(19) United States
(12) Patent Application Publication
(10) Pub. No.: US 2017/0051290 A1
(43) Pub. Date: Feb. 23, 2017

(54) METHODS FOR TREATMENT OF DISORDERS IN THE FRONT OF THE EYE UTILIZING NUCLEIC ACID MOLECULES

(71) Applicant: RXi Pharmaceuticals Corporation, Marlborough, MA (US)

(72) Inventors: Michael Byrne, Natick, MA (US); Pamela A. Pavco, Longmont, CO (US); Karen G. Bulock, Mendon, MA (US)

(73) Assignee: RXi Pharmaceuticals Corporation, Marlborough, MA (US)

(21) Appl. No.: 15/307,529

(22) PCT Filed: May 1, 2015
(86) PCT No.: PCT/US15/28860

§ 371 Date: Oct. 28, 2016

Related U.S. Application Data
(60) Provisional application No. 61/987,418, filed on May 1, 2014.

(51) Int. Cl. C12N 15/113; A61K 31/7088

(52) U.S. Cl. CPC C12N 15/1137; C12N 15/1136; A61K 31/7088; C12N 2310/351; C12N 2310/14

(57) ABSTRACT

Aspects of the invention relate to methods for treating an ocular disorder associated with the front of the eye, comprising administering to the eye of a subject in need thereof a therapeutic RNA molecule, in an effective amount to treat an ocular disorder associated with the front of the eye.

### RELATED APPLICATIONS

This application claims the benefit under 35 U.S.C. §119(e) of U.S. Provisional Application Ser. No. U.S. 61/987,418, entitled “METHODS FOR TREATMENT OF DISORDERS IN THE FRONT OF THE EYE UTILIZING NUCLEIC ACID MOLECULES,” filed on May 1, 2014, the entire disclosure of which is herein incorporated by reference in its entirety.

### FIELD OF INVENTION

The invention pertains to the treatment of ocular disorders in the front of the eye.

### BACKGROUND OF INVENTION

Complementary oligonucleotide sequences are promising therapeutic agents and useful research tools in elucidating gene functions. However, prior art oligonucleotide molecules suffer from several problems that may impede their clinical development, and frequently make it difficult to achieve intended efficient inhibition of gene expression (including protein synthesis) using such compositions in vivo.

A major problem has been the delivery of these compounds to cells and tissues. Conventional double-stranded RNAi compounds, 19-29 bases long, form a highly negatively-charged rigid helix of approximately 1.5 by 10-15 nm in size. This rod type molecule cannot get through the cell-membrane and as a result has very limited efficacy both in vitro and in vivo. As a result, all conventional RNAi compounds require some kind of a delivery vehicle to promote their tissue distribution and cellular uptake. This is considered to be a major limitation of the RNAi technology.

There have been previous attempts to apply chemical modifications to oligonucleotides to improve their cellular uptake properties. One such modification was the attachment of a cholesterol molecule to the oligonucleotide. A first report on this approach was by Letsinger et al., in 1989. Subsequently, ISIS Pharmaceuticals, Inc. (Carlsbad, Calif.) reported on more advanced techniques in attaching the cholesterol molecule to the oligonucleotide (Manoharan, 1992).

With the discovery of siRNAs in the late nineties, similar types of modifications were attempted on these molecules to enhance their delivery profiles. Cholesterol molecules conjugated to slightly modified (Soutschek, 2004) and heavily modified (Wolfrum, 2007) siRNAs appeared in the literature. Yamada et al., 2008 also reported on the use of advanced linker chemistries which further improved cholesterol mediated uptake of siRNAs. In spite of all this effort, the uptake of these types of compounds appears to be inhibited in the presence of biological fluids resulting in highly limited efficacy in gene silencing in vivo, limiting the applicability of these compounds in a clinical setting.

### SUMMARY OF INVENTION

Described herein are methods and compositions for efficient in vivo administration of therapeutic RNA molecules to the eye. Surprisingly, intravitreal administration of an sd-rxRNA molecule targeting CTGF resulted in effective gene silencing in the front of the eye. Therapeutic RNA molecules described herein have widespread applications for treatment of disorders or conditions associated with the front of the eye.

Aspects of the invention relate to methods for treating an ocular disorder associated with the front of the eye, comprising administering to the eye of a subject in need thereof a therapeutic RNA molecule, in an effective amount to treat an ocular disorder associated with the front of the eye.

In some embodiments, the ocular disorder associated with the front of the eye is selected from the group consisting of: Corneal scarring, corneal perforation, corneal dystrophies, corneal injury and/or trauma (including burns), corneal inflammation, corneal infection, opthalmia neonatorum, erythema multiform (Stevens-Johnson Syndrome), xerophthalmia (dry eye syndrome), trachoma, onchocerciasis (river blindness), corneal complications of leprosy, keratitis, persistent corneal epithelial defects, conjunctivitis, anterior uveitis, iridocorneal endothelial syndrome, Fuch's Dystrophy, trichiasis, ocular herpes, corneal grafting or transplant (including ex vivo treatment of a graft or transplant prior to surgery), corneal transplant failure and/or rejection.

In some embodiments, the therapeutic RNA molecule is delivered to an area of the eye other than the front of the eye. In some embodiments, the therapeutic RNA molecule is delivered to the front of the eye.

In some embodiments, the therapeutic RNA molecule is administered by a method selected from the group consisting of: intravitreal, subretinal, periocular (subconjunctival, sub-tenon, retrobulbar, peribulbar and posterior juxtascleral), topical, eye drops, corneal implants, biodegradable implants, non-biodegradable implants ocular inserts, thin-films, sustained release formulations, polymers and slow release polymers, iontophoresis, hydrogel contact lenses, reverse/thermal hydrogels and biodegradable pellets.

In some embodiments, the therapeutic RNA molecule is directed against a gene encoding a protein selected from the group consisting of: CTGF, VEGF, MAP4K4, PDGF-B, SDF-1, IGTA5, ANG2, HIF-1alpha, mTOR, SDF-1, PDGF-B, SPP1, PTGS2 (COX-2), TGFβ1, TGFβ2, complement factors 3 and 5, PDGFRa, PPIB, IL-1 alpha, IL-1 beta, Icam-1, Tie 1, Tie 2, ANg 1, Ang 2, and myc, or a combination thereof.

In some embodiments, the therapeutic RNA molecule is directed against a gene encoding CTGF. In some embodiments, the therapeutic RNA molecule is directed against a gene encoding VEGF. In some embodiments, the therapeutic RNA molecule is directed against a gene encoding Map4K4.

In some embodiments, two or more different therapeutic RNA molecules that are directed against genes encoding two or more different proteins are both administered to the eye of the subject. In some embodiments, two or more different therapeutic RNA molecules that are directed against genes encoding the same protein are both administered to the eye of the subject.

In some embodiments, the therapeutic RNA molecule is an sd-rxRNA.

In some embodiments, the sd-rxRNA comprises at least 12 contiguous nucleotides of a sequence selected from the sequences within Tables 3-8, 10 or 11. In some embodiments, the antisense strand of the sd-rxRNA comprises at least 12 contiguous nucleotides of the sequence of SEQ ID NO:948 or SEQ ID NO:964. In some embodiments, the sense strand of the sd-rxRNA comprises at least 12 contiguous nucleotides of the sequence of SEQ ID NO:947 or SEQ ID NO:963.

In some embodiments, the sense strand of the sd-rxRNA comprises SEQ ID NO:947 and the antisense strand of the sd-rxRNA comprises SEQ ID NO:948. In some embodiments, the sense strand of the sd-rxRNA comprises at least 12 contiguous nucleotides of the sequence of SEQ ID NO:1317 or SEQ ID NO:1357.

In some embodiments, the antisense strand of the sd-rxRNA comprises at least 12 contiguous nucleotides of the sequence of SEQ ID NO:1318 or SEQ ID NO:1358. In some embodiments, the sense strand of the sd-rxRNA comprises SEQ ID NO:1317 and the antisense strand of the sd-rxRNA comprises SEQ ID NO:1318. In some embodiments, the sense strand of the sd-rxRNA comprises SEQ ID NO:1357 and the antisense strand of the sd-rxRNA comprises SEQ ID NO:1358. In some embodiments, the sense strand of the sd-rxRNA comprises SEQ ID NO:1379 and the antisense strand of the sd-rxRNA comprises SEQ ID NO:1380. In some embodiments, the sense strand of the sd-rxRNA comprises SEQ ID NO:1397 and the antisense strand of the sd-rxRNA comprises SEQ ID NO:1398.

In some embodiments, the sd-rxRNA is hydrophobically modified. In some embodiments, the sd-rxRNA is linked to one or more hydrophobic conjugates.

In some embodiments, the therapeutic RNA molecule is an rxRNAori.

Aspects of the invention relate to an sd-rxRNA that is directed against a sequence comprising at least 12 contiguous nucleotides of a sequence within Table 11.

Aspects of the invention relate to an sd-rxRNA that comprises at least 12 contiguous nucleotides of a sequence within Table 11.

Aspects of the invention relate to methods of administering a therapeutic RNA molecule to the eye wherein the therapeutic RNA molecule is administered to an eye that is compromised and/or wounded. In some embodiments, the cornea is compromised and/or wounded. In some embodiments, the therapeutic RNA molecule is administered to the cornea. In some embodiments, the therapeutic RNA molecule is administered topically.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 demonstrates a significant reduction of CTGF protein levels in the cornea of monkeys intravitreally injected with a therapeutic RNA molecule targeting CTGF compared to the cornea of PBS-injected control monkeys.

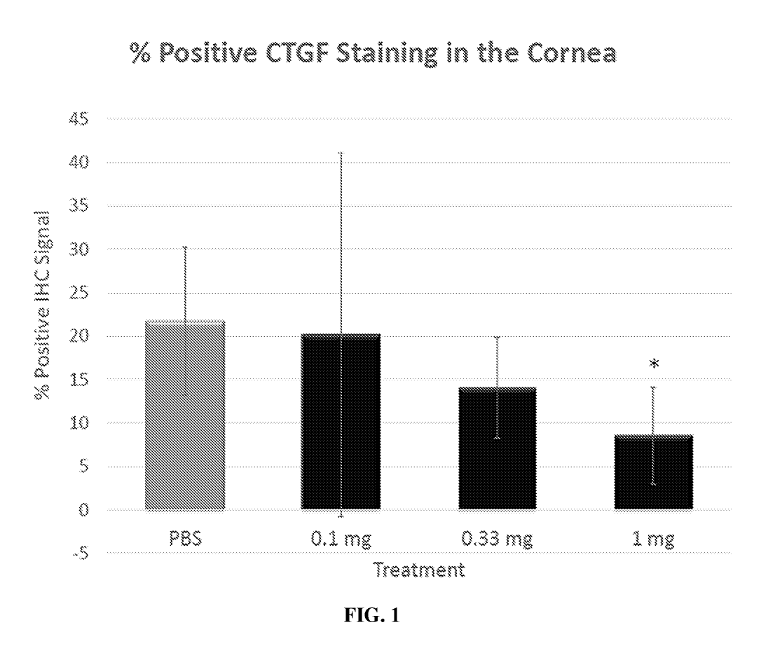

FIG. 2 demonstrates that the sd-rxRNA, RXI-109, penetrates all cell layers of the MatTek 3D epicorneal tissue model. Cells were treated with the sd-rxRNA by media exposure.

FIG. 3 demonstrates that the sd-rxRNA, RXI-109, penetrates all cell layers of the MatTek 3D epicorneal tissue model. Cells were treated by media exposure or by topical administration. Uptake of the sd-rxRNA using media exposure and topical administration was compared in the presence of a scratch to mimic a wound in the cornea. Cellular uptake of sd-rxRNA was observed following media exposure (intact or scratch model) or topical administration (scratch model).

FIG. 4 demonstrates sd-rxRNAs significantly reduce target gene mRNA levels in the epicorneal 3D model (human epithelia cells). Gene specific silencing was observed forty eight hours post-administration of Map4k4-targeting sd-rxRNA in the epicorneal model.

### DETAILED DESCRIPTION

Aspects of the invention relate to methods and compositions involved in gene silencing. The invention is based at least in part on the surprising discovery that intravitreal administration of a therapeutic RNA molecule to the eye led to reduced expression of a target gene in the front of the eye. Thus, methods described herein provide significant potential for treatment of ocular conditions or disorders affecting the front of the eye.

As used herein, “therapeutic RNA molecule” refers to an RNA molecule that can reduce expression of a target gene. A therapeutic RNA molecule includes but is not limited to: sd-rxRNA, rxRNAori, oligonucleotides, ASO, siRNA, shRNA, miRNA, ncRNA, cp-lasiRNA, aiRNA, BMT-101, RXI-109, EXC-001, and single-stranded nucleic acid molecules. In some embodiments, a therapeutic RNA molecule is a chemically modified nucleic acid molecule, such as a chemically modified oligonucleotide.

Aspects of the invention relate to the treatment of ocular disorders in the front of the eye. As used herein, the front of the eye includes but is not limited to the lens, iris, cornea, pupil, sclera, ciliary body and conjunctiva.

sd-rxRNA Molecules

Aspects of the invention relate to sd-rxRNA molecules. As used herein, an “sd-rxRNA” or an “sd-rxRNA molecule” refers to a self-delivering RNA molecule such as those described in, and incorporated by reference from, PCT Publication No. WO2010/033247 (Application No. PCT/US2009/005247), filed on Sep. 22, 2009, and entitled “REDUCED SIZE SELF-DELIVERING RNAI COMPOUNDS” and U.S. Pat. No. 8,796,443, which issued on Aug. 5, 2014, and published on Feb. 16, 2012 as US 2012/0040459, entitled “REDUCED SIZE SELF-DELIVERING RNAI COMPOUNDS.” Briefly, an sd-rxRNA, (also referred to as an sd-rxRNA^{nano}) is an isolated asymmetric double stranded nucleic acid molecule comprising a guide strand, with a minimal length of 16 nucleotides, and a passenger strand of 8-18 nucleotides in length, wherein the double stranded nucleic acid molecule has a double stranded region and a single stranded region, the single stranded region having 4-12 nucleotides in length and having at least three nucleotide backbone modifications. In preferred embodiments, the double stranded nucleic acid molecule has one end that is blunt or includes a one or two nucleotide overhang. sd-rxRNA molecules can be optimized through chemical modification, and in some instances through attachment of hydrophobic conjugates.

In some embodiments, an sd-rxRNA comprises an isolated double stranded nucleic acid molecule comprising a guide strand and a passenger strand, wherein the region of the molecule that is double stranded is from 8-15 nucleotides long, wherein the guide strand contains a single stranded region that is 4-12 nucleotides long, wherein the single stranded region of the guide strand contains 3, 4, 5, 6, 7, 8, 9, 10, 11 or 12 phosphorothioate modifications, and wherein at least 40% of the nucleotides of the double stranded nucleic acid are modified.

The polynucleotides of the invention are referred to herein as isolated double stranded or duplex nucleic acids, oligonucleotides or polynucleotides, nano molecules, nano RNA, sd-rxRNA′, sd-rxRNA or RNA molecules of the invention.

sd-rxRNAs are much more effectively taken up by cells compared to conventional siRNAs. These molecules are highly efficient in silencing of target gene expression and offer significant advantages over previously described RNAi molecules including high activity in the presence of serum, efficient self delivery, compatibility with a wide variety of linkers, and reduced presence or complete absence of chemical modifications that are associated with toxicity.

In contrast to single-stranded polynucleotides, duplex polynucleotides have traditionally been difficult to deliver to a cell as they have rigid structures and a large number of negative charges which makes membrane transfer difficult. sd-rxRNAs however, although partially double-stranded, are recognized in vivo as single-stranded and, as such, are capable of efficiently being delivered across cell membranes. As a result the polynucleotides of the invention are capable in many instances of self delivery. Thus, the polynucleotides of the invention may be formulated in a manner similar to conventional RNAi agents or they may be delivered to the cell or subject alone (or with non-delivery type carriers) and allowed to self deliver. In one embodiment of the present invention, self delivering asymmetric double-stranded RNA molecules are provided in which one portion of the molecule resembles a conventional RNA duplex and a second portion of the molecule is single stranded.

The oligonucleotides of the invention in some aspects have a combination of asymmetric structures including a double stranded region and a single stranded region of 5 nucleotides or longer, specific chemical modification patterns and are conjugated to lipophilic or hydrophobic molecules. This class of RNAi like compounds have superior efficacy in vitro and in vivo. It is believed that the reduction in the size of the rigid duplex region in combination with phosphorothioate modifications applied to a single stranded region contribute to the observed superior efficacy.

The invention is based, at least in part, on the surprising discovery that sd-rxRNAs can be delivered efficiently to the eye through either subretinal or intravitreal injection. Based on results generated in multiple different mammalian systems, including mouse, rat and rabbit, and as presented in the Examples section, drastically (several orders of magnitude) better ocular uptake and distribution is observed following administration of sd-rxRNAs than following administration of conventional RNAi compounds.

Another surprising aspect of the invention is that sd-rxRNA molecules are taken up by all cell layers in the retina, including the retinal pigment epithelium cell layer. Efficient sd-rxRNA distribution is achieved through both subretinal and intravitreal injection and both means of administration are compatible with aspects of the invention. In some embodiments, intravitreal administration is preferred due to technical ease and widespread use in intraocular drug delivery.

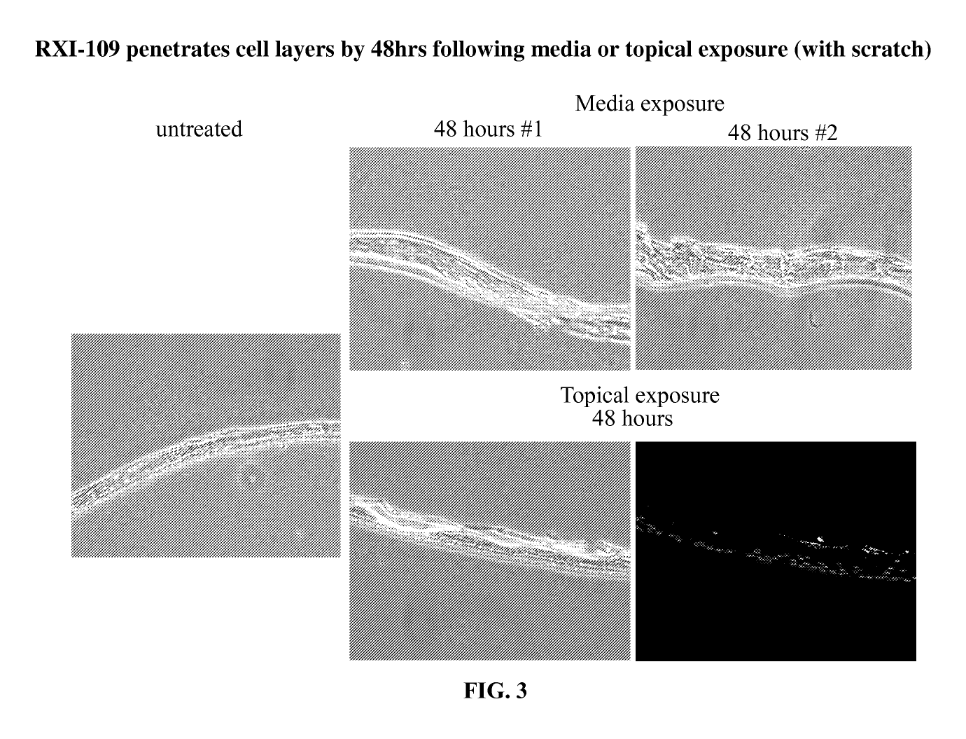

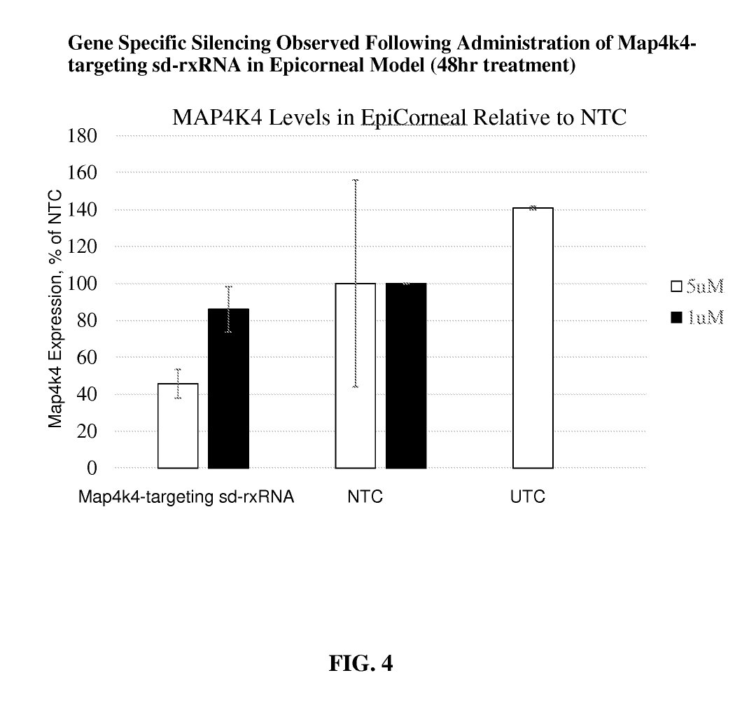

Another surprising aspect of the invention is that in a 3D epicorneal tissue culture model system (utilizing human corneal epithelial cells), when cells were treated with sd-rxRNA through media exposure or through topical administration, cellular uptake was observed (FIG. 3). Sd-rxRNAs also achieved significantly reduced expression of target genes in this model system (FIG. 4). In some embodiments, topical administration, such as topical administration to the cornea, is preferred.

As used herein, “ocular” refers to the eye, including any and all of its cells including muscles, nerves, blood vessels, tear ducts, membranes etc., as well as structures that are connected with the eye and its physiological functions. The terms ocular and eye are used interchangeably throughout this disclosure. Non-limiting examples of cell types within the eye include: cells located in the ganglion cell layer (GCL), the inner plexiform layer inner (IPL), the inner nuclear layer (INL), the outer plexiform layer (OPL), outer nuclear layer (ONL), outer segments (OS) of rods and cones, the retinal pigmented epithelium (RPE), the inner segments (IS) of rods and cones, the epithelium of the conjunctiva, the iris, the ciliary body, the corneum, and epithelium of ocular sebaceous glands.

In a preferred embodiment the RNAi compounds of the invention comprise an asymmetric compound comprising a duplex region (required for efficient RISC entry of 8-15 bases long) and single stranded region of 4-12 nucleotides long. In some embodiments, the duplex region is 13 or 14 nucleotides long. A 6 or 7 nucleotide single stranded region is preferred in some embodiments. The single stranded region of the new RNAi compounds also comprises 2-12 phosphorothioate internucleotide linkages (referred to as phosphorothioate modifications). 6-8 phosphorothioate internucleotide linkages are preferred in some embodiments. Additionally, the RNAi compounds of the invention also include a unique chemical modification pattern, which provides stability and is compatible with RISC entry. The combination of these elements has resulted in unexpected properties which are highly useful for delivery of RNAi reagents in vitro and in vivo.

The chemical modification pattern, which provides stability and is compatible with RISC entry includes modifications to the sense, or passenger, strand as well as the antisense, or guide, strand. For instance the passenger strand can be modified with any chemical entities which confirm stability and do not interfere with activity. Such modifications include 2′ ribo modifications (O-methyl, 2′ F, 2 deoxy and others) and backbone modification like phosphorothioate modifications. A preferred chemical modification pattern in the passenger strand includes Omethyl modification of C and U nucleotides within the passenger strand or alternatively the passenger strand may be completely Omethyl modified.

The guide strand, for example, may also be modified by any chemical modification which confirms stability without interfering with RISC entry. A preferred chemical modification pattern in the guide strand includes the majority of C and U nucleotides being 2′ F modified and the 5′ end being phosphorylated. Another preferred chemical modification pattern in the guide strand includes 2′Omethyl modification of position 1 and C/U in positions 11-18 and 5′ end chemical phosphorylation. Yet another preferred chemical modification pattern in the guide strand includes 2′Omethyl modification of position 1 and C/U in positions 11-18 and 5′ end chemical phosphorylation and 2′F modification of C/U in positions 2-10. In some embodiments the passenger strand and/or the guide strand contains at least one 5-methyl C or U modifications.

In some embodiments, at least 30% of the nucleotides in the sd-rxRNA are modified. For example, at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotides in the sd-rxRNA are modified. In some embodiments, 100% of the nucleotides in the sd-rxRNA are modified.

The above-described chemical modification patterns of the oligonucleotides of the invention are well tolerated and actually improved efficacy of asymmetric RNAi compounds. It was also demonstrated experimentally herein that the combination of modifications to RNAi when used together in a polynucleotide results in the achievement of optimal efficacy in passive uptake of the RNAi. Elimination of any of the described components (Guide strand stabilization, phosphorothioate stretch, sense strand stabilization and hydrophobic conjugate) or increase in size in some instances results in sub-optimal efficacy and in some instances complete lost of efficacy. The combination of elements results in development of a compound, which is fully active following passive delivery to cells such as HeLa cells. The data in the Examples presented below demonstrates high efficacy of the oligonucleotides of the invention in vivo upon ocular administration.

The sd-rxRNA can be further improved in some instances by improving the hydrophobicity of compounds using of novel types of chemistries. For example, one chemistry is related to use of hydrophobic base modifications. Any base in any position might be modified, as long as modification results in an increase of the partition coefficient of the base. The preferred locations for modification chemistries are positions 4 and 5 of the pyrimidines. The major advantage of these positions is (a) ease of synthesis and (b) lack of interference with base-pairing and A form helix formation, which are essential for RISC complex loading and target recognition. A version of sd-rxRNA compounds where multiple deoxy Uridines are present without interfering with overall compound efficacy was used. In addition major improvement in tissue distribution and cellular uptake might be obtained by optimizing the structure of the hydrophobic conjugate. In some of the preferred embodiment the structure of sterol is modified to alter (increase/decrease) C17 attached chain. This type of modification results in significant increase in cellular uptake and improvement of tissue uptake prosperities in vivo.

dsRNA formulated according to the invention also includes rxRNAori. rxRNAori refers to a class of RNA molecules described in and incorporated by reference from PCT Publication No. WO2009/102427 (Application No. PCT/US2009/000852), filed on Feb. 11, 2009, and entitled, “MODIFIED RNAI POLYNUCLEOTIDES AND USES THEREOF” and US Patent Publication No. 2011/0039914, published on Feb. 17, 2011 and entitled “MODIFIED RNAI POLYNUCLEOTIDES AND USES THEREOF”.

In some embodiments, an rxRNAori molecule comprises a double-stranded RNA (dsRNA) construct of 12-35 nucleotides in length, for inhibiting expression of a target gene, comprising: a sense strand having a 5′-end and a 3′-end, wherein the sense strand is highly modified with 2′-modified ribose sugars, and wherein 3-6 nucleotides in the central portion of the sense strand are not modified with 2′-modified ribose sugars and, an antisense strand having a 5′-end and a 3′-end, which hybridizes to the sense strand and to mRNA of the target gene, wherein the dsRNA inhibits expression of the target gene in a sequence-dependent manner.

rxRNAori can contain any of the modifications described herein. In some embodiments, at least 30% of the nucleotides in the rxRNAori are modified. For example, at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotides in the rxRNAori are modified. In some embodiments, 100% of the nucleotides in the sd-rxRNA are modified. In some embodiments, only the passenger strand of the rxRNAori contains modifications.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Thus, aspects of the invention relate to isolated double stranded nucleic acid molecules comprising a guide (antisense) strand and a passenger (sense) strand. As used herein, the term “double-stranded” refers to one or more nucleic acid molecules in which at least a portion of the nucleomonomers are complementary and hydrogen bond to form a double-stranded region. In some embodiments, the length of the guide strand ranges from 16-29 nucleotides long. In certain embodiments, the guide strand is 16, 17, 18, 19, 20, 21, 22, 23, 24, 25, 26, 27, 28, or 29 nucleotides long. The guide strand has complementarity to a target gene. Complementarity between the guide strand and the target gene may exist over any portion of the guide strand. Complementarity as used herein may be perfect complementarity or less than perfect complementarity as long as the guide strand is sufficiently complementary to the target that it mediates RNAi. In some embodiments complementarity refers to less than 25%, 20%, 15%, 10%, 5%, 4%, 3%, 2%, or 1% mismatch between the guide strand and the target. Perfect complementarity refers to 100% complementarity. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Moreover, not all positions of a siRNA contribute equally to target recognition. Mismatches in the center of the siRNA are most critical and essentially abolish target RNA cleavage. Mismatches upstream of the center or upstream of the cleavage site referencing the antisense strand are tolerated but significantly reduce target RNA cleavage. Mismatches downstream of the center or cleavage site referencing the antisense strand, preferably located near the 3′ end of the antisense strand, e.g. 1, 2, 3, 4, 5 or 6 nucleotides from the 3′ end of the antisense strand, are tolerated and reduce target RNA cleavage only slightly.

While not wishing to be bound by any particular theory, in some embodiments, the guide strand is at least 16 nucleotides in length and anchors the Argonaute protein in RISC. In some embodiments, when the guide strand loads into RISC it has a defined seed region and target mRNA cleavage takes place across from position 10-11 of the guide strand. In some embodiments, the 5′ end of the guide strand is or is able to be phosphorylated. The nucleic acid molecules described herein may be referred to as minimum trigger RNA.

In some embodiments, the length of the passenger strand ranges from 8-15 nucleotides long. In certain embodiments, the passenger strand is 8, 9, 10, 11, 12, 13, 14 or 15 nucleotides long. The passenger strand has complementarity to the guide strand. Complementarity between the passenger strand and the guide strand can exist over any portion of the passenger or guide strand. In some embodiments, there is 100% complementarity between the guide and passenger strands within the double stranded region of the molecule.

Aspects of the invention relate to double stranded nucleic acid molecules with minimal double stranded regions. In some embodiments the region of the molecule that is double stranded ranges from 8-15 nucleotides long. In certain embodiments, the region of the molecule that is double stranded is 8, 9, 10, 11, 12, 13, 14 or 15 nucleotides long. In certain embodiments the double stranded region is 13 or 14 nucleotides long. There can be 100% complementarity between the guide and passenger strands, or there may be one or more mismatches between the guide and passenger strands. In some embodiments, on one end of the double stranded molecule, the molecule is either blunt-ended or has a one-nucleotide overhang. The single stranded region of the molecule is in some embodiments between 4-12 nucleotides long. For example the single stranded region can be 4, 5, 6, 7, 8, 9, 10, 11 or 12 nucleotides long. However, in certain embodiments, the single stranded region can also be less than 4 or greater than 12 nucleotides long. In certain embodiments, the single stranded region is at least 6 or at least 7 nucleotides long.

RNAi constructs associated with the invention can have a thermodynamic stability (ΔG) of less than −13 kkal/mol. In some embodiments, the thermodynamic stability (ΔG) is less than −20 kkal/mol. In some embodiments there is a loss of efficacy when (ΔG) goes below −21 kkal/mol. In some embodiments a (ΔG) value higher than −13 kkal/mol is compatible with aspects of the invention. Without wishing to be bound by any theory, in some embodiments a molecule with a relatively higher (ΔG) value may become active at a relatively higher concentration, while a molecule with a relatively lower (ΔG) value may become active at a relatively lower concentration. In some embodiments, the (ΔG) value may be higher than −9 kkcal/mol. The gene silencing effects mediated by the RNAi constructs associated with the invention, containing minimal double stranded regions, are unexpected because molecules of almost identical design but lower thermodynamic stability have been demonstrated to be inactive (Rana et al 2004).

Without wishing to be bound by any theory, results described herein suggest that a stretch of 8-10 bp of dsRNA or dsDNA will be structurally recognized by protein components of RISC or co-factors of RISC. Additionally, there is a free energy requirement for the triggering compound that it may be either sensed by the protein components and/or stable enough to interact with such components so that it may be loaded into the Argonaute protein. If optimal thermodynamics are present and there is a double stranded portion that is preferably at least 8 nucleotides then the duplex will be recognized and loaded into the RNAi machinery.

In some embodiments, thermodynamic stability is increased through the use of LNA bases. In some embodiments, additional chemical modifications are introduced. Several non-limiting examples of chemical modifications include: 5′ Phosphate, 2′-O-methyl, 2′-O-ethyl, 2′-fluoro, ribothymidine, C-5 propynyl-dC (pdC) and C-5 propynyl-dU (pdU); C-5 propynyl-C(pC) and C-5 propynyl-U (pU); 5-methyl C, 5-methyl U, 5-methyl dC, 5-methyl dU methoxy, (2,6-diaminopurine), 5′-Dimethoxytrityl-N4-ethyl-2′-deoxyCytidine and MGB (minor groove binder). It should be appreciated that more than one chemical modification can be combined within the same molecule.

Molecules associated with the invention are optimized for increased potency and/or reduced toxicity. For example, nucleotide length of the guide and/or passenger strand, and/or the number of phosphorothioate modifications in the guide and/or passenger strand, can in some aspects influence potency of the RNA molecule, while replacing 2′-fluoro (2′F) modifications with 2′-O-methyl (2′OMe) modifications can in some aspects influence toxicity of the molecule. Specifically, reduction in 2′F content of a molecule is predicted to reduce toxicity of the molecule. The Examples section presents molecules in which 2′F modifications have been eliminated, offering an advantage over previously described RNAi compounds due to a predicted reduction in toxicity. Furthermore, the number of phosphorothioate modifications in an RNA molecule can influence the uptake of the molecule into a cell, for example the efficiency of passive uptake of the molecule into a cell. Preferred embodiments of molecules described herein have no 2′F modification and yet are characterized by equal efficacy in cellular uptake and tissue penetration. Such molecules represent a significant improvement over prior art, such as molecules described by Accell and Wolfrum, which are heavily modified with extensive use of 2′F.

In some embodiments, a guide strand is approximately 18-19 nucleotides in length and has approximately 2-14 phosphate modifications. For example, a guide strand can contain 2, 3, 4, 5, 6, 7, 8, 9, 10, 11, 12, 13, 14 or more than 14 nucleotides that are phosphate-modified. The guide strand may contain one or more modifications that confer increased stability without interfering with RISC entry. The phosphate modified nucleotides, such as phosphorothioate modified nucleotides, can be at the 3′ end, 5′ end or spread throughout the guide strand. In some embodiments, the 3′ terminal 10 nucleotides of the guide strand contains 1, 2, 3, 4, 5, 6, 7, 8, 9 or 10 phosphorothioate modified nucleotides. The guide strand can also contain 2′F and/or 2′OMe modifications, which can be located throughout the molecule. In some embodiments, the nucleotide in position one of the guide strand (the nucleotide in the most 5′ position of the guide strand) is 2′OMe modified and/or phosphorylated. C and U nucleotides within the guide strand can be 2′F modified. For example, C and U nucleotides in positions 2-10 of a 19 nt guide strand (or corresponding positions in a guide strand of a different length) can be 2′F modified. C and U nucleotides within the guide strand can also be 2′OMe modified. For example, C and U nucleotides in positions 11-18 of a 19 nt guide strand (or corresponding positions in a guide strand of a different length) can be 2′OMe modified. In some embodiments, the nucleotide at the most 3′ end of the guide strand is unmodified. In certain embodiments, the majority of Cs and Us within the guide strand are 2′F modified and the 5′ end of the guide strand is phosphorylated. In other embodiments, position 1 and the Cs or Us in positions 11-18 are 2′OMe modified and the 5′ end of the guide strand is phosphorylated. In other embodiments, position 1 and the Cs or Us in positions 11-18 are 2′OMe modified, the 5′ end of the guide strand is phosphorylated, and the Cs or Us in position 2-10 are 2′F modified.

In some aspects, an optimal passenger strand is approximately 11-14 nucleotides in length. The passenger strand may contain modifications that confer increased stability. One or more nucleotides in the passenger strand can be 2′OMe modified. In some embodiments, one or more of the C and/or U nucleotides in the passenger strand is 2′OMe modified, or all of the C and U nucleotides in the passenger strand are 2′OMe modified. In certain embodiments, all of the nucleotides in the passenger strand are 2′OMe modified. One or more of the nucleotides on the passenger strand can also be phosphate-modified such as phosphorothioate modified. The passenger strand can also contain 2′ ribo, 2′F and 2 deoxy modifications or any combination of the above. As demonstrated in the Examples, chemical modification patterns on both the guide and passenger strand are well tolerated and a combination of chemical modifications is shown herein to lead to increased efficacy and self-delivery of RNA molecules.

Aspects of the invention relate to RNAi constructs that have extended single-stranded regions relative to double stranded regions, as compared to molecules that have been used previously for RNAi. The single stranded region of the molecules may be modified to promote cellular uptake or gene silencing. In some embodiments, phosphorothioate modification of the single stranded region influences cellular uptake and/or gene silencing. The region of the guide strand that is phosphorothioate modified can include nucleotides within both the single stranded and double stranded regions of the molecule. In some embodiments, the single stranded region includes 2-12 phosphorothioate modifications. For example, the single stranded region can include 2, 3, 4, 5, 6, 7, 8, 9, 10, 11, or 12 phosphorothioate modifications. In some instances, the single stranded region contains 6-8 phosphorothioate modifications.

Molecules associated with the invention are also optimized for cellular uptake. In RNA molecules described herein, the guide and/or passenger strands can be attached to a conjugate. In certain embodiments the conjugate is hydrophobic. The hydrophobic conjugate can be a small molecule with a partition coefficient that is higher than 10. The conjugate can be a sterol-type molecule such as cholesterol, or a molecule with an increased length polycarbon chain attached to C17, and the presence of a conjugate can influence the ability of an RNA molecule to be taken into a cell with or without a lipid transfection reagent. The conjugate can be attached to the passenger or guide strand through a hydrophobic linker. In some embodiments, a hydrophobic linker is 5-12C in length, and/or is hydroxypyrrolidine-based. In some embodiments, a hydrophobic conjugate is attached to the passenger strand and the CU residues of either the passenger and/or guide strand are modified. In some embodiments, at least 50%, 55%, 60%, 65%, 70%, 75%, 80%, 85%, 90% or 95% of the CU residues on the passenger strand and/or the guide strand are modified. In some aspects, molecules associated with the invention are self-delivering (sd). As used herein, “self-delivery”refers to the ability of a molecule to be delivered into a cell without the need for an additional delivery vehicle such as a transfection reagent.

Aspects of the invention relate to selecting molecules for use in RNAi. In some embodiments, molecules that have a double stranded region of 8-15 nucleotides can be selected for use in RNAi. In some embodiments, molecules are selected based on their thermodynamic stability (ΔG). In some embodiments, molecules will be selected that have a (ΔG) of less than −13 kkal/mol. For example, the (ΔG) value may be −13, −14, −15, −16, −17, −18, −19, −21, −22 or less than −22 kkal/mol. In other embodiments, the (ΔG) value may be higher than −13 kkal/mol. For example, the (ΔG) value may be −12, −11, −10, −9, −8, −7 or more than −7 kkal/mol. It should be appreciated that ΔG can be calculated using any method known in the art. In some embodiments ΔG is calculated using Mfold, available through the Mfold internet site (mfold.bioinfo.rpi.edu/cgi-bin/rna-form1.cgi). Methods for calculating ΔG are described in, and are incorporated by reference from, the following references: Zuker, M. (2003) Nucleic Acids Res., 31(13):3406-15; Mathews, D. H., Sabina, J., Zuker, M. and Turner, D. H. (1999) J. Mol. Biol. 288:911-940; Mathews, D. H., Disney, M. D., Childs, J. L., Schroeder, S. J., Zuker, M., and Turner, D. H. (2004) Proc. Natl. Acad. Sci. 101:7287-7292; Duan, S., Mathews, D. H., and Turner, D. H. (2006) Biochemistry 45:9819-9832; Wuchty, S., Fontana, W., Hofacker, I. L., and Schuster, P. (1999) Biopolymers 49:145-165.

In certain embodiments, the polynucleotide contains 5′- and/or 3′-end overhangs. The number and/or sequence of nucleotides overhang on one end of the polynucleotide may be the same or different from the other end of the polynucleotide. In certain embodiments, one or more of the overhang nucleotides may contain chemical modification(s), such as phosphorothioate or 2′-OMe modification.

In certain embodiments, the polynucleotide is unmodified. In other embodiments, at least one nucleotide is modified. In further embodiments, the modification includes a 2′-H or 2′-modified ribose sugar at the 2nd nucleotide from the 5′-end of the guide sequence. The “2nd nucleotide” is defined as the second nucleotide from the 5′-end of the polynucleotide.

As used herein, “2′-modified ribose sugar” includes those ribose sugars that do not have a 2′-OH group. “2′-modified ribose sugar” does not include 2′-deoxyribose (found in unmodified canonical DNA nucleotides). For example, the 2′-modified ribose sugar may be 2′-O-alkyl nucleotides, 2′-deoxy-2′-fluoro nucleotides, 2′-deoxy nucleotides, or combination thereof.

In certain embodiments, the 2′-modified nucleotides are pyrimidine nucleotides (e.g., C/U). Examples of 2′-O-alkyl nucleotides include 2′-O-methyl nucleotides, or 2′-O-allyl nucleotides.

In certain embodiments, the sd-rxRNA polynucleotide of the invention with the above-referenced 5′-end modification exhibits significantly (e.g., at least about 25%, 30%, 35%, 40%, 45%, 50%, 55%, 60%, 65%, 70%, 75%, 80%, 85%, 90% or more) less “off-target” gene silencing when compared to similar constructs without the specified 5′-end modification, thus greatly improving the overall specificity of the RNAi reagent or therapeutics.

As used herein, “off-target” gene silencing refers to unintended gene silencing due to, for example, spurious sequence homology between the antisense (guide) sequence and the unintended target mRNA sequence.

According to this aspect of the invention, certain guide strand modifications further increase nuclease stability, and/or lower interferon induction, without significantly decreasing RNAi activity (or no decrease in RNAi activity at all).

In some embodiments, the 5′-stem sequence may comprise a 2′-modified ribose sugar, such as 2′-O-methyl modified nucleotide, at the 2^{nd} nucleotide on the 5′-end of the polynucleotide and, in some embodiments, no other modified nucleotides. The hairpin structure having such modification may have enhanced target specificity or reduced off-target silencing compared to a similar construct without the 2′-O-methyl modification at said position.

Certain combinations of specific 5′-stem sequence and 3′-stem sequence modifications may result in further unexpected advantages, as partly manifested by enhanced ability to inhibit target gene expression, enhanced serum stability, and/or increased target specificity, etc.

In certain embodiments, the guide strand comprises a 2′-O-methyl modified nucleotide at the 2^{nd} nucleotide on the 5′-end of the guide strand and no other modified nucleotides.

In other aspects, the sd-rxRNA structures of the present invention mediates sequence-dependent gene silencing by a microRNA mechanism. As used herein, the term “microRNA” (“miRNA”), also referred to in the art as “small temporal RNAs” (“stRNAs”), refers to a small (10-50 nucleotide) RNA which are genetically encoded (e.g., by viral, mammalian, or plant genomes) and are capable of directing or mediating RNA silencing. An “miRNA disorder” shall refer to a disease or disorder characterized by an aberrant expression or activity of an miRNA.

microRNAs are involved in down-regulating target genes in critical pathways, such as development and cancer, in mice, worms and mammals. Gene silencing through a microRNA mechanism is achieved by specific yet imperfect base-pairing of the miRNA and its target messenger RNA (mRNA). Various mechanisms may be used in microRNA-mediated down-regulation of target mRNA expression.

miRNAs are noncoding RNAs of approximately 22 nucleotides which can regulate gene expression at the post transcriptional or translational level during plant and animal development. One common feature of miRNAs is that they are all excised from an approximately 70 nucleotide precursor RNA stem-loop termed pre-miRNA, probably by Dicer, an RNase III-type enzyme, or a homolog thereof. Naturally-occurring miRNAs are expressed by endogenous genes in vivo and are processed from a hairpin or stem-loop precursor (pre-miRNA or pri-miRNAs) by Dicer or other RNAses. miRNAs can exist transiently in vivo as a double-stranded duplex but only one strand is taken up by the RISC complex to direct gene silencing.

In some embodiments a version of sd-rxRNA compounds, which are effective in cellular uptake and inhibiting of miRNA activity are described. Essentially the compounds are similar to RISC entering version but large strand chemical modification patterns are optimized in the way to block cleavage and act as an effective inhibitor of the RISC action. For example, the compound might be completely or mostly Omethyl modified with the PS content described previously. For these types of compounds the 5′ phosphorylation is not necessary. The presence of double stranded region is preferred as it is promotes cellular uptake and efficient RISC loading.

Another pathway that uses small RNAs as sequence-specific regulators is the RNA interference (RNAi) pathway, which is an evolutionarily conserved response to the presence of double-stranded RNA (dsRNA) in the cell. The dsRNAs are cleaved into ˜20-base pair (bp) duplexes of small-interfering RNAs (siRNAs) by Dicer. These small RNAs get assembled into multiprotein effector complexes called RNA-induced silencing complexes (RISCs). The siRNAs then guide the cleavage of target mRNAs with perfect complementarity.

Some aspects of biogenesis, protein complexes, and function are shared between the siRNA pathway and the miRNA pathway. The subject single-stranded polynucleotides may mimic the dsRNA in the siRNA mechanism, or the microRNA in the miRNA mechanism.

In certain embodiments, the modified RNAi constructs may have improved stability in serum and/or cerebral spinal fluid compared to an unmodified RNAi constructs having the same sequence.

In certain embodiments, the structure of the RNAi construct does not induce interferon response in primary cells, such as mammalian primary cells, including primary cells from human, mouse and other rodents, and other non-human mammals. In certain embodiments, the RNAi construct may also be used to inhibit expression of a target gene in an invertebrate organism.

To further increase the stability of the subject constructs in vivo, the 3′-end of the hairpin structure may be blocked by protective group(s). For example, protective groups such as inverted nucleotides, inverted abasic moieties, or amino-end modified nucleotides may be used. Inverted nucleotides may comprise an inverted deoxynucleotide. Inverted abasic moieties may comprise an inverted deoxyabasic moiety, such as a 3′,3′-linked or linked deoxyabasic moiety.

The RNAi constructs of the invention are capable of inhibiting the synthesis of any target protein encoded by target gene(s). The invention includes methods to inhibit expression of a target gene either in a cell in vitro, or in vivo. As such, the RNAi constructs of the invention are useful for treating a patient with a disease characterized by the overexpression of a target gene.

The target gene can be endogenous or exogenous (e.g., introduced into a cell by a virus or using recombinant DNA technology) to a cell. Such methods may include introduction of RNA into a cell in an amount sufficient to inhibit expression of the target gene. By way of example, such an RNA molecule may have a guide strand that is complementary to the nucleotide sequence of the target gene, such that the composition inhibits expression of the target gene.

The invention also relates to vectors expressing the nucleic acids of the invention, and cells comprising such vectors or the nucleic acids. The cell may be a mammalian cell in vivo or in culture, such as a human cell.

The invention further relates to compositions comprising the subject RNAi constructs, and a pharmaceutically acceptable carrier or diluent.

Another aspect of the invention provides a method for inhibiting the expression of a target gene in a mammalian cell, comprising contacting an eye cell with any of the subject RNAi constructs.

The method may be carried out in vitro, ex vivo, or in vivo, in, for example, mammalian cells in culture, such as a human cell in culture.

The target cells (e.g., mammalian cell) may be contacted in the presence of a delivery reagent, such as a lipid (e.g., a cationic lipid) or a liposome.

Another aspect of the invention provides a method for inhibiting the expression of a target gene in a mammalian cell, comprising contacting the mammalian cell with a vector expressing the subject RNAi constructs.

In one aspect of the invention, a longer duplex polynucleotide is provided, including a first polynucleotide that ranges in size from about 16 to about 30 nucleotides; a second polynucleotide that ranges in size from about 26 to about 46 nucleotides, wherein the first polynucleotide (the antisense strand) is complementary to both the second polynucleotide (the sense strand) and a target gene, and wherein both polynucleotides form a duplex and wherein the first polynucleotide contains a single stranded region longer than 6 bases in length and is modified with alternative chemical modification pattern, and/or includes a conjugate moiety that facilitates cellular delivery. In this embodiment, between about 40% to about 90% of the nucleotides of the passenger strand between about 40% to about 90% of the nucleotides of the guide strand, and between about 40% to about 90% of the nucleotides of the single stranded region of the first polynucleotide are chemically modified nucleotides.

In an embodiment, the chemically modified nucleotide in the polynucleotide duplex may be any chemically modified nucleotide known in the art, such as those discussed in detail above. In a particular embodiment, the chemically modified nucleotide is selected from the group consisting of 2′ F modified nucleotides, 2′-O-methyl modified and 2′deoxy nucleotides. In another particular embodiment, the chemically modified nucleotides results from “hydrophobic modifications” of the nucleotide base. In another particular embodiment, the chemically modified nucleotides are phosphorothioates. In an additional particular embodiment, chemically modified nucleotides are combination of phosphorothioates, 2′-O-methyl, 2′deoxy, hydrophobic modifications and phosphorothioates. As these groups of modifications refer to modification of the ribose ring, back bone and nucleotide, it is feasible that some modified nucleotides will carry a combination of all three modification types.

In another embodiment, the chemical modification is not the same across the various regions of the duplex. In a particular embodiment, the first polynucleotide (the passenger strand), has a large number of diverse chemical modifications in various positions. For this polynucleotide up to 90% of nucleotides might be chemically modified and/or have mismatches introduced.

In another embodiment, chemical modifications of the first or second polynucleotide include, but not limited to, 5′ position modification of Uridine and Cytosine (4-pyridyl, 2-pyridyl, indolyl, phenyl (C₆H₅OH); tryptophanyl (C8H6N)CH2CH(NH2)CO), isobutyl, butyl, aminobenzyl; phenyl; naphthyl, etc), where the chemical modification might alter base pairing capabilities of a nucleotide. For the guide strand an important feature of this aspect of the invention is the position of the chemical modification relative to the 5′ end of the antisense and sequence. For example, chemical phosphorylation of the 5′ end of the guide strand is usually beneficial for efficacy. O-methyl modifications in the seed region of the sense strand (position 2-7 relative to the 5′ end) are not generally well tolerated, whereas 2′F and deoxy are well tolerated. The mid part of the guide strand and the 3′ end of the guide strand are more permissive in a type of chemical modifications applied. Deoxy modifications are not tolerated at the 3′ end of the guide strand.

A unique feature of this aspect of the invention involves the use of hydrophobic modification on the bases. In one embodiment, the hydrophobic modifications are preferably positioned near the 5′ end of the guide strand, in other embodiments, they localized in the middle of the guides strand, in other embodiment they localized at the 3′ end of the guide strand and yet in another embodiment they are distributed thought the whole length of the polynucleotide. The same type of patterns is applicable to the passenger strand of the duplex.

The other part of the molecule is a single stranded region. The single stranded region is expected to range from 7 to 40 nucleotides.

In one embodiment, the single stranded region of the first polynucleotide contains modifications selected from the group consisting of between 40% and 90% hydrophobic base modifications, between 40%-90% phosphorothioates, between 40%-90% modification of the ribose moiety, and any combination of the preceding.

Efficiency of guide strand (first polynucleotide) loading into the RISC complex might be altered for heavily modified polynucleotides, so in one embodiment, the duplex polynucleotide includes a mismatch between nucleotide 9, 11, 12, 13, or 14 on the guide strand (first polynucleotide) and the opposite nucleotide on the sense strand (second polynucleotide) to promote efficient guide strand loading.

More detailed aspects of the invention are described in the sections below.

### Duplex Characteristics

Double-stranded oligonucleotides of the invention may be formed by two separate complementary nucleic acid strands. Duplex formation can occur either inside or outside the cell containing the target gene.

As used herein, the term “duplex” includes the region of the double-stranded nucleic acid molecule(s) that is (are) hydrogen bonded to a complementary sequence. Double-stranded oligonucleotides of the invention may comprise a nucleotide sequence that is sense to a target gene and a complementary sequence that is antisense to the target gene. The sense and antisense nucleotide sequences correspond to the target gene sequence, e.g., are identical or are sufficiently identical to effect target gene inhibition (e.g., are about at least about 98% identical, 96% identical, 94%, 90% identical, 85% identical, or 80% identical) to the target gene sequence.

In certain embodiments, the double-stranded oligonucleotide of the invention is double-stranded over its entire length, i.e., with no overhanging single-stranded sequence at either end of the molecule, i.e., is blunt-ended. In other embodiments, the individual nucleic acid molecules can be of different lengths. In other words, a double-stranded oligonucleotide of the invention is not double-stranded over its entire length. For instance, when two separate nucleic acid molecules are used, one of the molecules, e.g., the first molecule comprising an antisense sequence, can be longer than the second molecule hybridizing thereto (leaving a portion of the molecule single-stranded). Likewise, when a single nucleic acid molecule is used a portion of the molecule at either end can remain single-stranded.

In one embodiment, a double-stranded oligonucleotide of the invention contains mismatches and/or loops or bulges, but is double-stranded over at least about 70% of the length of the oligonucleotide. In another embodiment, a double-stranded oligonucleotide of the invention is double-stranded over at least about 80% of the length of the oligonucleotide. In another embodiment, a double-stranded oligonucleotide of the invention is double-stranded over at least about 90%-95% of the length of the oligonucleotide. In another embodiment, a double-stranded oligonucleotide of the invention is double-stranded over at least about 96%-98% of the length of the oligonucleotide. In certain embodiments, the double-stranded oligonucleotide of the invention contains at least or up to 1, 2, 3, 4, 5, 6, 7, 8, 9, 10, 11, 12, 13, 14, or 15 mismatches.

### Modifications

The nucleotides of the invention may be modified at various locations, including the sugar moiety, the phosphodiester linkage, and/or the base.

In some embodiments, the base moiety of a nucleoside may be modified. For example, a pyrimidine base may be modified at the 2, 3, 4, 5, and/or 6 position of the pyrimidine ring. In some embodiments, the exocyclic amine of cytosine may be modified. A purine base may also be modified. For example, a purine base may be modified at the 1, 2, 3, 6, 7, or 8 position. In some embodiments, the exocyclic amine of adenine may be modified. In some cases, a nitrogen atom in a ring of a base moiety may be substituted with another atom, such as carbon. A modification to a base moiety may be any suitable modification. Examples of modifications are known to those of ordinary skill in the art. In some embodiments, the base modifications include alkylated purines or pyrimidines, acylated purines or pyrimidines, or other heterocycles.

In some embodiments, a pyrimidine may be modified at the 5 position. For example, the 5 position of a pyrimidine may be modified with an alkyl group, an alkynyl group, an alkenyl group, an acyl group, or substituted derivatives thereof. In other examples, the 5 position of a pyrimidine may be modified with a hydroxyl group or an alkoxyl group or substituted derivative thereof. Also, the N⁴ position of a pyrimidine may be alkylated. In still further examples, the pyrimidine 5-6 bond may be saturated, a nitrogen atom within the pyrimidine ring may be substituted with a carbon atom, and/or the O² and O⁴ atoms may be substituted with sulfur atoms. It should be understood that other modifications are possible as well.

In other examples, the N⁷ position and/or N² and/or N³ position of a purine may be modified with an alkyl group or substituted derivative thereof. In further examples, a third ring may be fused to the purine bicyclic ring system and/or a nitrogen atom within the purine ring system may be substituted with a carbon atom. It should be understood that other modifications are possible as well.

Non-limiting examples of pyrimidines modified at the 5 position are disclosed in U.S. Pat. No. 5,591,843, U.S. Pat. No. 7,205,297, U.S. Pat. No. 6,432,963, and U.S. Pat. No. 6,020,483; non-limiting examples of pyrimidines modified at the N⁴ position are disclosed in U.S. Pat. No. 5,580,731; non-limiting examples of purines modified at the 8 position are disclosed in U.S. Pat. No. 6,355,787 and U.S. Pat. No. 5,580,972; non-limiting examples of purines modified at the N⁶ position are disclosed in U.S. Pat. No. 4,853,386, U.S. Pat. No. 5,789,416, and U.S. Pat. No. 7,041,824; and non-limiting examples of purines modified at the 2 position are disclosed in U.S. Pat. No. 4,201,860 and U.S. Pat. No. 5,587,469, all of which are incorporated herein by reference.

Non-limiting examples of modified bases include N⁴,N⁴-ethanocytosine, 7-deazaxanthosine, 7-deazaguanosine, 8-oxo-N⁶-methyladenine, 4-acetylcytosine, 5-(carboxyhydroxylmethyl) uracil, 5-fluorouracil, 5-bromouracil, 5-carboxymethylaminomethyl-2-thiouracil, 5-carboxymethylaminomethyl uracil, dihydrouracil, inosine, N⁶-isopentenyl-adenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N⁶-methyladenine, 7-methylguanine, 5-methylaminomethyl uracil, 5-methoxy aminomethyl-2-thiouracil, 5-methoxyuracil, 2-methylthio-N⁶-isopentenyladenine, pseudouracil, 5-methyl-2-thiouracil, 2-thiouracil, 4-thiouracil, 5-methyluracil, 2-thiocytosine, and 2,6-diaminopurine. In some embodiments, the base moiety may be a heterocyclic base other than a purine or pyrimidine. The heterocyclic base may be optionally modified and/or substituted.

Sugar moieties include natural, unmodified sugars, e.g., monosaccharide (such as pentose, e.g., ribose, deoxyribose), modified sugars and sugar analogs. In general, possible modifications of nucleomonomers, particularly of a sugar moiety, include, for example, replacement of one or more of the hydroxyl groups with a halogen, a heteroatom, an aliphatic group, or the functionalization of the hydroxyl group as an ether, an amine, a thiol, or the like.

One particularly useful group of modified nucleomonomers are 2′-O-methyl nucleotides. Such 2′-O-methyl nucleotides may be referred to as “methylated,” and the corresponding nucleotides may be made from unmethylated nucleotides followed by alkylation or directly from methylated nucleotide reagents. Modified nucleomonomers may be used in combination with unmodified nucleomonomers. For example, an oligonucleotide of the invention may contain both methylated and unmethylated nucleomonomers.

Some exemplary modified nucleomonomers include sugar- or backbone-modified ribonucleotides. Modified ribonucleotides may contain a non-naturally occurring base (instead of a naturally occurring base), such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides may have the 2′-OH group replaced by a H, alxoxy (or OR), R or alkyl, halogen, SH, SR, amino (such as NH₂, NHR, NR₂,), or CN group, wherein R is lower alkyl, alkenyl, or alkynyl.

Modified ribonucleotides may also have the phosphodiester group connecting to adjacent ribonucleotides replaced by a modified group, e.g., of phosphorothioate group. More generally, the various nucleotide modifications may be combined.

Although the antisense (guide) strand may be substantially identical to at least a portion of the target gene (or genes), at least with respect to the base pairing properties, the sequence need not be perfectly identical to be useful, e.g., to inhibit expression of a target gene's phenotype. Generally, higher homology can be used to compensate for the use of a shorter antisense gene. In some cases, the antisense strand generally will be substantially identical (although in antisense orientation) to the target gene.

The use of 2′-O-methyl modified RNA may also be beneficial in circumstances in which it is desirable to minimize cellular stress responses. RNA having 2′-O-methyl nucleomonomers may not be recognized by cellular machinery that is thought to recognize unmodified RNA. The use of 2′-O-methylated or partially 2′-O-methylated RNA may avoid the interferon response to double-stranded nucleic acids, while maintaining target RNA inhibition. This may be useful, for example, for avoiding the interferon or other cellular stress responses, both in short RNAi (e.g., siRNA) sequences that induce the interferon response, and in longer RNAi sequences that may induce the interferon response.

Overall, modified sugars may include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. In one embodiment, the sugar moiety can be a hexose and incorporated into an oligonucleotide as described (Augustyns, K., et al., Nucl. Acids. Res. 18:4711 (1992)). Exemplary nucleomonomers can be found, e.g., in U.S. Pat. No. 5,849,902, incorporated by reference herein.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

In certain embodiments, oligonucleotides of the invention comprise 3′ and 5′ termini (except for circular oligonucleotides). In one embodiment, the 3′ and 5′ termini of an oligonucleotide can be substantially protected from nucleases e.g., by modifying the 3′ or 5′ linkages (e.g., U.S. Pat. No. 5,849,902 and WO 98/13526). For example, oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

One of ordinary skill in the art will appreciate that the synthetic methods, as described herein, utilize a variety of protecting groups. By the term “protecting group,” as used herein, it is meant that a particular functional moiety, e.g., O, S, or N, is temporarily blocked so that a reaction can be carried out selectively at another reactive site in a multifunctional compound. In certain embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM),

p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, a-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein. However, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups. In certain embodiments, as used herein, “lower alkyl” is used to indicate those alkyl groups (cyclic, acyclic, substituted, unsubstituted, branched, or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

Some examples of substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂R; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments described herein.

The term “heteroaliphatic,” as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(R)₂; —OC(O)Rₓ; —OCO₂Rₓ; —OCON(Rₓ)₂; —N(R)₂; —S(O)₂R; —NR(CO)R, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments described herein.

The terms “halo” and “halogen” as used herein refer to an atom selected from fluorine, chlorine, bromine, and iodine.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids. The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, unless otherwise specified, the term alkenyl includes both “unsubstituted alkenyls” and “substituted alkenyls,” the latter of which refers to alkenyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, unless otherwise specified, the term alkynyl includes both “unsubstituted alkynyls” and “substituted alkynyls,” the latter of which refers to alkynyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with independently selected groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulffiydryl, alkylthio, arylthio, thiocarboxylate, sulfates, alkylsulfmyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “heteroatom” includes atoms of any element other than carbon or hydrogen. Preferred heteroatoms are nitrogen, oxygen, sulfur and phosphorus.

The term “hydroxy” or “hydroxyl” includes groups with an —OH or —O⁻ (with an appropriate counterion).

The term “halogen” includes fluorine, bromine, chlorine, iodine, etc. The term “perhalogenated” generally refers to a moiety wherein all hydrogens are replaced by halogen atoms.

The term “substituted” includes independently selected substituents which can be placed on the moiety and which allow the molecule to perform its intended function. Examples of substituents include alkyl, alkenyl, alkynyl, aryl, (CR′R″)₀₋₃NR′R″, (CR′R″)₀₋₃CN, NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂W, (CR′R″)₀₋₃O(CR′R″)₀₋₃H, (CR′R″)₀₋₃COR′, (CR′R″)₀₋₃CO₂R′, or (CR′R″)₀₋₃OR′ groups; wherein each R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups.

The term “ether” includes compounds or moieties which contain an oxygen bonded to two different carbon atoms or heteroatoms. For example, the term includes “alkoxyalkyl,” which refers to an alkyl, alkenyl, or alkynyl group covalently bonded to an oxygen atom which is covalently bonded to another alkyl group.

The terms “polynucleotide,” “nucleotide sequence,” “nucleic acid,” “nucleic acid molecule,” “nucleic acid sequence,” and “oligonucleotide” refer to a polymer of two or more nucleotides. The polynucleotides can be DNA, RNA, or derivatives or modified versions thereof. The polynucleotide may be single-stranded or double-stranded. The polynucleotide can be modified at the base moiety, sugar moiety, or phosphate backbone, for example, to improve stability of the molecule, its hybridization parameters, etc. The polynucleotide may comprise a modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine. The polynucleotide may comprise a modified sugar moiety (e.g., 2′-fluororibose, ribose, 2′-deoxyribose, 2′-O-methylcytidine, arabinose, and hexose), and/or a modified phosphate moiety (e.g., phosphorothioates and 5′-N-phosphoramidite linkages). A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA, and RNA-RNA hybrids, as well as “protein nucleic acids” (PNA) formed by conjugating bases to an amino acid backbone.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N⁶-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In a preferred embodiment, the nucleomonomers of an oligonucleotide of the invention are RNA nucleotides. In another preferred embodiment, the nucleomonomers of an oligonucleotide of the invention are modified RNA nucleotides. Thus, the oligonucleotides contain modified RNA nucleotides.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2^{nd} Ed., Wiley-Interscience, New York, 1999).

The term “nucleotide” includes nucleosides which further comprise a phosphate group or a phosphate analog.

The nucleic acid molecules may be associated with a hydrophobic moiety for targeting and/or delivery of the molecule to a cell. In certain embodiments, the hydrophobic moiety is associated with the nucleic acid molecule through a linker. In certain embodiments, the association is through non-covalent interactions. In other embodiments, the association is through a covalent bond. Any linker known in the art may be used to associate the nucleic acid with the hydrophobic moiety. Linkers known in the art are described in published international PCT applications, WO 92/03464, WO 95/23162, WO 2008/021157, WO 2009/021157, WO 2009/134487, WO 2009/126933, U.S. Patent Application Publication 2005/0107325, U.S. Pat. No. 5,414,077, U.S. Pat. No. 5,419,966, U.S. Pat. No. 5,512,667, U.S. Pat. No. 5,646,126, and U.S. Pat. No. 5,652,359, which are incorporated herein by reference. The linker may be as simple as a covalent bond to a multi-atom linker. The linker may be cyclic or acyclic. The linker may be optionally substituted. In certain embodiments, the linker is capable of being cleaved from the nucleic acid. In certain embodiments, the linker is capable of being hydrolyzed under physiological conditions. In certain embodiments, the linker is capable of being cleaved by an enzyme (e.g., an esterase or phosphodiesterase). In certain embodiments, the linker comprises a spacer element to separate the nucleic acid from the hydrophobic moiety. The spacer element may include one to thirty carbon or heteroatoms. In certain embodiments, the linker and/or spacer element comprises protonatable functional groups. Such protonatable functional groups may promote the endosomal escape of the nucleic acid molecule. The protonatable functional groups may also aid in the delivery of the nucleic acid to a cell, for example, neutralizing the overall charge of the molecule. In other embodiments, the linker and/or spacer element is biologically inert (that is, it does not impart biological activity or function to the resulting nucleic acid molecule).

In certain embodiments, the nucleic acid molecule with a linker and hydrophobic moiety is of the formulae described herein. In certain embodiments, the nucleic acid molecule is of the formula:

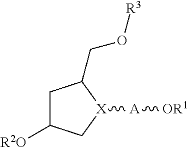

wherein

X is N or CH;

A is a bond; substituted or unsubstituted, cyclic or acyclic, branched or unbranched aliphatic; or substituted or unsubstituted, cyclic or acyclic, branched or unbranched heteroaliphatic;

R¹ is a hydrophobic moiety;

R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and

R³ is a nucleic acid.

In certain embodiments, the molecule is of the formula:

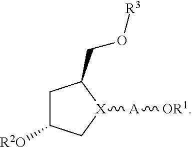

In certain embodiments, the molecule is of the formula:

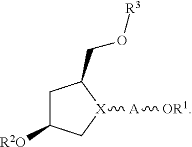

In certain embodiments, the molecule is of the formula:

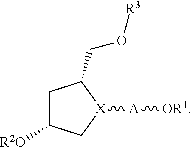

In certain embodiments, the molecule is of the formula:

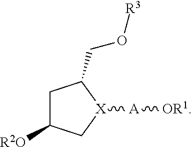

In certain embodiments, X is N. In certain embodiments, X is CH.

In certain embodiments, A is a bond. In certain embodiments, A is substituted or unsubstituted, cyclic or acyclic, branched or unbranched aliphatic. In certain embodiments, A is acyclic, substituted or unsubstituted, branched or unbranched aliphatic. In certain embodiments, A is acyclic, substituted, branched or unbranched aliphatic. In certain embodiments, A is acyclic, substituted, unbranched aliphatic. In certain embodiments, A is acyclic, substituted, unbranched alkyl. In certain embodiments, A is acyclic, substituted, unbranched C₁₋₂₀ alkyl. In certain embodiments, A is acyclic, substituted, unbranched C₁₋₁₂ alkyl. In certain embodiments, A is acyclic, substituted, unbranched C₁₋₁₀ alkyl. In certain embodiments, A is acyclic, substituted, unbranched C₁₋₈ alkyl. In certain embodiments, A is acyclic, substituted, unbranched C₁₋₆ alkyl. In certain embodiments, A is substituted or unsubstituted, cyclic or acyclic, branched or unbranched heteroaliphatic. In certain embodiments, A is acyclic, substituted or unsubstituted, branched or unbranched heteroaliphatic. In certain embodiments, A is acyclic, substituted, branched or unbranched heteroaliphatic. In certain embodiments, A is acyclic, substituted, unbranched heteroaliphatic.

In certain embodiments, A is of the formula:

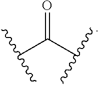 [SMILES: *C(*)=O]

In certain embodiments, A is of one of the formulae:

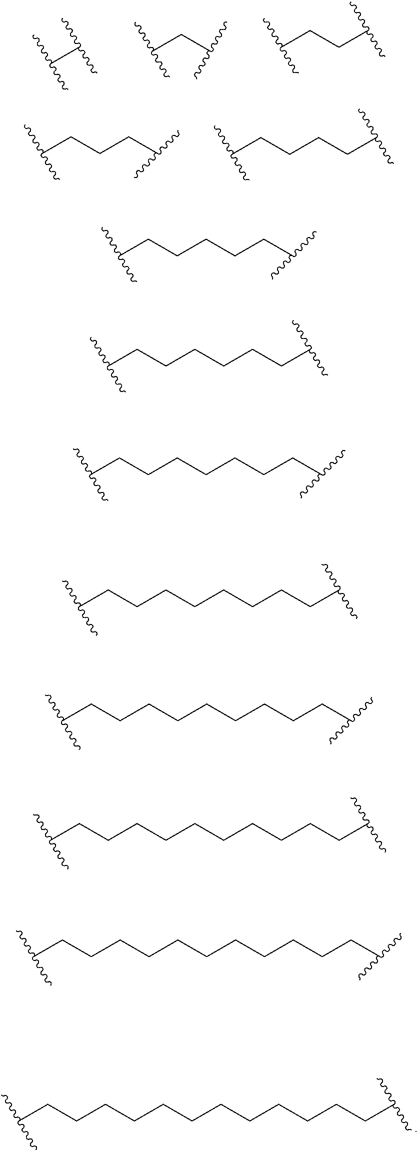

In certain embodiments, A is of one of the formulae:

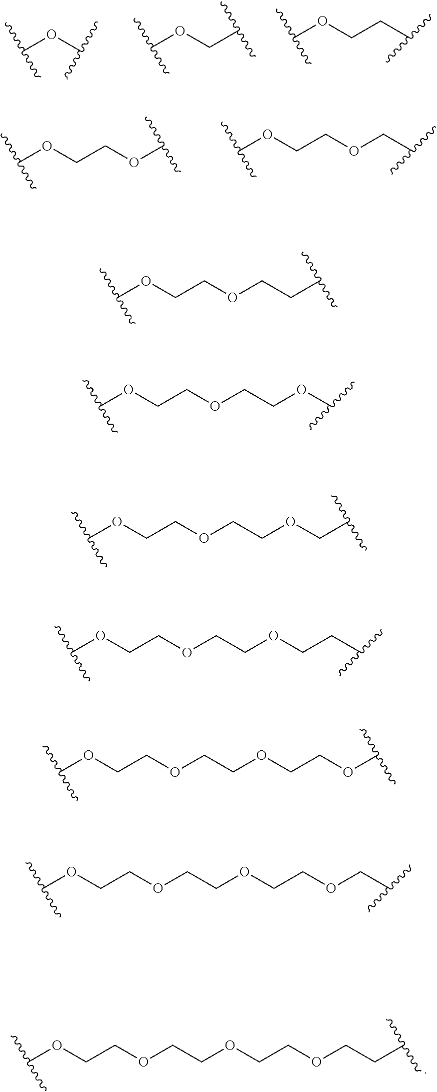

In certain embodiments, A is of one of the formulae:

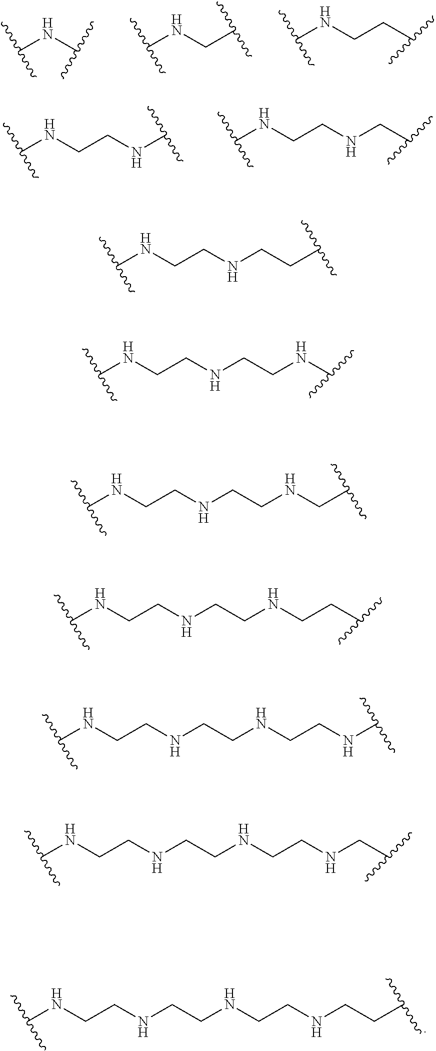

In certain embodiments, A is of the formula:

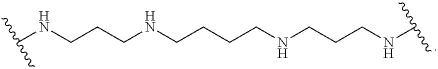 [SMILES: *NCCCNCCCCNCCCN*]

In certain embodiments A is of the formula:

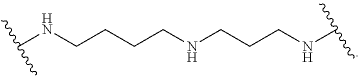 [SMILES: *NCCCCNCCCN*]

In certain embodiments, A is of the formula:

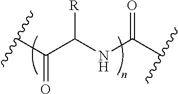

wherein

each occurrence of R is independently the side chain of a natural or unnatural amino acid; and

n is an integer between 1 and 20, inclusive. In certain embodiments, A is of the formula:

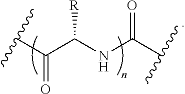

In certain embodiments, each occurrence of R is independently the side chain of a natural amino acid. In certain embodiments, n is an integer between 1 and 15, inclusive. In certain embodiments, n is an integer between 1 and 10, inclusive. In certain embodiments, n is an integer between 1 and 5, inclusive.

In certain embodiments, A is of the formula:

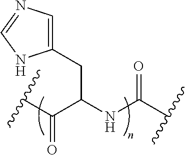

wherein n is an integer between 1 and 20, inclusive. In certain embodiments, A is of the formula:

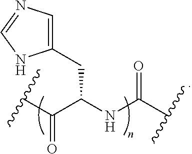

In certain embodiments, n is an integer between 1 and 15, inclusive. In certain embodiments, n is an integer between 1 and 10, inclusive. In certain embodiments, n is an integer between 1 and 5, inclusive.

In certain embodiments, A is of the formula:

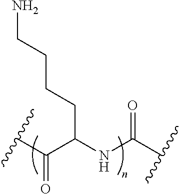

wherein n is an integer between 1 and 20, inclusive. In certain embodiments, A is of the formula:

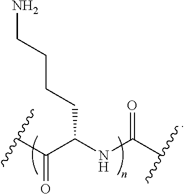

In certain embodiments, n is an integer between 1 and 15, inclusive. In certain embodiments, n is an integer between 1 and 10, inclusive. In certain embodiments, n is an integer between 1 and 5, inclusive.

In certain embodiments, the molecule is of the formula:

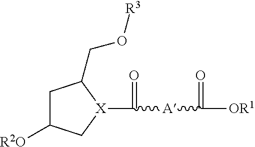

wherein X, R¹, R², and R³ are as defined herein; and

A′ is substituted or unsubstituted, cyclic or acyclic, branched or unbranched aliphatic; or substituted or unsubstituted, cyclic or acyclic, branched or unbranched heteroaliphatic.

In certain embodiments, A′ is of one of the formulae:

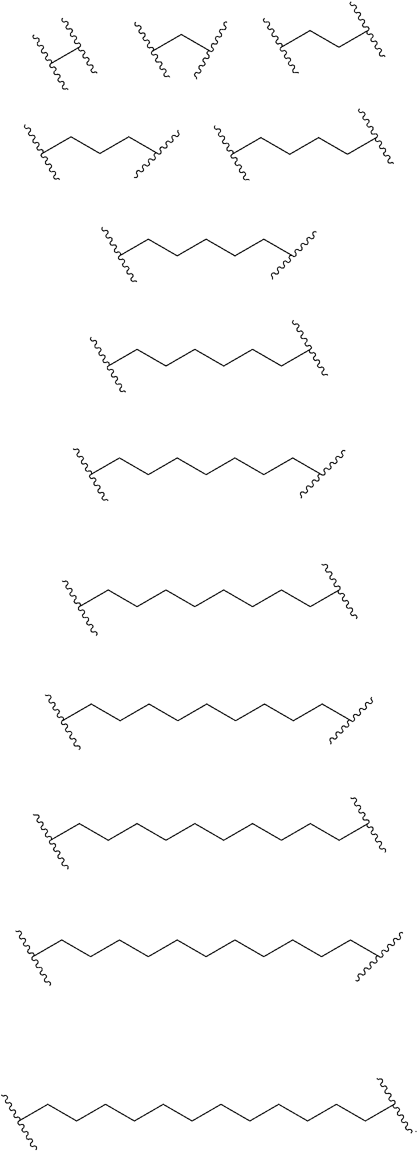

In certain embodiments, A is of one of the formulae:

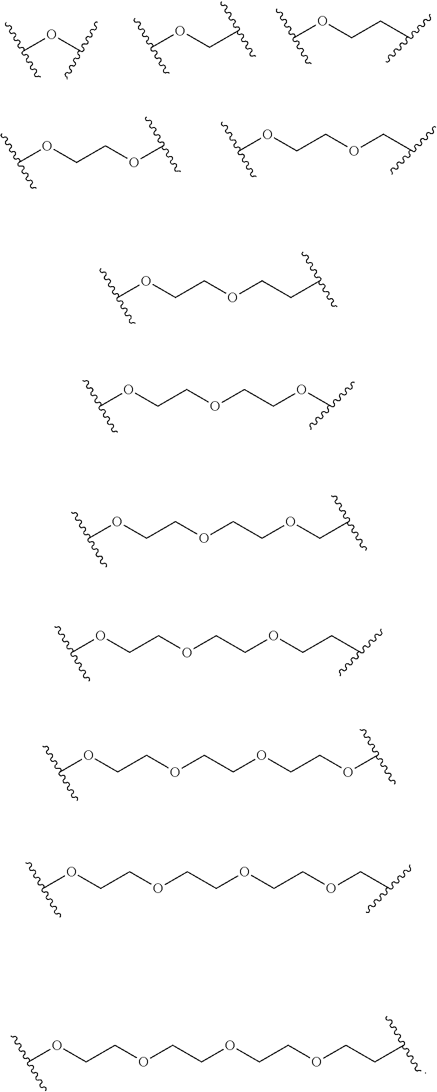

In certain embodiments, A is of one of the formulae:

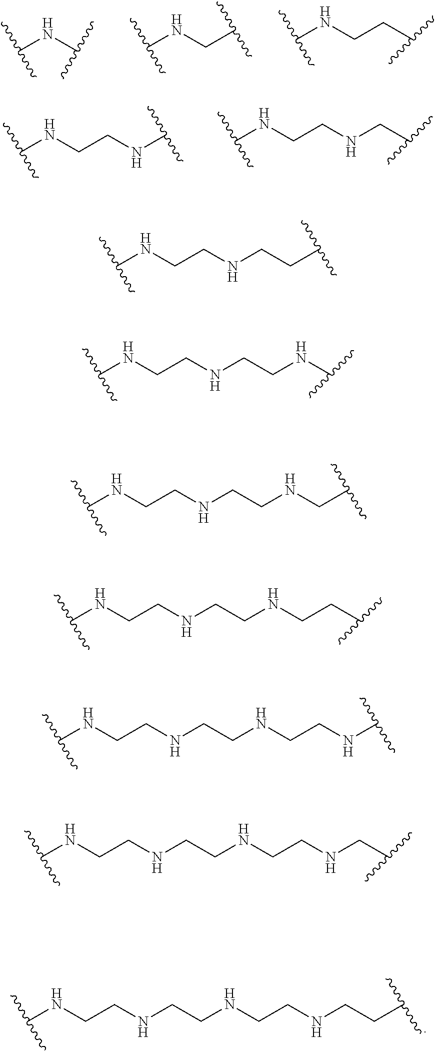

In certain embodiments, A is of the formula:

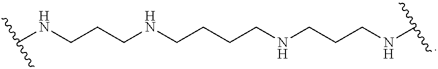 [SMILES: *NCCCNCCCCNCCCN*]

In certain embodiments, A is of the formula:

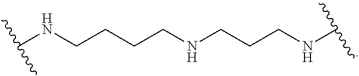 [SMILES: *NCCCCNCCCN*]

In certain embodiments, R¹ is a steroid. In certain embodiments, R¹ is a cholesterol. In certain embodiments, R¹ is a lipophilic vitamin. In certain embodiments, R¹ is a vitamin A. In certain embodiments, R¹ is a vitamin E.
In certain embodiments, R¹ is of the formula:

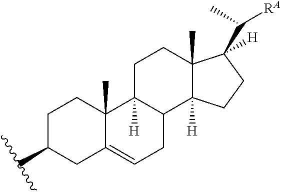

wherein R^{A} is substituted or unsubstituted, cyclic or acyclic, branched or unbranched aliphatic; or substituted or unsubstituted, cyclic or acyclic, branched or unbranched heteroaliphatic.
In certain embodiments, R¹ is of the formula:

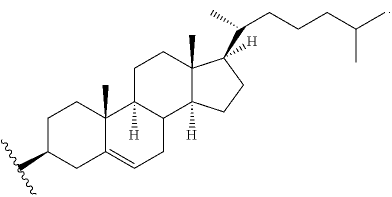

In certain embodiments, R¹ is of the formula:

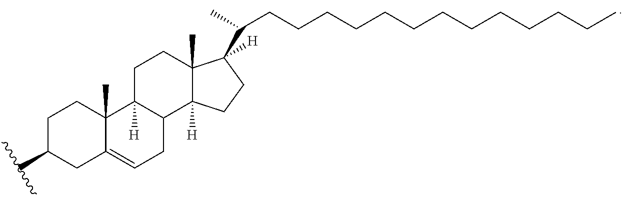

In certain embodiments, R¹ is of the formula:

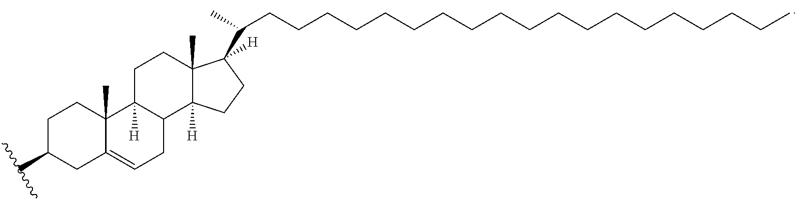

In certain embodiments, R¹ is of the formula:

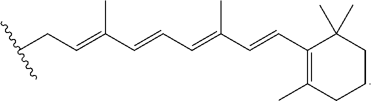 [SMILES: *C/C=C(C)/C=C/C=C(C)/C=C/C1=C(C)CCCC1(C)C]

In certain embodiments, R¹ is of the formula:

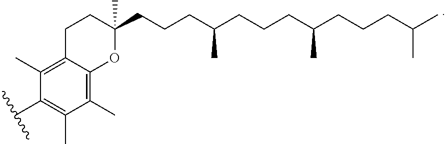 [SMILES: *c1c(C)c(C)c2c(c1C)CC[C@@](C)(CCC[C@H](C)CCC[C@H](C)CCCC(C)C)O2]

In certain embodiments, the nucleic acid molecule is of the formula:

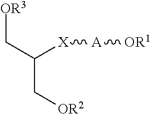

wherein

### X is N or CH;

A is a bond; substituted or unsubstituted, cyclic or acyclic, branched or unbranched aliphatic; or substituted or unsubstituted, cyclic or acyclic, branched or unbranched heteroaliphatic;
R¹ is a hydrophobic moiety;
R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and
R³ is a nucleic acid.
In certain embodiments, the nucleic acid molecule is of the formula:

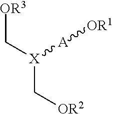

wherein

### X is N or CH;

A is a bond; substituted or unsubstituted, cyclic or acyclic, branched or unbranched aliphatic; or substituted or unsubstituted, cyclic or acyclic, branched or unbranched heteroaliphatic;
R¹ is a hydrophobic moiety;
R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and
R³ is a nucleic acid.
In certain embodiments, the nucleic acid molecule is of the formula:

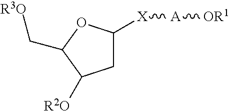

wherein

### X is N or CH;

A is a bond; substituted or unsubstituted, cyclic or acyclic, branched or unbranched aliphatic; or substituted or unsubstituted, cyclic or acyclic, branched or unbranched heteroaliphatic;
R¹ is a hydrophobic moiety;
R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and
R³ is a nucleic acid. In certain embodiments, the nucleic acid molecule is of the formula:

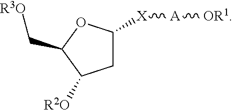

In certain embodiments, the nucleic acid molecule is of the formula:

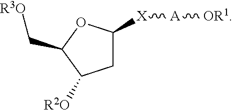

In certain embodiments, the nucleic acid molecule is of the formula:

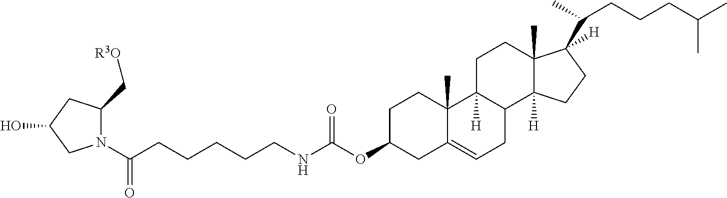

wherein R³ is a nucleic acid.
In certain embodiments, the nucleic acid molecule is of the formula:

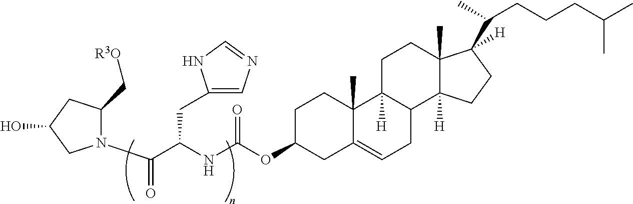

wherein R³ is a nucleic acid; and
n is an integer between 1 and 20, inclusive.
In certain embodiments, the nucleic acid molecule is of the formula:

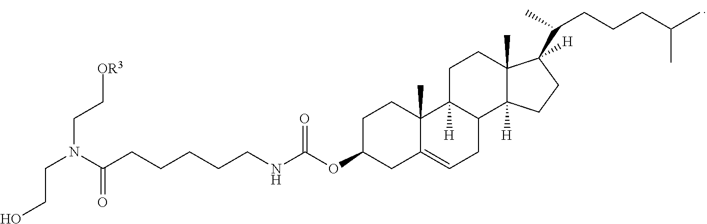

In certain embodiments, the nucleic acid molecule is of the formula:

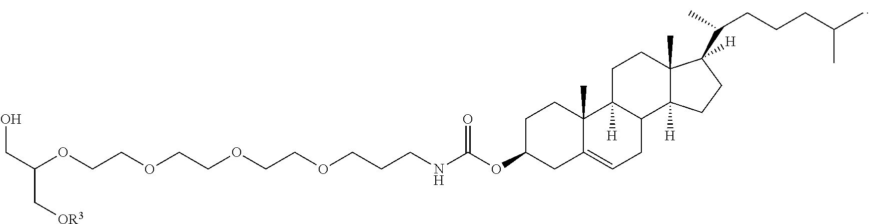

In certain embodiments, the nucleic acid molecule is of the formula:

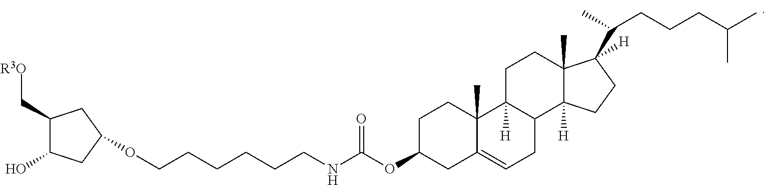

In certain embodiments, the nucleic acid molecule is of the formula:

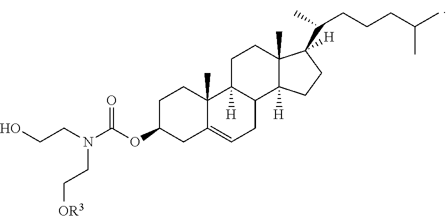

In certain embodiments, the nucleic acid molecule is of the formula:

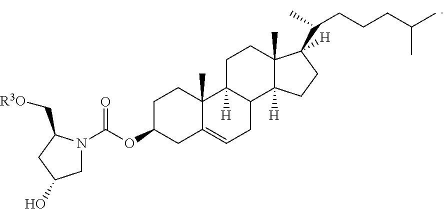

As used herein, the term “linkage” includes a naturally occurring, unmodified phosphodiester moiety (—O—(PO²⁻)—O—) that covalently couples adjacent nucleomonomers. As used herein, the term “substitute linkage” includes any analog or derivative of the native phosphodiester group that covalently couples adjacent nucleomonomers. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47). In certain embodiments, non-hydrolizable linkages are preferred, such as phosphorothiate linkages.

In certain embodiments, oligonucleotides of the invention comprise hydrophobicly modified nucleotides or “hydrophobic modifications.” As used herein “hydrophobic modifications” refers to bases that are modified such that (1) overall hydrophobicity of the base is significantly increased, and/or (2) the base is still capable of forming close to regular Watson-Crick interaction. Several non-limiting examples of base modifications include 5-position uridine and cytidine modifications such as phenyl, 4-pyridyl, 2-pyridyl, indolyl, and isobutyl, phenyl (C6H5OH); tryptophanyl (C8H6N)CH2CH(NH2)CO), Isobutyl, butyl, aminobenzyl; phenyl; and naphthyl.

Another type of conjugates that can be attached to the end (3′ or 5′ end), the loop region, or any other parts of the sd-rxRNA might include a sterol, sterol type molecule, peptide, small molecule, protein, etc. In some embodiments, a sdrxRNA may contain more than one conjugates (same or different chemical nature). In some embodiments, the conjugate is cholesterol.

Another way to increase target gene specificity, or to reduce off-target silencing effect, is to introduce a 2′-modification (such as the 2′-O methyl modification) at a position corresponding to the second 5′-end nucleotide of the guide sequence. This allows the positioning of this 2′-modification in the Dicer-resistant hairpin structure, thus enabling one to design better RNAi constructs with less or no off-target silencing.

In one embodiment, a hairpin polynucleotide of the invention can comprise one nucleic acid portion which is DNA and one nucleic acid portion which is RNA. Antisense (guide) sequences of the invention can be “chimeric oligonucleotides” which comprise an RNA-like and a DNA-like region.

The language “RNase H activating region” includes a region of an oligonucleotide, e.g., a chimeric oligonucleotide, that is capable of recruiting RNase H to cleave the target RNA strand to which the oligonucleotide binds. Typically, the RNase activating region contains a minimal core (of at least about 3-5, typically between about 3-12, more typically, between about 5-12, and more preferably between about 5-10 contiguous nucleomonomers) of DNA or DNA-like nucleomonomers. (See, e.g., U.S. Pat. No. 5,849,902). Preferably, the RNase H activating region comprises about nine contiguous deoxyribose containing nucleomonomers.

The language “non-activating region” includes a region of an antisense sequence, e.g., a chimeric oligonucleotide, that does not recruit or activate RNase H. Preferably, a non-activating region does not comprise phosphorothioate DNA. The oligonucleotides of the invention comprise at least one non-activating region. In one embodiment, the non-activating region can be stabilized against nucleases or can provide specificity for the target by being complementary to the target and forming hydrogen bonds with the target nucleic acid molecule, which is to be bound by the oligonucleotide.

In one embodiment, at least a portion of the contiguous polynucleotides are linked by a substitute linkage, e.g., a phosphorothioate linkage.

In certain embodiments, most or all of the nucleotides beyond the guide sequence (2′-modified or not) are linked by phosphorothioate linkages. Such constructs tend to have improved pharmacokinetics due to their higher affinity for serum proteins. The phosphorothioate linkages in the non-guide sequence portion of the polynucleotide generally do not interfere with guide strand activity, once the latter is loaded into RISC.

Antisense (guide) sequences of the present invention may include “morpholino oligonucleotides.” Morpholino oligonucleotides are non-ionic and function by an RNase H-independent mechanism. Each of the 4 genetic bases (Adenine, Cytosine, Guanine, and Thymine/Uracil) of the morpholino oligonucleotides is linked to a 6-membered morpholine ring. Morpholino oligonucleotides are made by joining the 4 different subunit types by, e.g., non-ionic phosphorodiamidate inter-subunit linkages. Morpholino oligonucleotides have many advantages including: complete resistance to nucleases (Antisense & Nucl. Acid Drug Dev. 1996. 6:267); predictable targeting (Biochemica Biophysica Acta. 1999. 1489:141); reliable activity in cells (Antisense & Nucl. Acid Drug Dev. 1997. 7:63); excellent sequence specificity (Antisense & Nucl. Acid Drug Dev. 1997. 7:151); minimal non-antisense activity (Biochemica Biophysica Acta. 1999. 1489:141); and simple osmotic or scrape delivery (Antisense & Nucl. Acid Drug Dev. 1997. 7:291). Morpholino oligonucleotides are also preferred because of their non-toxicity at high doses. A discussion of the preparation of morpholino oligonucleotides can be found in Antisense & Nucl. Acid Drug Dev. 1997. 7:187.

The chemical modifications described herein are believed, based on the data described herein, to promote single stranded polynucleotide loading into the RISC. Single stranded polynucleotides have been shown to be active in loading into RISC and inducing gene silencing. However, the level of activity for single stranded polynucleotides appears to be 2 to 4 orders of magnitude lower when compared to a duplex polynucleotide.

The present invention provides a description of the chemical modification patterns, which may (a) significantly increase stability of the single stranded polynucleotide (b) promote efficient loading of the polynucleotide into the RISC complex and (c) improve uptake of the single stranded nucleotide by the cell. FIG. 18 provides some non-limiting examples of the chemical modification patterns which may be beneficial for achieving single stranded polynucleotide efficacy inside the cell. The chemical modification patterns may include combination of ribose, backbone, hydrophobic nucleoside and conjugate type of modifications. In addition, in some of the embodiments, the 5′ end of the single polynucleotide may be chemically phosphorylated.

In yet another embodiment, the present invention provides a description of the chemical modifications patterns, which improve functionality of RISC inhibiting polynucleotides. Single stranded polynucleotides have been shown to inhibit activity of a preloaded RISC complex through the substrate competition mechanism. For these types of molecules, conventionally called antagomers, the activity usually requires high concentration and in vivo delivery is not very effective. The present invention provides a description of the chemical modification patterns, which may (a) significantly increase stability of the single stranded polynucleotide (b) promote efficient recognition of the polynucleotide by the RISC as a substrate and/or (c) improve uptake of the single stranded nucleotide by the cell. FIG. 6 provides some non-limiting examples of the chemical modification patterns that may be beneficial for achieving single stranded polynucleotide efficacy inside the cell. The chemical modification patterns may include combination of ribose, backbone, hydrophobic nucleoside and conjugate type of modifications.

The modifications provided by the present invention are applicable to all polynucleotides. This includes single stranded RISC entering polynucleotides, single stranded RISC inhibiting polynucleotides, conventional duplexed polynucleotides of variable length (15-40 bp), asymmetric duplexed polynucleotides, and the like. Polynucleotides may be modified with wide variety of chemical modification patterns, including 5′ end, ribose, backbone and hydrophobic nucleoside modifications.

### Synthesis

Oligonucleotides of the invention can be synthesized by any method known in the art, e.g., using enzymatic synthesis and/or chemical synthesis. The oligonucleotides can be synthesized in vitro (e.g., using enzymatic synthesis and chemical synthesis) or in vivo (using recombinant DNA technology well known in the art).

In a preferred embodiment, chemical synthesis is used for modified polynucleotides. Chemical synthesis of linear oligonucleotides is well known in the art and can be achieved by solution or solid phase techniques. Preferably, synthesis is by solid phase methods. Oligonucleotides can be made by any of several different synthetic procedures including the phosphoramidite, phosphite triester, H-phosphonate, and phosphotriester methods, typically by automated synthesis methods.

Oligonucleotide synthesis protocols are well known in the art and can be found, e.g., in U.S. Pat. No. 5,830,653; WO 98/13526; Stec et al. 1984. J. Am. Chem. Soc. 106:6077; Stec et al. 1985. J. Org. Chem. 50:3908; Stec et al. J. Chromatog. 1985. 326:263; LaPlanche et al. 1986. Nucl. Acid. Res. 1986. 14:9081; Fasman G. D., 1989. Practical Handbook of Biochemistry and Molecular Biology. 1989. CRC Press, Boca Raton, Fla.; Lamone. 1993. Biochem. Soc. Trans. 21:1; U.S. Pat. No. 5,013,830; U.S. Pat. No. 5,214,135; U.S. Pat. No. 5,525,719; Kawasaki et al. 1993. J. Med. Chem. 36:831; WO 92/03568; U.S. Pat. No. 5,276,019; and U.S. Pat. No. 5,264,423.

The synthesis method selected can depend on the length of the desired oligonucleotide and such choice is within the skill of the ordinary artisan. For example, the phosphoramidite and phosphite triester method can produce oligonucleotides having 175 or more nucleotides, while the H-phosphonate method works well for oligonucleotides of less than 100 nucleotides. If modified bases are incorporated into the oligonucleotide, and particularly if modified phosphodiester linkages are used, then the synthetic procedures are altered as needed according to known procedures. In this regard, Uhlmann et al. (1990, Chemical Reviews 90:543-584) provide references and outline procedures for making oligonucleotides with modified bases and modified phosphodiester linkages. Other exemplary methods for making oligonucleotides are taught in Sonveaux. 1994. “Protecting Groups in Oligonucleotide Synthesis”; Agrawal. Methods in Molecular Biology 26:1. Exemplary synthesis methods are also taught in “Oligonucleotide Synthesis—A Practical Approach” (Gait, M. J. IRL Press at Oxford University Press. 1984). Moreover, linear oligonucleotides of defined sequence, including some sequences with modified nucleotides, are readily available from several commercial sources.

The oligonucleotides may be purified by polyacrylamide gel electrophoresis, or by any of a number of chromatographic methods, including gel chromatography and high pressure liquid chromatography. To confirm a nucleotide sequence, especially unmodified nucleotide sequences, oligonucleotides may be subjected to DNA sequencing by any of the known procedures, including Maxam and Gilbert sequencing, Sanger sequencing, capillary electrophoresis sequencing, the wandering spot sequencing procedure or by using selective chemical degradation of oligonucleotides bound to Hybond paper. Sequences of short oligonucleotides can also be analyzed by laser desorption mass spectroscopy or by fast atom bombardment (McNeal, et al., 1982, J. Am. Chem. Soc. 104:976; Viari, et al., 1987, Biomed. Environ. Mass Spectrom. 14:83; Grotjahn et al., 1982, Nuc. Acid Res. 10:4671). Sequencing methods are also available for RNA oligonucleotides.

The quality of oligonucleotides synthesized can be verified by testing the oligonucleotide by capillary electrophoresis and denaturing strong anion HPLC (SAX-HPLC) using, e.g., the method of Bergot and Egan. 1992. J. Chrom. 599:35.

Other exemplary synthesis techniques are well known in the art (see, e.g., Sambrook et al., Molecular Cloning: a Laboratory Manual, Second Edition (1989); DNA Cloning, Volumes I and II (DN Glover Ed. 1985); Oligonucleotide Synthesis (M J Gait Ed, 1984; Nucleic Acid Hybridisation (B D Hames and S J Higgins eds. 1984); A Practical Guide to Molecular Cloning (1984); or the series, Methods in Enzymology (Academic Press, Inc.)).

In certain embodiments, the subject RNAi constructs or at least portions thereof are transcribed from expression vectors encoding the subject constructs. Any art recognized vectors may be use for this purpose. The transcribed RNAi constructs may be isolated and purified, before desired modifications (such as replacing an unmodified sense strand with a modified one, etc.) are carried out.

### Delivery/Carrier

### Uptake of Oligonucleotides by Cells

Oligonucleotides and oligonucleotide compositions are contacted with (i.e., brought into contact with, also referred to herein as administered or delivered to) and taken up by one or more cells or a cell lysate. The term “cells” includes prokaryotic and eukaryotic cells, preferably vertebrate cells, and, more preferably, mammalian cells. In a preferred embodiment, the oligonucleotide compositions of the invention are contacted with human cells.

Oligonucleotide compositions of the invention can be contacted with cells in vitro, e.g., in a test tube or culture dish, (and may or may not be introduced into a subject) or in vivo, e.g., in a subject such as a mammalian subject. In some embodiments, Oligonucleotides are administered topically or through electroporation. Oligonucleotides are taken up by cells at a slow rate by endocytosis, but endocytosed oligonucleotides are generally sequestered and not available, e.g., for hybridization to a target nucleic acid molecule. In one embodiment, cellular uptake can be facilitated by electroporation or calcium phosphate precipitation. However, these procedures are only useful for in vitro or ex vivo embodiments, are not convenient and, in some cases, are associated with cell toxicity.

In another embodiment, delivery of oligonucleotides into cells can be enhanced by suitable art recognized methods including calcium phosphate, DMSO, glycerol or dextran, electroporation, or by transfection, e.g., using cationic, anionic, or neutral lipid compositions or liposomes using methods known in the art (see e.g., WO 90/14074; WO 91/16024; WO 91/17424; U.S. Pat. No. 4,897,355; Bergan et al. 1993. Nucleic Acids Research. 21:3567). Enhanced delivery of oligonucleotides can also be mediated by the use of vectors (See e.g., Shi, Y. 2003. Trends Genet 2003 Jan. 19:9; Reichhart J M et al. Genesis. 2002. 34(1-2):1604, Yu et al. 2002. Proc. Natl. Acad Sci. USA 99:6047; Sui et al. 2002. Proc. Natl. Acad Sci. USA 99:5515) viruses, polyamine or polycation conjugates using compounds such as polylysine, protamine, or Ni, N12-bis (ethyl) spermine (see, e.g., Bartzatt, R. et al. 1989. Biotechnol. Appl. Biochem. 11:133; Wagner E. et al. 1992. Proc. Natl. Acad. Sci. 88:4255).

In certain embodiments, the sd-rxRNA of the invention may be delivered by using various beta-glucan containing particles, referred to as GeRPs (glucan encapsulated RNA loaded particle), described in, and incorporated by reference from, U.S. Provisional Application No. 61/310,611, filed on Mar. 4, 2010 and entitled “Formulations and Methods for Targeted Delivery to Phagocyte Cells.” Such particles are also described in, and incorporated by reference from US Patent Publications US 2005/0281781 A1, and US 2010/0040656, and in PCT publications WO 2006/007372, and WO 2007/050643. The sd-rxRNA molecule may be hydrophobically modified and optionally may be associated with a lipid and/or amphiphilic peptide. In certain embodiments, the beta-glucan particle is derived from yeast. In certain embodiments, the payload trapping molecule is a polymer, such as those with a molecular weight of at least about 1000 Da, 10,000 Da, 50,000 Da, 100 kDa, 500 kDa, etc. Preferred polymers include (without limitation) cationic polymers, chitosans, or PEI (polyethylenimine), etc.

Glucan particles can be derived from insoluble components of fungal cell walls such as yeast cell walls. In some embodiments, the yeast is Baker's yeast. Yeast-derived glucan molecules can include one or more of β-(1,3)-Glucan, β-(1,6)-Glucan, mannan and chitin. In some embodiments, a glucan particle comprises a hollow yeast cell wall whereby the particle maintains a three dimensional structure resembling a cell, within which it can complex with or encapsulate a molecule such as an RNA molecule. Some of the advantages associated with the use of yeast cell wall particles are availability of the components, their biodegradable nature, and their ability to be targeted to phagocytic cells.

In some embodiments, glucan particles can be prepared by extraction of insoluble components from cell walls, for example by extracting Baker's yeast (Fleischmann's) with 1M NaOH/pH 4.0 H2O, followed by washing and drying. Methods of preparing yeast cell wall particles are discussed in, and incorporated by reference from U.S. Pat. Nos. 4,810,646, 4,992,540, 5,082,936, 5,028,703, 5,032,401, 5,322,841, 5,401,727, 5,504,079, 5,607,677, 5,968,811, 6,242,594, 6,444,448, 6,476,003, US Patent Publications 2003/0216346, 2004/0014715 and 2010/0040656, and PCT published application WO02/12348.

Protocols for preparing glucan particles are also described in, and incorporated by reference from, the following references: Soto and Ostroff (2008), “Characterization of multilayered nanoparticles encapsulated in yeast cell wall particles for DNA delivery.” Bioconjug Chem 19(4):840-8; Soto and Ostroff (2007), “Oral Macrophage Mediated Gene Delivery System,” Nanotech, Volume 2, Chapter 5 (“Drug Delivery”), pages 378-381; and Li et al. (2007), “Yeast glucan particles activate murine resident macrophages to secrete proinflammatory cytokines via MyD88- and Syk kinase-dependent pathways.” Clinical Immunology 124(2):170-181.

Glucan containing particles such as yeast cell wall particles can also be obtained commercially. Several non-limiting examples include: Nutricell MOS 55 from Biorigin (Sao Paolo, Brazil), SAF-Mannan (SAF Agri, Minneapolis, Minn.), Nutrex (Sensient Technologies, Milwaukee, Wis.), alkali-extracted particles such as those produced by Nutricepts (Nutricepts Inc., Burnsville, Minn.) and ASA Biotech, acid-extracted WGP particles from Biopolymer Engineering, and organic solvent-extracted particles such as Adjuvaxtm from Alpha-beta Technology, Inc. (Worcester, Mass.) and microparticulate glucan from Novogen (Stamford, Conn.).

Glucan particles such as yeast cell wall particles can have varying levels of purity depending on the method of production and/or extraction. In some instances, particles are alkali-extracted, acid-extracted or organic solvent-extracted to remove intracellular components and/or the outer mannoprotein layer of the cell wall. Such protocols can produce particles that have a glucan (w/w) content in the range of 50%-90%. In some instances, a particle of lower purity, meaning lower glucan w/w content may be preferred, while in other embodiments, a particle of higher purity, meaning higher glucan w/w content may be preferred.

Glucan particles, such as yeast cell wall particles, can have a natural lipid content. For example, the particles can contain 1%, 2%, 3%, 4%, 5%, 6%, 7%, 8%, 9%, 10%, 11%, 12%, 13%, 14%, 15%, 16%, 17%, 18%, 19%, 20% or more than 20% w/w lipid. In the Examples section, the effectiveness of two glucan particle batches are tested: YGP SAF and YGP SAF+L (containing natural lipids). In some instances, the presence of natural lipids may assist in complexation or capture of RNA molecules.

Glucan containing particles typically have a diameter of approximately 2-4 microns, although particles with a diameter of less than 2 microns or greater than 4 microns are also compatible with aspects of the invention.

The RNA molecule(s) to be delivered are complexed or “trapped” within the shell of the glucan particle. The shell or RNA component of the particle can be labeled for visualization, as described in, and incorporated by reference from, Soto and Ostroff (2008) Bioconjug Chem 19:840. Methods of loading GeRPs are discussed further below.

The optimal protocol for uptake of oligonucleotides will depend upon a number of factors, the most crucial being the type of cells that are being used. Other factors that are important in uptake include, but are not limited to, the nature and concentration of the oligonucleotide, the confluence of the cells, the type of culture the cells are in (e.g., a suspension culture or plated) and the type of media in which the cells are grown.

### Encapsulating Agents

Encapsulating agents entrap oligonucleotides within vesicles. In another embodiment of the invention, an oligonucleotide may be associated with a carrier or vehicle, e.g., liposomes or micelles, although other carriers could be used, as would be appreciated by one skilled in the art. Liposomes are vesicles made of a lipid bilayer having a structure similar to biological membranes. Such carriers are used to facilitate the cellular uptake or targeting of the oligonucleotide, or improve the oligonucleotide's pharmacokinetic or toxicologic properties.

For example, the oligonucleotides of the present invention may also be administered encapsulated in liposomes, pharmaceutical compositions wherein the active ingredient is contained either dispersed or variously present in corpuscles consisting of aqueous concentric layers adherent to lipidic layers. The oligonucleotides, depending upon solubility, may be present both in the aqueous layer and in the lipidic layer, or in what is generally termed a liposomic suspension. The hydrophobic layer, generally but not exclusively, comprises phopholipids such as lecithin and sphingomyelin, steroids such as cholesterol, more or less ionic surfactants such as diacetylphosphate, stearylamine, or phosphatidic acid, or other materials of a hydrophobic nature. The diameters of the liposomes generally range from about 15 nm to about 5 microns.

The use of liposomes as drug delivery vehicles offers several advantages. Liposomes increase intracellular stability, increase uptake efficiency and improve biological activity. Liposomes are hollow spherical vesicles composed of lipids arranged in a similar fashion as those lipids which make up the cell membrane. They have an internal aqueous space for entrapping water soluble compounds and range in size from 0.05 to several microns in diameter. Several studies have shown that liposomes can deliver nucleic acids to cells and that the nucleic acids remain biologically active. For example, a lipid delivery vehicle originally designed as a research tool, such as Lipofectin or LIPOFECTAMINE™ 2000, can deliver intact nucleic acid molecules to cells.

Specific advantages of using liposomes include the following: they are non-toxic and biodegradable in composition; they display long circulation half-lives; and recognition molecules can be readily attached to their surface for targeting to tissues. Finally, cost-effective manufacture of liposome-based pharmaceuticals, either in a liquid suspension or lyophilized product, has demonstrated the viability of this technology as an acceptable drug delivery system.

In some aspects, formulations associated with the invention might be selected for a class of naturally occurring or chemically synthesized or modified saturated and unsaturated fatty acid residues. Fatty acids might exist in a form of triglycerides, diglycerides or individual fatty acids. In another embodiment, the use of well-validated mixtures of fatty acids and/or fat emulsions currently used in pharmacology for parenteral nutrition may be utilized.

Liposome based formulations are widely used for oligonucleotide delivery. However, most of commercially available lipid or liposome formulations contain at least one positively charged lipid (cationic lipids). The presence of this positively charged lipid is believed to be essential for obtaining a high degree of oligonucleotide loading and for enhancing liposome fusogenic properties. Several methods have been performed and published to identify optimal positively charged lipid chemistries. However, the commercially available liposome formulations containing cationic lipids are characterized by a high level of toxicity. In vivo limited therapeutic indexes have revealed that liposome formulations containing positive charged lipids are associated with toxicity (i.e. elevation in liver enzymes) at concentrations only slightly higher than concentration required to achieve RNA silencing.

Nucleic acids associated with the invention can be hydrophobically modified and can be encompassed within neutral nanotransporters. Further description of neutral nanotransporters is incorporated by reference from PCT Application PCT/US2009/005251, filed on Sep. 22, 2009, and entitled “Neutral Nanotransporters.” Such particles enable quantitative oligonucleotide incorporation into non-charged lipid mixtures. The lack of toxic levels of cationic lipids in such neutral nanotransporter compositions is an important feature.

As demonstrated in PCT/US2009/005251, oligonucleotides can effectively be incorporated into a lipid mixture that is free of cationic lipids and such a composition can effectively deliver a therapeutic oligonucleotide to a cell in a manner that it is functional. For example, a high level of activity was observed when the fatty mixture was composed of a phosphatidylcholine base fatty acid and a sterol such as a cholesterol. For instance, one preferred formulation of neutral fatty mixture is composed of at least 20% of DOPC or DSPC and at least 20% of sterol such as cholesterol. Even as low as 1:5 lipid to oligonucleotide ratio was shown to be sufficient to get complete encapsulation of the oligonucleotide in a non charged formulation.

The neutral nanotransporters compositions enable efficient loading of oligonucleotide into neutral fat formulation. The composition includes an oligonucleotide that is modified in a manner such that the hydrophobicity of the molecule is increased (for example a hydrophobic molecule is attached (covalently or no-covalently) to a hydrophobic molecule on the oligonucleotide terminus or a non-terminal nucleotide, base, sugar, or backbone), the modified oligonucleotide being mixed with a neutral fat formulation (for example containing at least 25% of cholesterol and 25% of DOPC or analogs thereof). A cargo molecule, such as another lipid can also be included in the composition. This composition, where part of the formulation is build into the oligonucleotide itself, enables efficient encapsulation of oligonucleotide in neutral lipid particles.

In some aspects, stable particles ranging in size from 50 to 140 nm can be formed upon complexing of hydrophobic oligonucleotides with preferred formulations. It is interesting to mention that the formulation by itself typically does not form small particles, but rather, forms agglomerates, which are transformed into stable 50-120 nm particles upon addition of the hydrophobic modified oligonucleotide.

The neutral nanotransporter compositions of the invention include a hydrophobic modified polynucleotide, a neutral fatty mixture, and optionally a cargo molecule. A “hydrophobic modified polynucleotide” as used herein is a polynucleotide of the invention (i.e. sd-rxRNA) that has at least one modification that renders the polynucleotide more hydrophobic than the polynucleotide was prior to modification. The modification may be achieved by attaching (covalently or non-covalently) a hydrophobic molecule to the polynucleotide. In some instances the hydrophobic molecule is or includes a lipophilic group.

The term “lipophilic group” means a group that has a higher affinity for lipids than its affinity for water. Examples of lipophilic groups include, but are not limited to, cholesterol, a cholesteryl or modified cholesteryl residue, adamantine, dihydrotesterone, long chain alkyl, long chain alkenyl, long chain alkynyl, olely-lithocholic, cholenic, oleoyl-cholenic, palmityl, heptadecyl, myrisityl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen. The cholesterol moiety may be reduced (e.g. as in cholestan) or may be substituted (e.g. by halogen). A combination of different lipophilic groups in one molecule is also possible.

The hydrophobic molecule may be attached at various positions of the polynucleotide. As described above, the hydrophobic molecule may be linked to the terminal residue of the polynucleotide such as the 3′ of 5′-end of the polynucleotide. Alternatively, it may be linked to an internal nucleotide or a nucleotide on a branch of the polynucleotide. The hydrophobic molecule may be attached, for instance to a 2′-position of the nucleotide. The hydrophobic molecule may also be linked to the heterocyclic base, the sugar or the backbone of a nucleotide of the polynucleotide.

The hydrophobic molecule may be connected to the polynucleotide by a linker moiety. Optionally the linker moiety is a non-nucleotidic linker moiety. Non-nucleotidic linkers are e.g. abasic residues (dSpacer), oligoethyleneglycol, such as triethyleneglycol (spacer 9) or hexaethylenegylcol (spacer 18), or alkane-diol, such as butanediol. The spacer units are preferably linked by phosphodiester or phosphorothioate bonds. The linker units may appear just once in the molecule or may be incorporated several times, e.g. via phosphodiester, phosphorothioate, methylphosphonate, or amide linkages.

Typical conjugation protocols involve the synthesis of polynucleotides bearing an aminolinker at one or more positions of the sequence, however, a linker is not required. The amino group is then reacted with the molecule being conjugated using appropriate coupling or activating reagents. The conjugation reaction may be performed either with the polynucleotide still bound to a solid support or following cleavage of the polynucleotide in solution phase. Purification of the modified polynucleotide by HPLC typically results in a pure material.

In some embodiments the hydrophobic molecule is a sterol type conjugate, a PhytoSterol conjugate, cholesterol conjugate, sterol type conjugate with altered side chain length, fatty acid conjugate, any other hydrophobic group conjugate, and/or hydrophobic modifications of the internal nucleoside, which provide sufficient hydrophobicity to be incorporated into micelles.

For purposes of the present invention, the term “sterols”, refers or steroid alcohols are a subgroup of steroids with a hydroxyl group at the 3-position of the A-ring. They are amphipathic lipids synthesized from acetyl-coenzyme A via the HMG-CoA reductase pathway. The overall molecule is quite flat. The hydroxyl group on the A ring is polar. The rest of the aliphatic chain is non-polar. Usually sterols are considered to have an 8 carbon chain at position 17.

For purposes of the present invention, the term “sterol type molecules”, refers to steroid alcohols, which are similar in structure to sterols. The main difference is the structure of the ring and number of carbons in a position 21 attached side chain.

For purposes of the present invention, the term “PhytoSterols” (also called plant sterols) are a group of steroid alcohols, phytochemicals naturally occurring in plants. There are more then 200 different known PhytoSterols

For purposes of the present invention, the term “Sterol side chain” refers to a chemical composition of a side chain attached at the position 17 of sterol-type molecule. In a standard definition sterols are limited to a 4 ring structure carrying a 8 carbon chain at position 17. In this invention, the sterol type molecules with side chain longer and shorter than conventional are described. The side chain may branched or contain double back bones.

Thus, sterols useful in the invention, for example, include cholesterols, as well as unique sterols in which position 17 has attached side chain of 2-7 or longer then 9 carbons. In a particular embodiment, the length of the polycarbon tail is varied between 5 and 9 carbons. Such conjugates may have significantly better in vivo efficacy, in particular delivery to liver. These types of molecules are expected to work at concentrations 5 to 9 fold lower then oligonucleotides conjugated to conventional cholesterols.

Alternatively the polynucleotide may be bound to a protein, peptide or positively charged chemical that functions as the hydrophobic molecule. The proteins may be selected from the group consisting of protamine, dsRNA binding domain, and arginine rich peptides. Exemplary positively charged chemicals include spermine, spermidine, cadaverine, and putrescine.

In another embodiment hydrophobic molecule conjugates may demonstrate even higher efficacy when it is combined with optimal chemical modification patterns of the polynucleotide (as described herein in detail), containing but not limited to hydrophobic modifications, phosphorothioate modifications, and 2′ ribo modifications.

In another embodiment the sterol type molecule may be a naturally occurring PhytoSterols. The polycarbon chain may be longer than 9 and may be linear, branched and/or contain double bonds. Some PhytoSterol containing polynucleotide conjugates may be significantly more potent and active in delivery of polynucleotides to various tissues. Some PhytoSterols may demonstrate tissue preference and thus be used as a way to delivery RNAi specifically to particular tissues.

The hydrophobic modified polynucleotide is mixed with a neutral fatty mixture to form a micelle. The neutral fatty acid mixture is a mixture of fats that has a net neutral or slightly net negative charge at or around physiological pH that can form a micelle with the hydrophobic modified polynucleotide. For purposes of the present invention, the term “micelle” refers to a small nanoparticle formed by a mixture of non charged fatty acids and phospholipids. The neutral fatty mixture may include cationic lipids as long as they are present in an amount that does not cause toxicity. In preferred embodiments the neutral fatty mixture is free of cationic lipids. A mixture that is free of cationic lipids is one that has less than 1% and preferably 0% of the total lipid being cationic lipid. The term “cationic lipid” includes lipids and synthetic lipids having a net positive charge at or around physiological pH. The term “anionic lipid” includes lipids and synthetic lipids having a net negative charge at or around physiological pH.

The neutral fats bind to the oligonucleotides of the invention by a strong but non-covalent attraction (e.g., an electrostatic, van der Waals, pi-stacking, etc. interaction).

The neutral fat mixture may include formulations selected from a class of naturally occurring or chemically synthesized or modified saturated and unsaturated fatty acid residues. Fatty acids might exist in a form of triglycerides, diglycerides or individual fatty acids. In another embodiment the use of well-validated mixtures of fatty acids and/or fat emulsions currently used in pharmacology for parenteral nutrition may be utilized.

The neutral fatty mixture is preferably a mixture of a choline based fatty acid and a sterol. Choline based fatty acids include for instance, synthetic phosphocholine derivatives such as DDPC, DLPC, DMPC, DPPC, DSPC, DOPC, POPC, and DEPC. DOPC (chemical registry number 4235-95-4) is dioleoylphosphatidylcholine (also known as dielaidoylphosphatidylcholine, dioleoyl-PC, dioleoylphosphocholine, dioleoyl-sn-glycero-3-phosphocholine, dioleylphosphatidylcholine). DSPC (chemical registry number 816-94-4) is distearoylphosphatidylcholine (also known as 1,2-Distearoyl-sn-Glycero-3-phosphocholine).

The sterol in the neutral fatty mixture may be for instance cholesterol. The neutral fatty mixture may be made up completely of a choline based fatty acid and a sterol or it may optionally include a cargo molecule. For instance, the neutral fatty mixture may have at least 20% or 25% fatty acid and 20% or 25% sterol.

For purposes of the present invention, the term “Fatty acids” relates to conventional description of fatty acid. They may exist as individual entities or in a form of two- and triglycerides. For purposes of the present invention, the term “fat emulsions” refers to safe fat formulations given intravenously to subjects who are unable to get enough fat in their diet. It is an emulsion of soy bean oil (or other naturally occurring oils) and egg phospholipids. Fat emulsions are being used for formulation of some insoluble anesthetics. In this disclosure, fat emulsions might be part of commercially available preparations like Intralipid, Liposyn, Nutrilipid, modified commercial preparations, where they are enriched with particular fatty acids or fully de novo-formulated combinations of fatty acids and phospholipids.

In one embodiment, the cells to be contacted with an oligonucleotide composition of the invention are contacted with a mixture comprising the oligonucleotide and a mixture comprising a lipid, e.g., one of the lipids or lipid compositions described supra for between about 12 hours to about 24 hours. In another embodiment, the cells to be contacted with an oligonucleotide composition are contacted with a mixture comprising the oligonucleotide and a mixture comprising a lipid, e.g., one of the lipids or lipid compositions described supra for between about 1 and about five days. In one embodiment, the cells are contacted with a mixture comprising a lipid and the oligonucleotide for between about three days to as long as about 30 days. In another embodiment, a mixture comprising a lipid is left in contact with the cells for at least about five to about 20 days. In another embodiment, a mixture comprising a lipid is left in contact with the cells for at least about seven to about 15 days.

50%-60% of the formulation can optionally be any other lipid or molecule. Such a lipid or molecule is referred to herein as a cargo lipid or cargo molecule. Cargo molecules include but are not limited to intralipid, small molecules, fusogenic peptides or lipids or other small molecules might be added to alter cellular uptake, endosomal release or tissue distribution properties. The ability to tolerate cargo molecules is important for modulation of properties of these particles, if such properties are desirable. For instance the presence of some tissue specific metabolites might drastically alter tissue distribution profiles. For example use of Intralipid type formulation enriched in shorter or longer fatty chains with various degrees of saturation affects tissue distribution profiles of these type of formulations (and their loads).

An example of a cargo lipid useful according to the invention is a fusogenic lipid. For instance, the zwiterionic lipid DOPE (chemical registry number 4004-5-1, 1,2-Dioleoyl-sn-Glycero-3-phosphoethanolamine) is a preferred cargo lipid.

Intralipid may be comprised of the following composition: 1 000 mL contain: purified soybean oil 90 g, purified egg phospholipids 12 g, glycerol anhydrous 22 g, water for injection q.s. ad 1 000 mL. pH is adjusted with sodium hydroxide to pH approximately 8. Energy content/L: 4.6 MJ (190 kcal). Osmolality (approx.): 300 mOsm/kg water. In another embodiment fat emulsion is Liposyn that contains 5% safflower oil, 5% soybean oil, up to 1.2% egg phosphatides added as an emulsifier and 2.5% glycerin in water for injection. It may also contain sodium hydroxide for pH adjustment. pH 8.0 (6.0-9.0). Liposyn has an osmolarity of 276 m Osmol/liter (actual).

Variation in the identity, amounts and ratios of cargo lipids affects the cellular uptake and tissue distribution characteristics of these compounds. For example, the length of lipid tails and level of saturability will affect differential uptake to liver, lung, fat and cardiomyocytes. Addition of special hydrophobic molecules like vitamins or different forms of sterols can favor distribution to special tissues which are involved in the metabolism of particular compounds. In some embodiments, vitamin A or E is used. Complexes are formed at different oligonucleotide concentrations, with higher concentrations favoring more efficient complex formation.

In another embodiment, the fat emulsion is based on a mixture of lipids. Such lipids may include natural compounds, chemically synthesized compounds, purified fatty acids or any other lipids. In yet another embodiment the composition of fat emulsion is entirely artificial. In a particular embodiment, the fat emulsion is more then 70% linoleic acid. In yet another particular embodiment the fat emulsion is at least 1% of cardiolipin. Linoleic acid (LA) is an unsaturated omega-6 fatty acid. It is a colorless liquid made of a carboxylic acid with an 18-carbon chain and two cis double bonds.

In yet another embodiment of the present invention, the alteration of the composition of the fat emulsion is used as a way to alter tissue distribution of hydrophobicly modified polynucleotides. This methodology provides for the specific delivery of the polynucleotides to particular tissues.

In another embodiment the fat emulsions of the cargo molecule contain more then 70% of Linoleic acid (C18H32O2) and/or cardiolipin.

Fat emulsions, like intralipid have been used before as a delivery formulation for some non-water soluble drugs (such as Propofol, re-formulated as Diprivan). Unique features of the present invention include (a) the concept of combining modified polynucleotides with the hydrophobic compound(s), so it can be incorporated in the fat micelles and (b) mixing it with the fat emulsions to provide a reversible carrier. After injection into a blood stream, micelles usually bind to serum proteins, including albumin, HDL, LDL and other. This binding is reversible and eventually the fat is absorbed by cells. The polynucleotide, incorporated as a part of the micelle will then be delivered closely to the surface of the cells. After that cellular uptake might be happening though variable mechanisms, including but not limited to sterol type delivery.

### Complexing Agents

Complexing agents bind to the oligonucleotides of the invention by a strong but non-covalent attraction (e.g., an electrostatic, van der Waals, pi-stacking, etc. interaction). In one embodiment, oligonucleotides of the invention can be complexed with a complexing agent to increase cellular uptake of oligonucleotides. An example of a complexing agent includes cationic lipids. Cationic lipids can be used to deliver oligonucleotides to cells. However, as discussed above, formulations free in cationic lipids are preferred in some embodiments.

The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Preferred straight chain or branched alkyl or alkene groups have six or more carbon atoms. Alicyclic groups include cholesterol and other steroid groups. Cationic lipids can be prepared with a variety of counterions (anions) including, e.g., Cl⁻, Br⁻, I⁻, F⁻, acetate, trifluoroacetate, sulfate, nitrite, and nitrate.

Examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N—(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). The cationic lipid N-(1-(2,3-dioleyloxy)propyl)-N,N,N-trimethylammonium chloride (DOTMA), for example, was found to increase 1000-fold the antisense effect of a phosphorothioate oligonucleotide. (Vlassov et al., 1994, Biochimica et Biophysica Acta 1197:95-108). Oligonucleotides can also be complexed with, e.g., poly (L-lysine) or avidin and lipids may, or may not, be included in this mixture, e.g., steryl-poly (L-lysine).

Cationic lipids have been used in the art to deliver oligonucleotides to cells (see, e.g., U.S. Pat. Nos. 5,855,910; 5,851,548; 5,830,430; 5,780,053; 5,767,099; Lewis et al. 1996. Proc. Natl. Acad. Sci. USA 93:3176; Hope et al. 1998. Molecular Membrane Biology 15:1). Other lipid compositions which can be used to facilitate uptake of the instant oligonucleotides can be used in connection with the claimed methods. In addition to those listed supra, other lipid compositions are also known in the art and include, e.g., those taught in U.S. Pat. No. 4,235,871; U.S. Pat. Nos. 4,501,728; 4,837,028; 4,737,323.

In one embodiment lipid compositions can further comprise agents, e.g., viral proteins to enhance lipid-mediated transfections of oligonucleotides (Kamata, et al., 1994. Nucl. Acids. Res. 22:536). In another embodiment, oligonucleotides are contacted with cells as part of a composition comprising an oligonucleotide, a peptide, and a lipid as taught, e.g., in U.S. Pat. No. 5,736,392. Improved lipids have also been described which are serum resistant (Lewis, et al., 1996. Proc. Natl. Acad. Sci. 93:3176). Cationic lipids and other complexing agents act to increase the number of oligonucleotides carried into the cell through endocytosis.

In another embodiment N-substituted glycine oligonucleotides (peptoids) can be used to optimize uptake of oligonucleotides. Peptoids have been used to create cationic lipid-like compounds for transfection (Murphy, et al., 1998. Proc. Natl. Acad. Sci. 95:1517). Peptoids can be synthesized using standard methods (e.g., Zuckermann, R. N., et al. 1992. J. Am. Chem. Soc. 114:10646; Zuckermann, R. N., et al. 1992. Int. J. Peptide Protein Res. 40:497). Combinations of cationic lipids and peptoids, liptoids, can also be used to optimize uptake of the subject oligonucleotides (Hunag, et al., 1998. Chemistry and Biology. 5:345). Liptoids can be synthesized by elaborating peptoid oligonucleotides and coupling the amino terminal submonomer to a lipid via its amino group (Hunag, et al., 1998. Chemistry and Biology. 5:345).

It is known in the art that positively charged amino acids can be used for creating highly active cationic lipids (Lewis et al. 1996. Proc. Natl. Acad. Sci. US.A. 93:3176). In one embodiment, a composition for delivering oligonucleotides of the invention comprises a number of arginine, lysine, histidine or ornithine residues linked to a lipophilic moiety (see e.g., U.S. Pat. No. 5,777,153).

In another embodiment, a composition for delivering oligonucleotides of the invention comprises a peptide having from between about one to about four basic residues. These basic residues can be located, e.g., on the amino terminal, C-terminal, or internal region of the peptide. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine (can also be considered non-polar), asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Apart from the basic amino acids, a majority or all of the other residues of the peptide can be selected from the non-basic amino acids, e.g., amino acids other than lysine, arginine, or histidine. Preferably a preponderance of neutral amino acids with long neutral side chains are used.

In one embodiment, a composition for delivering oligonucleotides of the invention comprises a natural or synthetic polypeptide having one or more gamma carboxyglutamic acid residues, or γ-Gla residues. These gamma carboxyglutamic acid residues may enable the polypeptide to bind to each other and to membrane surfaces. In other words, a polypeptide having a series of γ-Gla may be used as a general delivery modality that helps an RNAi construct to stick to whatever membrane to which it comes in contact. This may at least slow RNAi constructs from being cleared from the blood stream and enhance their chance of homing to the target.

The gamma carboxyglutamic acid residues may exist in natural proteins (for example, prothrombin has 10 γ-Gla residues). Alternatively, they can be introduced into the purified, recombinantly produced, or chemically synthesized polypeptides by carboxylation using, for example, a vitamin K-dependent carboxylase. The gamma carboxyglutamic acid residues may be consecutive or non-consecutive, and the total number and location of such gamma carboxyglutamic acid residues in the polypeptide can be regulated/fine tuned to achieve different levels of “stickiness” of the polypeptide.

In one embodiment, the cells to be contacted with an oligonucleotide composition of the invention are contacted with a mixture comprising the oligonucleotide and a mixture comprising a lipid, e.g., one of the lipids or lipid compositions described supra for between about 12 hours to about 24 hours. In another embodiment, the cells to be contacted with an oligonucleotide composition are contacted with a mixture comprising the oligonucleotide and a mixture comprising a lipid, e.g., one of the lipids or lipid compositions described supra for between about 1 and about five days. In one embodiment, the cells are contacted with a mixture comprising a lipid and the oligonucleotide for between about three days to as long as about 30 days. In another embodiment, a mixture comprising a lipid is left in contact with the cells for at least about five to about 20 days. In another embodiment, a mixture comprising a lipid is left in contact with the cells for at least about seven to about 15 days.

For example, in one embodiment, an oligonucleotide composition can be contacted with cells in the presence of a lipid such as cytofectin CS or GSV (available from Glen Research; Sterling, Va.), GS3815, GS2888 for prolonged incubation periods as described herein.

In one embodiment, the incubation of the cells with the mixture comprising a lipid and an oligonucleotide composition does not reduce the viability of the cells. Preferably, after the transfection period the cells are substantially viable. In one embodiment, after transfection, the cells are between at least about 70% and at least about 100% viable. In another embodiment, the cells are between at least about 80% and at least about 95% viable. In yet another embodiment, the cells are between at least about 85% and at least about 90% viable.

In one embodiment, oligonucleotides are modified by attaching a peptide sequence that transports the oligonucleotide into a cell, referred to herein as a “transporting peptide.” In one embodiment, the composition includes an oligonucleotide which is complementary to a target nucleic acid molecule encoding the protein, and a covalently attached transporting peptide.

The language “transporting peptide” includes an amino acid sequence that facilitates the transport of an oligonucleotide into a cell. Exemplary peptides which facilitate the transport of the moieties to which they are linked into cells are known in the art, and include, e.g., HIV TAT transcription factor, lactoferrin, Herpes VP22 protein, and fibroblast growth factor 2 (Pooga et al. 1998. Nature Biotechnology. 16:857; and Derossi et al. 1998. Trends in Cell Biology. 8:84; Elliott and O'Hare. 1997. Cell 88:223).

Oligonucleotides can be attached to the transporting peptide using known techniques, e.g., (Prochiantz, A. 1996. Curr. Opin. Neurobiol. 6:629; Derossi et al. 1998. Trends Cell Biol. 8:84; Troy et al. 1996. J. Neurosci. 16:253), Vives et al. 1997. J. Biol. Chem. 272:16010). For example, in one embodiment, oligonucleotides bearing an activated thiol group are linked via that thiol group to a cysteine present in a transport peptide (e.g., to the cysteine present in the β turn between the second and the third helix of the antennapedia homeodomain as taught, e.g., in Derossi et al. 1998. Trends Cell Biol. 8:84; Prochiantz. 1996. Current Opinion in Neurobiol. 6:629; Allinquant et al. 1995. J Cell Biol. 128:919). In another embodiment, a Boc-Cys-(Npys)OH group can be coupled to the transport peptide as the last (N-terminal) amino acid and an oligonucleotide bearing an SH group can be coupled to the peptide (Troy et al. 1996. J. Neurosci. 16:253).

In one embodiment, a linking group can be attached to a nucleomonomer and the transporting peptide can be covalently attached to the linker. In one embodiment, a linker can function as both an attachment site for a transporting peptide and can provide stability against nucleases. Examples of suitable linkers include substituted or unsubstituted C₁-C₂₀ alkyl chains, C₂-C₂₀ alkenyl chains, C₂-C₂₀alkynyl chains, peptides, and heteroatoms (e.g., S, O, NH, etc.). Other exemplary linkers include bifunctional crosslinking agents such as sulfosuccinimidyl-4-(maleimidophenyl)-butyrate (SMPB) (see, e.g., Smith et al. Biochem J 1991.276: 417-2).

In one embodiment, oligonucleotides of the invention are synthesized as molecular conjugates which utilize receptor-mediated endocytotic mechanisms for delivering genes into cells (see, e.g., Bunnell et al. 1992. Somatic Cell and Molecular Genetics. 18:559, and the references cited therein).

### Targeting Agents

The delivery of oligonucleotides can also be improved by targeting the oligonucleotides to a cellular receptor. The targeting moieties can be conjugated to the oligonucleotides or attached to a carrier group (i.e., poly(L-lysine) or liposomes) linked to the oligonucleotides. This method is well suited to cells that display specific receptor-mediated endocytosis.

For instance, oligonucleotide conjugates to 6-phosphomannosylated proteins are internalized 20-fold more efficiently by cells expressing mannose 6-phosphate specific receptors than free oligonucleotides. The oligonucleotides may also be coupled to a ligand for a cellular receptor using a biodegradable linker. In another example, the delivery construct is mannosylated streptavidin which forms a tight complex with biotinylated oligonucleotides. Mannosylated streptavidin was found to increase 20-fold the internalization of biotinylated oligonucleotides. (Vlassov et al. 1994. Biochimica et Biophysica Acta 1197:95-108).

In addition specific ligands can be conjugated to the polylysine component of polylysine-based delivery systems. For example, transferrin-polylysine, adenovirus-polylysine, and influenza virus hemagglutinin HA-2 N-terminal fusogenic peptides-polylysine conjugates greatly enhance receptor-mediated DNA delivery in eucaryotic cells. Mannosylated glycoprotein conjugated to poly(L-lysine) in aveolar macrophages has been employed to enhance the cellular uptake of oligonucleotides. Liang et al. 1999. Pharmazie 54:559-566.

Because malignant cells have an increased need for essential nutrients such as folic acid and transferrin, these nutrients can be used to target oligonucleotides to cancerous cells. For example, when folic acid is linked to poly(L-lysine) enhanced oligonucleotide uptake is seen in promyelocytic leukaemia (HL-60) cells and human melanoma (M-14) cells. Ginobbi et al. 1997. Anticancer Res. 17:29. In another example, liposomes coated with maleylated bovine serum albumin, folic acid, or ferric protoporphyrin IX, show enhanced cellular uptake of oligonucleotides in murine macrophages, KB cells, and 2.2.15 human hepatoma cells. Liang et al. 1999. Pharmazie 54:559-566.

Liposomes naturally accumulate in the liver, spleen, and reticuloendothelial system (so-called, passive targeting). By coupling liposomes to various ligands such as antibodies are protein A, they can be actively targeted to specific cell populations. For example, protein A-bearing liposomes may be pretreated with H-2K specific antibodies which are targeted to the mouse major histocompatibility complex-encoded H-2K protein expressed on L cells. (Vlassov et al. 1994. Biochimica et Biophysica Acta 1197:95-108).

Other in vitro and/or in vivo delivery of RNAi reagents are known in the art, and can be used to deliver the subject RNAi constructs. See, for example, U.S. patent application publications 20080152661, 20080112916, 20080107694, 20080038296, 20070231392, 20060240093, 20060178327, 20060008910, 20050265957, 20050064595, 20050042227, 20050037496, 20050026286, 20040162235, 20040072785, 20040063654, 20030157030, WO 2008/036825, WO04/065601, and AU2004206255B2, just to name a few (all incorporated by reference).

### Administration

The optimal course of administration or delivery of the oligonucleotides or therapeutic RNA molecules may vary depending upon the desired result and/or on the subject to be treated. As used herein “administration” refers to contacting cells with oligonucleotides and can be performed in vitro, in vivo or ex vivo.

Non-limiting examples of methods of administration include intravitreal, subretinal, periocular (subconjunctival, sub-tenon, retrobulbar, peribulbar, and post juxtascleral), topical, eye drops, corneal implants, biodegradable implants, non-biodegradable implants, ocular inserts, thin-films, sustained release formulations, polymers, iontophoresis, hydrogel contact lenses, reverse-thermal hydrogels and biodegradable pellets.

In some embodiments, the therapeutic RNA molecule is administered to an area of the eye other than the front of the eye. Surprisingly, it was found herein that administration of a therapeutic RNA molecule to an area of the eye other than the front of the eye led to significant reduction of gene expression in the front of the eye. In some embodiments, the method of administration of the therapeutic RNA molecule is intravitreal. It was unexpected that intravitreal administration of a therapeutic RNA molecule would lead to reduced expression of a target gene in the front of the eye, such as the cornea.

In other embodiments, the therapeutic RNA molecule is administered to the front of the eye, such as through topical administration. In some embodiments, the therapeutic RNA molecule is administered to the cornea by topical administration.

The dosage of oligonucleotides may be adjusted to optimally reduce expression of a protein translated from a target nucleic acid molecule, e.g., as measured by a readout of RNA stability or by a therapeutic response, without undue experimentation.

For example, expression of the protein encoded by the nucleic acid target can be measured to determine whether or not the dosage regimen needs to be adjusted accordingly. In addition, an increase or decrease in RNA or protein levels in a cell or produced by a cell can be measured using any art recognized technique. By determining whether transcription has been decreased, the effectiveness of the oligonucleotide in inducing the cleavage of a target RNA can be determined.

Any of the above-described oligonucleotide compositions can be used alone or in conjunction with a pharmaceutically acceptable carrier. As used herein, “pharmaceutically acceptable carrier” includes appropriate solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, it can be used in the therapeutic compositions. Supplementary active ingredients can also be incorporated into the compositions.

Oligonucleotides may be incorporated into liposomes or liposomes modified with polyethylene glycol or admixed with cationic lipids for parenteral administration. Incorporation of additional substances into the liposome, for example, antibodies reactive against membrane proteins found on specific target cells, can help target the oligonucleotides to specific cell types.

With respect to in vivo applications, the formulations of the present invention can be administered to a patient in a variety of forms adapted to deliver the construct to the eye. In preferred embodiments, parenteral administration is ocular. Ocular administration can be intravitreal, intracameral, subretinal, subconjunctival, or subtenon.

Pharmaceutical preparations for parenteral administration include aqueous solutions of the active compounds in water-soluble or water-dispersible form. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, or dextran, optionally, the suspension may also contain stabilizers. The oligonucleotides of the invention can be formulated in liquid solutions, preferably in physiologically compatible buffers such as Hank's solution or Ringer's solution. In addition, the oligonucleotides may be formulated in solid form and redissolved or suspended immediately prior to use. Lyophilized forms are also included in the invention.

The chosen method of delivery will result in entry into cells. In some embodiments, preferred delivery methods include liposomes (10-400 nm), hydrogels, controlled-release polymers, and other pharmaceutically applicable vehicles, and microinjection or electroporation (for ex vivo treatments).

The pharmaceutical preparations of the present invention may be prepared and formulated as emulsions. Emulsions are usually heterogeneous systems of one liquid dispersed in another in the form of droplets usually exceeding 0.1 μm in diameter. The emulsions of the present invention may contain excipients such as emulsifiers, stabilizers, dyes, fats, oils, waxes, fatty acids, fatty alcohols, fatty esters, humectants, hydrophilic colloids, preservatives, and anti-oxidants may also be present in emulsions as needed. These excipients may be present as a solution in either the aqueous phase, oily phase or itself as a separate phase.

Examples of naturally occurring emulsifiers that may be used in emulsion formulations of the present invention include lanolin, beeswax, phosphatides, lecithin and acacia. Finely divided solids have also been used as good emulsifiers especially in combination with surfactants and in viscous preparations. Examples of finely divided solids that may be used as emulsifiers include polar inorganic solids, such as heavy metal hydroxides, nonswelling clays such as bentonite, attapulgite, hectorite, kaolin, montrnorillonite, colloidal aluminum silicate and colloidal magnesium aluminum silicate, pigments and nonpolar solids such as carbon or glyceryl tristearate.

Examples of preservatives that may be included in the emulsion formulations include methyl paraben, propyl paraben, quaternary ammonium salts, benzalkonium chloride, esters of p-hydroxybenzoic acid, and boric acid. Examples of antioxidants that may be included in the emulsion formulations include free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

In one embodiment, the compositions of oligonucleotides are formulated as microemulsions. A microemulsion is a system of water, oil and amphiphile which is a single optically isotropic and thermodynamically stable liquid solution. Typically microemulsions are prepared by first dispersing an oil in an aqueous surfactant solution and then adding a sufficient amount of a 4th component, generally an intermediate chain-length alcohol to form a transparent system.

Surfactants that may be used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C₈-C₁₂) mono, di, and tri-glycerides, polyoxyethylated glyceryl fatty acid esters, fatty alcohols, polyglycolized glycerides, saturated polyglycolized C₈-C₁₀ glycerides, vegetable oils and silicone oil.

Microemulsions are particularly of interest from the standpoint of drug solubilization and the enhanced absorption of drugs. Lipid based microemulsions (both oil/water and water/oil) have been proposed to enhance the oral bioavailability of drugs.

Microemulsions offer improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11:1385; Ho et al., J. Pharm. Sci., 1996, 85:138-143). Microemulsions have also been effective in the transdermal delivery of active components in both cosmetic and pharmaceutical applications. It is expected that the microemulsion compositions and formulations of the present invention will facilitate the increased systemic absorption of oligonucleotides from the gastrointestinal tract, as well as improve the local cellular uptake of oligonucleotides within the gastrointestinal tract, vagina, buccal cavity and other areas of administration.

The useful dosage to be administered and the particular mode of administration will vary depending upon such factors as the cell type, or for in vivo use, the age, weight and the particular animal and region thereof to be treated, the particular oligonucleotide and delivery method used, the therapeutic or diagnostic use contemplated, and the form of the formulation, for example, suspension, emulsion, micelle or liposome, as will be readily apparent to those skilled in the art. Typically, dosage is administered at lower levels and increased until the desired effect is achieved. When lipids are used to deliver the oligonucleotides, the amount of lipid compound that is administered can vary and generally depends upon the amount of oligonucleotide agent being administered. For example, the weight ratio of lipid compound to oligonucleotide agent is preferably from about 1:1 to about 15:1, with a weight ratio of about 5:1 to about 10:1 being more preferred. Generally, the amount of cationic lipid compound which is administered will vary from between about 0.1 milligram (mg) to about 1 gram (g). By way of general guidance, typically between about 0.1 mg and about 10 mg of the particular oligonucleotide agent, and about 1 mg to about 100 mg of the lipid compositions, each per kilogram of patient body weight, is administered, although higher and lower amounts can be used.

The agents of the invention are administered to subjects or contacted with cells in a biologically compatible form suitable for pharmaceutical administration. By “biologically compatible form suitable for administration” is meant that the oligonucleotide is administered in a form in which any toxic effects are outweighed by the therapeutic effects of the oligonucleotide. In one embodiment, oligonucleotides can be administered to subjects. Examples of subjects include mammals, e.g., humans and other primates; cows, pigs, horses, and farming (agricultural) animals; dogs, cats, and other domesticated pets; mice, rats, and transgenic non-human animals.

Administration of an active amount of an oligonucleotide of the present invention is defined as an amount effective, at dosages and for periods of time necessary to achieve the desired result. For example, an active amount of an oligonucleotide may vary according to factors such as the type of cell, the oligonucleotide used, and for in vivo uses the disease state, age, sex, and weight of the individual, and the ability of the oligonucleotide to elicit a desired response in the individual. Establishment of therapeutic levels of oligonucleotides within the cell is dependent upon the rates of uptake and efflux or degradation. Decreasing the degree of degradation prolongs the intracellular half-life of the oligonucleotide. Thus, chemically-modified oligonucleotides, e.g., with modification of the phosphate backbone, may require different dosing.

The exact dosage of an oligonucleotide and number of doses administered will depend upon the data generated experimentally and in clinical trials. Several factors such as the desired effect, the delivery vehicle, disease indication, and the route of administration, will affect the dosage. Dosages can be readily determined by one of ordinary skill in the art and formulated into the subject pharmaceutical compositions. Preferably, the duration of treatment will extend at least through the course of the disease symptoms.

Dosage regimens may be adjusted to provide the optimum therapeutic response. For example, the oligonucleotide may be repeatedly administered, e.g., several doses may be administered daily or the dose may be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

Ocular administration of sd-rxRNAs, including topical, intravitreal, intracameral, subretinal, subconjunctival, and subtenon administration, can be optimized through testing of dosing regimens. In some embodiments, a single administration is sufficient. To further prolong the effect of the administered sd-rxRNA, the sd-rxRNA can be administered in a slow-release formulation or device, as would be familiar to one of ordinary skill in the art. The hydrophobic nature of sd-rxRNA compounds can enable use of a wide variety of polymers, some of which are not compatible with conventional oligonucleotide delivery.

In other embodiments, the sd-rxRNA is administered multiple times. In some instances it is administered daily, bi-weekly, weekly, every two weeks, every three weeks, monthly, every two months, every three months, every four months, every five months, every six months or less frequently than every six months. In some instances, it is administered multiple times per day, week, month and/or year. For example, it can be administered approximately every hour, 2 hours, 3 hours, 4 hours, 5 hours, 6 hours, 7 hours, 8 hours, 9 hours 10 hours, 12 hours or more than twelve hours. It can be administered 1, 2, 3, 4, 5, 6, 7, 8, 9, 10 or more than 10 times per day.

Aspects of the invention relate to administering sd-rxRNA or rxRNA on molecules to a subject. In some instances the subject is a patient and administering the sd-rxRNA molecule involves administering the sd-rxRNA molecule in a doctor's office.

In some instances, the effective amount of sd-rxRNA that is delivered through ocular administration is at least approximately 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 μg including any intermediate values.

sd-rxRNA molecules administered through methods described herein are effectively targeted to all the cell types in the eye.

Physical methods of introducing nucleic acids include injection of a solution containing the nucleic acid, bombardment by particles covered by the nucleic acid, soaking the cell or organism in a solution of the nucleic acid, electroporation of cell membranes in the presence of the nucleic acid or topical application of a composition comprising the nucleic acid to the eye. A viral construct packaged into a viral particle would accomplish both efficient introduction of an expression construct into the cell and transcription of nucleic acid encoded by the expression construct. Other methods known in the art for introducing nucleic acids to cells may be used, such as lipid-mediated carrier transport, chemical-mediated transport, such as calcium phosphate, and the like. Thus the nucleic acid may be introduced along with components that perform one or more of the following activities: enhance nucleic acid uptake by the cell, inhibit annealing of single strands, stabilize the single strands, or other-wise increase inhibition of the target gene.

### Assays of Oligonucleotide Stability

In some embodiments, the oligonucleotides of the invention are stabilized, i.e., substantially resistant to endonuclease and exonuclease degradation. An oligonucleotide is defined as being substantially resistant to nucleases when it is at least about 3-fold more resistant to attack by an endogenous cellular nuclease, and is highly nuclease resistant when it is at least about 6-fold more resistant than a corresponding oligonucleotide. This can be demonstrated by showing that the oligonucleotides of the invention are substantially resistant to nucleases using techniques which are known in the art.

One way in which substantial stability can be demonstrated is by showing that the oligonucleotides of the invention function when delivered to a cell, e.g., that they reduce transcription or translation of target nucleic acid molecules, e.g., by measuring protein levels or by measuring cleavage of mRNA. Assays which measure the stability of target RNA can be performed at about 24 hours post-transfection (e.g., using Northern blot techniques, RNase Protection Assays, or QC-PCR assays as known in the art). Alternatively, levels of the target protein can be measured. Preferably, in addition to testing the RNA or protein levels of interest, the RNA or protein levels of a control, non-targeted gene will be measured (e.g., actin, or preferably a control with sequence similarity to the target) as a specificity control. RNA or protein measurements can be made using any art-recognized technique. Preferably, measurements will be made beginning at about 16-24 hours post transfection. (M. Y. Chiang, et al. 1991. J Biol Chem. 266:18162-71; T. Fisher, et al. 1993. Nucleic Acids Research. 21 3857).

The ability of an oligonucleotide composition of the invention to inhibit protein synthesis can be measured using techniques which are known in the art, for example, by detecting an inhibition in gene transcription or protein synthesis. For example, Nuclease S1 mapping can be performed. In another example, Northern blot analysis can be used to measure the presence of RNA encoding a particular protein. For example, total RNA can be prepared over a cesium chloride cushion (see, e.g., Ausebel et al., 1987. Current Protocols in Molecular Biology (Greene & Wiley, New York)). Northern blots can then be made using the RNA and probed (see, e.g., Id.). In another example, the level of the specific mRNA produced by the target protein can be measured, e.g., using PCR. In yet another example, Western blots can be used to measure the amount of target protein present. In still another embodiment, a phenotype influenced by the amount of the protein can be detected. Techniques for performing Western blots are well known in the art, see, e.g., Chen et al. J. Biol. Chem. 271:28259.

In another example, the promoter sequence of a target gene can be linked to a reporter gene and reporter gene transcription (e.g., as described in more detail below) can be monitored. Alternatively, oligonucleotide compositions that do not target a promoter can be identified by fusing a portion of the target nucleic acid molecule with a reporter gene so that the reporter gene is transcribed. By monitoring a change in the expression of the reporter gene in the presence of the oligonucleotide composition, it is possible to determine the effectiveness of the oligonucleotide composition in inhibiting the expression of the reporter gene. For example, in one embodiment, an effective oligonucleotide composition will reduce the expression of the reporter gene.

A “reporter gene” is a nucleic acid that expresses a detectable gene product, which may be RNA or protein. Detection of mRNA expression may be accomplished by Northern blotting and detection of protein may be accomplished by staining with antibodies specific to the protein. Preferred reporter genes produce a readily detectable product. A reporter gene may be operably linked with a regulatory DNA sequence such that detection of the reporter gene product provides a measure of the transcriptional activity of the regulatory sequence. In preferred embodiments, the gene product of the reporter gene is detected by an intrinsic activity associated with that product. For instance, the reporter gene may encode a gene product that, by enzymatic activity, gives rise to a detectable signal based on color, fluorescence, or luminescence. Examples of reporter genes include, but are not limited to, those coding for chloramphenicol acetyl transferase (CAT), luciferase, beta-galactosidase, and alkaline phosphatase.

One skilled in the art would readily recognize numerous reporter genes suitable for use in the present invention. These include, but are not limited to, chloramphenicol acetyltransferase (CAT), luciferase, human growth hormone (hGH), and beta-galactosidase. Examples of such reporter genes can be found in F. A. Ausubel et al., Eds., Current Protocols in Molecular Biology, John Wiley & Sons, New York, (1989). Any gene that encodes a detectable product, e.g., any product having detectable enzymatic activity or against which a specific antibody can be raised, can be used as a reporter gene in the present methods.

One reporter gene system is the firefly luciferase reporter system. (Gould, S. J., and Subramani, S. 1988. Anal. Biochem., 7:404-408 incorporated herein by reference). The luciferase assay is fast and sensitive. In this assay, a lysate of the test cell is prepared and combined with ATP and the substrate luciferin. The encoded enzyme luciferase catalyzes a rapid, ATP dependent oxidation of the substrate to generate a light-emitting product. The total light output is measured and is proportional to the amount of luciferase present over a wide range of enzyme concentrations.

CAT is another frequently used reporter gene system; a major advantage of this system is that it has been an extensively validated and is widely accepted as a measure of promoter activity. (Gorman C. M., Moffat, L. F., and Howard, B. H. 1982. Mol. Cell. Biol., 2:1044-1051). In this system, test cells are transfected with CAT expression vectors and incubated with the candidate substance within 2-3 days of the initial transfection. Thereafter, cell extracts are prepared. The extracts are incubated with acetyl CoA and radioactive chloramphenicol. Following the incubation, acetylated chloramphenicol is separated from nonacetylated form by thin layer chromatography. In this assay, the degree of acetylation reflects the CAT gene activity with the particular promoter.

Another suitable reporter gene system is based on immunologic detection of hGH. This system is also quick and easy to use. (Selden, R., Burke-Howie, K. Rowe, M. E., Goodman, H. M., and Moore, D. D. (1986), Mol. Cell, Biol., 6:3173-3179 incorporated herein by reference). The hGH system is advantageous in that the expressed hGH polypeptide is assayed in the media, rather than in a cell extract. Thus, this system does not require the destruction of the test cells. It will be appreciated that the principle of this reporter gene system is not limited to hGH but rather adapted for use with any polypeptide for which an antibody of acceptable specificity is available or can be prepared.

In one embodiment, nuclease stability of a double-stranded oligonucleotide of the invention is measured and compared to a control, e.g., an RNAi molecule typically used in the art (e.g., a duplex oligonucleotide of less than 25 nucleotides in length and comprising 2 nucleotide base overhangs) or an unmodified RNA duplex with blunt ends.

The target RNA cleavage reaction achieved using the siRNAs of the invention is highly sequence specific. Sequence identity may determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10. Additionally, numerous commercial entities, such as Dharmacon, and Invitrogen provide access to algorithms on their website. The Whitehead Institute also offers a free siRNA Selection Program. Greater than 90% sequence identity, e.g., 91%, 92%, 93%, 94%, 95%, 96%, 97%, 98%, 99% or even 100% sequence identity, between the siRNA and the portion of the target gene is preferred. Alternatively, the siRNA may be defined functionally as a nucleotide sequence (or oligonucleotide sequence) that is capable of hybridizing with a portion of the target gene transcript. Examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

### Therapeutic Use

By inhibiting the expression of a gene, the oligonucleotide compositions of the present invention can be used to treat any disease involving the expression of a protein. Examples of diseases that can be treated by oligonucleotide compositions, just to illustrate, include: cancer, retinopathies, autoimmune diseases, inflammatory diseases (i.e., ICAM-1 related disorders, Psoriasis, Ulcerative Colitus, Crohn's disease), viral diseases (i.e., HIV, Hepatitis C), miRNA disorders, and cardiovascular diseases.

As discussed above, sd-rxRNA molecules administered by methods described herein are effectively targeted to all the cell types in the eye.

Aspects of the invention relate to targeting sd-rxRNA to various cell types in the eye, including, but not limited to, cells located in the ganglion cell layer (GCL), the inner plexiform layer inner (IPL), the inner nuclear layer (INL), the outer plexiform layer (OPL), outer nuclear layer (ONL), outer segments (OS) of rods and cones, the retinal pigmented epithelium (RPE), the inner segments (IS) of rods and cones, the epithelium of the conjunctiva, the iris, the ciliary body, the corneum, and epithelium of ocular sebaceous glands.

The sd-rxRNA that is targeted to the eye may, in some instances target an eye-specific gene or a gene that is expressed at higher levels in the eye than in other tissues. As one of ordinary skill in the art would appreciate, publicly accessible databases can be used to identify genes that have eye-specific expression or increased expression in the eye relative to other tissues. Several non-limiting examples of such databases include TISGED (Tissue-Specific Genes Database) and the TiGER database for tissue-specific gene expression and regulation. In other embodiments, the sd-rxRNA does not target an eye-specific gene. In other embodiments, the gene that is targeted does not have eye-specific expression or increased expression in the eye.

In some instances, an sd-rxRNA that is targeted to the eye is used to ameliorate at least one symptom of a condition or disorder associated with the eye.

Aspects of the invention relate to treatment of ocular disorders affecting the front of the eye.

Non-limiting examples of ocular conditions or disorders associated with the front of the eye include: corneal scarring, corneal perforation, corneal dystrophies, corneal injury and or trauma (including burns), corneal inflammation, corneal infection, opthalmia neonatorum, erythema multiform (Stevens-Johnson Syndrome), xerophthalmia (dry eye syndrome), trachoma, onchocerciasis (river blindness), corneal complications of leprosy, keratitis, persistent corneal epithelial defects, iridocorneal endothelial syndrome, Fuch's Dystrophy, trichiasis, ocular herpes, corneal transplant failure and or rejection.

In some embodiments, the condition or disorder is corneal grafting or transplant. In some embodiments, the therapeutic RNA molecule is administered as an ex vivo treatment of the graft or transplant prior to surgery.

In some embodiments, the condition or disorder is a wound or scratch on the cornea. It should be appreciated that any disorder or damage to the cornea is encompassed by conditions and disorders associated with aspects of the invention.

In some embodiments, the therapeutic RNA is administered to an eye that is compromised or wounded. In some embodiments, the cornea is compromised or wounded and the therapeutic RNA is administered to the cornea that is compromised or wounded. In some embodiments, the therapeutic RNA is administered topically to the cornea.

Several other non-limiting examples of conditions or disorders associated with the eye include: vascular leakage/neovascularization (e.g., angiographic cystoid macular edema, macular edema secondary to retinal vein occlusion (RVO), glaucoma or neovascular glaucoma (NVG), retinopathy of prematurity (ROP); fibroproliferative diseases (e.g., proliferative vitreoretinopathy (PVR), epiretinal membranes/vitreomacular adhesions; age-related macular degeneration (AMD) (e.g., choroidal neovascularization (wet AMD), geographic atrophy (advanced dry AMD), early-to-intermediate dry AMD); diabetic retinopathy (e.g., nonproliferative diabetic retinopathy (NPDR), diabetic macular edema (DME), proliferative diabetic retinopathy (PDR); retinal degenerative diseases (and related diseases); retinal vascular occlusive diseases (e.g., retinal vein occlusion, retinal artery occlusion) and other retinal diseases; retinal detachment; inflammatory diseases such as uveitis (including panuveitis) or choroiditis (including multifocal choroiditis) of unknown cause (idiopathic) or associated with a systemic (e.g., autoimmune) disease; episcleritis or scleritis; Birdshot retinochoroidopathy; vascular diseases (retinal ischemia, retinal vasculitis, choroidal vascular insufficiency, choroidal thrombosis); neovascularization of the optic nerve; optic neuritis; blepharitis; keratitis; rubeosis iritis; Fuchs' heterochromic iridocyclitis; chronic uveitis or anterior uveitis; conjunctivitis; allergic conjunctivitis (including seasonal or perennial, vernal, atopic, and giant papillary); keratoconjunctivitis sicca (dry eye syndrome); iridocyclitis; iritis; scleritis; episcleritis; corneal edema; scleral disease; ocular cicatrcial pemphigoid; pars planitis; Posner Schlossman syndrome; Behcet's disease; Vogt-Koyanagi-Harada syndrome; hypersensitivity reactions; conjunctival edema; conjunctival venous congestion; periorbital cellulitis; acute dacryocystitis; non-specific vasculitis; sarcoidosis; keratoconjunctivitis sicca, a condition also known as dry-eye, keratitis sicca, sicca syndrome, xeropthalmia, and dry eye syndrome (DES), which can arise from decreased tear production and/or increased tear film evaporation due to abnormal tear composition; a disorder associated with the autoimmune diseases rheumatoid arthritis, lupus erythematosus, diabetes mellitus, and Sjogren's syndrome. In some embodiments, sd-rxRNA is administered as a method of wound healing. Non-limiting examples of conditions or disorders associated with the eye are incorporated by reference from US Patent Publication 20100010082 and U.S. Pat. No. 6,331,313.

### Neovascularization/Vascular Leakage

Aspects of the invention relate to treating diseases and conditions associated with neovascularization and/or vascular leakage. Of these conditions, wet AMD and DME are most prevalent, PDR and macular edema secondary to RVO are of lower prevalence, and rare neovascular conditions include ROP and neovascular glaucoma. Vascular leakage is considered to be the driving force behind DME, while both vascular leakage and neovascularization drive PDR. Oligonucleotide compositions of the present invention can be selected based on the etiology of a particular disease or condition. For example, a composition comprising an anti-angiogenic oligonucleotide affecting vascular permeability may be chosen to treat DME, while one affecting proliferation may be chosen to treat PDR. Alternatively, oligonucleotide compositions may comprise a combination of anti-angiogenic agents, for example, an sd-rxRNA that inhibits function of a target that affects vascular permeability and an sd-rxRNA that inhibits function of a target that affects proliferation, such that both etiological aspects of the condition are targeted.

In certain embodiments, the sd-rxRNA is used to treat neovascularization and/or vascular permeability. In some embodiments, the sd-rxRNA targets Vascular Endothelial Growth Factor (VEGF), an inhibitor of vascular permeability. VEGF is a canonical and clinically validated target for treatment of wet AMD and approval is expected for DME and RVO-associated ME. VEGF proteins are growth factors that bind to tyrosine kinase receptors and are implicated in multiple disorders such as cancer, age-related macular degeneration, rheumatoid arthritis and diabetic retinopathy. Members of this protein family include VEGF-A, VEGF-B, VEGF-C and VEGF-D. Representative Genbank accession numbers providing DNA and protein sequence information for human VEGF proteins are NM_001171623.1 (VEGF-A), U43368 (VEGF-B), X94216 (VEGF-C), and D89630 (VEGF-D).

Aspects of the invention relate to rxRNAori directed against VEGF. As described in the Examples section, over 100 optimal rxRNA ori sequences for VEGF were identified herein (Tables 2 and 9). An rxRNAori can be directed against a sequence comprising at least 12 contiguous nucleotides of a sequence within Table 2 or 9. For example, an rxRNAori can be directed against a sequence comprising 12, 13, 14, 15, 16, 17, 18, 19, 20, 21, 22, 23, 24, or 25 contiguous nucleotides of a sequence within Table 2 or 9. In some embodiments, an rxRNAori is directed against a sequence comprising at least 12 contiguous nucleotides of SEQ ID NO:13 (AUCACCAUCGACAGAACAGUCCUUA) or SEQ ID NO: 28 (CCAUGCAGAUUAUGCGGAUCAAACA). The sense strand of the rxRNAori molecule can comprise at least 12 contiguous nucleotides of a sequence selected from the sequences presented in Table 2. In some embodiments, the sense strand of the rxRNAori comprises at least 12 contiguous nucleotides of the sequence of SEQ ID NO:13 or SEQ ID NO: 28. The antisense strand of the rxRNAori can be complementary to at least 12 contiguous nucleotides of a sequence selected from the sequences within Table 2. In some embodiments, the antisense strand of the rxRNAori comprises at least 12 contiguous nucleotides of SEQ ID NO:1377 (UAAGGACUGUUCUGUCGAUGGUGAU) or SEQ ID NO:1378 (UGUUUGAUCCGCAUAAUCUGCAUGG).

Non-limiting examples of an rxRNAori directed against VEGF include an rxRNAori comprising a sense strand that comprises the sequence of SEQ ID NO:13 and an antisense strand that comprises the sequence of SEQ ID NO:1377 or an rxRNAori comprising a sense strand that comprises the sequence of SEQ ID NO:28 and an antisense strand that comprises the sequence of SEQ ID NO:1378. It should be appreciated that a variety of modifications patterns are compatible with rxRNAori. Aspects of the invention encompass rxRNAori directed against VEGF, wherein the rxRNAori is modified or unmodified. In some embodiments, the rxRNAori is adminstered to the eye.

Ori sequences can also be converted to sd-rxRNA molecules to target VEGF in the eye. It should be appreciated that the disclosed ori sequences represent non-limiting examples of sequences within VEGF for sd-rxRNA development. Variations in length and modifications of these sequences, as well as other sequences within VEGF are also compatible with development of sd-rxRNA molecules. An sd-rxRNA can be directed against a sequence selected from the sequences within Table 2 or 9. For example, an sd-rxRNA can be directed against a sequence comprising at least 12 contiguous nucleotides of a sequence selected from the sequences within Table 2 or 9. In some embodiments, an sd-rxRNA can be directed against a sequence comprising 12, 13, 14, 15, 16, 17, 18, 19, 20, 21, 22, 23, 24 or 25 contiguous nucleotides of a sequence selected from the sequences within Table 2 or 9.

In some embodiments, an sd-rxRNA directed against VEGF comprises at least 12 nucleotides of a sequence selected from the sequences within Table 8. In some embodiments, the sense strand of the sd-rxRNA comprises at least 12 contiguous nucleotides of the sequence of SEQ ID NO:1317 (AGAACAGUCCUUA) or SEQ ID NO:1357 (UGCGGAUCAAACA) and/or the antisense strand of the sd-rxRNA comprises at least 12 contiguous nucleotides of the sequence of SEQ ID NO:1318 (UAAGGACUGUUCUGUCGAU) or SEQ ID NO:1358 (UGUUUGAUCCGCAUAAUCU). In certain embodiments, an sd-rxRNA directed against VEGF includes a sense strand comprising SEQ ID NO:1317 and an antisense strand comprising SEQ ID NO:1318. Various chemical modification patterns are compatible with sd-rxRNA. Non-limiting examples of modified forms of SEQ ID NO:1317 and SEQ ID NO:1318 are represented by SEQ ID NOs 1379 (A. G. A. A.mC. A. G.mU.mC.mC.mU.mU. A.Chl) and 1380 (P.mU. A. A. G. G. A.fC.fU. G.fU.fU.fC.fU*G*fU*fC*G*A*U), respectively.

In certain embodiments, an sd-rxRNA directed against VEGF includes a sense strand comprising SEQ ID NO:1357 and an antisense strand comprising SEQ ID NO:1358. Non-limiting examples of modified forms of SEQ ID NO:1357 and SEQ ID NO:1358 are represented by SEQ ID NOs 1397 (mU. G.mC. G. G. A.mU.mC. A. A. A.mC. A.Chl) and 1398 (P.mU. G.fU.fU.fU. G. A.fU.fC.fC. G.fC. A*fU*A*A*fU*fC*U), respectively. In certain embodiments, the sd-rxRNA comprises SEQ ID NOs 1397 and 1398. It should be appreciated that other modifications patterns of sd-rxRNAs disclosed herein are also compatible with aspects of the invention.

Described herein are also sd-rxRNAs directed against genes that encode for proteins other than VEGF. Non-limiting examples of such sd-rxRNAs are provided in Tables 3-7. In some embodiments, an sd-rxRNA comprises at least 12 contiguous nucleotides of a sequence selected from the sequences within Tables 3-7.

In some embodiments, the sd-rxRNA is directed against CTGF. Non-limiting examples of sd-rxRNAs directed against CTGF are provided in Table 5. In some embodiments, the sense strand of an sd-rxRNA directed against CTGF comprises at least 12 contiguous nucleotides of the sequence of SEQ ID NO:1431 (GCACCUUUCUAGA) and an antisense strand of an sd-rxRNA directed against CTGF comprises at least 12 contiguous nucleotides of the sequence of SEQ ID NO:1432 (UCUAGAAAGGUGCAAACAU). Non-limiting examples of modified forms of SEQ ID NOs 1431 and 1432 are represented by SEQ ID NOs:947 (G.mC. A.mC.mC.mU.mU.mU.mC.mU. A*mG*mA.TEG-Chl) and 948 (P.mU.fC.fU. A. G.mA. A.mA. G. G.fU. G.mC*A*A*A*mC*A*U.), respectively. In some embodiments, the sense strand of an sd-rxRNA directed against CTGF comprises at least 12 contiguous nucleotides of the sequence of SEQ ID NO:1433 (UUGCACCUUUCUAA) and an antisense strand of an sd-rxRNA directed against CTGF comprises at least 12 contiguous nucleotides of the sequence of SEQ ID NO:1434 (UUAGAAAGGUGCAAACAAGG). Non-limiting examples of modified forms of SEQ ID Nos 1433 and 1434 and represented by SEQ ID NOs:963 (mU.mU. G.mC. A.mC.mC.mU.mU.mU.mC.mU*mA*mA.TEG-Chl) and 964 (P.mU.fU. A. G. A.mA. A. G. G.fU. G.fC.mA.mA*mA*fC*mA*mA*mG*G.).

In some embodiments, the sense strand of the sd-rxRNA directed against CTGF comprises at least 12 contiguous nucleotides of the sequence of SEQ ID NO:947 or SEQ ID NO:963. In certain embodiments, the sd-rxRNA directed against CTGF includes a sense strand comprising the sequence of SEQ ID NO:963 and an antisense strand comprising the sequence of SEQ ID NO:964. In other embodiments, the sd-rxRNA directed against CTGF includes a sense strand comprising the sequence of SEQ ID NO:947 and an antisense strand comprising the sequence of SEQ ID NO:948.

sd-rxRNA can be hydrophobically modified. For example, the sd-rxRNA can be linked to one or more hydrophobic conjugates. In some embodiments, the sd-rxRNA includes at least one 5-methyl C or U modifications.

Aspects of the invention relate to compositions comprising rxRNAori and/or sd-rxRNA nucleic acids described herein. A composition can comprise one or more rxRNAori and/or sd-rxRNA. In some embodiments, a composition comprises multiple different rxRNAoris that are directed to genes encoding for different proteins and/or multiple different sd-rxRNAs that are directed to genes encoding for different proteins. In some embodiments, a composition comprises sd-rxRNA directed to VEGF as well as sd-rxRNA directed against another gene such as a gene encoding for CTGF or PTGS2 (COX-2).

In some embodiments, one or more sd-rxRNA targets IGTA5, ANG2, CTGF, COX-2, complement factors 3 or 5, or a combination thereof.

In some embodiments, the sd-rxRNA targets Connective tissue growth factor (CTGF), also known as Hypertrophic chondrocyte-specific protein 24. CTGF is a secreted heparin-binding protein that has been implicated in wound healing and scleroderma. Connective tissue growth factor is active in many cell types including fibroblasts, myofibroblasts, endothelial and epithelial cells. Representative Genbank accession number providing DNA and protein sequence information for human CTGF are NM_001901.2 and M92934.

In some embodiments, the sd-rxRNA targets Osteopontin (OPN), also known as Secreted phosphoprotein 1 (SPP1), Bone Sinaloprotein 1 (BSP-1), and early T-lymphocyte activation (ETA-1). SPP1 is a secreted glycoprotein protein that binds to hydroxyapatite. OPN has been implicated in a variety of biological processes including bone remodeling, immune functions, chemotaxis, cell activation and apoptosis. Osteopontin is produced by a variety of cell types including fibroblasts, preosteoblasts, osteoblasts, osteocytes, odontoblasts, bone marrow cells, hypertrophic chondrocytes, dendritic cells, macrophages, smooth muscle, skeletal muscle myoblasts, endothelial cells, and extraosseous (non-bone) cells in the inner ear, brain, kidney, deciduum, and placenta. Representative Genbank accession number providing DNA and protein sequence information for human Osteopontin are NM_000582.2 and X13694.

In some embodiments, the sd-rxRNA targets Transforming growth factor 13 (TGFβ) proteins, for which three isoforms exist in mammals (TGFβ1, TGFβ2, TGFβ3). TGFβ proteins are secreted proteins belonging to a superfamily of growth factors involved in the regulation of many cellular processes including proliferation, migration, apoptosis, adhesion, differentiation, inflammation, immuno-suppression and expression of extracellular proteins. These proteins are produced by a wide range of cell types including epithelial, endothelial, hematopoietic, neuronal, and connective tissue cells. Representative Genbank accession numbers providing DNA and protein sequence information for human TGFβ1, TGFβ2 and TGFβ3 are BT007245, BC096235, and X14149, respectively. Within the TGFβ family, TGFβ1 and TGFβ2 but not TGFβ3 represent suitable targets. In some embodiments, the sd-rxRNA targets Cyclooxygenase-2 (COX-2), also called Prostaglandin G/H synthase 2 (PTGS2). COX-2 is involved in lipid metabolism and biosynthesis of prostanoids and is implicated in inflammatory disorders such as rheumatoid arthritis. A representative Genbank accession number providing DNA and protein sequence information for human COX-2 is AY462100.

In other embodiments, the sd-rxRNA targets HIF-1α, a component of the HIF-1 transcription factor. HIF-1α is a key regulator of the cellular response to hypoxia, acting upstream of VEGF-dependent and VEGF-independent pro-angiogenic pathways and pro-fibrotic pathways. HIF-1α inhibitors are effective in laser CNV and OIR models. A representative Genbank accession number providing DNA and protein sequence information for human HIF1α is U22431.

In some embodiments, the sd-rxRNA targets mTOR. mTOR is a serine/threonine kinase component of the PI3K/Akt/mTOR pathway, and is a regulator or cell growth, proliferation, survival, transcription and translation. mTOR inhibitors have both anti-angiogenic (effective in laser CNV and OIR models) and anti-fibrotic activity. Rapamycin and other mTOR inhibitors are being used in clinical trials for AMD and DME. A representative Genbank accession number providing DNA and protein sequence information for human mTOR is L34075.

In some embodiments, the sd-rxRNA targets SDF-1 (stromal derived factor-1), which is a soluble factor that stimulates homing of hematopoietic stem cells and endothelial progenitor cells to tissues. SDF-1 acts synergistically with VEGF to drive pathologic neovascularization, and inhibition of SDF-1 signaling suppresses neovascularization in OIR, laser CNV, and VEGF-induced rodent models.

In certain embodiments, the sd-rxRNA targets PDGF-B (platelet-derived growth factor B). Retinal overexpression of PDGF-B in transgenic mice leads to fibrovascular proliferation, and inhibition of PDGF-B signaling enhances efficacy of anti-VEGF treatment in laser CNV model. Dual inhibition of PDGF-B and VEGF can promote regression of NV. Representative Genbank accession numbers providing DNA and protein sequence information for human PDGF genes and proteins include X03795 (PDGFA), X02811 (PDGFB), AF091434 (PDGFC), AB033832 (PDGFD).

In some embodiments, the therapeutic RNA targets TIE1 (tyrosine kinase with immunoglobulin-like and EGF-like domains). In some embodiments, the therapeutic RNA targets TIE2 (TEK tyrosine kinase). In some embodiments, the therapeutic RNA targets angiopoietins. In some embodiments, the therapeutic RNA targets ANG1 (angiopoietin 1). In some embodiments, the therapeutic RNA targets ANG2 (angiopoietin 2).

In other embodiments, the sd-rxRNA targets VEGFR1 (vascular endothelial growth factor receptor 1), also referred to as FLT1 (fms-related tyrosine kinase 1). This gene encodes a member of the vascular endothelial growth factor receptor (VEGFR) family. VEGFR family members are receptor tyrosine kinases (RTKs) which contain an extracellular ligand-binding region with seven immunoglobulin (Ig)-like domains, a transmembrane segment, and a tyrosine kinase (TK) domain within the cytoplasmic domain. This protein binds to VEGFR-A, VEGFR-B and placental growth factor and plays an important role in angiogenesis and vasculogenesis. Representative Genbank accession numbers providing DNA and protein sequence information for human VEGFR1 genes and proteins include NM_001159920, NP_001153392, NM_001160030, NP_001153502, NM_001160031, NP_001153503, NM_002019, and NP_002010.

In certain embodiments, the sd-rxRNA targets VEGFR2 (vascular endothelial growth factor receptor 2), also referred to as KDR (kinase insert domain receptor). This receptor, known as kinase insert domain receptor, is a type III receptor tyrosine kinase. It functions as the main mediator of VEGF-induced endothelial proliferation, survival, migration, tubular morphogenesis and sprouting. The signaling and trafficking of this receptor are regulated by multiple factors, including Rab GTPase, P2Y purine nucleotide receptor, integrin alphaVbeta3, T-cell protein tyrosine phosphatase, etc. Representative Genbank accession numbers providing DNA and protein sequence information for human VEGFR2 genes and proteins include NM_002253 and NP_002244. In some embodiments, treatment of neovascularization and/or vascular leakage may include the use of a combination of sd-rxRNAs, each sd-rxRNA targeting a different gene. For example, an sd-rRNA targeting VEGF and an sd-rxRNA targeting HIF-1α can be used. As another example, an sd-rRNA targeting mTOR and an sd-rRNA targeting SDF-1 can be used. As yet another example, an sd-rRNA targeting VEGF, an sd-rRNA targeting mTOR, and an sd-rRNA targeting PDGF-B can be used.

### Wet AMD (Choroidal Neovascularization (CNV))

Aspects of the invention relate to treating choroidal vascularization, the fastest progressing form of AMD (˜1 million cases in the U.S.), which results from inappropriate growth of new blood vessels from the choroid into the subretinal space and leakage of fluid from these vessels. If untreated, 75% of patients will progress to legal blindness within three years. Intravitreal anti-VEGF agents can rapidly improve vision by inhibiting CNV lesion growth and vascular leakage from CNV lesions; however, existing anti-VEGFs may not cause regression of existing lesions in most patients.

In certain embodiments, the sd-rxRNA is used to treat CNV. In some embodiments, the sd-rxRNA targets VEGF. In other embodiments, the sd-rxRNA targets HIF-1α, mTOR, PDGF-B, SDF-1, IGTA5, ANG2, CTGF, COX-2, or complement factors 3 or 5. In some embodiments, treatment of CNV includes the use of a combination of sd-rxRNAs, each sd-rxRNA targeting a different gene.

### Diabetic Macular Edema (DME)

DME results from vascular leakage from retinal vessels leading to vision-threatening buildup of fluid in the macula, occurring in ˜2-5% of diabetic patients. The current standard of care is focal or grid laser photocoagulation. Intravitreal anti-VEGF agents and corticosteroids have been shown to be effective, but are not yet approved.

In certain embodiments, the sd-rxRNA is used to treat DMA. In some embodiments, the sd-rxRNA targets VEGF. In other embodiments, the sd-rxRNA targets HIF-1α, mTOR, PDGF-B, SDF-1, IGTA5, ANG2, CTGF, COX-2, or complement factors 3 or 5. In some embodiments, treatment of DME includes the use of a combination of sd-rxRNAs, each sd-rxRNA targeting a different gene.

### Proliferative Diabetic Retinopathy (PDR)

PDR is associated with chronic retinal ischemia. Retinal neovascularization occurs secondary to retinal ischemia and can lead to vitreous hemorrhage, fibrovascular proliferation, and traction retinal detachment.

In certain embodiments, the sd-rxRNA is used to treat PDR. In some embodiments, the sd-rxRNA targets VEGF. In other embodiments, the sd-rxRNA targets HIF-1α, mTOR, PDGF-B, SDF-1, IGTA5, ANG2, CTGF, COX-2, or complement factors 3 or 5. In some embodiments, treatment of PDR includes the use of a combination of sd-rxRNAs, each sd-rxRNA targeting a different gene.

### Macular Edema Secondary to RVO

RVO can occur in ischemic and non-ischemic forms. Ischemic RVO can lead to several vision threatening complications, including macular edema, retinal ischemia, and neovascularization. Non-ischemic RVO has a more favorable prognosis and the most common vision-threatening complication is macular edema.

In certain embodiments, the sd-rxRNA is used to treat macular edema secondary to RVO. In some embodiments, the sd-rxRNA targets VEGF. In other embodiments, the sd-rxRNA targets HIF-1α, mTOR, PDGF-B, SDF-1, IGTA5, ANG2, CTGF, COX-2, or complement factors 3 or 5. In some embodiments, treatment of macular edema secondary to RVO includes the use of a combination of sd-rxRNAs, each sd-rxRNA targeting a different gene.

### Iris Neovascularization/Neovascular Glaucoma (NVG)

NVG is a rare disorder that develops in eyes suffering from severe, chronic ocular ischemia. The most common causes are advanced PDR or ischemic CRVO. Iris neovascularization occurs due to ischemia, and eventually obstructs trabecular meshwork leading to a severe secondary glaucoma.

In certain embodiments, the sd-rxRNA is used to treat iris neovascularization and/or NVG. In some embodiments, the sd-rxRNA targets VEGF. In other embodiments, the sd-rxRNA targets HIF-1α, mTOR, PDGF-B, SDF-1, IGTA5, ANG2, CTGF, COX-2, or complement factors 3 or 5. In some embodiments, treatment of iris neovascularization and/or NVG includes the use of a combination of sd-rxRNAs, each sd-rxRNA targeting a different gene.

### Proliferative Retinal Diseases

Proliferative retinal diseases include proliferative vitreoretinopathy, proliferative diabetic retinopathy (PDR), epiretinal membranes (transparent layers of cells that can grow over the surface of the macula, causing retinal traction), and wet AMD.

In certain embodiment, the sd-rxRNA is used to treat proliferative retinal diseases. In some embodiments, the sd-rxRNA targets TGFβ, while in other embodiments, the sd-rxRNA targets CTGF. In still other embodiments, multiple sd-rxRNAs target PDGFRα, mTOR, IGTA5, or a combination thereof. In yet other embodiments, multiple sd-rxRNAs targets TGFβ and at least one of CTGF, PDGFRα, mTOR, IGTA5, or a combination thereof. In further embodiments, multiple sd-rxRNAs target CTGF and at least one of TGFβ, PDGFRα, mTOR, IGTA5, or a combination thereof. In certain embodiments, treatment of proliferative retinal diseases includes the use of a combination of sd-rxRNAs, each sd-rxRNA targeting a different gene.

### Dry AMD

In certain embodiments, the sd-rxRNA is used to treat dry AMD, including geographic atrophy (GA) (a form of advanced AMD that progresses more slowly than wet AMD) and early-to-intermediate dry AMD (early stages of dry AMD that precedes GA or CNV). In some embodiments, the sd-rxRNA targets Alu transcription. In other embodiments, the sd-rxRNA targets transcription factors or other molecules that inhibit or regulate expression of DICER (an endoribonuclease in the RNase III family that cleaves double-stranded RNA (dsRNA) and pre-microRNA (miRNA) into short double-stranded RNA fragments called small interfering RNA (siRNA) about 20-25 nucleotides long).

### Cystoid Macular Edema

Cystoid macular edema is an accumulation of intraretinal fluid in erofoveal cysts following surgery. In certain embodiments, the sd-rxRNA is used to treat cystoid macular edema. In some embodiments, the sd-rxRNA targets COX-2 (cyclooxygenase-2) enzyme.

### Retinitis Pigmentosa

Retinitis pigmentosa is an inherited retinal degenerative disease caused by mutations in several known genes. In certain embodiments, the sd-rxRNA is used to treat retinitis pigmentosa. In some embodiments, the sd-rxRNA targets NADPH oxidase.

### Glaucoma

Glaucoma is a slowly progressive disease characterized by degeneration of the optic nerve. There is an initial vision loss in the periphery with central vision loss at advanced stages of the disease. The best understood risk factor for glaucoma-related vision loss is intraocular pressure (TOP). Trabeculectomy is a surgical procedure designed to create a channel or bleb though the sclera to allow excess fluid to drain from the anterior of the eye, leading to reduced IOP. The most common cause of trabeculectomy failure is blockage of the bleb by scar tissue.

In certain embodiments, the sd-rxRNA is used to prevent formation of scar tissue resulting from a trabeculectomy. In some embodiments, the sd-rxRNA targets CTGF, while in other embodiments, the sd-rxRNA targets TGFβ. In still other embodiments, multiple sd-rxRNAs target both CTGF and TGFβ. In some embodiments, scar tissue formation is prevented by the use of a combination of sd-rxRNAs, one targeting CTGF and one targeting TGFβ.

### Uveitis

Uveitis is a broad group of disorders characterized by inflammation of the middle layer of the eye, called the uvea, which is composed of the choroid, ciliary body, and iris. The disorders are categorized anatomically as anterior, intermediate, posterior, or panuveitis, and are categorized pathologically as infectious or non-infectious.

In certain embodiments, the sd-rxRNA is used to treat uveitis. In some embodiments, the sd-rxRNA targets a cytokine, for example TNFα. In other embodiments, the sd-rxRNA targets IL-1, IL-6, IL-15, IL-17, IL-2R, or CTLA-4. In still other embodiments, the sd-rxRNA targets adhesion molecules, including VLA-4, VCAM-1, LFA-1, ICAM-1, CD44, or osteopontin. In yet another embodiment, the sd-rxRNA targets at least one of TNFα, IL-1, IL-6, IL-15, IL-17, IL-2R, CTLA-4, VLA-4, VCAM-1, LFA-1, ICAM-1, CD44, and osteopontin. In some embodiments, scar tissue formation is prevented by the use of a combination of sd-rxRNAs, each targeting a different gene.

### Retinoblastoma (Rb)

Retinoblastoma is a rapidly developing cancer in the cells of retina. In certain embodiments, the sd-rxRNA is used to treat retinoblastoma. In some embodiments, the sd-rxRNA targets HMGA2, a nuclear protein thought to have a role in neoplastic transformation.

In certain embodiments, sd-rxRNAs of the present invention can be used for multi-gene silencing. In some embodiments, a combination of sd-rxRNAs is used to target multiple, different genes. For example, when used for the treatment of a neovascular disorder, a sd-rxRNA targeting VEGF can be used together with a sd-rxRNA targeting HIF-1α. As another example, when used for the treatment of uveitis, a sd-rxRNA targeting TNFα, a sd-rxRNA targeting VCAM-1, and a sd-rxRNA targeting IL-2R can be used in combination.

In some embodiments, multiple sd-rxRNAs can be used to target VEGF, IGTA5, ANG2, CTGF, COX-2, complement factor 3, complement factor 5, HIF-1α, mTOR, SDF-1, PDGF-β, Alu, NADPH oxidase, TGF-β, IL-1, IL-6, IL-15, IL-17, IL-2R, CTLA-4, VLA-4, VCAM-1, LFA-1, ICAM-1, CD44, osteopontin (SPP1), or any combination thereof. In some embodiments, such multi-target gene silencing can be used to treat more than one disease or condition, if so needed.

In some embodiments, the sd-rxRNA targets MAP4K4. MAP4K4 is a mammalian serine/threonine protein kinase that belongs to a group of protein kinases related to Saccharomyces cerevisiae Sterile 20 (STE20). MAP4K4 (also known as NIK for Nck interacting kinase) was first identified in a mouse screen for proteins that interact with the SH3 domain of Nck (Su et al. (1997). Since its discovery, MAP4K4 has been and continues to be linked to wide range of physiological functions.

Approaches for RNAi-mediated inhibition of MAP4K4 expression are described in, and incorporated by reference from, U.S. Provisional Application Ser. No. 61/199,661, entitled “Inhibition of MAP4K4 through RNAi,” filed on Nov. 19, 2008, and PCT application PCT/US2009/006211, filed on Nov. 19, 2009 and entitled “Inhibition of MAP4K4 through RNAi.” sd-rxRNA molecules targeting MAP4K4 are compatible with aspects of the invention. In some embodiments an sd-rxRNA molecule targeting VEGF and an sd-rxRNA molecule targeting MAP4K4 can be administered together.

Table 1 presents non-limiting examples of sd-rxRNA targets and areas in which they can be applied.

TABLE 1
Examples of sd-rxRNA targets and applications

| Target | Area of Interest | Possible Indications |
|---|---|---|
| VEGF | Neovascularization | i) AMD/DME |
| Map4K4 | Inflammation | i) Geographic Atrophy |
| CTGF | Angiogenesis, Fibrosis/Scarring | i) AMD/DME |
| | | ii) Proliferative Vitreoretinopathy |
| | | iii) Prevention of Trabeculectomy Failure |
| PTGS2 | Inflammation | i) Cystoid Macular Edema (Post Surgery), |
| (COX-2) | | ii) Geographic Atrophy |
| TGFβ | Fibrosis/Scarring | i) Proliferative Vitreoretinopathy |
| | | ii) Prevention of Trabeculectomy Failure |
| | | iii) Diabetic Retinopathy |
| VEGF/ | Neovascularization/inflamation | i) AMD/DME |
| COX-2 | | ii) Geographic Atrophy |
| | | iii) Proliferative Vitreoretinopathy |
| | | iv) Prevention of Trabeculectomy |
| | | Failure |
| VEGF/ | Neovascularization/fibrosis | i) AMD/DME |
| CTGF | | ii) Geographic Atrophy |
| | | iii) Proliferative Vitreoretinopathy |
| | | iv) Prevention of Trabeculectomy |
| | | Failure |
| VEGF/ | Neovascularization/inflamation | i) AMD/DME |
| MAP4K4 | | ii) Geographic Atrophy |
| | | iii) Proliferative Vitreoretinopathy |
| | | iv) Prevention of Trabeculectomy |
| | | Failure |

In one embodiment, in vitro treatment of cells with oligonucleotides can be used for ex vivo therapy of cells removed from a subject or for treatment of cells which did not originate in the subject, but are to be administered to the subject (e.g., to eliminate transplantation antigen expression on cells to be transplanted into a subject). In addition, in vitro treatment of cells can be used in non-therapeutic settings, e.g., to evaluate gene function, to study gene regulation and protein synthesis or to evaluate improvements made to oligonucleotides designed to modulate gene expression or protein synthesis. In vivo treatment of cells can be useful in certain clinical settings where it is desirable to inhibit the expression of a protein. The subject nucleic acids can be used in RNAi-based therapy in any animal having RNAi pathway, such as human, non-human primate, non-human mammal, non-human vertebrates, rodents (mice, rats, hamsters, rabbits, etc.), domestic livestock animals, pets (cats, dogs, etc.), Xenopus, fish, insects (Drosophila, etc.), and worms (C. elegans), etc.

The invention provides methods for inhibiting or preventing in a subject, a disease or condition associated with an aberrant or unwanted target gene expression or activity, by administering to the subject a nucleic acid of the invention. If appropriate, subjects are first treated with a priming agent so as to be more responsive to the subsequent RNAi therapy. Subjects at risk for a disease which is caused or contributed to by aberrant or unwanted target gene expression or activity can be identified by, for example, any or a combination of diagnostic or prognostic assays known in the art. Administration of a prophylactic agent can occur prior to the manifestation of symptoms characteristic of the target gene aberrancy, such that a disease or disorder is prevented or, alternatively, delayed in its progression. Depending on the type of target gene aberrancy, for example, a target gene, target gene agonist or target gene antagonist agent can be used for treating the subject.

In another aspect, the invention pertains to methods of modulating target gene expression, protein expression or activity for therapeutic purposes. Accordingly, in an exemplary embodiment, the methods of the invention involve contacting a cell capable of expressing target gene with a nucleic acid of the invention that is specific for the target gene or protein (e.g., is specific for the mRNA encoded by said gene or specifying the amino acid sequence of said protein) such that expression or one or more of the activities of target protein is modulated. These methods can be performed in vitro (e.g., by culturing the cell with the agent), in vivo (e.g., by administering the agent to a subject), or ex vivo. The subjects may be first treated with a priming agent so as to be more responsive to the subsequent RNAi therapy if desired. As such, the present invention provides methods of treating a subject afflicted with a disease or disorder characterized by aberrant or unwanted expression or activity of a target gene polypeptide or nucleic acid molecule. Inhibition of target gene activity is desirable in situations in which target gene is abnormally unregulated and/or in which decreased target gene activity is likely to have a beneficial effect.

Thus the therapeutic agents of the invention can be administered to subjects to treat (prophylactically or therapeutically) disorders associated with aberrant or unwanted target gene activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a therapeutic agent as well as tailoring the dosage and/or therapeutic regimen of treatment with a therapeutic agent. Pharmacogenomics deals with clinically significant hereditary variations in the response to drugs due to altered drug disposition and abnormal action in affected persons.

For the purposes of the invention, ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Moreover, for the purposes of the present invention, the term “a” or “an” entity refers to one or more of that entity; for example, “a protein” or “a nucleic acid molecule” refers to one or more of those compounds or at least one compound. As such, the terms “a” (or “an”), “one or more” and “at least one” can be used interchangeably herein. It is also to be noted that the terms “comprising”, “including”, and “having” can be used interchangeably. Furthermore, a compound “selected from the group consisting of” refers to one or more of the compounds in the list that follows, including mixtures (i.e., combinations) of two or more of the compounds. According to the present invention, an isolated, or biologically pure, protein or nucleic acid molecule is a compound that has been removed from its natural milieu. As such, “isolated” and “biologically pure” do not necessarily reflect the extent to which the compound has been purified. An isolated compound of the present invention can be obtained from its natural source, can be produced using molecular biology techniques or can be produced by chemical synthesis.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### Ocular Administration of RXI-109

Cynomolgus monkeys received single bilateral intravitreal injections (50 μl) of phosphate buffered saline or 0.1, 0.33, or 1 mg/eye of RXI-109 on Day 1. Whole eyes were collected seven days following intravitreal injection. CTGF protein levels were determined by immunohistochemistry detection with an anti-CTGF antibody and quantified by digital image analysis of stained slides. CTGF protein levels were reduced in a dose-dependent manner in the cornea tissue following administration of RXI-109. A statistically significant reduction of CTGF protein levels was found between the 1 mg/eye group and the PBS injected group; *p<0.05.

The sequence of RXI-109 corresponds to a sense strand sequence of: SEQ ID NO:947 (G.mC. A.mC.mC.mU.mU.mU.mC.mU. A*mG*mA.TEG-Chl) and an antisense sequence of SEQ ID NO:948 (P.mU.fC.fU. A. G.mA. A.mA. G. G.fU. G.mC*A*A*A*mC*A*U.).

### Example 2

### sd-rxRNAs Penetrate all Cell Layers in a 3D Epicorneal Tissue Culture Model

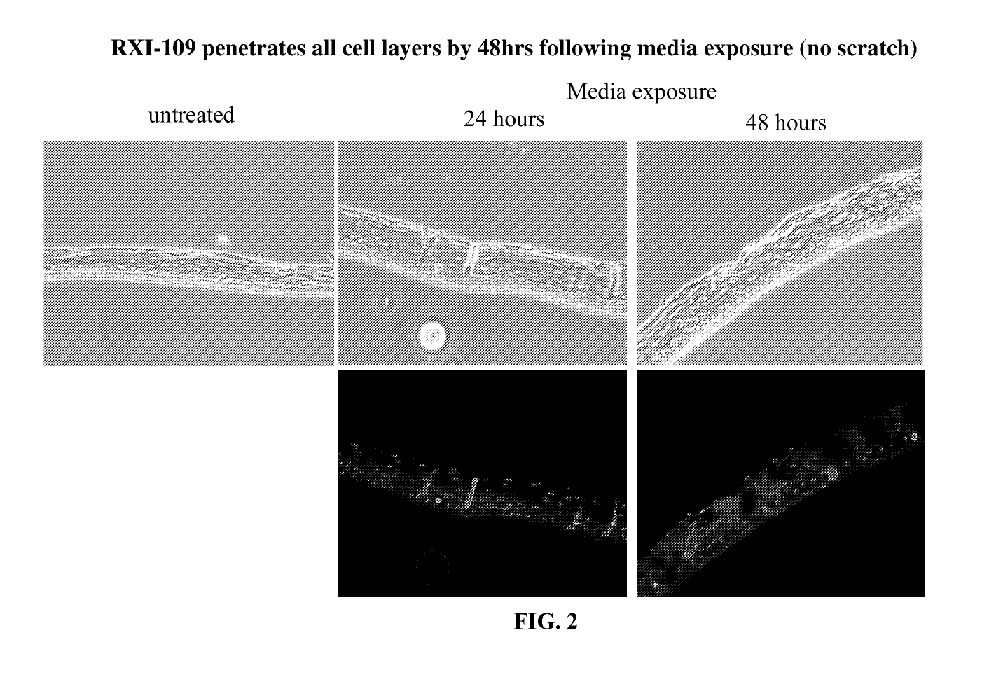

The MatTek Epicorneal model, a 3D tissue culture model utilizing human corneal epithelial cells, was used to determine if sd-rxRNAs are able to penetrate the cornea. This model is used to determine drug permeability in the cornea since the model is comparable to the permeability barrier in vivo and expresses major corneal markers. Cells were treated with fluorescently-labeled sd-rxRNA (5 uM) by media exposure (FIG. 2) or topically (FIG. 3, bottom row). In addition, uptake of the sd-rxRNA was compared in the presence of a scratch (to mimic a wound) (FIG. 3). Twenty four and forty eight hours post sd-rxRNA exposure, cells were transferred, formalin fixed and paraffin embedded and sections were cut. Fluorescent microscopy was used to detect cellular uptake of the sd-rxRNA in the corneal epithelia cells. Cellular uptake of the sd-rxRNA was observed in the epicorneal model following media exposure (intact or scratch model) or topical administration (scratch model).

### Example 3

### sd-rxRNAs Significantly Reduce Target Gene mRNA Levels in a 3D Epicorneal Tissue Culture Model

Map4k4 targeting sd-rxRNAs were tested for activity in the epicorneal model (human corneal epithelial cells). Corneal epithelial cells in 3D culture were treated with varying concentrations of a Map4k4-targeting sd-rxRNAs or non-targeting control (#21204) in serum-free media. Concentrations tested were 5 and 1 μM. The non-targeting control sd-rxRNA (#21204) is of similar structure to the Map4k4-targeting sd-rxRNA and contains similar stabilizing modifications throughout both strands. Forty eight hours post administration, cells were lysed and mRNA levels determined by qPCR according to manufacturer's protocol using gene-specific TaqMan probes (Life Technologies, Carlsbad, Calif.). Data were normalized to a house keeping gene (PPIB) and graphed with respect to the non-targeting control. Error bars represent the standard deviation from the mean of biological duplicates. (FIG. 4.)

TABLE 2
hVEGF stealth sequences

| | | | SEQ | 25-mer Sense Strand |
|---|---|---|---|---|
| Oligo | Gene | Ref | ID | (position 25 of SS, |
| ID | Region | Pos | NO | replaced with A) |
| 18832 | 3′UTR | 3471 | 1 | UAUCAUUUAUUUAUUGGUGCUACUA |
| 18811 | 3′UTR | 3199 | 2 | UUAAUUUUGCUAACACUCAGCUCUA |
| 18902 | 3′UTR | 2792 | 3 | CCUCACACCAUUGAAACCACUAGUA |
| 18830 | 3′UTR | 3429 | 4 | CUACAUACUAAAUCUCUCUCCUUUA |
| 18880 | CDS | 1343 | 5 | CCAACAUCACCAUGCAGAUUAUGCA |
| 18756 | CDS | 1389 | 6 | GCACAUAGGAGAGAUGAGCUUCCUA |
| 18913 | 3′UTR | 3163 | 7 | AUCGGUGACAGUCACUAGCUUAUCA |
| 18909 | 3′UTR | 3073 | 8 | UUUAUGAGAUGUAUCUUUUGCUCUA |
| 18831 | 3′UTR | 3430 | 9 | UACAUACUAAAUCUCUCUCCUUUUA |
| 18778 | 3′UTR | 2183 | 10 | UAACAGUGCUAAUGUUAUUGGUGUA |
| 18793 | 3′UTR | 2932 | 11 | UUGUGGAGGCAGAGAAAAGAGAAAA |
| 18898 | 3′UTR | 2210 | 12 | CACUGGAUGUAUUUGACUGCUGUGA |
| 18760 | 3′UTR | 1853 | 13 | AUCACCAUCGACAGAACAGUCCUUA |
| 18766 | 3′UTR | 1859 | 14 | AUCGACAGAACAGUCCUUAAUCCAA |
| 18908 | 3′UTR | 3072 | 15 | AUUUAUGAGAUGUAUCUUUUGCUCA |
| 18903 | 3′UTR | 2794 | 16 | UCACACCAUUGAAACCACUAGUUCA |
| 18834 | 3′UTR | 3476 | 17 | UUUAUUUAUUGGUGCUACUGUUUAA |
| 18828 | 3′UTR | 3427 | 18 | UUCUACAUACUAAAUCUCUCUCCUA |
| 18761 | 3′UTR | 1854 | 19 | UCACCAUCGACAGAACAGUCCUUAA |
| 18892 | 3′UTR | 1985 | 20 | CCUCUUGGAAUUGGAUUCGCCAUUA |
| 18764 | 3′UTR | 1857 | 21 | CCAUCGACAGAACAGUCCUUAAUCA |
| 18883 | CDS | 1347 | 22 | CAUCACCAUGCAGAUUAUGCGGAUA |
| 18790 | 3′UTR | 2790 | 23 | GUCCUCACACCAUUGAAACCACUAA |
| 18912 | 3′UTR | 3162 | 24 | GAUCGGUGACAGUCACUAGCUUAUA |
| 18794 | 3′UTR | 2933 | 25 | UGUGGAGGCAGAGAAAAGAGAAAGA |
| 18900 | 3′UTR | 2447 | 26 | AGGUCAGACGGACAGAAAGACAGAA |
| 18792 | 3′UTR | 2931 | 27 | AUUGUGGAGGCAGAGAAAAGAGAAA |
| 18886 | CDS | 1352 | 28 | CCAUGCAGAUUAUGCGGAUCAAACA |
| 18769 | 3′UTR | 1863 | 29 | ACAGAACAGUCCUUAAUCCAGAAAA |
| 18817 | 3′UTR | 3252 | 30 | CACAUUCCUUUGAAAUAAGGUUUCA |
| 18865 | 3′UTR | 1852 | 31 | CAUCACCAUCGACAGAACAGUCCUA |
| 18879 | CDS | 1342 | 32 | UCCAACAUCACCAUGCAGAUUAUGA |
| 18866 | 3′UTR | 2926 | 33 | UGCCCAUUGUGGAGGCAGAGAAAAA |
| 18751 | CDS | 1356 | 34 | GCAGAUUAUGCGGAUCAAACCUCAA |
| 18899 | 3′UTR | 2211 | 35 | ACUGGAUGUAUUUGACUGCUGUGGA |
| 18762 | 3′UTR | 1855 | 36 | CACCAUCGACAGAACAGUCCUUAAA |
| 18777 | 3′UTR | 2182 | 37 | UUAACAGUGCUAAUGUUAUUGGUGA |
| 18887 | CDS | 1353 | 38 | CAUGCAGAUUAUGCGGAUCAAACCA |
| 18846 | 3′UTR | 3516 | 39 | GGAAAAGAUAUUAACAUCACGUCUA |
| 18877 | CDS | 1340 | 40 | AGUCCAACAUCACCAUGCAGAUUAA |
| 18813 | 3′UTR | 3246 | 41 | CCAGCACACAUUCCUUUGAAAUAAA |
| 18810 | 3′UTR | 3197 | 42 | AUUUAAUUUUGCUAACACUCAGCUA |
| 18798 | 3′UTR | 2949 | 43 | AGAGAAAGUGUUUUAUAUACGGUAA |
| 18759 | CDS | 1396 | 44 | GGAGAGAUGAGCUUCCUACAGCACA |
| 18795 | 3′UTR | 2935 | 45 | UGGAGGCAGAGAAAAGAGAAAGUGA |
| 18819 | 3′UTR | 3363 | 46 | UGAUAAAAUAGACAUUGCUAUUCUA |
| 18916 | 3′UTR | 3167 | 47 | GUGACAGUCACUAGCUUAUCUUGAA |
| 18836 | 3′UTR | 3478 | 48 | UAUUUAUUGGUGCUACUGUUUAUCA |
| 18785 | 3′UTR | 2191 | 49 | CUAAUGUUAUUGGUGUCUUCACUGA |
| 18874 | CDS | 1337 | 50 | AGGAGUCCAACAUCACCAUGCAGAA |
| 18750 | CDS | 1354 | 51 | AUGCAGAUUAUGCGGAUCAAACCUA |
| 18878 | CDS | 1341 | 52 | GUCCAACAUCACCAUGCAGAUUAUA |
| 18791 | 3′UTR | 2930 | 53 | CAUUGUGGAGGCAGAGAAAAGAGAA |
| 18770 | 3′UTR | 1884 | 54 | AAACCUGAAAUGAAGGAAGAGGAGA |
| 18776 | 3′UTR | 2181 | 55 | AUUAACAGUGCUAAUGUUAUUGGUA |
| 18780 | 3′UTR | 2185 | 56 | ACAGUGCUAAUGUUAUUGGUGUCUA |
| 18805 | 3′UTR | 3155 | 57 | UCUCCCUGAUCGGUGACAGUCACUA |
| 18829 | 3′UTR | 3428 | 58 | UCUACAUACUAAAUCUCUCUCCUUA |
| 18767 | 3′UTR | 1860 | 59 | UCGACAGAACAGUCCUUAAUCCAGA |
| 18809 | 3′UTR | 3196 | 60 | UAUUUAAUUUUGCUAACACUCAGCA |
| 18816 | 3′UTR | 3251 | 61 | ACACAUUCCUUUGAAAUAAGGUUUA |
| 18867 | CDS | 1214 | 62 | CCCUGGUGGACAUCUUCCAGGAGUA |
| 18774 | 3′UTR | 1987 | 63 | UCUUGGAAUUGGAUUCGCCAUUUUA |
| 18882 | CDS | 1346 | 64 | ACAUCACCAUGCAGAUUAUGCGGAA |
| 18905 | 3′UTR | 2797 | 65 | CACCAUUGAAACCACUAGUUCUGUA |
| 18754 | CDS | 1385 | 66 | GCCAGCACAUAGGAGAGAUGAGCUA |
| 18822 | 3′UTR | 3366 | 67 | UAAAAUAGACAUUGCUAUUCUGUUA |
| 18763 | 3′UTR | 1856 | 68 | ACCAUCGACAGAACAGUCCUUAAUA |
| 18863 | 3′UTR | 3589 | 69 | UAAACAACGACAAAGAAAUACAGAA |
| 18835 | 3′UTR | 3477 | 70 | UUAUUUAUUGGUGCUACUGUUUAUA |
| 18893 | 3′UTR | 2009 | 71 | UUAUUUUUCUUGCUGCUAAAUCACA |
| 18771 | 3′UTR | 1885 | 72 | AACCUGAAAUGAAGGAAGAGGAGAA |
| 18894 | 3′UTR | 2010 | 73 | UAUUUUUCUUGCUGCUAAAUCACCA |
| 18765 | 3′UTR | 1858 | 74 | CAUCGACAGAACAGUCCUUAAUCCA |
| 18796 | 3′UTR | 2936 | 75 | GGAGGCAGAGAAAAGAGAAAGUGUA |
| 18797 | 3′UTR | 2946 | 76 | AAAAGAGAAAGUGUUUUAUAUACGA |
| 18821 | 3′UTR | 3365 | 77 | AUAAAAUAGACAUUGCUAUUCUGUA |
| 18823 | 3′UTR | 3367 | 78 | AAAAUAGACAUUGCUAUUCUGUUUA |
| 18869 | CDS | 1231 | 79 | CAGGAGUACCCUGAUGAGAUCGAGA |
| 18781 | 3′UTR | 2187 | 80 | AGUGCUAAUGUUAUUGGUGUCUUCA |
| 18775 | 3′UTR | 2180 | 81 | AAUUAACAGUGCUAAUGUUAUUGGA |
| 18870 | CDS | 1232 | 82 | AGGAGUACCCUGAUGAGAUCGAGUA |
| 18815 | 3′UTR | 3248 | 83 | AGCACACAUUCCUUUGAAAUAAGGA |
| 18804 | 3′UTR | 3135 | 84 | AUUCAUGUUUCCAAUCUCUCUCUCA |
| 18799 | 3′UTR | 2950 | 85 | GAGAAAGUGUUUUAUAUACGGUACA |
| 18779 | 3′UTR | 2184 | 86 | AACAGUGCUAAUGUUAUUGGUGUCA |
| 18924 | 3′UTR | 3545 | 87 | UCUAGUGCAGUUUUUCGAGAUAUUA |
| 18758 | CDS | 1394 | 88 | UAGGAGAGAUGAGCUUCCUACAGCA |
| 18782 | 3′UTR | 2188 | 89 | GUGCUAAUGUUAUUGGUGUCUUCAA |
| 18833 | 3′UTR | 3475 | 90 | AUUUAUUUAUUGGUGCUACUGUUUA |
| 18800 | 3′UTR | 3094 | 91 | UCUCUCUUGCUCUCUUAUUUGUACA |
| 18904 | 3′UTR | 2795 | 92 | CACACCAUUGAAACCACUAGUUCUA |
| 18845 | 3′UTR | 3515 | 93 | GGGAAAAGAUAUUAACAUCACGUCA |
| 18884 | CDS | 1348 | 94 | AUCACCAUGCAGAUUAUGCGGAUCA |
| 18818 | 3′UTR | 3356 | 95 | GUGAUUCUGAUAAAAUAGACAUUGA |
| 18814 | 3′UTR | 3247 | 96 | CAGCACACAUUCCUUUGAAAUAAGA |
| 18801 | 3′UTR | 3131 | 97 | UAAAAUUCAUGUUUCCAAUCUCUCA |
| 18873 | CDS | 1236 | 98 | GUACCCUGAUGAGAUCGAGUACAUA |
| 18802 | 3′UTR | 3133 | 99 | AAAUUCAUGUUUCCAAUCUCUCUCA |
| 18787 | 3′UTR | 2212 | 100 | CUGGAUGUAUUUGACUGCUGUGGAA |
| 18854 | 3′UTR | 3525 | 101 | AUUAACAUCACGUCUUUGUCUCUAA |
| 18901 | 3′UTR | 2791 | 102 | UCCUCACACCAUUGAAACCACUAGA |
| 18753 | CDS | 1384 | 103 | GGCCAGCACAUAGGAGAGAUGAGCA |
| 18820 | 3′UTR | 3364 | 104 | GAUAAAAUAGACAUUGCUAUUCUGA |
| 18807 | 3′UTR | 3194 | 105 | GAUAUUUAAUUUUGCUAACACUCAA |
| 18772 | 3′UTR | 1886 | 106 | ACCUGAAAUGAAGGAAGAGGAGACA |
| 18803 | 3′UTR | 3134 | 107 | AAUUCAUGUUUCCAAUCUCUCUCUA |
| 18844 | 3′UTR | 3514 | 108 | GGGGAAAAGAUAUUAACAUCACGUA |
| 18888 | CDS | 1411 | 109 | CUACAGCACAACAAAUGUGAAUGCA |
| 18895 | 3′UTR | 2077 | 110 | ACACACCCACCCACAUACAUACAUA |
| 18858 | 3′UTR | 3553 | 111 | AGUUUUUCGAGAUAUUCCGUAGUAA |
| 18889 | 3′UTR | 1981 | 112 | GGUCCCUCUUGGAAUUGGAUUCGCA |
| 18856 | 3′UTR | 3551 | 113 | GCAGUUUUUCGAGAUAUUCCGUAGA |
| 18931 | 3′UTR | 3588 | 114 | UUAAACAACGACAAAGAAAUACAGA |
| 18808 | 3′UTR | 3195 | 115 | AUAUUUAAUUUUGCUAACACUCAGA |
| 18825 | 3′UTR | 3423 | 116 | AGAAUUCUACAUACUAAAUCUCUCA |
| 18864 | 3′UTR | 3590 | 117 | AAACAACGACAAAGAAAUACAGAUA |
| 18881 | CDS | 1345 | 118 | AACAUCACCAUGCAGAUUAUGCGGA |
| 18906 | 3′UTR | 2798 | 119 | ACCAUUGAAACCACUAGUUCUGUCA |
| 18868 | CDS | 1229 | 120 | UCCAGGAGUACCCUGAUGAGAUCGA |
| 18897 | 3′UTR | 2196 | 121 | GUUAUUGGUGUCUUCACUGGAUGUA |
| 18788 | 3′UTR | 2213 | 122 | UGGAUGUAUUUGACUGCUGUGGACA |
| 18896 | 3′UTR | 2195 | 123 | UGUUAUUGGUGUCUUCACUGGAUGA |
| 18784 | 3′UTR | 2190 | 124 | GCUAAUGUUAUUGGUGUCUUCACUA |
| 18847 | 3′UTR | 3518 | 125 | AAAAGAUAUUAACAUCACGUCUUUA |
| 18852 | 3′UTR | 3523 | 126 | AUAUUAACAUCACGUCUUUGUCUCA |
| 18850 | 3′UTR | 3521 | 127 | AGAUAUUAACAUCACGUCUUUGUCA |
| 18917 | 3′UTR | 3264 | 128 | AAAUAAGGUUUCAAUAUACAUCUAA |
| 18871 | CDS | 1234 | 129 | GAGUACCCUGAUGAGAUCGAGUACA |
| 18837 | 3′UTR | 3479 | 130 | AUUUAUUGGUGCUACUGUUUAUCCA |
| 18910 | 3′UTR | 3130 | 131 | AUAAAAUUCAUGUUUCCAAUCUCUA |
| 18875 | CDS | 1338 | 132 | GGAGUCCAACAUCACCAUGCAGAUA |
| 18923 | 3′UTR | 3544 | 133 | CUCUAGUGCAGUUUUUCGAGAUAUA |
| 18853 | 3′UTR | 3524 | 134 | UAUUAACAUCACGUCUUUGUCUCUA |
| 18876 | CDS | 1339 | 135 | GAGUCCAACAUCACCAUGCAGAUUA |
| 18824 | 3′UTR | 3422 | 136 | GAGAAUUCUACAUACUAAAUCUCUA |
| 18768 | 3′UTR | 1862 | 137 | GACAGAACAGUCCUUAAUCCAGAAA |
| 18891 | 3′UTR | 1983 | 138 | UCCCUCUUGGAAUUGGAUUCGCCAA |
| 18842 | 3′UTR | 3484 | 139 | UUGGUGCUACUGUUUAUCCGUAAUA |
| 18838 | 3′UTR | 3480 | 140 | UUUAUUGGUGCUACUGUUUAUCCGA |
| 18925 | 3′UTR | 3546 | 141 | CUAGUGCAGUUUUUCGAGAUAUUCA |
| 18859 | 3′UTR | 3554 | 142 | GUUUUUCGAGAUAUUCCGUAGUACA |
| 18885 | CDS | 1351 | 143 | ACCAUGCAGAUUAUGCGGAUCAAAA |
| 18857 | 3′UTR | 3552 | 144 | CAGUUUUUCGAGAUAUUCCGUAGUA |
| 18849 | 3′UTR | 3520 | 145 | AAGAUAUUAACAUCACGUCUUUGUA |
| 18755 | CDS | 1387 | 146 | CAGCACAUAGGAGAGAUGAGCUUCA |
| 18927 | 3′UTR | 3548 | 147 | AGUGCAGUUUUUCGAGAUAUUCCGA |
| 18786 | 3′UTR | 2194 | 148 | AUGUUAUUGGUGUCUUCACUGGAUA |
| 18926 | 3′UTR | 3547 | 149 | UAGUGCAGUUUUUCGAGAUAUUCCA |
| 18928 | 3′UTR | 3549 | 150 | GUGCAGUUUUUCGAGAUAUUCCGUA |
| 18757 | CDS | 1391 | 151 | ACAUAGGAGAGAUGAGCUUCCUACA |
| 18848 | 3′UTR | 3519 | 152 | AAAGAUAUUAACAUCACGUCUUUGA |
| 18921 | 3′UTR | 3542 | 153 | GUCUCUAGUGCAGUUUUUCGAGAUA |
| 18907 | 3′UTR | 3070 | 154 | CUAUUUAUGAGAUGUAUCUUUUGCA |
| 18783 | 3′UTR | 2189 | 155 | UGCUAAUGUUAUUGGUGUCUUCACA |
| 18918 | 3′UTR | 3296 | 156 | AUAUAUAUUUGGCAACUUGUAUUUA |
| 18851 | 3′UTR | 3522 | 157 | GAUAUUAACAUCACGUCUUUGUCUA |
| 18890 | 3′UTR | 1982 | 158 | GUCCCUCUUGGAAUUGGAUUCGCCA |
| 18827 | 3′UTR | 3425 | 159 | AAUUCUACAUACUAAAUCUCUCUCA |
| 18812 | 3′UTR | 3241 | 160 | GCUCCCCAGCACACAUUCCUUUGAA |
| 18773 | 3′UTR | 1887 | 161 | CCUGAAAUGAAGGAAGAGGAGACUA |
| 18855 | 3′UTR | 3526 | 162 | UUAACAUCACGUCUUUGUCUCUAGA |
| 18789 | 3′UTR | 2214 | 163 | GGAUGUAUUUGACUGCUGUGGACUA |
| 18826 | 3′UTR | 3424 | 164 | GAAUUCUACAUACUAAAUCUCUCUA |
| 18919 | 3′UTR | 3297 | 165 | UAUAUAUUUGGCAACUUGUAUUUGA |
| 18752 | CDS | 1381 | 166 | CAAGGCCAGCACAUAGGAGAGAUGA |
| 18914 | 3′UTR | 3165 | 167 | CGGUGACAGUCACUAGCUUAUCUUA |
| 18930 | 3′UTR | 3587 | 168 | UUUAAACAACGACAAAGAAAUACAA |
| 18911 | 3′UTR | 3161 | 169 | UGAUCGGUGACAGUCACUAGCUUAA |
| 18872 | CDS | 1235 | 170 | AGUACCCUGAUGAGAUCGAGUACAA |
| 18929 | 3′UTR | 3550 | 171 | UGCAGUUUUUCGAGAUAUUCCGUAA |
| 18860 | 3′UTR | 3555 | 172 | UUUUUCGAGAUAUUCCGUAGUACAA |
| 18839 | 3′UTR | 3481 | 173 | UUAUUGGUGCUACUGUUUAUCCGUA |
| 18806 | 3′UTR | 3160 | 174 | CUGAUCGGUGACAGUCACUAGCUUA |
| 18843 | 3′UTR | 3491 | 175 | UACUGUUUAUCCGUAAUAAUUGUGA |
| 18861 | 3′UTR | 3556 | 176 | UUUUCGAGAUAUUCCGUAGUACAUA |
| 18841 | 3′UTR | 3483 | 177 | AUUGGUGCUACUGUUUAUCCGUAAA |
| 18922 | 3′UTR | 3543 | 178 | UCUCUAGUGCAGUUUUUCGAGAUAA |
| 18915 | 3′UTR | 3166 | 179 | GGUGACAGUCACUAGCUUAUCUUGA |
| 18920 | 3′UTR | 3298 | 180 | AUAUAUUUGGCAACUUGUAUUUGUA |
| 18840 | 3′UTR | 3482 | 181 | UAUUGGUGCUACUGUUUAUCCGUAA |
| 18862 | 3′UTR | 3557 | 182 | UUUCGAGAUAUUCCGUAGUACAUAA |

TABLE 3
SPP1 (Accession Number NM_000582.2) sd-rxRNA sequences

| Oligo | Start | SEQ ID | | SEQ ID | |
|---|---|---|---|---|---|
| Number | Site | NO | Sense sequence | NO | Antisense sequence |
| 14084 | 1025 | 183 | mC.mU.mC. A.mU. G. A. | 184 | P.mU.fC.fU. A. A.fU.fU.fC. |
| | | | A.mU.mU. A. G. A.Chl | | A.fU. G. A. G* A* A* |
| | | | | | A*mU* A* C. |
| 14085 | 1049 | 185 | mC.mU. G. A. G. | 186 | P.mU. A. A.fU.fU. G. |
| | | | G.mU.mC. A. A.mU.mU. | | A.fC.fC.fU.mC. A. G* A* |
| | | | A.Chl | | A* G* A*mU* G. |
| 14086 | 1051 | 187 | G. A. G. G.mU.mC. A. | 188 | P.mU.fU.fU. A. A.fU.fU. G. |
| | | | A.mU.mU. A. A. A.Chl | | A.fC.mC.mU.mC* A* G* |
| | | | | | A* A* G* A. |
| 14087 | 1048 | 189 | mU.mC.mU. G. A. G. | 190 | P.mA. A.fU.fU. G. |
| | | | G.mU.mC. A. | | A.fC.fC.fU.fC. A. G. A* A* |
| | | | A.mU.mU.Chl | | G* A*mU* G* C. |
| 14088 | 1050 | 191 | mU. G. A. G. G.mU.mC. | 192 | P.mU.fU. A. A.fU.fU. G. |
| | | | A. A.mU.mU. A. A.Chl | | A.fC.fC.mU.mC. A* G* A* |
| | | | | | A* G* A* U. |
| 14089 | 1047 | 193 | mU.mU.mC.mU. G. A. | 194 | P.mA.fU.fU. G. |
| | | | G. G.mU.mC. A. | | A.fC.fC.fU.fC. A. G. A. A* |
| | | | A.mU.Chl | | G* A*mU* G*mC* A. |
| 14090 | 800 | 195 | G.mU.mC. A. G.mC.mU. | 196 | P.mU.fC. A.fU.fC.fC. A. |
| | | | G. G. A.mU. G. A.Chl | | G.fC.fU. G. |
| | | | | | A.mC*mU*mC* |
| | | | | | G*mU*mU* U. |
| 14091 | 492 | 197 | mU.mU.mC.mU. G. | 198 | P.mA. G. A.fU.fU.fC. |
| | | | A.mU. G. A. | | A.fU.fC. A. G. A. A*mU* |
| | | | A.mU.mC.mU.Chl | | G* G*mU* G* A. |
| 14092 | 612 | 199 | mU. G. G. A.mC.mU. G. | 200 | P.mU. G. A.fC.fC.fU.fC. A. |
| | | | A. G. G.mU.mC. A.Chl | | G.fU.mC.mC. A*mU* A* |
| | | | | | A* A*mC* C. |
| 14093 | 481 | 201 | G. A. G.mU.mC.mU.mC. | 202 | P.mA. A.fU. G. G.fU. G. A. |
| | | | A.mC.mC. A.mU.mU.Chl | | G. A.mC.mU.mC* |
| | | | | | A*mU*mC* A* G* A. |
| 14094 | 614 | 203 | G. A.mC.mU. G. A. G. | 204 | P.mU.fU.fU. G. |
| | | | G.mU.mC. A. A. A.Chl | | A.fC.fC.fU.fC. A. |
| | | | | | G.mU.mC*mC* A*mU* |
| | | | | | A* A* A. |
| 14095 | 951 | 205 | mU.mC. A.mC. A. | 206 | P.mU.fU.fC. A.fU. G. |
| | | | G.mC.mC. A.mU. G. A. | | G.fC.fU. G.mU. G. A* A* |
| | | | A.Chl | | A*mU*mU*mC* A. |
| 14096 | 482 | 207 | A. G.mU.mC.mU.mC. | 208 | P.mG. A. A.fU. G. G.fU. G. |
| | | | A.mC.mC. | | A. G. A.mC.mU*mC* |
| | | | A.mU.mU.mC.Chl | | A*mU*mC* A* G. |
| 14097 | 856 | 209 | A. A. G.mC. G. G. A. A. | 210 | P.mU. G. |
| | | | A. G.mC.mC. A.Chl | | G.fC.fU.fU.fU.fC.fC. |
| | | | | | G.mC.mU.mU* A*mU* |
| | | | | | A*mU* A* A. |
| 14098 | 857 | 211 | A. G.mC. G. G. A. A. A. | 212 | P.mU.fU. G. |
| | | | G.mC.mC. A. A.Chl | | G.fC.fU.fU.fU.fC.fC. |
| | | | | | G.mC.mU*mU* A*mU* |
| | | | | | A*mU* A. |
| 14099 | 365 | 213 | A.mC.mC. A.mC. A.mU. | 214 | P.mU.fC. A.fU.fC.fC. A.fU. |
| | | | G. G. A.mU. G. A.Chl | | G.fU. G. G.mU*mC* |
| | | | | | A*mU* G* G* C. |
| 14100 | 359 | 215 | G.mC.mC. A.mU. G. | 216 | P.mA.fU. G.fU. G. G.fU.fC. |
| | | | A.mC.mC. A.mC. | | A.fU. G. |
| | | | A.mU.Chl | | G.mC*mU*mU*mU*mC* |
| | | | | | G* U. |
| 14101 | 357 | 217 | A. A. G.mC.mC. A.mU. | 218 | P.mG.fU. G. G.fU.fC. A.fU. |
| | | | G. A.mC.mC. A.mC.Chl | | G. |
| | | | | | G.mC.mU.mU*mU*mC* |
| | | | | | G*mU*mU* G. |
| 14102 | 858 | 219 | G.mC. G. G. A. A. A. | 220 | P.mA.fU.fU. G. |
| | | | G.mC.mC. A. A.mU.Chl | | G.fC.fU.fU.fU.fC.mC. |
| | | | | | G.mC*mU*mU* A*mU* |
| | | | | | A* U. |
| 14103 | 1012 | 221 | A. A. A.mU.mU.mU.mC. | 222 | P.mA. A. A.fU.A.fC. G.A. A. |
| | | | G.mU. | | A.mU.mU.mU*mC* A* |
| | | | A.mU.mU.mU.Chl | | G* G*mU* G. |
| 14104 | 1014 | 223 | A.mU.mU.mU.mC. | 224 | P.mA. G. A. A. A.fU. A.fC. |
| | | | G.mU. | | G.A. A. |
| | | | A.mU.mU.mU.mC.mU.Chl | | A.mU*mU*mU*mC* A* |
| | | | | | G* G. |
| 14105 | 356 | 225 | A. A. A. G.mC.mC. | 226 | P.mU. G. G.fU.fC. A.fU. G. |
| | | | A.mU. G. A.mC.mC. | | G.fC.mU.mU.mU*mC* |
| | | | A.Chl | | G*mU*mU* G* G. |
| 14106 | 368 | 227 | A.mC. A.mU. G. G. | 228 | P.mA.fU. A.fU.fC. |
| | | | A.mU. G. A.mU. | | A.fU.fC.fC. A.mU. G.mU* |
| | | | A.mU.Chl | | G* G*mU*mC* A* U. |
| 14107 | 1011 | 229 | G. A. A. | 230 | P.mA. A.fU.A.fC. G.A. A. |
| | | | A.mU.mU.mU.mC. | | A.fU.mU.mU.mC* A* G* |
| | | | G.mU. A.mU.mU.Chl | | G*mU* G* U. |
| 14108 | 754 | 231 | G.mC. | 232 | P.mA. A.fU.fC. A. G.A. A. |
| | | | G.mC.mC.mU.mU.mC.mU. | | G. G.mC. G.mC* |
| | | | G. A.mU.mU.Chl | | G*mU*mU*mC* A* G. |
| 14109 | 1021 | 233 | A.mU.mU.mU.mC.mU. | 234 | P.mA.fU.fU.fC. A.fU. G. A. |
| | | | mC. A.mU. G. A. | | G.A. A. A.mU*A*mC*G* |
| | | | A.mU.Chl | | A* A* A. |
| 14110 | 1330 | 235 | mC.mU.mC.mU.mC. | 236 | P.mC.fU. A.fU.fU.fC. A.fU. |
| | | | A.mU. G. A. A.mU. A. | | G. A. G. A. G* A* A*mU* |
| | | | G.Chl | | A* A* C. |
| 14111 | 346 | 237 | A. A. G.mU.mC.mC. A. | 238 | P.mU.fU.fU.fC. G.fU.fU. |
| | | | A.mC. G. A. A. A.Chl | | G. G. A.mC.mU.mU* |
| | | | | | A*mC*mU*mU* G* G. |
| 14112 | 869 | 239 | A.mU. G. A.mU. G. A. G. | 240 | P.mU.fU. G.fC.fU.fC.fU.fC. |
| | | | A. G.mC. A. A.Chl | | A.fU.mC. A.mU*mU* G* |
| | | | | | G*mC*mU* U. |
| 14113 | 701 | 241 | G.mC. G. A. G. G. A. | 242 | P.mU.fU.fC. A. |
| | | | G.mU.mU. G. A. A.Chl | | A.fC.fU.fC.fC.fU.mC. |
| | | | | | G.mC*mU*mU*mU*mC* |
| | | | | | mC* A. |
| 14114 | 896 | 243 | mU. G. A.mU.mU. G. | 244 | P.mU. G. A.fC.fU. A.fU.fC. |
| | | | A.mU. A. G.mU.mC. | | A. A.mU.mC. A*mC* |
| | | | A.Chl | | A*mU*mC* G* G. |
| 14115 | 1035 | 245 | A. G. A.mU. A. G.mU. | 246 | P.mA. G. A.fU. G.fC. |
| | | | G.mC. A.mU.mC.mU.Chl | | A.fC.fU. A.mU.mC.mU* |
| | | | | | A* A*mU*mU*mC* A. |
| 14116 | 1170 | 247 | A.mU. G.mU. G.mU. | 248 | P.mA. A.fU. A. G. A.fU. |
| | | | A.mU.mC.mU. | | A.fC. A.mC. |
| | | | A.mU.mU.Chl | | A.mU*mU*mC* A* |
| | | | | | A*mC* C. |
| 14117 | 1282 | 249 | mU.mU.mC.mU. A.mU. | 250 | P.mU.fU.fC.fU.fU.fC.fU. |
| | | | A. G. A. A. G. A. A.Chl | | A.fU. A. G. A. A*mU* G* |
| | | | | | A* A*mC* A. |
| 14118 | 1537 | 251 | mU.mU. G.mU.mC.mC. | 252 | P.mA. A.fU.fU. G.fC.fU. G. |
| | | | A. G.mC. A. | | G.A.mC. A. A*mC*mC* |
| | | | A.mU.mU.Chl | | G*mU* G* G. |
| 14119 | 692 | 253 | A.mC. A.mU. G. G. A. A. | 254 | P.mU.fC. |
| | | | A. G. C.mG. A.Chl | | G.fC.fU.fU.fU.fC.fC. |
| | | | | | A.mU. G.mU* G*mU* G* |
| | | | | | A* G* G. |
| 14120 | 840 | 255 | G.mC. A. G.mU.mC.mC. | 256 | P.mU. A. A.fU.fC.fU. G. G. |
| | | | A. G. A.mU.mU. A.Chl | | A.fC.mU. G.mC*mU*mU* |
| | | | | | G*mU* G* G. |
| 14121 | 1163 | 257 | mU. G. G.mU.mU. G. A. | 258 | P.mA.fC. A.fC. A.fU.fU.fC. |
| | | | A.mU. G.mU. G.mU.Chl | | A. A.mC.mC. A* A*mU* |
| | | | | | A* A* A* C. |
| 14122 | 789 | 259 | mU.mU. A.mU. G. A. A. | 260 | P.mA.fC.fU.fC. |
| | | | A.mC. G. A. G.mU.Chl | | G.fU.fU.fU.fC. A.mU. A. |
| | | | | | A*mC*mU* G*mU*mC* |
| | | | | | C. |
| 14123 | 841 | 261 | mC. A. G.mU.mC.mC. A. | 262 | P.mA.fU.A. A.fU.fC.fU. G. |
| | | | G. A.mU.mU. A.mU.Chl | | G. A.mC.mU. |
| | | | | | G*mC*mU*mU* G*mU* |
| | | | | | G. |
| 14124 | 852 | 263 | A.mU. A.mU. A. A. | 264 | P.mU.fU.fU.fC.fC. |
| | | | G.mC. G. G. A. A. A.Chl | | G.fC.fU.fU. A.mU. A.mU* |
| | | | | | A* A*mU*mC*mU* G. |
| 14125 | 209 | 265 | mU. A.mC.mC. A. | 266 | P.mU. G.fU.fU.fU. A. |
| | | | G.mU.mU. A. A. A.mC. | | A.fC.fU. G. G.mU. A*mU* |
| | | | A.Chl | | G* G*mC* A* C. |
| 14126 | 1276 | 267 | mU. G.mU.mU.mC. | 268 | P.mU. A.fU. A. G. A. A.fU. |
| | | | A.mU.mU.mC.mU. | | G. A. A.mC. A*mU* A* G* |
| | | | A.mU. A.Chl | | A*mC* A. |
| 14127 | 137 | 269 | mC.mC. G. A.mC.mC. A. | 270 | P.mU.fU.fU.fC.fC.fU.fU. |
| | | | A. G. G. A. A. A.Chl | | G. G.fU.mC. G. G*mC* |
| | | | | | G*mU*mU*mU* G. |
| 14128 | 711 | 271 | G. A. A.mU. G. G.mU. | 272 | P.mG.fU. A.fU. G.fC. |
| | | | G.mC. A.mU. A.mC.Chl | | A.fC.fC. A.mU.mU.mC* |
| | | | | | A* A*mC*mU*mC* C. |
| 14129 | 582 | 273 | A.mU. A.mU. G. A.mU. | 274 | P.mU.fC. G. G.fC.fC. |
| | | | G. G.mC.mC. G. A.Chl | | A.fU.fC. A.mU. A.mU* |
| | | | | | G*mU* G*mU*mC* U. |
| 14130 | 839 | 275 | A. G.mC. A. | 276 | P.mA. A.fU.fC.fU. G. G. |
| | | | G.mU.mC.mC. A. G. | | A.fC.fU. G.mC.mU*mU* |
| | | | A.mU.mU.Chl | | G*mU* G* G* C. |
| 14131 | 1091 | 277 | G.mC. A.mU.mU.mU. A. | 278 | P.mU.fU.fU. G. A.fC.fU. A. |
| | | | G.mU.mC. A. A. A.Chl | | A. A.mU. G.mC* A* A* A* |
| | | | | | G*mU* G. |
| 14132 | 884 | 279 | A. G.mC. | 280 | P.mA.fC. A.fU.fC. G. G. A. |
| | | | A.mU.mU.mC.mC. G. | | A.fU. G.mC.mU*mC* |
| | | | A.mU. G.mU.Chl | | A*mU*mU* G* C. |
| 14133 | 903 | 281 | mU. A. G.mU.mC. A. G. | 282 | P.mA. A. G.fU.fU.fC.fC.fU. |
| | | | G. A. A.mC.mU.mU.Chl | | G. A.mC.mU. A*mU*mC* |
| | | | | | A* A*mU* C. |
| 14134 | 1090 | 283 | mU. G.mC. | 284 | P.mU.fU. G. A.fC.fU. A. A. |
| | | | A.mU.mU.mU. A. | | A.fU. G.mC. A* A* A* |
| | | | G.mU.mC. A. A.Chl | | G*mU* G* A. |
| 14135 | 474 | 285 | G.mU.mC.mU. G. | 286 | P.mA. G. A.fC.fU.fC. |
| | | | A.mU. G. A. | | A.fU.fC. A. G. A.mC*mU* |
| | | | G.mU.mC.mU.Chl | | G* G*mU* G* A. |
| 14136 | 575 | 287 | mU. A. G. A.mC. A.mC. | 288 | P.mU.fC. A.fU. A.fU. G.fU. |
| | | | A.mU. A.mU. G. A.Chl | | G.fU.mC.mU. A*mC*mU* |
| | | | | | G*mU* G* G. |
| 14137 | 671 | 289 | mC. A. G. A.mC. G. A. G. | 290 | P.mA.fU. G.fU.fC.fC.fU.fC. |
| | | | G. A.mC. A.mU.Chl | | G.fU.mC.mU. G*mU* A* |
| | | | | | G*mC* A* U. |
| 14138 | 924 | 291 | mC. A. G.mC.mC. G.mU. | 292 | P.mG. A. A.fU.fU.fC. A.fC. |
| | | | G. A. A.mU.mU.mC.Chl | | G. G.mC.mU. G* |
| | | | | | A*mC*mU*mU*mU* G. |
| 14139 | 1185 | 293 | A. G.mU.mC.mU. G. G. | 294 | P.mU.fU. |
| | | | A. A. A.mU. A. A.Chl | | A.fU.fU.fU.fC.fC. A. G. |
| | | | | | A.mC.mU*mC* A* A* |
| | | | | | A*mU* A. |
| 14140 | 1221 | 295 | A. G.mU.mU.mU. | 296 | P.mG. A. A. G.fC.fC. A.fC. |
| | | | G.mU. G. | | A. A. A.mC.mU* A* A* |
| | | | G.mC.mU.mU.mC.Chl | | A*mC*mU* A. |
| 14141 | 347 | 297 | A. G.mU.mC.mC. A. | 298 | P.mC.fU.fU.fU.fC. |
| | | | A.mC. G. A. A. A. G.Chl | | G.fU.fU. G. G. |
| | | | | | A.mC. mU*mU* |
| | | | | | A*mC*mU*mU* G. |
| 14142 | 634 | 299 | A. A. G.mU.mU.mU.mC. | 300 | P.mG.fU.fC.fU. G.fC. G. A. |
| | | | G.mC. A. G. A.mC.Chl | | A. |
| | | | | | A.mC.mU.mU*mC*mU* |
| | | | | | mU* A* G* A. |
| 14143 | 877 | 301 | A. G.mC. A. A.mU. G. A. | 302 | P.mA. A.fU. G.fC.fU.fC. |
| | | | G.mC. A.mU.mU.Chl | | A.fU.fU. |
| | | | | | G.mC.mU*mC*mU*mC* |
| | | | | | A*mU* C. |
| 14144 | 1033 | 303 | mU.mU. A. G. A.mU. A. | 304 | P.mA.fU. G.fC. A.fC.fU. |
| | | | G.mU. G.mC. A.mU.Chl | | A.fU.fC.mU. A. |
| | | | | | A*mU*mU*mC* A*mU* |
| | | | | | G. |
| 14145 | 714 | 305 | mU. G. G.mU. G.mC. | 306 | P.mC.fU.fU. G.fU. A.fU. |
| | | | A.mU. A.mC. A. A. G.Chl | | G.fC. A.mC.mC. |
| | | | | | A*mU*mU*mC* A* A* C. |
| 14146 | 791 | 307 | A.mU. G. A. A. A.mC. G. | 308 | P.mU. G. A.fC.fU.fC. |
| | | | A. G.mU.mC. A.Chl | | G.fU.fU.fU.mC. A.mU* A* |
| | | | | | A*mC*mU* G* U. |
| 14147 | 813 | 309 | mC.mC. A. G. A. G.mU. | 310 | P.mU.fU.fC. A. G.fC. |
| | | | G.mC.mU. G. A. A.Chl | | A.fC.fU.fC.mU. G. |
| | | | | | G*mU*mC* A*mU*mC* |
| | | | C. | | |
| 14148 | 939 | 311 | mC. A. G.mC.mC. A.mU. | 312 | P.mA. A. A.fU.fU.fC. A.fU. |
| | | | G. A. A.mU.mU.mU.Chl | | G. G.mC.mU. G*mU* G* |
| | | | | | G* A* A* U. |
| 14149 | 1161 | 313 | A.mU.mU. G. | 314 | P.mA.fC. A.fU.fU.fC. A. |
| | | | G.mU.mU. G. A. A.mU. | | A.fC.fC. A. A.mU* A* A* |
| | | | G.mU.Chl | | A*mC*mU* G. |
| 14150 | 1164 | 315 | G. G.mU.mU. G. A. | 316 | P.mU. A.fC. A.fC. |
| | | | A.mU. G.mU. G.mU. | | A.fU.fU.fC. A. A.mC.mC* |
| | | | A.Chl | | A* A*mU* A* A* A. |
| 14151 | 1190 | 317 | G. G. A. A. A.mU. A. | 318 | P.mA.fU.fU. A. G.fU.fU. |
| | | | A.mC.mU. A. A.mU.Chl | | A.fU.fU.mU.mC.mC* A* |
| | | | | | G* A*mC*mU* C. |
| 14152 | 1333 | 319 | mU.mC. A.mU. G. A. | 320 | P.mU.fU.fU.fC.fU. |
| | | | A.mU. A. G. A. A. A.Chl | | A.fU.fU.fC. A.mU. G. A* |
| | | | | | G* A* G* A* A* U. |
| 14153 | 537 | 321 | G.mC.mC. A. G.mC. A. | 322 | P.mU.fU.fC. G. G.fU.fU. |
| | | | A.mC.mC. G. A. A.Chl | | G.fC.fU. G. G.mC* A* G* |
| | | | | | G*mU*mC* C. |
| 14154 | 684 | 323 | mC. A.mC.mC.mU.mC. | 324 | P.mC. A.fU. G.fU. G.fU. G. |
| | | | A.mC. A.mC. A.mU. | | A. G. G.mU. G* A*mU* |
| | | | G.Chl | | G*mU*mC* C. |
| 14155 | 707 | 325 | A. G.mU.mU. G. A. | 326 | P.mG.fC. A.fC.fC. |
| | | | A.mU. G. G.mU. | | A.fU.fU.fC. A. |
| | | | G.mC.Chl | | A.mC.mU*mC*mC*mU* |
| | | | | | mC* G* C. |
| 14156 | 799 | 327 | A. G.mU.mC. A. | 328 | P.mC. A.fU.fC.fC. A. |
| | | | G.mC.mU. G. G. A.mU. | | G.fC.fU. G. |
| | | | G.Chl | | A.mC.mU*mC* |
| | | | | | G*mU*mU*mU* C. |
| 14157 | 853 | 329 | mU. A.mU. A. A. G.mC. | 330 | P.mC.fU.fU.fU.fC.fC. |
| | | | G. G. A. A. A. G.Chl | | G.fC.fU.fU. A.mU. A*mU* |
| | | | | | A* A*mU*mC* U. |
| 14158 | 888 | 331 | mU.mU.mC.mC. G. | 332 | P.mA. A.fU.fC. A.fC. |
| | | | A.mU. G.mU. G. | | A.fU.fC. G. G. A. A*mU* |
| | | | A.mU.mU.Chl | | G*mC*mU*mC* A. |
| 14159 | 1194 | 333 | A.mU. A. A.mC.mU. A. | 334 | P.mA.fC. A.fC. A.fU.fU. A. |
| | | | A.mU. G.mU. G.mU.Chl | | G.fU.mU. |
| | | | | | A.mU*mU*mU*mC*mC* |
| | | | | | A* G. |
| 14160 | 1279 | 335 | mU.mC. | 336 | P.mU.fU.fC.fU. A.fU. A. G. |
| | | | A.mU.mU.mC.mU. | | A. A.mU. G.A* A*mC* |
| | | | A.mU. A. G. A. A.Chl | | A*mU* A* G. |
| 14161 | 1300 | 337 | A. A.mC.mU. A.mU.mC. | 338 | P.mU. A.fC. A. G.fU. G. |
| | | | A.mC.mU. G.mU. A.Chl | | A.fU. A. G.mU.mU*mU* |
| | | | | | G*mC* A*mU* U. |
| 14162 | 1510 | 339 | G.mU.mC. A. A.mU.mU. | 340 | P.mA.fU.A. A. G.fC. A. |
| | | | G.mC.mU.mU. A.mU.Chl | | A.fU.fU. G. A.mC* |
| | | | | | A*mC*mC* A*mC* C. |
| 14163 | 1543 | 341 | A. G.mC. A. A.mU.mU. | 342 | P.mU.fU.fU. A.fU.fU. A. |
| | | | A. A.mU. A. A. A.Chl | | A.fU.fU. G.mC.mU* G* |
| | | | | | G* A*mC* A* A. |
| 14164 | 434 | 343 | A.mC. G. | 344 | P.mU.fC. A.fU.fC. A. G. A. |
| | | | A.mC.mU.mC.mU. G. | | G.fU.mC. G.mU*mU*mC* |
| | | | A.mU. G. A.Chl | | G* A* G* U. |
| 14165 | 600 | 345 | mU. A. G.mU. G.mU. G. | 346 | P.mA.fU. A. A. A.fC.fC. |
| | | | G.mU.mU.mU. | | A.fC. A.mC.mU. |
| | | | A.mU.Chl | | A*mU*mC* A*mC*mC* |
| | | | | | U. |
| 14166 | 863 | 347 | A. A. G.mC.mC. A. | 348 | P.mU.fC. A.fU.fC. A.fU.fU. |
| | | | A.mU. G. A.mU. G. A.Chl | | G. |
| | | | | | G.mC.mU.mU*mU*mC* |
| | | | | | mC* G*mC* U. |
| 14167 | 902 | 349 | A.mU. A. G.mU.mC. A. | 350 | P.mA. G.fU.fU.fC.fC.fU. G. |
| | | | G. G. A. A.mC.mU.Chl | | A.fC.mU. A.mU*mC* A* |
| | | | | | A*mU*mC* A. |
| 14168 | 921 | 351 | A. G.mU.mC. A. | 352 | P.mU.fU.fC. A.fC. G. |
| | | | G.mC.mC. G.mU. G. A. | | G.fC.fU. G. |
| | | | A.Chl | | A.mC.mU*mU*mU* G* |
| | | | | | G* A* A. |
| 14169 | 154 | 353 | A.mC.mU. A.mC.mC. | 354 | P.mU.fU.fC.fU.fC. A.fU. G. |
| | | | A.mU. G. A. G. A. A.Chl | | G.fU. A. G.mU* G* A* |
| | | | | | G*mU*mU* U. |
| 14170 | 217 | 355 | A. A. A.mC. A. G. | 356 | P.mA. A.fU.fC. A. |
| | | | G.mC.mU. G. | | G.fC.fC.fU. |
| | | | A.mU.mU.Chl | | G.mU.mU.mU* A* |
| | | | | | A*mC*mU* G* G. |
| 14171 | 816 | 357 | G. A. G.mU. G.mC.mU. | 358 | P.mG. G.fU.fU.fU.fC. A. |
| | | | G. A. A. A.mC.mC.Chl | | G.fC. A.mC.mU.mC*mU* |
| | | | | | G* G*mU*mC* A. |
| 14172 | 882 | 359 | mU. G. A. G.mC. | 360 | P.mA.fU.fC. G. G. A. A.fU. |
| | | | A.mU.mU.mC.mC. G. | | G.fC.mU.mC. A*mU*mU* |
| | | | A.mU.Chl | | G*mC*mU* C. |
| 14173 | 932 | 361 | A. A.mU.mU.mC.mC. | 362 | P.mU. G. G.fC.fU. G.fU. G. |
| | | | A.mC. A. G.mC.mC. | | G.A. A.mU.mU*mC* |
| | | | A.Chl | | A*mC* G* G* C. |
| 14174 | 1509 | 363 | mU. G.mU.mC. A. | 364 | P.mU. A. A. G.fC. A. |
| | | | A.mU.mU. | | A.fU.fU. G. A.mC. |
| | | | G.mC.mU.mU. A.Chl | | A*mC*mC* A*mC*mC* |
| | | | | | A. |
| 14175 | 157 | 365 | A.mC.mC. A.mU. G. A. | 366 | P.mC. A. A.fU.fU.fC.fU.fC. |
| | | | G. A. A.mU.mU. G.Chl | | A.fU. G. G.mU* A* |
| | | | | | G*mU* G* A* G. |
| 14176 | 350 | 367 | mC.mC. A. A.mC. G. A. | 368 | P.mU. G. G.fC.fU.fU.fU.fC. |
| | | | A. A. G.mC.mC. A.Chl | | G.fU.mU. G. G* |
| | | | | | A*mC*mU*mU* A* C. |
| 14177 | 511 | 369 | mC.mU. G. G.mU.mC. | 370 | P.mA. A.fU.fC. A. G.fU. G. |
| | | | A.mC.mU. G. | | A.fC.mC. A. |
| | | | A.mU.mU.Chl | | G*mU*mU*mC* A*mU* |
| | | | | | C. |
| 14178 | 605 | 371 | mU. G. G.mU.mU.mU. | 372 | P.mA. G.fU.fC.fC. A.fU. A. |
| | | | A.mU. G. G. | | A. A.mC.mC. A*mC* |
| | | | A.mC.mU.Chl | | A*mC*mU* A* U. |
| 14179 | 811 | 373 | G. A.mC.mC. A. G. A. | 374 | P.mC. A. G.fC. |
| | | | G.mU. G.mC.mU. G.Chl | | A.fC.fU.fC.fU. G. |
| | | | | | G.mU.mC* |
| | | | | | A*mU*mC*mC* A* G. |
| 14180 | 892 | 375 | G. A.mU. G.mU. G. | 376 | P.mU. A.fU.fC. A. A.fU.fC. |
| | | | A.mU.mU. G. A.mU. | | A.fC. A.mU.mC* G* G* |
| | | | A.Chl | | A* A*mU* G. |
| 14181 | 922 | 377 | G.mU.mC. A. G.mC.mC. | 378 | P.mA.fU.fU.fC. A.fC. G. |
| | | | G.mU. G. A. A.mU.Chl | | G.fC.fU. G. |
| | | | | | A.mC*mU*mU*mU* G* |
| | | | | | G* A. |
| 14182 | 1169 | 379 | A. A.mU. G.mU. G.mU. | 380 | P.mA.fU.A. G. A.fU. A.fC. |
| | | | A.mU.mC.mU. A.mU.Chl | | A.fC. A.mU.mU*mC* A* |
| | | | | | A*mC*mC* A. |
| 14183 | 1182 | 381 | mU.mU. G. A. | 382 | P.mU.fU.fU.fC.fC. A. G. |
| | | | G.mU.mC.mU. G. G. A. | | A.fC.fU.mC. A. A* A*mU* |
| | | | A. A.Chl | | A* G* A* U. |
| 14184 | 1539 | 383 | G.mU.mC.mC. A. G.mC. | 384 | P.mU.fU. A. A.fU.fU. |
| | | | A. A.mU.mU. A. A.Chl | | G.fC.fU. G. G. A.mC* A* |
| | | | | | A*mC*mC* G* U. |
| 14185 | 1541 | 385 | mC.mC. A. G.mC. A. | 386 | P.mU. A.fU.fU. A. A.fU.fU. |
| | | | A.mU.mU. A. A.mU. | | G.fC.mU. G. G* A*mC* |
| | | | A.Chl | | A* A*mC* C. |
| 14186 | 427 | 387 | G. A.mC.mU.mC. G. A. | 388 | P.mA. G.fU.fC. G.fU.fU.fC. |
| | | | A.mC. G. A.mC.mU.Chl | | G.A. G.mU.mC* A* |
| | | | | | A*mU* G* G* A. |
| 14187 | 533 | 389 | A.mC.mC.mU. | 390 | P.mG.fU.fU. G.fC.fU. G. |
| | | | G.mC.mC. A. G.mC. A. | | G.fC. A. G. |
| | | | A.mC.Chl | | G.mU*mC*mC* G*mU* |
| | | | | | G* G. |
| 18538 | 496 | 391 | G. A.mU. G. A. | 392 | P.mU. A.fU.fC. A. G. |
| | | | A.mU.mC.mU. G. A.mU. | | A.fU.fU.fC. A.fU.fC* A* |
| | | | A.Chl | | G* A* A*fU* G. |
| 18539 | 496 | 393 | mU. G. A.mU. G. A. | 394 | P.mU. A.fU.fC. A. G. |
| | | | A.mU.mC.mU. G. A.mU. | | A.fU.fU.fC. A.fU.fC* A* |
| | | | A.Chl | | G* A* A*fU* G. |
| 18540 | 175 | 395 | A.mU.mU.mU. | 396 | P.mU. G.fC. A. A. A. A. |
| | | | G.mC.mU.mU.mU.mU. | | G.fC. A. A. A.fU*fC* |
| | | | G.mC. A.Chl | | A*fC*fU*fG* C. |
| 18541 | 175 | 397 | G. A.mU.mU.mU. | 398 | P.mU. G.fC. A. A. A. A. |
| | | | G.mC.mU.mU.mU.mU. | | G.fC. A. A. A.fU*fC* |
| | | | G.mC. A.Chl | | A*fC*fU*fG* C. |
| 18542 | 172 | 399 | G.mU. G. | 400 | P.mU. A. A. A. G.fC. A. A. |
| | | | A.mU.mU.mU. | | A.fU.fC. A.fC*fU* G*fC* |
| | | | G.mC.mU.mU.mU. A.Chl | | A* A* U. |
| 18543 | 172 | 401 | A. G.mU. G. | 402 | P.mU. A. A. A. G.fC. A. A. |
| | | | A.mU.mU.mU. | | A.fU.fC. A.fC*fU* G*fC* |
| | | | G.mC.mU.mU.mU. A.Chl | | A* A* U. |
| 18544 | 1013 | 403 | A. A.mU.mU.mU.mC. | 404 | P.mU. A. A. A.fU. A.fC. G. |
| | | | G.mU. A.mU.mU.mU. | | A. A. A.fU.fU*fU*fC* A* |
| | | | A.Chl | | G* G* U. |
| 18545 | 1013 | 405 | A. A. A.mU.mU.mU.mC. | 406 | P.mU. A. A. A.fU. A.fC. G. |
| | | | G.mU. A.mU.mU.mU. | | A. A. A.fU.fU*fU*fC* A* |
| | | | A.Chl | | G* G* U. |
| 18546 | 952 | 407 | mC. A.mC. A. G.mC.mC. | 408 | P.mU.fU.fU. C. A.fU. G. |
| | | | A.mU. G. A. A. A.Chl | | G.fC.fU. G.fU. G* A* A* |
| | | | | | A*fU*fU* C. |
| 18547 | 952 | 409 | mU.mC. A.mC. A. | 410 | P.mU.fU.fU. C. A.fU. G. |
| | | | G.mC.mC. A.mU. G. A. | | G.fC.fU. G.fU. G* A* A* |
| | | | A. A.Chl | | A*fU*fU* C. |
| 18548 | 174 | 411 | G. A.mU.mU.mU. | 412 | P.mU.fC. A. A. A. A. G.fC. |
| | | | G.mC.mU.mU.mU.mU. | | A. A. A.fU.fC* A*fC*fU* |
| | | | G. A.Chl | | G*fC* A. |
| 18549 | 174 | 413 | mU. G. A.mU.mU.mU. | 414 | P.mU.fC. A. A. A. A. G.fC. |
| | | | G.mC.mU.mU.mU.mU. | | A. A. A.fU.fC* A*fC*fU* |
| | | | G. A.Chl | | G*fC* A. |
| 18550 | 177 | 415 | mU.mU. | 416 | P.mU. A. G. G.fC. A. A. A. |
| | | | G.mC.mU.mU.mU.mU. | | A. G.fC. A. A* A*fU*fC* |
| | | | G.mC.mC.mU. A.Chl | | A*fC* U. |
| 18551 | 177 | 417 | mU.mU.mU. | 418 | P.mU. A. G. G.fC. A. A. A. |
| | | | G.mC.mU.mU.mU.mU. | | A. G.fC. A. A* A*fU*fC* |
| | | | G.mC.mC.mU. A.Chl | | A*fC* U. |
| 18552 | 1150 | 419 | mU.mU.mU.mC.mU.mC. | 420 | P.mU.fU. A. A. A.fC.fU. G. |
| | | | A. G.mU.mU.mU. A. | | A. G. A. A. A* G* A* A* |
| | | | A.Chl | | G*fC* A. |
| 18553 | 1089 | 421 | mU.mU. G.mC. | 422 | P.mU. G. A.fC.fU. A. A. |
| | | | A.mU.mU.mU. A. | | A.fU. G.fC. A. A* A* |
| | | | G.mU.mC. A.Chl | | G*fU* G* A* G. |
| 18554 | 1086 | 423 | A.mC.mU.mU.mU. | 424 | P.mU.fU. A. A. A.fU. G.fC. |
| | | | G.mC. A.mU.mU.mU. A. | | A. A. A. G.fU* G* A* G* |
| | | | A.Chl | | A* A* A. |
| 18555 | 1093 | 425 | A.mU.mU.mU. A. | 426 | P.mU.fU.fU.fU.fU. G. |
| | | | G.mU.mC. A. A. A. A. | | A.fC.fU. A. A. A.fU* G*fC* |
| | | | A.Chl | | A* A* A* G. |
| 18556 | 1147 | 427 | mU.mU.mC.mU.mU.mU. | 428 | P.mU. A.fC.fU. G. A. G. A. |
| | | | mC.mU.mC. A. G.mU. | | A. A. G. A. A* G*fC* |
| | | | A.Chl | | A*fU*fU* U. |
| 18557 | 1148 | 429 | mU.mC.mU.mU.mU.mC. | 430 | P.mU. A. A.fC.fU. G. A. G. |
| | | | mU.mC. A. G.mU.mU. | | A. A. A. G. A* A* G*fC* |
| | | | A.Chl | | A*fU* U. |
| 18558 | 1128 | 431 | G. A. A. A. G. A. G. A. | 432 | P.mU. A.fU. |
| | | | A.mC. A.mU. A.Chl | | G.fU.fU.fC.fU.fC.fU.fU.fU. |
| | | | | | fC* A*fU*fU*fU*fU* G. |
| 18559 | 1087 | 433 | mC.mU.mU.mU. G.mC. | 434 | P.mU.fC.fU. A. A. A.fU. |
| | | | A.mU.mU.mU. A. G. | | G.fC. A. A. A. G*fU* G* |
| | | | A.Chl | | A* G* A* A. |
| 18560 | 1088 | 435 | mU.mU.mU. G.mC. | 436 | P.mU. A.fC.fU. A. A. A.fU. |
| | | | A.mU.mU.mU. A. G.mU. | | G.fC. A. A. A* G*fU* G* |
| | | | A.Chl | | A* G* A. |
| 18561 | 1083 | 437 | mC.mU.mC. | 438 | P.mU. A.fU. G.fC. A. A. A. |
| | | | A.mC.mU.mU.mU. | | G.fU. G. A. G* A* A* |
| | | | G.mC. A.mU. A.Chl | | A*fU*fU* G. |
| 18562 | 1081 | 439 | mU.mU.mC.mU.mC. | 440 | P.mU. G.fC. A. A. A. G.fU. |
| | | | A.mC.mU.mU.mU. | | G. A. G. A. A* A*fU*fU* |
| | | | G.mC. A.Chl | | G*fU* A. |
| 18563 | 555 | 441 | mC. A.mC.mU.mC.mC. | 442 | P.mU. A.fC. A. A.fC.fU. G. |
| | | | A. G.mU.mU. G.mU. | | G. A. G.fU. G* A* A* A* |
| | | | A.Chl | | A*fC*fU. |
| 18564 | 1125 | 443 | A. A.mU. G. A. A. A. G. | 444 | P.mU.fU.fU.fC.fU.fC.fU.fU. |
| | | | A. G. A. A. A.Chl | | fU.fC. A.fU.fU*fU*fU* |
| | | | | | G*fC*fU* A. |
| 18565 | 168 | 445 | mU. G.mC. A. G.mU. G. | 446 | P.mU.fC. A. A. A.fU.fC. |
| | | | A.mU.mU.mU.mG. | | A.fC.fU. G.fC. A* |
| | | | A.Chl | | A*fU*fU*fC*fU* C. |
| 18566 | 1127 | 447 | mU. G. A. A. A. G. A. G. | 448 | P.mU.fU. |
| | | | A. A.mC. A. A.Chl | | G.fU.fU.fC.fU.fC.fU.fU.fU. |
| | | | | | fC. A*fU*fU*fU*fU* G* |
| | | | | | C. |
| 18567 | 1007 | 449 | A.mC.mC.mU. G. A. A. | 450 | P.mU. G. A. A. |
| | | | A.mU.mU.mU.mC. A.Chl | | A.fU.fU.fU.fC. A. G. G.fU* |
| | | | | | G*fU*fU*fU* A* U. |
| 18568 | 164 | 451 | G. A. A.mU.mU. G.mC. | 452 | P.mU.fU.fC. A.fC.fU. G.fC. |
| | | | A. G.mU. G. A. A.Chl | | A. A.fU.fU.fC*fU*fC* |
| | | | | | A*fU* G* G. |
| 18569 | 222 | 453 | G. G.mC.mU. G. | 454 | P.mU.fC.fC. A. G. A. |
| | | | A.mU.mU.mC.mU. G. G. | | A.fU.fC. A. G.fC.fC*fU* |
| | | | A.Chl | | G*fU*fU*fU* A. |
| 20612 | 172 | 455 | A. G.mU. G. | 456 | P.mU. A. A. A. G.fC. A. A. |
| | | | A.mU.mU.mU. | | A.fU.mC. A.mC*mU* |
| | | | G.mC.mU.mU.mU. A.Chl | | G*mC* A* A* U. |
| 20613 | 172 | 457 | A. G.mU. G. | 458 | P.mU. A. A. A. G.fC. A. A. |
| | | | A.mU.mU.mU. | | A.fU.fC. A.mC*fU* |
| | | | G.mC.mU.mU.mU. A.Chl | | G*mC* A* A* U. |
| 20614 | 172 | 459 | A. G.mU. G. | 460 | P.mU. A. A. A. G. C. A. A. |
| | | | A.mU.mU.mU. | | A. U.mC. A.mC*mU* |
| | | | G.mC.mU.mU.mU. A.Chl | | G*mC* A* A* U. |
| 20615 | 172 | 461 | A. G.mU. G. | 462 | P.mU. A. A. A. G.fC. A. A. |
| | | | A.mU.mU.mU. | | A.fU.mC. |
| | | | G.mC.mU.mU.mU. A.Chl | | A.mC*mU*mG*mC*mA* |
| | | | | | mA* U. |

Key
Chl = cholesterol with hydroxyprolinol linker
TEG-chl = cholesterol with TEG linker
m = 2′Ome
f = 2′fluoro
* = phosphorothioate llinkage
. = phosphodiester linkage

TABLE 4
PTGS2 (Accession Number NM_000963.2) sd-rxRNA sequences

| Oligo | Start | SEQ ID | | SEQ ID | |
|---|---|---|---|---|---|
| Number | Site | NO | Sense sequence | NO | Antisense sequence |
| 14422 | 451 | 463 | mC. A.mC. | 464 | P.mU.fC. A. A.fU.fC. A. |
| | | | A.mU.mU.mU. G. | | A. A.fU. G.mU. G* |
| | | | A.mU.mU. G. A.Chl | | A*mU*mC*mU* G* G. |
| 14423 | 1769 | 465 | mC. A.mC.mU. | 466 | P.mA. A.fU.fU. G. A. G. |
| | | | G.mC.mC.mU.mC. | | G.fC. A. G.mU. |
| | | | A. A.mU.mU.Chl | | G*mU*mU* G* A*mU* |
| | | | | | G. |
| 14424 | 1464 | 467 | A. A. A.mU. | 468 | P.mA. A. G. A.fC.fU. G. |
| | | | A.mC.mC. A. | | G.fU. |
| | | | G.mU.mC.mU.mU. | | A.mU.mU.mU*mC* |
| | | | Chl | | A*mU*mC*mU* G. |
| 14425 | 453 | 469 | mC. A.mU.mU.mU. | 470 | P.mU. G.fU.fC. A. |
| | | | G. A.mU.mU. G. | | A.fU.fC. A. A. A.mU. |
| | | | A.mC. A.Chl | | G*mU* G* A*mU*mC* |
| | | | | | U. |
| 17388 | 285 | 471 | G. A. A. A. | 472 | P.mU.fU. G. A. G.fC. A. |
| | | | A.mC.mU. | | G.fU.fU.fU.fU.fC*fU*fC* |
| | | | G.mC.mU.mC. A. | | fC* A*fU* A. |
| | | | A.Chl | | |
| 17389 | 520 | 473 | A.mC.mC.mU.mC. | 474 | P.mU. A. A.fU. A. G. G. |
| | | | mU.mC.mC.mU. | | A. G. A. G. G.fU*fU* A* |
| | | | A.mU.mU. A.Chl | | G* A* G* A. |
| 17390 | 467 | 475 | mU.mC.mC. | 476 | P.mU.fU. A. A. G.fU.fU. |
| | | | A.mC.mC. A. | | G. G.fU. G. G. A*fC*fU* |
| | | | A.mC.mU.mU. A. | | G*fU*fC* A. |
| | | | A.Chl | | |
| 17391 | 467 | 477 | G.mU.mC.mC. | 478 | P.mU.fU. A. A. G.fU.fU. |
| | | | A.mC.mC. A. | | G. G.fU. G. G. A*fC*fU* |
| | | | A.mC.mU.mU. A. | | G*fU*fC* A. |
| | | | A.Chl | | |
| 17392 | 524 | 479 | mC.mU.mC.mC.mU. | 480 | P.mU. G.fU. A.fU. A. |
| | | | A.mU.mU. A.mU. | | A.fU. A. G. G. A. G* A* |
| | | | A.mC. A.Chl | | G* G*fU*fU* A. |
| 17393 | 448 | 481 | G. A.mU.mC. | 482 | P.mU.fU.fC. A. A. A.fU. |
| | | | A.mC. | | G.fU. G. A.fU.fC*fU* G* |
| | | | A.mU.mU.mU. G. | | G* A*fU* G. |
| | | | A. A.Chl | | |
| 17394 | 448 | 483 | A. G. A.mU.mC. | 484 | P.mU.fU.fC. A. A. A.fU. |
| | | | A.mC. | | G.fU. G. A.fU.fC*fU* G* |
| | | | A.mU.mU.mU. G. | | G* A*fU* G. |
| | | | A. A.Chl | | |
| 17395 | 519 | 485 | A. | 486 | P.mU. A.fU. A. G. G. A. |
| | | | A.mC.mC.mU.mC. | | G. A. G. G.fU.fU* A* G* |
| | | | mU.mC.mC.mU. | | A* G* A* A. |
| | | | A.mU. A.Chl | | |
| 17396 | 437 | 487 | G.mU.mU. G. | 488 | P.mU.fC.fU. G. G. A.fU. |
| | | | A.mC. | | G.fU.fC. A. A.fC* A*fC* |
| | | | A.mU.mC.mC. A. G. | | A*fU* A* A. |
| | | | A.Chl | | |
| 17397 | 406 | 489 | mC.mC.mU.mU.mC. | 490 | P.mU.fU.fU.fC. G. A. A. |
| | | | mC.mU.mU.mC. G. | | G. G. A. A. G. G* G* A* |
| | | | A. A. A.Chl | | A*fU* G* U. |
| 17398 | 339 | 491 | A.mC.mU.mC.mC. | 492 | P.mU.fU. G.fU. |
| | | | A. A. A.mC. A.mC. | | G.fU.fU.fU. G. G. A. |
| | | | A. A.Chl | | G.fU* G* G* G*fU*fU* |
| | | | | | U. |
| 17399 | 339 | 493 | mC. | 494 | P.mU.fU. G.fU. |
| | | | A.mC.mU.mC.mC. | | G.fU.fU.fU. G. G. A. |
| | | | A. A. A.mC. A.mC. | | G.fU* G* G* G*fU*fU* |
| | | | A. A.Chl | | U. |
| 17400 | 338 | 495 | mC. | 496 | P.mU. G.fU. G.fU.fU.fU. |
| | | | A.mC.mU.mC.mC. | | G. G. A. G.fU. G* G* |
| | | | A. A. A.mC. A.mC. | | G*fU*fU*fU* C. |
| | | | A.Chl | | |
| 17401 | 468 | 497 | mC.mC. A.mC.mC. | 498 | P.mU. G.fU. A. A. |
| | | | A. A.mC.mU.mU. | | G.fU.fU. G. G.fU. G. G* |
| | | | A.mC. A.Chl | | A*fC*fU* G*fU* C. |
| 17402 | 468 | 499 | mU.mC.mC. | 500 | P.mU. G.fU. A. A. |
| | | | A.mC.mC. A. | | G.fU.fU. G. G.fU. G. G* |
| | | | A.mC.mU.mU. | | A*fC*fU* G*fU* C. |
| | | | A.mC. A.Chl | | |
| 17403 | 1465 | 501 | A. A.mU. A.mC.mC. | 502 | P.mU. A. A. G. A.fC.fU. |
| | | | A. | | G. G.fU. A.fU.fU*fU*fC* |
| | | | G.mU.mC.mU.mU. | | A*fU*fC* U. |
| | | | A.Chl | | |
| 17404 | 243 | 503 | G. A.mC.mC. A. | 504 | P.mU.fC.fU.fU. A.fU. |
| | | | G.mU. A.mU. A. A. | | A.fC.fU. G. G.fU.fC* A* |
| | | | G. A.Chl | | A* A*fU*fC* C. |
| 17405 | 1472 | 505 | G.mU.mC.mU.mU. | 506 | P.mU.fU.fC. A.fU.fU. A. |
| | | | mU.mU. A. A.mU. | | A. A. A. G.A.fC*fU*G* |
| | | | G. A. A.Chl | | G*fU* A* U. |
| 17406 | 2446 | 507 | A. | 508 | P.mU. A. G. A.fC. A.fU. |
| | | | A.mU.mU.mU.mC. | | G. A. A. A.fU.fU* |
| | | | A.mU. | | A*fC*fU* G* G* U. |
| | | | G.mU.mC.mU. | | |
| | | | A.Chl | | |
| 17407 | 449 | 509 | A.mU.mC. A.mC. | 510 | P.mU. A.fU.fC. A. A. |
| | | | A.mU.mU.mU. G. | | A.fU. G.fU. G. |
| | | | A.mU. A.Chl | | A.fU*fC*fU* G* G* A* |
| | | | | | U. |
| 17408 | 449 | 511 | G. A.mU.mC. | 512 | P.mU. A.fU.fC. A. A. |
| | | | A.mC. | | A.fU. G.fU. G. |
| | | | A.mU.mU.mU. G. | | A.fU*fC*fU* G* G* A* |
| | | | A.mU. A.Chl | | U. |
| 17409 | 444 | 513 | mU.mC.mC. A. G. | 514 | P.mU. A.fU. G.fU. G. |
| | | | A.mU.mC. A.mC. | | A.fU.fC.fU. G. G. A*fU* |
| | | | A.mU. A.Chl | | G*fU*fC* A* A. |
| 17410 | 1093 | 515 | mU. A.mC.mU. G. | 516 | P.mU.fC.fU.fC.fC.fU. |
| | | | A.mU. A. G. G. A. G. | | A.fU.fC. A. G.fU. |
| | | | A.Chl | | A*fU*fU* A* G*fC* C. |
| 17411 | 1134 | 517 | G.mU. G.mC. A. | 518 | P.mU.fC. A. A. G.fU. |
| | | | A.mC. A.mC.mU.fU. | | G.fU.fU. G.mC. A.fC* |
| | | | G. A.Chl | | A*fU* A* A*fU* C. |
| 17412 | 244 | 519 | A.mC.mC. A. | 520 | P.mU. A.fC.fU.fU. A.fU. |
| | | | G.mU. A.mU. A. A. | | A.fC.fU. G. G.fU*fC* A* |
| | | | G.mU. A.Chl | | A* A*fU* C. |
| 17413 | 1946 | 521 | G. A. A. | 522 | P.mU.fU.fC. A.fU.fU. A. |
| | | | G.mU.mC.mU. A. | | G. A.fC.mU.fU.fC*fU* |
| | | | A.mU. G. A. A.Chl | | A*fC* A* G* U. |
| 17414 | 638 | 523 | A. A. G. A. A. G. A. | 524 | P.mU. A. |
| | | | A. A. G.mU.mU. | | A.fC.fU.fU.fU.fC.fU.fU.fC. |
| | | | A.Chl | | fU.fU* A* G* A* A* |
| | | | | | G* C. |
| 17415 | 450 | 525 | mU.mC. A.mC. | 526 | P.mU. A. A.fU.fC. A. A. |
| | | | A.mU.mU.mU. G. | | A.fU. G.fU. G. |
| | | | A.mU.mU. A.Chl | | A*fU*fC*fU* G* G* A. |
| 17416 | 450 | 527 | A.mU.mC. A.mC. | 528 | P.mU. A. A.fU.fC. A. A. |
| | | | A.mU.mU.mU. G. | | A.fU. G.fU. G. |
| | | | A.mU.mU. A.Chl | | A*fU*fC*fU* G* G* A. |
| 17417 | 452 | 529 | A.mC. | 530 | P.mU.fU.fC. A. A.fU.fC. |
| | | | A.mU.mU.mU. G. | | A. A. A.fU. G.fU* G* |
| | | | A.mU.mU. G. A. | | A*fU*fC*fU* G. |
| | | | A.Chl | | |
| 17418 | 452 | 531 | mC. A.mC. | 532 | P.mU.fU.fC. A. A.fU.fC. |
| | | | A.mU.mU.mU. G. | | A. A. A.fU. G.fU* G* |
| | | | A.mU.mU. G. A. | | A*fU*fC*fU* G. |
| | | | A.Chl | | |
| 17419 | 454 | 533 | A.mU.mU.mU. G. | 534 | P.mU.fU. G.fU.fC. A. |
| | | | A.mU.mU. G. A.mC. | | A.fU.fC. A. A. A.fU* |
| | | | A. A.Chl | | G*fU* G* A*fU* C. |
| 17420 | 454 | 535 | mC. A.mU.mU.mU. | 536 | P.mU.fU. G.fU.fC. A. |
| | | | G. A.mU.mU. G. | | A.fU.fC. A. A. A.fU* |
| | | | A.mC. A. A.Chl | | G*fU* G* A*fU* C. |
| 17421 | 1790 | 537 | mC. A.mU.mC.mU. | 538 | P.mU.fU.fU. A.fU.fU. |
| | | | G.mC. A. A.mU. A. | | G.fC. A. G. A.fU. G* A* |
| | | | A. A.Chl | | G* A* G* A* C. |
| 17422 | 1790 | 539 | mU.mC. | 540 | P.mU.fU.fU. A.fU.fU. |
| | | | A.mU.mC.mU. | | G.fC. A. G. A.fU. G* A* |
| | | | G.mC. A. A.mU. A. | | G* A* G* A* C. |
| | | | A. A.Chl | | |
| 21180 | 448 | 541 | G. A.mU.mC. | 542 | P.mU.fU.fC. A.mA. A.fU. |
| | | | A.mC. | | G.fU. G. A.mU.mC*mU* |
| | | | A.mU.mU.mU. G. | | G* G* A*mU* G. |
| | | | A. A.TEG-Chl | | |
| 21181 | 448 | 543 | G. A.mU.mC. | 544 | P.mU.fU.fC. A.mA. A.fU. |
| | | | A.mC. | | G.fU. G. |
| | | | A.mU.mU.mU. G. | | A.fU.fC*fU*mG*mG*mA* |
| | | | A. A.TEG-Chl | | fU* G. |
| 21182 | 448 | 545 | G. A.mU.mC. | 546 | P.mU.fU.fC. A. A. A.fU. |
| | | | A.mC. | | G.fU. G. A.fU.fC*fU* G* |
| | | | A.mU.mU.mU. | | G* A*fU* G. |
| | | | G*mA*mA.TEG-Chl | | |
| 21183 | 448 | 547 | mG*mA*mU.mC. | 548 | P.mU.fU.fC. A. A. A.fU. |
| | | | A.mC. | | G.fU. G. A.fU.fC*fU* G* |
| | | | A.mU.mU.mU. | | G* A*fU* G. |
| | | | G*mA*mA.TEG-Chl | | |
| 21184 | 448 | 549 | mG*mA*mU.mC.mA. | 550 | P.mU.fU.fC. A. A. A.fU. |
| | | | mC.mA.mU.mU. | | G.fU. G. A.fU.fC*fU* G* |
| | | | mU.mG*mA*mA.TEG- | | G* A*fU* G. |
| | | | Chl | | |
| 21185 | 449 | 551 | G. A.mU.mC. | 552 | P.mU. A.fU.fC. A. A. |
| | | | A.mC. | | A.fU. G.fU. G. |
| | | | A.mU.mU.mU. G. | | A.fU.fCfU* G* G* |
| | | | A.mU. A.TEG-Chl | | A*fU* G. |
| 21186 | 449 | 553 | G. A.mU.mC. | 554 | P.mU. A.fU.fC. A. A. |
| | | | A.mC. | | A.fU. G.fU. G. |
| | | | A.mU.mU.mU. G. | | A.mU.mC*mU* G* G* |
| | | | A.mU. A.TEG-Chl | | A*mU* G. |
| 21187 | 449 | 555 | G. A.mU.mC. | 556 | P.mU. A.fU.fC. A.mA. |
| | | | A.mC. | | A.fU. G.fU. G. |
| | | | A.mU.mU.mU. G. | | A.mU.mC*mU* G* G* |
| | | | A.mU. A.TEG-Chl | | A*mU* G. |
| 21188 | 449 | 557 | G. A.mU.mC. | 558 | P.mU. A.fU.fC. A. A. |
| | | | A.mC. | | A.fU. G.fU. G. |
| | | | A.mU.mU.mU. G. | | A.mU.mC*mU*mG*mG* |
| | | | A.mU. A.TEG-Chl | | mA*mU* G. |
| 21189 | 449 | 559 | G. A.mU.mC. | 560 | P.mU. A.fU.fC. A.mA. |
| | | | A.mC. | | A.fU. G.fU. G. |
| | | | A.mU.mU.mU. G. | | A.mU.mC*mU*mG*mG* |
| | | | A.mU. A.TEG-Chl | | mA*mU* G. |
| 21190 | 449 | 561 | G. A.mU.mC. | 562 | P.mU. A.fU.fC. A. A. |
| | | | A.mC. | | A.fU. G.fU. G. |
| | | | A.mU.mU.mU. G. | | A.fU.fC*fU*mG*mG*mA* |
| | | | A.mU. A.TEG-Chl | | fU* G. |
| 21191 | 449 | 563 | G. A.mU.mC. | 564 | P.mU. A.fU.fC. A.mA. |
| | | | A.mC. | | A.fU. G.fU. G. |
| | | | A.mU.mU.mU. G. | | A.fU.fC*fU*mG*mG*mA* |
| | | | A.mU. A.TEG-Chl | | fU* G. |
| 21192 | 449 | 565 | G. A.mU.mC. | 566 | P.mU. A.fU.fC. A. A. |
| | | | A.mC. | | A.fU. G.fU. G. |
| | | | A.mU.mU.mU. G. | | A.fU.mC*fU*mG*mG* |
| | | | A.mU. A.TEG-Chl | | mA*fU* G. |
| 21193 | 449 | 567 | G. A.mU.mC. | 568 | P.mU. A.fU.fC. A. A. |
| | | | A.mC. | | A.fU. G.fU. G. |
| | | | A.mU.mU.mU. G. | | A.fU*fC*fU* G* G* A* |
| | | | A*mU*mA.TEG-Chl | | U. |
| 21194 | 449 | 569 | mG*mA*mU.mC. | 570 | P.mU. A.fU.fC. A. A. |
| | | | A.mC. | | A.fU. G.fU. G. |
| | | | A.mU.mU.mU. G. | | A.fU*fC*fU* G* G* A* |
| | | | A*mU*mA.TEG-Chl | | U. |
| 21195 | 449 | 571 | mG*mA*mU.mC.mA. | 572 | P.mU. A.fU.fC. A. A. |
| | | | mC.mA.mU.mU. | | A.fU. G.fU. G. |
| | | | mU.mG.mA*mU*mA. | | A.fU*fC*fU* G* G* A* |
| | | | TEG-Chl | | U. |
| 20620 | 449 | 573 | G. A.mU.mC. | 574 | P.mU. A.fU.fC. A. A. |
| | | | A.mC. | | A.fU. G.fU. G. |
| | | | A.mU.mU.mU. G. | | A.mU*mC*mU* G* G* |
| | | | A.mU. A.Chl-TEG | | A* U. |
| 20621 | 449 | 575 | G. A.mU.mC. | 576 | P.mU. A.fU.fC. A. A. |
| | | | A.mC. | | A.fU. G.fU. G. |
| | | | A.mU.mU.mU. G. | | A.mU*fC*mU* G* G* |
| | | | A.mU. A.Chl-TEG | | A* U. |
| 20622 | 449 | 577 | G. A.mU.mC. | 578 | P.mU. A. U. C. A. A. A. |
| | | | A.mC. | | U. G. U. G. |
| | | | A.mU.mU.mU. G. | | A.mU*mC*mU* G* G* |
| | | | A.mU. A.Chl-TEG | | A* U. |
| 20623 | 449 | 579 | G. A.mU.mC. | 580 | P.mU. A.fU.fC. A. A. |
| | | | A.mC. | | A.fU. G.fU. G. |
| | | | A.mU.mU.mU. G. | | A.mU*mC*mU*mG*mG* |
| | | | A.mU. A.Chl-TEG | | mA* U. |
| 20588 | 448 | 581 | G. A.mU.mC. | 582 | P.mU.fU.fC. A. A. A.fU. |
| | | | A.mC. | | G.fU. G. A.mU.mC*mU* |
| | | | A.mU.mU.mU. G. | | G* G* A*mU* G. |
| | | | A. A.Chl-TEG | | |
| 20589 | 448 | 583 | G. A.mU.mC. | 584 | P.mU.fU.fC. A. A. A.fU. |
| | | | A.mC. | | G.fU. G. A.mU.fC*mU* |
| | | | A.mU.mU.mU. G. | | G* G* A*fU* G. |
| | | | A. A.Chl-TEG | | |
| 20590 | 448 | 585 | G. A.mU.mC. | 586 | P.mU. U. C. A. A. A. U. |
| | | | A.mC. | | G. U. G. A.mU.mC*mU* |
| | | | A.mU.mU.mU. G. | | G* G* A*mU* G. |
| | | | A. A.Chl-TEG | | |
| 20591 | 448 | 587 | G. A.mU.mC. | 588 | P.mU.fU.fC. A. A. A.fU. |
| | | | A.mC. | | G.fU. G. |
| | | | A.mU.mU.mU. G. | | A.fU.fC*fU*mG*mG*mA* |
| | | | A. A.Chl-TEG | | fU* G. |

Key
Chl = cholesterol with hydroxyprolinol linker
TEG-chl = cholesterol with TEG linker
m = 2′Ome
f = 2′fluoro
* = phosphorothioate llinkage
. = phosphodiester linkage

TABLE 5
CTGF (Accession Number: NM_001901.2) sd-rxRNA sequences

| Oligo | Start | SEQ ID | | SEQ ID | |
|---|---|---|---|---|---|
| Number | Site | NO | Sense sequence | NO | Antisense sequence |
| 13980 | 1222 | 589 | A.mC. A. G. G. A. | 590 | P.mU. A.fC. |
| | | | A. G. A.mU. G.mU. | | A.fU.fC.fU.fU.fC.fC.mU. |
| | | | A.Chl | | G.mU* A* G*mU* |
| | | | | | A*mC* A. |
| 13981 | 813 | 591 | G. A. G.mU. G. G. | 592 | P.mA. G. G.fC. |
| | | | A. G.mC. | | G.fC.fU.fC.fC. |
| | | | G.mC.mC.mU.Chl | | A.mC.mU.mC*mU* |
| | | | | | G*mU* G* G* U. |
| 13982 | 747 | 593 | mC. G. A.mC.mU. | 594 | P.mU. |
| | | | G. G. A. A. G. A.mC. | | G.fU.fC.fU.fU.fC.fC. A. |
| | | | A.Chl | | G.mU.mC. G* G*mU* |
| | | | | | A* A* G* C. |
| 13983 | 817 | 595 | G. G. A. G.mC. | 596 | P.mG. A. A.fC. A. G. |
| | | | G.mC.mC.mU. | | G.fC. G.fC.mU.mC.mC* |
| | | | G.mU.mU.mC.Chl | | A*mC*mU*mC*mU* |
| | | | | | G. |
| 13984 | 1174 | 597 | G.mC.mC. | 598 | P.mC. A. G.fU.fU. G.fU. |
| | | | A.mU.mU. A.mC. A. | | A. A.fU. G. G.mC* A* |
| | | | A.mC.mU. G.Chl | | G* G*mC* A* C. |
| 13985 | 1005 | 599 | G. A. | 600 | P.mA. G.fC.fC. A. G. A. |
| | | | G.mC.mU.mU.mU. | | A. A. G.mC.mU.mC* A* |
| | | | mC.mU. G. | | A* A*mC*mU* U. |
| | | | G.mC.mU.Chl | | |
| 13986 | 814 | 601 | A. G.mU. G. G. A. | 602 | P.mC. A. G. G.fC. |
| | | | G.mC. | | G.fC.fU.fC.fC. |
| | | | G.mC.mC.mU. | | A.mC.mU*mC*mU* |
| | | | G.Chl | | G*mU* G* G. |
| 13987 | 816 | 603 | mU. G. G. A. G.mC. | 604 | P.mA. A.fC. A. G. G.fC. |
| | | | G.mC.mC.mU. | | G.fC.fU.mC.mC. |
| | | | G.mU.mU.Chl | | A*mC*mU*mC*mU* |
| | | | | | G* U. |
| 13988 | 1001 | 605 | G.mU.mU.mU. G. | 606 | P.mA. G. A. A. A. |
| | | | A. | | G.fC.fU.fC. A. A. |
| | | | G.mC.mU.mU.mU. | | A.mC*mU*mU* G* |
| | | | mC.mU.Chl | | A*mU* A. |
| 13989 | 1173 | 607 | mU. G.mC.mC. | 608 | P.mA. G.fU.fU. G.fU. A. |
| | | | A.mU.mU. A.mC. A. | | A.fU. G. G.mC. A* G* |
| | | | A.mC.mU.Chl | | G*mC* A*mC* A. |
| 13990 | 749 | 609 | A.mC.mU. G. G. A. | 610 | P.mC. G.fU. |
| | | | A. G. A.mC. A.mC. | | G.fU.fC.fU.fU.fC.fC. A. |
| | | | G.Chl | | G.mU*mC* G* G*mU* |
| | | | | | A* A. |
| 13991 | 792 | 611 | A. A.mC.mU. | 612 | P.mG. G. A.fC.fC. A. G. |
| | | | G.mC.mC.mU. G. | | G.fC. A. G.mU.mU* G* |
| | | | G.mU.mC.mC.Chl | | G*mC*mU*mC* U. |
| 13992 | 1162 | 613 | A. G. | 614 | P.mC. A. G. G.fC. A.fC. |
| | | | A.mC.mC.mU. | | A. G. |
| | | | G.mU. | | G.mU.mC.mU*mU* G* |
| | | | G.mC.mC.mU. | | A*mU* G* A. |
| | | | G.Chl | | |
| 13993 | 811 | 615 | mC. A. G. A. G.mU. | 616 | P.mG.fC. G.fC.fU.fC.fC. |
| | | | G. G. A. G.mC. | | A.fC.fU.mC.mU. G*mU* |
| | | | G.mC.Chl | | G* G*mU*mC* U. |
| 13994 | 797 | 617 | mC.mC.mU. G. | 618 | P.mG. G.fU.fC.fU. G. G. |
| | | | G.mU.mC.mC. A. G. | | A.fC.fC. A. G. G*mC* A* |
| | | | A.mC.mC.Chl | | G*mU*mU* G. |
| 13995 | 1175 | 619 | mC.mC. A.mU.mU. | 620 | P.mA.fC. A. G.fU.fU. |
| | | | A.mC. A. A.mC.mU. | | G.fU. A. A.mU. G. |
| | | | G.mU.Chl | | G*mC* A* G* G*mC* |
| | | | | | A. |
| 13996 | 1172 | 621 | mC.mU. G.mC.mC. | 622 | P.mG.fU.fU. G.fU. A. |
| | | | A.mU.mU. A.mC. A. | | A.fU. G. G.mC. A. G* |
| | | | A.mC.Chl | | G*mC* A*mC* A* G. |
| 13997 | 1177 | 623 | A.mU.mU. A.mC. | 624 | P.mG. G. A.fC. A. |
| | | | A. A.mC.mU. | | G.fU.fU. G.fU. A. A.mU* |
| | | | G.mU.mC.mC.Chl | | G* G*mC* A* G* G. |
| 13998 | 1176 | 625 | mC. A.mU.mU. | 626 | P.mG. A.fC. A. G.fU.fU. |
| | | | A.mC. A. A.mC.mU. | | G.fU. A. A.mU. G* |
| | | | G.mU.mC.Chl | | G*mC* A* G* G* C. |
| 13999 | 812 | 627 | A. G. A. G.mU. G. | 628 | P.mG. G.fC. |
| | | | G. A. G.mC. | | G.fC.fU.fC.fC. |
| | | | G.mC.mC.Chl | | A.fC.mU.mC.mU* |
| | | | | | G*mU* G* G*mU* C. |
| 14000 | 745 | 629 | A.mC.mC. G. | 630 | P.mU.fC.fU.fU.fC.fC. A. |
| | | | A.mC.mU. G. G. A. | | G.fU.fC. G. G.mU* A* |
| | | | A. G. A.Chl | | A* G*mC*mC* G. |
| 14001 | 1230 | 631 | A.mU. G.mU. | 632 | P.mU. G.fU.fC.fU.fC.fC. |
| | | | A.mC. G. G. A. G. | | G.fU. A.mC. |
| | | | A.mC. A.Chl | | A.mU*mC*mU*mU*mC* |
| | | | | | mC* U. |
| 14002 | 920 | 633 | G.mC.mC.mU.mU. | 634 | P.mA. G.fC.fU.fU.fC. |
| | | | G.mC. G. A. A. | | G.fC. A. A. G. |
| | | | G.mC.mU.Chl | | G.mC*mC*mU* G* |
| | | | | | A*mC* C. |
| 14003 | 679 | 635 | G.mC.mU. G.mC. | 636 | P.mC. |
| | | | G. A. G. G. A. | | A.fC.fU.fC.fC.fU.fC. |
| | | | G.mU. G.Chl | | G.fC. A. G.mC* |
| | | | | | A*mU*mU*mU*mC* |
| | | | | | C. |
| 14004 | 992 | 637 | G.mC.mC.mU. | 638 | P.mA. A. A.fC.fU.fU. G. |
| | | | A.mU.mC. A. A. | | A.fU. A. G. |
| | | | G.mU.mU.mU.Chl | | G.mC*mU*mU* G* G* |
| | | | | | A* G. |
| 14005 | 1045 | 639 | A. | 640 | P.mA.fC.fU.fC.fC. A.fC. |
| | | | A.mU.mU.mC.mU. | | A. G. A. A.mU.mU*mU* |
| | | | G.mU. G. G. A. | | A* G*mC*mU* C. |
| | | | G.mU.Chl | | |
| 14006 | 1231 | 641 | mU. G.mU. A.mC. | 642 | P.mA.fU. |
| | | | G. G. A. G. A.mC. | | G.fU.fC.fU.fC.fC. G.fU. |
| | | | A.mU.Chl | | A.mC. |
| | | | | | A*mU*mC*mU*mU*mC* |
| | | | | | C. |
| 14007 | 991 | 643 | A. G.mC.mC.mU. | 644 | P.mA. A.fC.fU.fU. G. |
| | | | A.mU.mC. A. A. | | A.fU. A. G. |
| | | | G.mU.mU.Chl | | G.mC.mU*mU* G* G* |
| | | | | | A* G* A. |
| 14008 | 998 | 645 | mC. A. A. | 646 | P.mA. A. G.fC.fU.fC. A. |
| | | | G.mU.mU.mU. G. | | A. A.fC.mU.mU. G* |
| | | | A. | | A*mU* A* G* G* C. |
| | | | G.mC.mU.mU.Chl | | |
| 14009 | 1049 | 647 | mC.mU. G.mU. G. | 648 | P.mA.fC. A.fU. |
| | | | G. A. G.mU. A.mU. | | A.fC.fU.fC.fC. A.mC. A. |
| | | | G.mU.Chl | | G* A* A*mU*mU*mU* |
| | | | | | A. |
| 14010 | 1044 | 649 | A. A. | 650 | P.mC.fU.fC.fC. A.fC. A. |
| | | | A.mU.mU.mC.mU. | | G. A. A.mU.mU.mU* A* |
| | | | G.mU. G. G. A. | | G*mC*mU*mC* G. |
| | | | G.Chl | | |
| 14011 | 1327 | 651 | mU.mU.mU.mC. A. | 652 | P.mU. G.fU. G.fC.fU. |
| | | | G.mU. A. G.mC. | | A.fC.fU. G. A. A. |
| | | | A.mC. A.Chl | | A*mU*mC* |
| | | | | | A*mU*mU* U. |
| 14012 | 1196 | 653 | mC. A. A.mU. G. | 654 | P.mA. A. A. G. A.fU. |
| | | | A.mC. | | G.fU.fC. A.mU.mU. |
| | | | A.mU.mC.mU.mU. | | G*mU*mC*mU*mC*mC* |
| | | | mU.Chl | | G. |
| 14013 | 562 | 655 | A. G.mU. | 656 | P.mG.fU. G.fC. A.fC.fU. |
| | | | A.mC.mC. A. G.mU. | | G. G.fU. |
| | | | G.mC. A.mC.Chl | | A.mC.mU*mU* G*mC* |
| | | | | | A* G* C. |
| 14014 | 752 | 657 | G. G. A. A. G. | 658 | P.mA. A. A.fC. G.fU. |
| | | | A.mC. A.mC. | | G.fU.fC.fU.mU.mC.mC* |
| | | | G.mU.mU.mU.Chl | | A* G*mU*mC* G* G. |
| 14015 | 994 | 659 | mC.mU. A.mU.mC. | 660 | P.mU.fC. A. A. |
| | | | A. A. | | A.fC.fU.fU. G. A.mU. A. |
| | | | G.mU.mU.mU. G. | | G* G*mC*mU*mU* G* |
| | | | A.Chl | | G. |
| 14016 | 1040 | 661 | A. G.mC.mU. A. A. | 662 | P.mA.fC. A. G. A. |
| | | | A.mU.mU.mC.mU. | | A.fU.fU.fU. A. |
| | | | G.mU.Chl | | G.mC.mU*mC* G* |
| | | | | | G*mU* A* U. |
| 14017 | 1984 | 663 | A. G. G.mU. A. G. | 664 | P.mU.fU. A.fC. |
| | | | A. A.mU. G.mU. A. | | A.fU.fU.fC.fU. |
| | | | A.Chl | | A.mC.mC.mU* A*mU* |
| | | | | | G* G*mU* G. |
| 14018 | 2195 | 665 | A. G.mC.mU. G. | 666 | P.mA. A. A.fC.fU. G. |
| | | | A.mU.mC. A. | | A.fU.fC. A. G.mC.mU* |
| | | | G.mU.mU.mU.Chl | | A*mU* A*mU* A* G. |
| 14019 | 2043 | 667 | mU.mU.mC.mU. | 668 | P.mU. A.fU.fC.fU. G. A. |
| | | | G.mC.mU.mC. A. G. | | G.fC. A. G. A. |
| | | | A.mU. A.Chl | | A*mU*mU*mU*mC*mC* |
| | | | | | A. |
| 14020 | 1892 | 669 | mU.mU. | 670 | P.mU.fU. A. A.fC.fU.fU. |
| | | | A.mU.mC.mU. A. A. | | A. G. A.mU. A. |
| | | | G.mU.mU. A. A.Chl | | A*mC*mU* G*mU* A* |
| | | | | | C. |
| 14021 | 1567 | 671 | mU. A.mU. A.mC. | 672 | P.mU. A.fU.fU. |
| | | | G. A. G.mU. A. | | A.fC.fU.fC. G.fU. A.mU. |
| | | | A.mU. A.Chl | | A* A* G* A*mU* G* C. |
| 14022 | 1780 | 673 | G. A.mC.mU. G. G. | 674 | P.mA. A. G.fC.fU. |
| | | | A.mC. A. | | G.fU.fC.fC. A. |
| | | | G.mC.mU.mU.Chl | | G.mU.mC*mU* A* |
| | | | | | A*mU*mC* G. |
| 14023 | 2162 | 675 | A.mU. G. | 676 | P.mU. A. A.fU. A. A. A. |
| | | | G.mC.mC.mU.mU. | | G. G.fC.mC. |
| | | | mU. A.mU.mU. | | A.mU*mU*mU* |
| | | | A.Chl | | G*mU*mU* C. |
| 14024 | 1034 | 677 | A.mU. A.mC.mC. | 678 | P.mU.fU.fU. A. |
| | | | G. A. G.mC.mU. A. | | G.fC.fU.fC. G. G.mU. |
| | | | A. A.Chl | | A.mU* |
| | | | | | G*mU*mC*mU*mU* |
| | | | | | C. |
| 14025 | 2264 | 679 | mU.mU. G.mU.mU. | 680 | P.mA.fC. |
| | | | G. A. G. A. G.mU. | | A.fC.fU.fC.fU.fC. A. |
| | | | G.mU.Chl | | A.mC. A. A* A*mU* A* |
| | | | | | A* A* C. |
| 14026 | 1032 | 681 | A.mC. A.mU. | 682 | P.mU. A. G.fC.fU.fC. G. |
| | | | A.mC.mC. G. A. | | G.fU. A.mU. |
| | | | G.mC.mU. A.Chl | | G.mU*mC*mU*mU*mC* |
| | | | | | A* U. |
| 14027 | 1535 | 683 | A. G.mC. A. G. A. | 684 | P.mU. A. |
| | | | A. A. G. G.mU.mU. | | A.fC.fC.fU.fU.fU.fC.fU. |
| | | | A.Chl | | G.mC.mU* G* G*mU* |
| | | | | | A*mC* C. |
| 14028 | 1694 | 685 | A. G.mU.mU. | 686 | P.mU.fU. A. A. G. G. A. |
| | | | G.mU.mU.mC.mC. | | A.fC. A. A.mC.mU*mU* |
| | | | mU.mU. A. A.Chl | | G* A*mC*mU* C. |
| 14029 | 1588 | 687 | A.mU.mU.mU. G. | 688 | P.mU.fU. A.fC. |
| | | | A. A. G.mU. G.mU. | | A.fC.fU.fU.fC. A. A. |
| | | | A. A.Chl | | A.mU* A* G*mC* A* |
| | | | | | G* G. |
| 14030 | 928 | 689 | A. A. G.mC.mU. G. | 690 | P.mU.fC.fC. A. G. |
| | | | A.mC.mC.mU. G. G. | | G.fU.fC. A. |
| | | | A.Chl | | G.mC.mU.mU*mC* |
| | | | | | G*mC* A* A* G. |
| 14031 | 1133 | 691 | G. G.mU.mC. | 692 | P.mC.fU.fU.fC.fU.fU.fC. |
| | | | A.mU. G. A. A. G. A. | | A.fU. G. |
| | | | A. G.Chl | | A.mC.mC*mU*mC* |
| | | | | | G*mC*mC* G. |
| 14032 | 912 | 693 | A.mU. G. | 694 | P.mA. A. G. G.fC.fC.fU. |
| | | | G.mU.mC. A. G. | | G. A.fC.mC. A.mU* |
| | | | G.mC.mC.mU.mU. | | G*mC* A*mC* A* G. |
| | | | Chl | | |
| 14033 | 753 | 695 | G. A. A. G. A.mC. | 696 | P.mC. A. A. A.fC. G.fU. |
| | | | A.mC. | | G.fU.fC.mU.mU.mC*mC* |
| | | | G.mU.mU.mU. | | A* G*mU*mC* G. |
| | | | G.Chl | | |
| 14034 | 918 | 697 | A. G. | 698 | P.mC.fU.fU.fC. G.fC. A. |
| | | | G.mC.mC.mU.mU. | | A. G. G.mC.mC.mU* G* |
| | | | G.mC. G. A. A. | | A*mC*mC* A* U. |
| | | | G.Chl | | |
| 14035 | 744 | 699 | mU. A.mC.mC. G. | 700 | P.mC.fU.fU.fC.fC. A. |
| | | | A.mC.mU. G. G. A. | | G.fU.fC. G. G.mU. A* A* |
| | | | A. G.Chl | | G*mC*mC* G* C. |
| 14036 | 466 | 701 | A.mC.mC. G.mC. | 702 | P.mC.fC. G. |
| | | | A. A. G. A.mU.mC. | | A.fU.fC.fU.fU. G.fC. G. |
| | | | G. G.Chl | | G.mU*mU* G* |
| | | | | | G*mC*mC* G. |
| 14037 | 917 | 703 | mC. A. G. | 704 | P.mU.fU.fC. G.fC. A. A. |
| | | | G.mC.mC.mU.mU. | | G. G.fC.mC.mU. G* |
| | | | G.mC. G. A. A.Chl | | A*mC*mC* A*mU* G. |
| 14038 | 1038 | 705 | mC. G. A. | 706 | P.mA. G. A. A.fU.fU.fU. |
| | | | G.mC.mU. A. A. | | A. G.fC.mU.mC. G* |
| | | | A.mU.mU.mC.mU. | | G*mU* A*mU* G* U. |
| | | | Chl | | |
| 14039 | 1048 | 707 | mU.mC.mU. G.mU. | 708 | P.mC. A.fU. |
| | | | G. G. A. G.mU. | | A.fC.fU.fC.fC. A.fC. A. G. |
| | | | A.mU. G.Chl | | A* A*mU*mU*mU* A* |
| | | | | | G. |
| 14040 | 1235 | 709 | mC. G. G. A. G. | 710 | P.mU. G.fC.fC. A.fU. |
| | | | A.mC. A.mU. G. | | G.fU.fC.fU.mC.mC. |
| | | | G.mC. A.Chl | | G*mU* A*mC* A*mU* |
| | | | | | C. |
| 14041 | 868 | 711 | A.mU. G. A.mC. A. | 712 | P.mG. A. G. G.fC. |
| | | | A.mC. | | G.fU.fU. G.fU.mC. |
| | | | G.mC.mC.mU.mC.Chl | | A.mU*mU* G* G*mU* |
| | | | | | A* A. |
| 14042 | 1131 | 713 | G. A. G. G.mU.mC. | 714 | P.mU.fC.fU.fU.fC. A.fU. |
| | | | A.mU. G. A. A. G. | | G. A.fC.mC.mU.mC* |
| | | | A.Chl | | G*mC*mC* G*mU* C. |
| 14043 | 1043 | 715 | mU. A. A. | 716 | P.mU.fC.fC. A.fC. A. G. |
| | | | A.mU.mU.mC.mU. | | A. A.fU.mU.mU. A* |
| | | | G.mU. G. G. A.Chl | | G*mC*mU*mC* G* G. |
| 14044 | 751 | 717 | mU. G. G. A. A. G. | 718 | P.mA. A.fC. G.fU. |
| | | | A.mC. A.mC. | | G.fU.fC.fU.fU.mC.mC. |
| | | | G.mU.mU.Chl | | A* G*mU*mC* G* G* |
| | | | | | U. |
| 14045 | 1227 | 719 | A. A. G. A.mU. | 720 | P.mC.fU.fC.fC. G.fU. |
| | | | G.mU. A.mC. G. G. | | A.fC. |
| | | | A. G.Chl | | A.fU.mC.mU.mU*mC* |
| | | | | | mC*mU* G*mU* A. |
| 14046 | 867 | 721 | A. A.mU. G. A.mC. | 722 | P.mA. G. G.fC. G.fU.fU. |
| | | | A. A.mC. | | G.fU.fC. A.mU.mU* G* |
| | | | G.mC.mC.mU.Chl | | G*mU* A* A* C. |
| 14047 | 1128 | 723 | G. G.mC. G. A. G. | 724 | P.mU.fC. A.fU. G. |
| | | | G.mU.mC. A.mU. | | A.fC.fC.fU.fC. |
| | | | G. A.Chl | | G.mC.mC* G*mU*mC* |
| | | | | | A* G* G. |
| 14048 | 756 | 725 | G. A.mC. A.mC. | 726 | P.mG. G.fC.fC. A. A. |
| | | | G.mU.mU.mU. G. | | A.fC. G.fU. |
| | | | G.mC.mC.Chl | | G.mU.mC*mU*mU*mC* |
| | | | | | mC* A* G. |
| 14049 | 1234 | 727 | A.mC. G. G. A. G. | 728 | P.mG.fC.fC. A.fU. |
| | | | A.mC. A.mU. G. | | G.fU.fC.fU.fC.mC. |
| | | | G.mC.Chl | | G.mU* A*mC* |
| | | | | | A*mU*mC* U. |
| 14050 | 916 | 729 | mU.mC. A. G. | 730 | P.mU.fC. G.fC. A. A. G. |
| | | | G.mC.mC.mU.mU. | | G.fC.fC.mU. G. |
| | | | G.mC. G. A.Chl | | A*mC*mC* A*mU* G* |
| | | | | | C. |
| 14051 | 925 | 731 | G.mC. G. A. A. | 732 | P.mA. G. G.fU.fC. A. |
| | | | G.mC.mU. G. | | G.fC.fU.fU.mC. G.mC* |
| | | | A.mC.mC.mU.Chl | | A* A* G* G*mC* C. |
| 14052 | 1225 | 733 | G. G. A. A. G. | 734 | P.mC.fC. G.fU. A.fC. |
| | | | A.mU. G.mU. | | A.fU.fC.fU.mU.mC.mC* |
| | | | A.mC. G. G.Chl | | mU* G*mU* A* G* U. |
| 14053 | 445 | 735 | G.mU. G. | 736 | P.mG. A. G.fC.fC. G. A. |
| | | | A.mC.mU.mU.mC. | | A. G.fU.mC. A.mC* A* |
| | | | G. | | G* A* A* G* A. |
| | | | G.mC.mU.mC.Chl | | |
| 14054 | 446 | 737 | mU. G. | 738 | P.mG. G. A. G.fC.fC. G. |
| | | | A.mC.mU.mU.mC. | | A. A. G.mU.mC. A*mC* |
| | | | G. | | A* G* A* A* G. |
| | | | G.mC.mU.mC.mC.Chl | | |
| 14055 | 913 | 739 | mU. G. G.mU.mC. | 740 | P.mC. A. A. G. |
| | | | A. G. | | G.fC.fC.fU. G. A.mC.mC. |
| | | | G.mC.mC.mU.mU. | | A*mU* G*mC* A*mC* |
| | | | G.Chl | | A. |
| 14056 | 997 | 741 | mU.mC. A. A. | 742 | P.mA. G.fC.fU.fC. A. A. |
| | | | G.mU.mU.mU. G. | | A.fC.fU.mU. G. A*mU* |
| | | | A. G.mC.mU.Chl | | A* G* G*mC* U. |
| 14057 | 277 | 743 | G.mC.mC. A. G. A. | 744 | P.mC.fU. G.fC. A. |
| | | | A.mC.mU. G.mC. A. | | G.fU.fU.fC.fU. G. |
| | | | G.Chl | | G.mC*mC* G* A*mC* |
| | | | | | G* G. |
| 14058 | 1052 | 745 | mU. G. G. A. G.mU. | 746 | P.mG. G.fU. A.fC. A.fU. |
| | | | A.mU. G.mU. | | A.fC.fU.mC.mC. A*mC* |
| | | | A.mC.mC.Chl | | A* G* A* A* U. |
| 14059 | 887 | 747 | G.mC.mU. A. G. A. | 748 | P.mC.fU. |
| | | | G. A. A. G.mC. A. | | G.fC.fU.fU.fC.fU.fC.fU. |
| | | | G.Chl | | A. G.mC*mC*mU* |
| | | | | | G*mC* A* G. |
| 14060 | 914 | 749 | G. G.mU.mC. A. G. | 750 | P.mG.fC. A. A. G. |
| | | | G.mC.mC.mU.mU. | | G.fC.fC.fU. G. |
| | | | G.mC.Chl | | A.mC.mC* A*mU* |
| | | | | | G*mC* A* C. |
| 14061 | 1039 | 751 | G. A. G.mC.mU. A. | 752 | P.mC. A. G. A. |
| | | | A. | | A.fU.fU.fU. A. |
| | | | A.mU.mU.mC.mU. | | G.mC.mU.mC* G* |
| | | | G.Chl | | G*mU* A*mU* G. |
| 14062 | 754 | 753 | A. A. G. A.mC. | 754 | P.mC.fC. A. A. A.fC. |
| | | | A.mC. | | G.fU. |
| | | | G.mU.mU.mU. G. | | G.fU.mC.mU.mU*mC* |
| | | | G.Chl | | mC* A* G*mU* C. |
| 14063 | 1130 | 755 | mC. G. A. G. | 756 | P.mC.fU.fU.fC. A.fU. G. |
| | | | G.mU.mC. A.mU. | | A.fC.fC.mU.mC. |
| | | | G. A. A. G.Chl | | G*mC*mC* |
| | | | | | G*mU*mC* A. |
| 14064 | 919 | 757 | G. | 758 | P.mG.fC.fU.fU.fC. G.fC. |
| | | | G.mC.mC.mU.mU. | | A. A. G. G.mC.mC*mU* |
| | | | G.mC. G. A. A. | | G* A*mC*mC* A. |
| | | | G.mC.Chl | | |
| 14065 | 922 | 759 | mC.mU.mU. G.mC. | 760 | P.mU.fC. A. |
| | | | G. A. A. G.mC.mU. | | G.fC.fU.fU.fC. G.fC. A. |
| | | | G. A.Chl | | A. G* G*mC*mC*mU* |
| | | | | | G* A. |
| 14066 | 746 | 761 | mC.mC. G. | 762 | P.mG.fU.fC.fU.fU.fC.fC. |
| | | | A.mC.mU. G. G. A. | | A. G.fU.mC. G. G*mU* |
| | | | A. G. A.mC.Chl | | A* A* G*mC* C. |
| 14067 | 993 | 763 | mC.mC.mU. | 764 | P.mC. A. A. A.fC.fU.fU. |
| | | | A.mU.mC. A. A. | | G. A.fU. A. G. |
| | | | G.mU.mU.mU. | | G*mC*mU*mU* G* G* |
| | | | G.Chl | | A. |
| 14068 | 825 | 765 | mU. | 766 | P.mA. G. G.fU.fC.fU.fU. |
| | | | G.mU.mU.mC.mC. | | G. G. A. A.mC. A* G* |
| | | | A. A. G. | | G*mC* G*mC* U. |
| | | | A.mC.mC.mU.Chl | | |
| 14069 | 926 | 767 | mC. G. A. A. | 768 | P.mC. A. G. G.fU.fC. A. |
| | | | G.mC.mU. G. | | G.fC.fU.mU.mC. G*mC* |
| | | | A.mC.mC.mU. | | A* A* G* G* C. |
| | | | G.Chl | | |
| 14070 | 923 | 769 | mU.mU. G.mC. G. | 770 | P.mG.fU.fC. A. |
| | | | A. A. G.mC.mU. G. | | G.fC.fU.fU.fC. G.mC. A. |
| | | | A.mC.Chl | | A* G* G*mC*mC*mU* |
| | | | | | G. |
| 14071 | 866 | 771 | mC. A. A.mU. G. | 772 | P.mG. G.fC. G.fU.fU. |
| | | | A.mC. A. A.mC. | | G.fU.fC. A.mU.mU. G* |
| | | | G.mC.mC.Chl | | G*mU* A* A*mC* C. |
| 14072 | 563 | 773 | G.mU. A.mC.mC. | 774 | P.mC. G.fU. G.fC. |
| | | | A. G.mU. G.mC. | | A.fC.fU. G. G.mU. |
| | | | A.mC. G.Chl | | A.mC*mU*mU* |
| | | | | | G*mC* A* G. |
| 14073 | 823 | 775 | mC.mC.mU. | 776 | P.mG.fU.fC.fU.fU. G. G. |
| | | | G.mU.mU.mC.mC. | | A. A.fC. A. G. G*mC* |
| | | | A. A. G. A.mC.Chl | | G*mC*mU*mC* C. |
| 14074 | 1233 | 777 | mU. A.mC. G. G. A. | 778 | P.mC.fC. A.fU. |
| | | | G. A.mC. A.mU. G. | | G.fU.fC.fU.fC.fC. G.mU. |
| | | | G.Chl | | A*mC* |
| | | | | | A*mU*mC*mU* U. |
| 14075 | 924 | 779 | mU. G.mC. G. A. A. | 780 | P.mG. G.fU.fC. A. |
| | | | G.mC.mU. G. | | G.fC.fU.fU.fC. G.mC. A* |
| | | | A.mC.mC.Chl | | A* G* G*mC*mC* U. |
| 14076 | 921 | 781 | mC.mC.mU.mU. | 782 | P.mC. A. G.fC.fU.fU.fC. |
| | | | G.mC. G. A. A. | | G.fC. A. A. G. |
| | | | G.mC.mU. G.Chl | | G*mC*mC*mU* G* A* |
| | | | | | C. |
| 14077 | 443 | 783 | mC.mU. G.mU. G. | 784 | P.mG.fC.fC. G. A. A. |
| | | | A.mC.mU.mU.mC. | | G.fU.fC. A.mC. A. G* A* |
| | | | G. G.mC.Chl | | A* G* A* G* G. |
| 14078 | 1041 | 785 | G.mC.mU. A. A. | 786 | P.mC. A.fC. A. G. A. |
| | | | A.mU.mU.mC.mU. | | A.fU.fU.fU. A. |
| | | | G.mU. G.Chl | | G.mC*mU*mC* G* |
| | | | | | G*mU* A. |
| 14079 | 1042 | 787 | mC.mU. A. A. | 788 | P.mC.fC. A.fC. A. G. A. |
| | | | A.mU.mU.mC.mU. | | A.fU.fU.mU. A. |
| | | | G.mU. G. G.Chl | | G*mC*mU*mC* G* G* |
| | | | | | U. |
| 14080 | 755 | 789 | A. G. A.mC. A.mC. | 790 | P.mG.fC.fC. A. A. A.fC. |
| | | | G.mU.mU.mU. G. | | G.fU. |
| | | | G.mC.Chl | | G.mU.mC.mU*mU*mC* |
| | | | | | mC* A* G* U. |
| 14081 | 467 | 791 | mC.mC. G.mC. A. | 792 | P.mG.fC. C.fG. A. |
| | | | A. G. A.mU.mC. G. | | U.fC.fU.fU.fG. C.mG. |
| | | | G.mC.Chl | | G*mU*mU* G* G*mC* |
| | | | | | C. |
| 14082 | 995 | 793 | mU. A.mU.mC. A. | 794 | P.mC.fU.fC. A. A. |
| | | | A. G.mU.mU.mU. | | A.fC.fU.fU. G. A.mU. A* |
| | | | G. A. G.Chl | | G* G*mC*mU*mU* G. |
| 14083 | 927 | 795 | G. A. A. G.mC.mU. | 796 | P.mC.fC. A. G. G.fU.fC. |
| | | | G. A.mC.mC.mU. G. | | A. G.fC.mU.mU.mC* |
| | | | G.Chl | | G*mC* A* A* G* G. |
| 17356 | 1267 | 797 | A.mC. A.mU.mU. | 798 | P.mU. A.fU. G. A. |
| | | | A. A.mC.mU.mC. | | G.mU.fU. A. A.fU. |
| | | | A.mU. A.Chl | | G.fU*fC*fU*fC*fU*fC* |
| | | | | | A. |
| 17357 | 1267 | 799 | G. A.mC. | 800 | P.mU. A.fU. G. A. |
| | | | A.mU.mU. A. | | G.mU.fU. A. A.fU. |
| | | | A.mC.mU.mC. | | G.fU*fC*fU*fC*fU*fC* |
| | | | A.mU. A.Chl | | A. |
| 17358 | 1442 | 801 | mU. G. A. A. G. A. | 802 | P.mU.fU. A. A.fC. |
| | | | A.mU. G.mU.mU. | | A.fU.fU.fC.fU.fU.fC. A* |
| | | | A. A.Chl | | A* A*fC*fC* A* G. |
| 17359 | 1442 | 803 | mU.mU. G. A. A. G. | 804 | P.mU.fU. A. A.fC. |
| | | | A. A.mU. | | A.fU.fU.fC.fU.fU.fC. A* |
| | | | G.mU.mU. A. A.Chl | | A* A*fC*fC* A* G. |
| 17360 | 1557 | 805 | G. A.mU. A. G.mC. | 806 | P.mU.fU. A. A. G. A.fU. |
| | | | A.mU.mC.mU.mU. | | G.fC.fU. A.fU.fC*fU* G* |
| | | | A. A.Chl | | A*fU* G* A. |
| 17361 | 1557 | 807 | A. G. A.mU. A. | 808 | P.mU.fU. A. A. G. A.fU. |
| | | | G.mC. | | G.fC.fU. A.fU.fC*fU* G* |
| | | | A.mU.mC.mU.mU. | | A*fU* G* A. |
| | | | A. A.Chl | | |
| 17362 | 1591 | 809 | mU. G. A. A. G.mU. | 810 | P.mU. A. A.fU.fU. A.fC. |
| | | | G.mU. A. | | A.fC.fU.fU.fC. A* A* |
| | | | A.mU.mU. A.Chl | | A*fU* A* G* C. |
| 17363 | 1599 | 811 | A. A.mU.mU. G. A. | 812 | P.mU.fU.fC.fC.fU.fU.fC.fU. |
| | | | G. A. A. G. G. A. | | fC. A. A.fU.fU* A*fC* |
| | | | A.Chl | | A*fC*fU* U. |
| 17364 | 1601 | 813 | mU.mU. G. A. G. A. | 814 | P.mU.fU.fU.fU.fC.fC.fU. |
| | | | A. G. G. A. A. A. | | fU.fC.fU.fC. A. |
| | | | A.Chl | | A*fU*fU* A*fC* A* C. |
| 17365 | 1732 | 815 | mC. | 816 | P.mU.fC. G. A. A.fU.fC. |
| | | | A.mU.mU.mC.mU. | | A. G. A. A.fU. G*fU*fC* |
| | | | G. A.mU.mU.mC. | | A* G* A* G. |
| | | | G. A.Chl | | |
| 17366 | 1734 | 817 | mU.mU.mC.mU. G. | 818 | P.mU.fU.fU.fC. G. A. |
| | | | A.mU.mU.mC. G. | | A.fU.fC. A. G. A. A*fU* |
| | | | A. A. A.Chl | | G*fU*fC* A* G. |
| 17367 | 1770 | 819 | mC.mU. G.mU.mC. | 820 | P.mU.fU.fC.fU. A. |
| | | | G. A.mU.mU. A. G. | | A.fU.fC. G. A.fC. A. G* |
| | | | A. A.Chl | | G* A*fU*fU*fC* C. |
| 17368 | 1805 | 821 | mU.mU.mU. | 822 | P.mU. G.fU.fU. A.fC. A. |
| | | | G.mC.mC.mU. | | G. G.fC. A. A. |
| | | | G.mU. A. A.mC. | | A*fU*fU*fC* A*fC* U. |
| | | | A.Chl | | |
| 17369 | 1805 | 823 | A.mU.mU.mU. | 824 | P.mU. G.fU.fU. A.fC. A. |
| | | | G.mC.mC.mU. | | G. G.fC. A. A. |
| | | | G.mU. A. A.mC. | | A*fU*fU*fC* A*fC* U. |
| | | | A.Chl | | |
| 17370 | 1815 | 825 | A.mC. A. A. | 826 | P.mU. A. A.fU.fC.fU. G. |
| | | | G.mC.mC. A. G. | | G.fC.fU.fU. G.fU*fU* |
| | | | A.mU.mU. A.Chl | | A*fC* A* G* G. |
| 17371 | 1815 | 827 | A. A.mC. A. A. | 828 | P.mU. A. A.fU.fC.fU. G. |
| | | | G.mC.mC. A. G. | | G.fC.fU.fU. G.fU*fU* |
| | | | A.mU.mU. A.Chl | | A*fC* A* G* G. |
| 17372 | 2256 | 829 | mC. A. | 830 | P.mU. A.fC. A. A. A.fU. |
| | | | G.mU.mU.mU. | | A. A. A.fC.fU. |
| | | | A.mU.mU.mU. | | G*fU*fC*fC* G* A* A. |
| | | | G.mU. A.Chl | | |
| 17373 | 2265 | 831 | mU. G.mU.mU. G. | 832 | P.mU. A.fC. |
| | | | A. G. A. G.mU. | | A.fC.fU.fC.fU.fC. A. A.fC. |
| | | | G.mU. A.Chl | | A* A* A*fU* A* A* A. |
| 17374 | 2265 | 833 | mU.mU. G.mU.mU. | 834 | P.mU. A.fC. |
| | | | G. A. G. A. G.mU. | | A.fC.fU.fC.fU.fC. A. A.fC. |
| | | | G.mU. A.Chl | | A* A* A*fU* A* A* A. |
| 17375 | 2295 | 835 | mU. G.mC. | 836 | P.mU.fU. A. G. A. A. A. |
| | | | A.mC.mC.mU.mU. | | G. G.fU. G.fC. A* A* |
| | | | mU.mC.mU. A. | | A*fC* A*fU* G. |
| | | | A.Chl | | |
| 17376 | 2295 | 837 | mU.mU. G.mC. | 838 | P.mU.fU. A. G. A. A. A. |
| | | | A.mC.mC.mU.mU. | | G. G.fU. G.fC. A* A* |
| | | | mU.mC.mU. A. | | A*fC* A*fU* G. |
| | | | A.Chl | | |
| 17377 | 1003 | 839 | mU.mU. G. A. | 840 | P.mU.fC. A. G. A. A. A. |
| | | | G.mC.mU.mU.mU. | | G.fC.fU.fC. A. A* |
| | | | mC.mU. G. A.Chl | | A*fC*fU*fU* G* A. |
| 17378 | 2268 | 841 | mU. G. A. G. A. | 842 | P.mU. G.fU.fC. A.fC. |
| | | | G.mU. G.mU. G. | | A.fC.fU.fC.fU.fC. A* |
| | | | A.mC. A.Chl | | A*fC* A* A* A* U. |
| 17379 | 2272 | 843 | A. G.mU. G.mU. G. | 844 | P.mU.fU.fU.fU. G. |
| | | | A.mC.mC. A. A. A. | | G.fU.fC. A.fC. |
| | | | A.Chl | | A.fC.fU*fC*fU*fC* A* |
| | | | | | A* C. |
| 17380 | 2272 | 845 | G. A. G.mU. G.mU. | 846 | P.mU.fU.fU.fU. G. |
| | | | G. A.mC.mC. A. A. | | G.fU.fC. A.fC. |
| | | | A. A.Chl | | A.fC.fU*fC*fU*fC* A* |
| | | | | | A* C. |
| 17381 | 2273 | 847 | G.mU. G.mU. G. | 848 | P.mU.fU.fU.fU.fU. G. |
| | | | A.mC.mC. A. A. A. | | G.fU.fC. A.fC. |
| | | | A. A.Chl | | A.fC*fU*fC*fU*fC* A* |
| | | | | | A. |
| 17382 | 2274 | 849 | mU. G.mU. G. | 850 | P.mU.fC.fU.fU.fU.fU. G. |
| | | | A.mC.mC. A. A. A. | | G.fU.fC. A.fC. |
| | | | A. G. A.Chl | | A*fC*fU*fC*fU*fC* A. |
| 17383 | 2274 | 851 | G.mU. G.mU. G. | 852 | P.mU.fC.fU.fU.fU.fU. G. |
| | | | A.mC.mC. A. A. A. | | G.fU.fC. A.fC. |
| | | | A. G. A.Chl | | A*fC*fU*fC*fU*fC* A. |
| 17384 | 2275 | 853 | G.mU. G. | 854 | P.mU. A.fC.fU.fU.fU.fU. |
| | | | A.mC.mC. A. A. A. | | G. G.fU.fC. A.fC* |
| | | | A. G.mU. A.Chl | | A*fC*fU*fC*fU* C. |
| 17385 | 2277 | 855 | G. A.mC.mC. A. A. | 856 | P.mU.fU. A. |
| | | | A. A. G.mU.mU. A. | | A.fC.fU.fU.fU.fU. G. |
| | | | A.Chl | | G.fU.fC* A*fC* |
| | | | | | A*fC*fU* C. |
| 17386 | 2296 | 857 | G.mC. | 858 | P.mU.fC.fU. A. G. A. A. |
| | | | A.mC.mC.mU.mU. | | A. G. G.fU. G.fC* A* A* |
| | | | mU.mC.mU. A. G. | | A*fC* A* U. |
| | | | A.Chl | | |
| 17387 | 2299 | 859 | mC.mC.mU.mU.mU. | 860 | P.mU.fC. A. A.fC.fU. A. |
| | | | mC.mU. A. | | G. A. A. A. G. G*fU* |
| | | | G.mU.mU. G. A.Chl | | G*fC* A* A* A. |
| 21138 | 2296 | 861 | G.mC. | 862 | P.mU.fC.fU. A. G. A.mA. |
| | | | A.mC.mC.mU.mU. | | A. G. G.fU. G.mC* A* |
| | | | mU.mC.mU. A. G. | | A* A*mC* A* U. |
| | | | A.TEG-Chl | | |
| 21139 | 2296 | 863 | G.mC. | 864 | P.mU.fC.fU. A. G.mA. |
| | | | A.mC.mC.mU.mU. | | A.mA. G. G.fU. G.mC* |
| | | | mU.mC.mU. A. G. | | A* A* A*mC* A* U. |
| | | | A.TEG-Chl | | |
| 21140 | 2296 | 865 | G.mC. | 866 | P.mU.fC.fU. A. G. A. A. |
| | | | A.mC.mC.mU.mU. | | A. G. G.fU. G.mC* |
| | | | mU.mC.mU. A. G. | | A*mA* A*mC* A* U. |
| | | | A.TEG-Chl | | |
| 21141 | 2296 | 867 | G.mC. | 868 | P.mU.fC.fU. A. G. A.mA. |
| | | | A.mC.mC.mU.mU. | | A. G. G.fU. G.mC* |
| | | | mU.mC.mU. A. G. | | A*mA* A*mC* A* U. |
| | | | A.TEG-Chl | | |
| 21142 | 2296 | 869 | G.mC. | 870 | P.mU.fC.fU. A. G.mA. |
| | | | A.mC.mC.mU.mU. | | A.mA. G. G.fU. G.mC* |
| | | | mU.mC.mU. A. G. | | A*mA* A*mC* A* U. |
| | | | A.TEG-Chl | | |
| 21143 | 2296 | 871 | G.mC. | 872 | P.mU.fC.fU. A. G. A. A. |
| | | | A.mC.mC.mU.mU. | | A. G. G.fU. |
| | | | mU.mC.mU. A. G. | | G.fC*mA*mA*mA*fC* |
| | | | A.TEG-Chl | | mA* U. |
| 21144 | 2296 | 873 | G.mC. | 874 | P.mU.fC.fU. A. G. A.mA. |
| | | | A.mC.mC.mU.mU. | | A. G. G.fU. |
| | | | mU.mC.mU. A. G. | | G.fC*mA*mA*mA*fC* |
| | | | A.TEG-Chl | | mA* U. |
| 21145 | 2296 | 875 | G.mC. | 876 | P.mU.fC.fU. A. G.mA. |
| | | | A.mC.mC.mU.mU. | | A.mA. G. G.fU. |
| | | | mU.mC.mU. A. G. | | G.fC*mA*mA*mA*fC* |
| | | | A.TEG-Chl | | mA* U. |
| 21146 | 2296 | 877 | G.mC. | 878 | P.mU.fC.fU. A. G. A. A. |
| | | | A.mC.mC.mU.mU. | | A. G. G.fU. G.fC* A* A* |
| | | | mU.mC.mU. | | A*fC* A* U. |
| | | | A*mG*mA.TEG-Chl | | |
| 21147 | 2296 | 879 | mG*mC* | 880 | P.mU.fC.fU. A. G. A. A. |
| | | | A.mC.mC.mU.mU. | | A. G. G.fU. G.fC* A* A* |
| | | | mU.mC.mU. | | A*fC* A* U. |
| | | | A*mG*mA.TEG-Chl | | |
| 21148 | 2296 | 881 | mG*mC*mA.mC.mC. | 882 | P.mU.fC.fU. A. G. A. A. |
| | | | mU.mU.mU.mC. | | A. G. G.fU. G.fC* A* A* |
| | | | mU.mA*mG*mA.TEG- | | A*fC* A* U. |
| | | | Chl | | |
| 21149 | 2275 | 883 | G.mU. G. | 884 | P.mU. A.fC.fU.fU.fU.fU. |
| | | | A.mC.mC. A. A. A. | | G. G.fU.fC. A.fC* |
| | | | A. G*mU*mA.TEG- | | A*fC*fU*fC*fU* C. |
| | | | Chl | | |
| 21150 | 2275 | 885 | mG*mU* G. | 886 | P.mU. A.fC.fU.fU.fU.fU. |
| | | | A.mC.mC. A. A.mA. | | G. G.fU.fC. A.fC* |
| | | | A. G*mU*mA.TEG- | | A*fC*fU*fC*fU* C. |
| | | | Chl | | |
| 21151 | 2275 | 887 | mG*mU*mG.mA. | 888 | P.mU. A.fC.fU.fU.fU.fU. |
| | | | mC.mC.mA.mA.mA. | | G. G.fU.fC. A.fC* |
| | | | mA.mG*mU*mA.TEG- | | A*fC*fU*fC*fU* C. |
| | | | Chl | | |
| 21152 | 2295 | 889 | mU.mU. G.mC. | 890 | P.mU.fU. A. G. A.mA. A. |
| | | | A.mC.mC.mU.mU. | | G. G.fU. G.fC. A. A* |
| | | | mU.mC.mU. A. | | A*fC* A*fA* G* G. |
| | | | A.TEG-Chl | | |
| 21153 | 2295 | 891 | mU.mU. G.mC. | 892 | P.mU.fU. A. G.mA. |
| | | | A.mC.mC.mU.mU. | | A.mA. G. G.fU. G.fC. A. |
| | | | mU.mC.mU. A. | | A* A*fC* A*fA* G* G. |
| | | | A.TEG-Chl | | |
| 21154 | 2295 | 893 | mU.mU. G.mC. | 894 | P.mU.fU.mA. G.mA. |
| | | | A.mC.mC.mU.mU. | | A.mA. G.mG.fU. G.fC. A. |
| | | | mU.mC.mU. A. | | A* A*fC* A*fA* G* G. |
| | | | A.TEG-Chl | | |
| 21155 | 2295 | 895 | mU.mU. G.mC. | 896 | P.mU.fU. A. G. A.mA. A. |
| | | | A.mC.mC.mU.mU. | | G. G.fU. G.mC. A. A* |
| | | | mU.mC.mU. A. | | A*mC* A*mA* G* G. |
| | | | A.TEG-Chl | | |
| 21156 | 2295 | 897 | mU.mU. G.mC. | 898 | P.mU.fU. A. G. A.mA. A. |
| | | | A.mC.mC.mU.mU. | | G. G.fU. G.fC. |
| | | | mU.mC.mU. A. | | A.mA*mA*fC*mA*fA* |
| | | | A.TEG-Chl | | mG* G. |
| 21157 | 2295 | 899 | mU.mU. G.mC. | 900 | P.mU.fU. A. G. A.mA. A. |
| | | | A.mC.mC.mU.mU. | | G. G.fU. |
| | | | mU.mC.mU. A. | | G.fC.mA.mA*mA*fC*mA* |
| | | | A.TEG-Chl | | fA*mG* G. |
| 21158 | 2295 | 901 | mU.mU. G.mC. | 902 | P.mU.fU. A. G. A.mA. A. |
| | | | A.mC.mC.mU.mU. | | G. G.fU. G.fC. |
| | | | mU.mC.mU. A. | | A.mA*mA*fC*mA*mA* |
| | | | A.TEG-Chl | | mG* G. |
| 21159 | 2295 | 903 | mU.mU. G.mC. | 904 | P.mU.fU. A. G. A.mA. A. |
| | | | A.mC.mC.mU.mU. | | G. G.fU. G.fC. |
| | | | mU.mC.mU. A. | | A.mA*mA*mC*mA*mA* |
| | | | A.TEG-Chl | | mG* G. |
| 21160 | 2295 | 905 | mU.mU. G.mC. | 906 | P.mU.fU. A. G. A.mA. A. |
| | | | A.mC.mC.mU.mU. | | G. G.fU. G.fC.mA. |
| | | | mU.mC.mU. A. | | A*mA*mC*mA*mA*mG* |
| | | | A.Chl-TEG | | mG. |
| 21161 | 2295 | 907 | mU.mU. G.mC. | 908 | P.mU.fU. A. G. A.mA. A. |
| | | | A.mC.mC.mU.mU. | | G. G.fU. G.fC. A. A* |
| | | | mU.mC.mU. A. | | A*fC* A*mA*mG* G. |
| | | | A.TEG-Chl | | |
| 21162 | 2295 | 909 | mU.mU. G.mC. | 910 | P.mU.fU. A. G. A.mA. A. |
| | | | A.mC.mC.mU.mU. | | G. G.fU. G.fC.mA. |
| | | | mU.mC.mU. A. | | A*mA*fC* A*mA*mG* |
| | | | A.TEG-Chl | | G. |
| 21163 | 2295 | 911 | mU.mU. G.mC. | 912 | P.mU.fU. A. G. A. A. A. |
| | | | A.mC.mC.mU.mU. | | G. G.fU. G.fC. A. A* |
| | | | mU.mC.mU. A* | | A*fC* A* A* G* G. |
| | | | A*TEG-Chl | | |
| 21164 | 2295 | 913 | mU.mU. G.mC. | 914 | P.mU.fU. A. G. A. A. A. |
| | | | A.mC.mC.mU.mU. | | G. G.fU. G.fC. A. A* |
| | | | mU.mC.mU.mA*mA* | | A*fC* A* A* G* G. |
| | | | TEG-Chl | | |
| 21165 | 2295 | 915 | mU*mU* G.mC. | 916 | P.mU.fU. A. G. A. A. A. |
| | | | A.mC.mC.mU.mU. | | G. G.fU. G.fC. A. A* |
| | | | mU.mC.mU.mA*mA* | | A*fC* A* A* G* G. |
| | | | TEG-Chl | | |
| 21166 | 2295 | 917 | mU.mU.mG.mC.mA. | 918 | P.mU.fU. A. G. A. A. A. |
| | | | mC.mC.mU.mU. | | G. G.fU. G.fC. A. A* |
| | | | mU.mC.mU.mA*mA* | | A*fC* A* A* G* G. |
| | | | TEG-Chl | | |
| 21167 | 2299 | 919 | mC.mC.mU.mU.mU. | 920 | P.mU.fC. A. A.fC.fU. A. |
| | | | mC.mU. A. | | G. A.mA. A. G. G*fU* |
| | | | G.mU.mU. G. | | G*fC* A* A* A. |
| | | | A.TEG-Chl | | |
| 21168 | 2299 | 921 | mC.mC.mU.mU.mU. | 922 | P.mU.fC. A. A.fC.fU. A. |
| | | | mC.mU. A. | | G. A.mA. A. G. G*mU* |
| | | | G.mU.mU. G. | | G*mC* A* A* A. |
| | | | A.TEG-Chl | | |
| 21169 | 2299 | 923 | mC.mC.mU.mU.mU. | 924 | P.mU.fC. A. A.fC.fU. A. |
| | | | mC.mU. A. | | G.mA. A. A.mG. G*fU* |
| | | | G.mU.mU. G. | | G*fC* A* A* A. |
| | | | A.TEG-Chl | | |
| 21170 | 2299 | 925 | mC.mC.mU.mU.mU. | 926 | P.mU.fC. A. A.fC.fU. A. |
| | | | mC.mU. A. | | G.mA. A. A.mG. G*mU* |
| | | | G.mU.mU. G. | | G*mC* A* A* A. |
| | | | A.TEG-Chl | | |
| 21171 | 2299 | 927 | mC.mC.mU.mU.mU. | 928 | P.mU.fC. A. A.fC.fU. A. |
| | | | mC.mU. A. | | G. A.mA. A. G. G*mU* |
| | | | G.mU.mU. G. | | G*mC* A*mA* A. |
| | | | A.TEG-Chl | | |
| 21172 | 2299 | 929 | mC.mC.mU.mU.mU. | 930 | P.mU.fC. A. A.fC.fU. A. |
| | | | mC.mU. A. | | G. A.mA. A. G. G*mU* |
| | | | G.mU.mU. G. | | G*mC*mA*mA* A. |
| | | | A.TEG-Chl | | |
| 21173 | 2299 | 931 | mC.mC.mU.mU.mU. | 932 | P.mU.fC. A. A.fC.fU. A. |
| | | | mC.mU. A. | | G. A.mA. A. |
| | | | G.mU.mU. G. | | G.mG*mU*mG*mC*mA* |
| | | | A.TEG-Chl | | mA* A. |
| 21174 | 2299 | 933 | mC.mC.mU.mU.mU. | 934 | P.mU.fC. A. A.fC.fU. A. |
| | | | mC.mU. A. | | G. A.mA. A. G. |
| | | | G.mU.mU. G. | | G*mU*mG*mC*mA*mA* |
| | | | A.TEG-Chl | | A. |
| 21175 | 2299 | 935 | mC.mC.mU.mU.mU. | 936 | P.mU.fC. A. A.fC.fU. A. |
| | | | mC.mU. A. | | G. A.mA. A. G. |
| | | | G.mU.mU. G. | | G*fU*mG*fC*mA*mA* |
| | | | A.TEG-Chl | | A. |
| 21176 | 2299 | 937 | mC.mC.mU.mU.mU. | 938 | P.mU.fC. A. A.fC.fU. A. |
| | | | mC.mU. A. | | G.mA. A. A.mG. |
| | | | G.mU.mU. G. | | G*fU*mG*fC*mA*mA* |
| | | | A.TEG-Chl | | A. |
| 21177 | 2299 | 939 | mC.mC.mU.mU.mU. | 940 | P.mU.fC. A. A.fC.fU. A. |
| | | | mC.mU. A. | | G. A. A. A. G. G*fU* |
| | | | G.mU.mU*mG*mA. | | G*fC* A* A* A. |
| | | | TEG-Chl | | |
| 21178 | 2299 | 941 | mC*mC*mU.mU.mU. | 942 | P.mU.fC. A. A.fC.fU. A. |
| | | | mC.mU. A. | | G. A. A. A. G. G*fU* |
| | | | G.mU.mU*mG*mA. | | G*fC* A* A* A. |
| | | | TEG-Chl | | |
| 21179 | 2299 | 943 | mC*mC*mU.mU.mU. | 944 | P.mU.fC. A. A.fC.fU. A. |
| | | | mC.mU.mA.mG. | | G. A. A. A. G. G*fU* |
| | | | mU.mU*mG*mA.TEG- | | G*fC* A* A* A. |
| | | | Chl | | |
| 21203 | 2296 | 945 | G.mC. | 946 | P.mU.fC.fU. A. G. A.mA. |
| | | | A.mC.mC.mU.mU. | | A. G. G.fU. G.mC* A* |
| | | | mU.mC.mU. | | A* A*mC* A* U. |
| | | | A*mG*mA.TEG-Chl | | |
| 21204 | 2296 | 947 | G.mC. | 948 | P.mU.fC.fU. A. G.mA. |
| | | | A.mC.mC.mU.mU. | | A.mA. G. G.fU. G.mC* |
| | | | mU.mC.mU. | | A* A* A*mC* A* U. |
| | | | A*mG*mA.TEG-Chl | | |
| 21205 | 2296 | 949 | G.mC. | 950 | P.mU.fC.fU. A. G.mA. |
| | | | A.mC.mC.mU.mU. | | A.mA. G. G.fU. G.mC* |
| | | | mU.mC.mU. | | A*mA* A*mC* A* U. |
| | | | A*mG*mA.TEG-Chl | | |
| 21206 | 2296 | 951 | mG*mC* | 952 | P.mU.fC.fU. A. G. A.mA. |
| | | | A.mC.mC.mU.mU. | | A. G. G.fU. G.mC* A* |
| | | | mU.mC.mU. | | A* A*mC* A* U. |
| | | | A*mG*mA.TEG-Chl | | |
| 21207 | 2296 | 953 | mG*mC* | 954 | P.mU.fC.fU. A. G.mA. |
| | | | A.mC.mC.mU.mU. | | A.mA. G. G.fU. G.mC* |
| | | | mU.mC.mU. | | A* A* A*mC* A* U. |
| | | | A*mG*mA.TEG-Chl | | |
| 21208 | 2296 | 955 | mG*mC* | 956 | P.mU.fC.fU. A. G.mA. |
| | | | A.mC.mC.mU.mU. | | A.mA. G. G.fU. G.mC* |
| | | | mU.mC.mU. | | A*mA* A*mC* A* U. |
| | | | A*mG*mA.TEG-Chl | | |
| 21209 | 2296 | 957 | mG*mC*mA.mC.mC. | 958 | P.mU.fC.fU. A. G. A.mA. |
| | | | mU.mU.mU.mC. | | A. G. G.fU. G.mC* A* |
| | | | mU.mA*mG*mA.TEG- | | A* A*mC* A* U. |
| | | | Chl | | |
| 21210 | 2296 | 959 | mG*mC*mA.mC.mC. | 960 | P.mU.fC.fU. A. G.mA. |
| | | | mU.mU.mU.mC. | | A.mA. G. G.fU. G.mC* |
| | | | mU.mA*mG*mA.TEG- | | A* A* A*mC* A* U. |
| | | | Chl | | |
| 21211 | 2296 | 961 | mG*mC*mA.mC.mC. | 962 | P.mU.fC.fU. A. G.mA. |
| | | | mU.mU.mU.mC. | | A.mA. G. G.fU. G.mC* |
| | | | mU.mA*mG*mA.TEG- | | A*mA* A*mC* A* U. |
| | | | Chl | | |
| 21212 | 2295 | 963 | mU.mU. G.mC. | 964 | P.mU.fU. A. G. A.mA. A. |
| | | | A.mC.mC.mU.mU. | | G. G.fU. |
| | | | mU.mC.mU*mA*mA. | | G.fC.mA.mA*mA*fC*m |
| | | | TEG-Chl | | A*mA*mG* G. |
| 21213 | 2295 | 965 | mU.mU. G.mC. | 966 | P.mU.fU. A. G. A.mA. A. |
| | | | A.mC.mC.mU.mU. | | G. G.fU. G.fC. |
| | | | mU.mC.mU*mA*mA. | | A.mA*mA*mC*mA*mA* |
| | | | TEG-Chl | | mG* G. |
| 21214 | 2295 | 967 | mU.mU. G.mC. | 968 | P.mU.fU. A. G. A.mA. A. |
| | | | A.mC.mC.mU.mU. | | G. G.fU. G.fC. A. A* |
| | | | mU.mC.mU*mA*mA. | | A*fC* A*mA*mG* G. |
| | | | TEG-Chl | | |
| 21215 | 2295 | 969 | mU.mU. G.mC. | 970 | P.mU.fU. A. G. A.mA. A. |
| | | | A.mC.mC.mU.mU. | | G. G.fU. G.fC.mA. |
| | | | mU.mC.mU*mA*mA. | | A*mA*fC* A*mA*mG* |
| | | | TEG-Chl | | G. |
| 21216 | 2295 | 971 | mU*mU* G.mC. | 972 | P.mU.fU. A. G. A.mA. A. |
| | | | A.mC.mC.mU.mU. | | G. G.fU. |
| | | | mU.mC.mU*mA*mA. | | G.fC.mA.mA*mA*fC*m |
| | | | TEG-Chl | | A*mA*mG* G. |
| 21217 | 2295 | 973 | mU*mU* G.mC. | 974 | P.mU.fU. A. G. A.mA. A. |
| | | | A.mC.mC.mU.mU. | | G. G.fU. G.fC. |
| | | | mU.mC.mU*mA*mA. | | A.mA*mA*mC*mA*mA* |
| | | | TEG-Chl | | mG* G. |
| 21218 | 2295 | 975 | mU*mU* G.mC. | 976 | P.mU.fU. A. G. A.mA. A. |
| | | | A.mC.mC.mU.mU. | | G. G.fU. G.fC. A. A* |
| | | | mU.mC.mU*mA*mA. | | A*fC* A*mA*mG* G. |
| | | | TEG-Chl | | |
| 21219 | 2295 | 977 | mU*mU* G.mC. | 978 | P.mU.fU. A. G. A.mA. A. |
| | | | A.mC.mC.mU.mU. | | G. G.fU. G.fC.mA. |
| | | | mU.mC.mU*mA*mA. | | A*mA*fC* A*mA*mG* |
| | | | TEG-Chl | | G. |
| 21220 | 2295 | 979 | mU.mU.mG.mC.mA. | 980 | P.mU.fU. A. G. A.mA. A. |
| | | | mC.mC.mU.mU. | | G. G.fU. |
| | | | mU.mC.mU*mA*mA. | | G.fC.mA.mA*mA*fC*mA* |
| | | | TEG-Chl | | mA*mG* G. |
| 21221 | 2295 | 981 | mU.mU.mG.mC.mA. | 982 | P.mU.fU. A. G. A.mA. A. |
| | | | mC.mC.mU.mU. | | G. G.fU. G.fC. |
| | | | mU.mC.mU*mA*mA. | | A.mA*mA*mC*mA*mA* |
| | | | TEG-Chl | | mG* G. |
| 21222 | 2295 | 983 | mU.mU.mG.mC.mA. | 984 | P.mU.fU. A. G. A.mA. A. |
| | | | mC.mC.mU.mU. | | G. G.fU. G.fC. A. A* |
| | | | mU.mC.mU*mA*mA. | | A*fC* A*mA*mG* G. |
| | | | TEG-Chl | | |
| 21223 | 2295 | 985 | mU.mU.mG.mC.mA. | 986 | P.mU.fU. A. G. A.mA. A. |
| | | | mC.mC.mU.mU. | | G. G.fU. G.fC.mA. |
| | | | mU.mC.mU*mA*mA. | | A*mA*fC* A*mA*mG* |
| | | | TEG-Chl | | G. |
| 21224 | 2299 | 987 | mC.mC.mU.mU.mU. | 988 | P.mU.fC. A. A.fC.fU. A. |
| | | | mC.mU. A. | | G. A.mA. A. G. |
| | | | G.mU.mU*mG*mA. | | G*fU*mG*fC*mA*mA* |
| | | | TEG-Chl | | A. |
| 21225 | 2299 | 989 | mC*mC*mU.mU.mU. | 990 | P.mU.fC. A. A.fC.fU. A. |
| | | | mC.mU. A. | | G. A.mA. A. G. |
| | | | G.mU.mU*mG*mA. | | G*fU*mG*fC*mA*mA* |
| | | | TEG-Chl | | A. |
| 21226 | 2299 | 991 | mC*mC*mU.mU.mU. | 992 | P.mU.fC. A. A.fC.fU. A. |
| | | | mC.mU.mA.mG. | | G. A.mA. A. G. |
| | | | mU.mU*mG*mA.TEG- | | G*fU*mG*fC*mA*mA* |
| | | | Chl | | A. |
| 21227 | 2296 | 993 | G.mC. | 994 | P.mU.fC.fU. A. G.mA. |
| | | | A.mC.mC.mU.mU. | | A.mA. G. G.fU. |
| | | | mU.mC.mU. | | G.fC*mA*mA*mA*fC* |
| | | | A*mG*mA.TEG-Chl | | mA* U. |
| 20584 | 2296 | 995 | G.mC. | 996 | P.mU.fC.fU. A. G. A. A. |
| | | | A.mC.mC.mU.mU. | | A. G. G.mU. G.mC* A* |
| | | | mU.mC.mU. A. G. | | A* A*mC* A* U. |
| | | | A.Chl-TEG | | |
| 20585 | 2296 | 997 | G.mC. | 998 | P.mU.fC.fU. A. G. A. A. |
| | | | A.mC.mC.mU.mU. | | A. G. G.fU. G.mC* A* |
| | | | mU.mC.mU. A. G. | | A* A*mC* A* U. |
| | | | A.Chl-TEG | | |
| 20586 | 2296 | 999 | G.mC. | 1000 | P.mU. C. U. A. G. A. A. |
| | | | A.mC.mC.mU.mU. | | A. G. G.mU. G.mC* A* |
| | | | mU.mC.mU. A. G. | | A* A*mC* A* U. |
| | | | A.Chl-TEG | | |
| 20587 | 2296 | 1001 | G.mC. | 1002 | P.mU.fC.fU. A. G. A. A. |
| | | | A.mC.mC.mU.mU. | | A. G. G.fU. |
| | | | mU.mC.mU. A. G. | | G.fC*mA*mA*mA*fC* |
| | | | A.Chl-TEG | | mA* U. |
| 20616 | 2275 | 1003 | G.mU. G. | 1004 | P.mU. A.fC.fU.fU.fU.fU. |
| | | | A.mC.mC. A. A. A. | | G. G.fU.mC. A.mC* |
| | | | A. G.mU. A.Chl-TEG | | A*mC*mU*mC*mU* C. |
| 20617 | 2275 | 1005 | G.mU. G. | 1006 | P.mU. A.fC.fU.fU.fU.fU. |
| | | | A.mC.mC. A. A. A. | | G. G.fU.fC. A.mC* |
| | | | A. G.mU. A.Chl-TEG | | A*fC*mU*fC*mU* C. |
| 20618 | 2275 | 1007 | G.mU. G. | 1008 | P.mU. A. C. U. U. U. U. |
| | | | A.mC.mC. A. A. A. | | G. G. U.mC. A.mC* |
| | | | A. G.mU. A.Chl-TEG | | A*mC*mU*mC*mU* C. |
| 20619 | 2275 | 1009 | G.mU. G. | 1010 | P.mU. A.fC.fU.fU.fU.fU. |
| | | | A.mC.mC. A. A. A. | | G. G.fU.fC. |
| | | | A. G.mU. A.Chl-TEG | | A.mC*mA*mC*mU*mC* |
| | | | | | mU* C. |
| 21381 | 2275 | 1011 | G.mU. G. | 1012 | P.mU. A.fC.fU.fU.fU.fU. |
| | | | A.mC.mC. A. A. A. | | G. G.fU.mC. A.mC* |
| | | | A. G*mU*mA.TEG- | | A*mC*mU*mC*mU* C. |
| | | | Chl | | |
| 21382 | 2275 | 1013 | G.mU. G. | 1014 | P.mU. A.fC.fU.fU.fU.fU. |
| | | | A.mC.mC. A. A. A. | | G. G.fU.fC. A.mC* |
| | | | A. G*mU*mA.TEG- | | A*fC*mU*fC*mU* C. |
| | | | Chl | | |
| 21383 | 2275 | 1015 | mG*mU*mG.mA. | 1016 | P.mU. A.fC.fU.fU.fU.fU. |
| | | | mC.mC.mA.mA.mA. | | G. G.fU.mC. A.mC* |
| | | | mA.mG*mU*mA.TEG- | | A*mC*mU*mC*mU* C. |
| | | | Chl | | |
| 21384 | 2275 | 1017 | mG*mU*mG.mA. | 1018 | P.mU. A.fC.fU.fU.fU.fU. |
| | | | mC.mC.mA.mA.mA. | | G. G.fU.fC. A.mC* |
| | | | mA.mG*mU*mA.TEG- | | A*fC*mU*fC*mU* C. |
| | | | Chl | | |
| 20392 | 2275 | 1019 | G.mU. G. | 1020 | P.mU. A.fC.fU.fU.fU.fU. |
| | | | A.mC.mC. A. A. A. | | G. G.fU.fC. A.fC* |
| | | | A. G.mU. A.TEG-Chl | | A*fC*fU*fC*fU* C. |
| 20393 | 2296 | 1021 | G.mC. | 1022 | P.mU.fC.fU. A. G. A. A. |
| | | | A.mC.mC.mU.mU. | | A. G. G.fU. G.fC* A* A* |
| | | | mU.mC.mU. A. G. | | A*fC* A* U. |
| | | | A.TEG-Chl | | |
| 21429 | 2275 | 1023 | G.mU. G. | 1024 | P.mU. A.fC.fU.fU.fU.fU. |
| | | | A.mC.mC. A. A. A. | | G. G.fU.fC. A.mC* |
| | | | A. G*mU*mA.Teg- | | A*fC*mU*fC*mU* C. |
| | | | Chl | | |
| 21430 | 2275 | 1025 | G.mU. G. | 1026 | P.mU. A.fC.fU.fU.fU.fU. |
| | | | A.mC.mC. A. A.mA. | | G. G.fU.mC. A.mC* |
| | | | A. G*mU*mA.Teg- | | A*mC*mU*mC*mU* C. |
| | | | Chl | | |

Key
Chl = cholesterol with hydroxyprolinol linker
TEG-chl = cholesterol with TEG linker
m = 2′Ome
f = 2′fluoro
* = phosphorothioate llinkage
. = phosphodiester linkage

TABLE 6
TGFβ2 (Accession Number: NM_001135599.1) sd-rxRNA sequences

| Oligo | Start | SEQ ID | | SEQ ID | |
|---|---|---|---|---|---|
| Number | Site | NO | Sense sequence | NO | Antisense sequence |
| 14408 | 1324 | 1027 | G. | 1028 | P.mU.fC. G. A. A. G. G. |
| | | | G.mC.mU.mC.mU. | | A. G. A. G.mC.mC* |
| | | | mC.mC.mU.mU.mC | | A*mU*mU*mC* G* C. |
| | | | . G. A.Chl | | |
| 14409 | 1374 | 1029 | G. A.mC. A. G. G. | 1030 | P.mC.fC. A. G. |
| | | | A. A.mC.mC.mU. G. | | G.fU.fU.fC.fC.fU. |
| | | | G.Chl | | G.mU.mC*mU*mU*m |
| | | | | | U* A*mU* G. |
| 14410 | 946 | 1031 | mC.mC. A. A. G. G. | 1032 | P.mU. A. A. |
| | | | A. G. | | A.fC.fC.fU.fC.fC.fU.mU. |
| | | | G.mU.mU.mU. | | G. G*mC* G*mU* A* |
| | | | A.Chl | | G* U. |
| 14411 | 849 | 1033 | A.mU.mU.mU.mC. | 1034 | P.mU. G.fU. A. G. A.fU. |
| | | | mC. A.mU.mC.mU. | | G. G. A. A. A.mU*mC* |
| | | | A.mC. A.Chl | | A*mC*mC*mU* C. |
| 14412 | 852 | 1035 | mU.mC.mC. | 1036 | P.mU. G.fU.fU. G.fU. A. |
| | | | A.mU.mC.mU. | | G. A.fU. G. G. A* A* |
| | | | A.mC. A. A.mC. | | A*mU*mC* A* C. |
| | | | A.Chl | | |
| 14413 | 850 | 1037 | mU.mU.mU.mC.m | 1038 | P.mU.fU. G.fU. A. G. |
| | | | C. A.mU.mC.mU. | | A.fU. G. G. A. A. |
| | | | A.mC. A. A. Chl | | A*mU*mC* |
| | | | | | A*mC*mC* U. |
| 14414 | 944 | 1039 | mC. G.mC.mC. A. | 1040 | P.mA. |
| | | | A. G. G. A. G. | | A.fC.fC.fU.fC.fC.fU.fU. |
| | | | G.mU.mU.Chl | | G. G.mC. G*mU* A* |
| | | | | | G*mU* A* C. |
| 14415 | 1513 | 1041 | G.mU. G. G.mU. G. | 1042 | P.mU.fU.fC.fU. G. |
| | | | A.mU.mC. A. G. A. | | A.fU.fC. A.fC.mC. |
| | | | A.Chl | | A.mC*mU* G* G*mU* |
| | | | | | A* U. |
| 14416 | 1572 | 1043 | mC.mU.mC.mC.mU | 1044 | P.mA.fC. A.fU.fU. A. |
| | | | . G.mC.mU. A. | | G.fC. A. G. G. A. G* |
| | | | A.mU. G.mU.Chl | | A*mU* G*mU* G* G. |
| 14417 | 1497 | 1045 | A.mC.mC.mU.mC. | 1046 | P.mU. A.fU. A.fU. G.fU. |
| | | | mC. A.mC. A.mU. | | G. G. A. G. G.mU* |
| | | | A.mU. A.Chl | | G*mC*mC* A*mU* C. |
| 14418 | 1533 | 1047 | A. A. | 1048 | P.mU.fC.fC.fU. A. G.fU. |
| | | | G.mU.mC.mC. | | G. G. |
| | | | A.mC.mU. A. G. G. | | A.mC.mU.mU*mU* |
| | | | A.Chl | | A*mU* A* G* U. |
| 14419 | 1514 | 1049 | mU. G. G.mU. G. | 1050 | P.mU.fU.fU.fC.fU. G. |
| | | | A.mU.mC. A. G. A. | | A.fU.fC. A.mC.mC. |
| | | | A. A.Chl | | A*mC*mU* G* G*mU* |
| | | | | | A. |
| 14420 | 1534 | 1051 | A. G.mU.mC.mC. | 1052 | P.mU.fU.fC.fC.fU. A. |
| | | | A.mC.mU. A. G. G. | | G.fU. G. G. |
| | | | A. A.Chl | | A.mC.mU*mU*mU* |
| | | | | | A*mU* A* G. |
| 14421 | 943 | 1053 | A.mC. G.mC.mC. | 1054 | P.mA.fC.fC.fU.fC.fC.fU.f |
| | | | A. A. G. G. A. G. | | U. G. G.mC. G.mU* A* |
| | | | G.mU.Chl | | G*mU* A*mC* U. |
| 18570 | 2445 | 1055 | mU. A.mU.mU.mU. | 1056 | P.mU. A.fC. A.fC. A. |
| | | | A.mU.mU. G.mU. | | A.fU. A. A. A.fU. A* |
| | | | G.mU. A.Chl | | A*fC*fU*fC* A* C. |
| 18571 | 2445 | 1057 | mU.mU. | 1058 | P.mU. A.fC. A.fC. A. |
| | | | A.mU.mU.mU. | | A.fU. A. A. A.fU. A* |
| | | | A.mU.mU. G.mU. | | A*fC*fU*fC* A* C. |
| | | | G.mU. A.Chl | | |
| 18572 | 2083 | 1059 | A.mU. C. A. G.mU. | 1060 | P.mU.fU.fU.fU. A. A.fC. |
| | | | G.mU.mU. A. A. A. | | A.fC.fU. G. A.fU* G* A* |
| | | | A.Chl | | A*fC*fC* A. |
| 18573 | 2083 | 1061 | mC. A.mU.mC. A. | 1062 | P.mU.fU.fU.fU. A. A.fC. |
| | | | G.mU. G.mU.mU. | | A.fC.fU. G. A.fU* G* A* |
| | | | A. A. A. A.Chl | | A*fC*fC* A. |
| 18574 | 2544 | 1063 | A.mU. G. | 1064 | P.mU.fU.fC.fC.fU.fU. A. |
| | | | G.mC.mU.mU. A. | | A. G.fC.fC. A. U*fC*fC* |
| | | | A. G. G. A. A.Chl | | A*fU* G* A. |
| 18575 | 2544 | 1065 | G. A.mU. G. | 1066 | P.mU.fU.fC.fC.fU.fU. A. |
| | | | G.mC.mU.mU. A. | | A. G.fC.fC. A. U*fC*fC* |
| | | | A. G. G. A. A.Chl | | A*fU* G* A. |
| 18576 | 2137 | 1067 | mU.mU. G.mU. | 1068 | P.mU. A. A.fC. A. G. A. |
| | | | G.mU.mU.mC.mU. | | A.fC. A.fC. A. A* |
| | | | G.mU.mU. A.Chl | | A*fC*fU*fU*fC* C. |
| 18577 | 2137 | 1069 | mU.mU.mU. G.mU. | 1070 | P.mU. A. A.fC. A. G. A. |
| | | | G.mU.mU.mC.mU. | | A.fC. A.fC. A. A* |
| | | | G.mU.mU. A.Chl | | A*fC*fU*fU*fC* C. |
| 18578 | 2520 | 1071 | A. A. A.mU. | 1072 | P.mU. G. G.fC. A. A. A. |
| | | | A.mC.mU.mU.mU. | | G.fU. A.fU.fU.fU* G* |
| | | | G.mC.mC. A.Chl | | G*fU*fC*fU* C. |
| 18579 | 2520 | 1073 | mC. A. A. A.mU. | 1074 | P.mU. G. G.fC. A. A. A. |
| | | | A.mC.mU.mU.mU. | | G.fU. A.fU.fU.fU* G* |
| | | | G.mC.mC. A.Chl | | G*fU*fC*fU* C. |
| 18580 | 3183 | 1075 | mC.mU.mU. G.mC. | 1076 | P.mU.fU.fU. G.fU. A. |
| | | | A.mC.mU. A.mC. A. | | G.fU. G.fC. A. A. |
| | | | A. A.Chl | | G*fU*fC* A* A* A* C. |
| 18581 | 3183 | 1077 | A.mC.mU.mU. | 1078 | P.mU.fU.fU. G.fU. A. |
| | | | G.mC. A.mC.mU. | | G.fU. G.fC. A. A. |
| | | | A.mC. A. A. A.Chl | | G*fU*fC* A* A* A* C. |
| 18582 | 2267 | 1079 | G. A. | 1080 | P.mU. A.fC.fU. A. A.fU. |
| | | | A.mU.mU.mU. | | A. A. |
| | | | A.mU.mU. A. | | A.fU.fU.fC*fU*fU*fC*fC |
| | | | G.mU. A.Chl | | * A* G. |
| 18583 | 2267 | 1081 | A. G. A. | 1082 | P.mU. A.fC.fU. A. A.fU. |
| | | | A.mU.mU.mU. | | A. A. |
| | | | A.mU.mU. A. | | A.fU.fU.fC*fU*fU*fC*fC |
| | | | G.mU. A.Chl | | * A* G. |
| 18584 | 3184 | 1083 | mU.mU. G.mC. | 1084 | P.mU.fU.fU.fU. G.fU. A. |
| | | | A.mC.mU. A.mC. A. | | G.fU. G.fC. A. A* |
| | | | A. A. A.Chl | | G*fU*fC* A* A* A. |
| 18585 | 3184 | 1085 | mC.mU.mU. G.mC. | 1086 | P.mU.fU.fU.fU. G.fU. A. |
| | | | A.mC.mU. A.mC. A. | | G.fU. G.fC. A. A* |
| | | | A. A. A.Chl | | G*fU*fC* A* A* A. |
| 18586 | 2493 | 1087 | A.mU. A. A. A. | 1088 | P.mU.fC. A.fC.fC.fU. |
| | | | A.mC. A. G. G.mU. | | G.fU.fU.fU.fU. |
| | | | G. A.Chl | | A.fU*fU*fU*fU*fC*fC* |
| | | | | | A. |
| 18587 | 2493 | 1089 | A. A.mU. A. A. A. | 1090 | P.mU.fC. A.fC.fC.fU. |
| | | | A.mC. A. G. G.mU. | | G.fU.fU.fU.fU. |
| | | | G. A.Chl | | A.fU*fU*fU*fU*fC*fC* |
| | | | | | A. |
| 18588 | 2297 | 1091 | G. A.mC. A. A.mC. | 1092 | P.mU. G.fU.fU. G.fU.fU. |
| | | | A. A.mC. A. A.mC. | | G.fU.fU. G.fU.fC* |
| | | | A.Chl | | G*fU*fU* G*fU* U. |
| 18589 | 2046 | 1093 | A.mU. G. | 1094 | P.mU.fU. G.fU.fU. A.fC. |
| | | | C.mU.mU. G.mU. | | A. A. G.fC. A.fU*fC* |
| | | | A. A.mC. A. A.Chl | | A*fU*fC* G* U. |
| 18590 | 2531 | 1095 | mC. A. G. A. A. | 1096 | P.mU.fC. A.fU. G. A. |
| | | | A.mC.mU.mC. | | G.fU.fU.fU.fC.fU. G* |
| | | | A.mU. G. A.Chl | | G*fC* A* A* A* G. |
| 18591 | 2389 | 1097 | G.mU. A.mU.mU. | 1098 | P.mU. G.fC. A.fU. A. |
| | | | G.mC.mU. A.mU. | | G.fC. A. A.fU. A.fC* A* |
| | | | G.mC. A.Chl | | G* A* A* A* A. |
| 18592 | 2530 | 1099 | mC.mC. A. G. A. A. | 1100 | P.mU. A.fU. G. A. |
| | | | A.mC.mU.mC. | | G.fU.fU.fU.fC.fU. G. |
| | | | A.mU. A.Chl | | G*fC* A* A* A* G* U. |
| 18593 | 2562 | 1101 | A.mC.mU.mC. A. | 1102 | P.mU. G.fC.fU.fC. |
| | | | A. A.mC. G. A. | | G.fU.fU.fU. G. A. |
| | | | G.mC. A.Chl | | G.fU*fU*fC* A* A* G* |
| | | | | | U. |
| 18594 | 2623 | 1103 | A.mU. A.mU. G. | 1104 | P.mU.fU.fC.fU.fC. G. |
| | | | A.mC.mC. G. A. G. | | G.fU.fC. A.fU. A.fU* A* |
| | | | A. A.Chl | | A*fU* A* A* C. |
| 18595 | 2032 | 1105 | mC. G. A.mC. G. | 1106 | P.mU.fU.fC. G.fU.fU. |
| | | | A.mC. A. A.mC. G. | | G.fU.fC. G.fU.fC. |
| | | | A. A.Chl | | G*fU*fC* A*fU*fC* A. |
| 18596 | 2809 | 1107 | G.mU. A. A. | 1108 | P.mU.fU.fC. A.fC.fU. G. |
| | | | A.mC.mC. A. G.mU. | | G.fU.fU.fU. A.fC*fU* A* |
| | | | G. A. A.Chl | | A* A*fC* U. |
| 18597 | 2798 | 1109 | mU.mU. G.mU.mC. | 1110 | P.mU.fC.fU. A. A. |
| | | | A. G.mU.mU.mU. | | A.fC.fU. G. A.fC. A. A* |
| | | | A. G. A.Chl | | A* G* A* A*fC* C. |
| 18598 | 2081 | 1111 | mU.mC. A.mU.mC. | 1112 | P.mU.fU. A. A.fC. |
| | | | A. G.mU. | | A.fC.fU. G. A.fU. G. A* |
| | | | G.mU.mU. A. A.Chl | | A*fC*fC* A* A* G. |
| 18599 | 2561 | 1113 | A. A.mC.mU.mC. | 1114 | P.mU.fC.fU.fC. |
| | | | A. A. A.mC. G. A. G. | | G.fU.fU.fU. G. A. |
| | | | A.Chl | | G.fU.fU*fC* A* A* |
| | | | | | G*fU* U. |
| 18600 | 2296 | 1115 | mC. G. A.mC. A. | 1116 | P.mU.fU.fU. G.fU.fU. |
| | | | A.mC. A. A.mC. A. | | G.fU.fU. G.fU.fC. |
| | | | A. A.Chl | | G*fU*fU* G*fU*fU* C. |
| 18601 | 2034 | 1117 | A.mC. G. A.mC. A. | 1118 | P.mU.fC. A.fU.fC. |
| | | | A.mC. G. A.mU. G. | | G.fU.fU. G.fU.fC. |
| | | | A.Chl | | G.fU*fC* G*fU*fC* |
| | | | | | A*fU. |
| 18602 | 2681 | 1119 | G.mC.mU. | 1120 | P.mU.fU.fC.fC.fU.fU. A. |
| | | | G.mC.mC.mU. A. A. | | G. G.fC. A. G.fC*fU* G* |
| | | | G. G. A. A.Chl | | A*fU* A* C. |
| 18603 | 2190 | 1121 | A.mU.mU.mC.mU. | 1122 | P.mU. G. A. A. A.fU. |
| | | | A.mC. | | G.fU. A. G. A. A.fU* A* |
| | | | A.mU.mU.mU.mC. | | A* G* G*fC* C. |
| | | | A.Chl | | |
| 20604 | 2083 | 1123 | mC. A.mU.mC. A. | 1124 | P.mU.fU.fU.fU. A. A.fC. |
| | | | G.mU. G.mU.mU. | | A.fC.fU. G. A.mU* G* |
| | | | A. A. A. A.Chl | | A* A*mC*mC* A. |
| 20605 | 2083 | 1125 | mC. A.mU.mC. A. | 1126 | P.mU.fU.fU.fU. A. A.fC. |
| | | | G.mU. G.mU.mU. | | A.fC.fU. G. A.mU* G* |
| | | | A. A. A. A.Chl | | A* A*fC*mC* A. |
| 20606 | 2083 | 1127 | mC. A.mU.mC. A. | 1128 | P.mU. U. U. U. A. A. C. |
| | | | G.mU. G.mU.mU. | | A. C. U. G. A.mU* G* |
| | | | A. A. A. A.Chl | | A* A*mC*mC* A. |
| 20607 | 2083 | 1129 | mC. A.mU.mC. A. | 1130 | P.mU.fU.fU.fU. A. A.fC. |
| | | | G.mU. G.mU.mU. | | A.fC.fU. G. |
| | | | A. A. A. A.Chl | | A.fU*mG*mA*mA*fC*f |
| | | | | | C* A. |
| 21722 | 2081 | 1131 | mU.mC. A.mU.mC. | 1132 | P.mU.fU. A. A.fC. |
| | | | A. G.mU. | | A.fC.fU. G. A.fU. G. A* |
| | | | G.mU.mU. A. A.Chl | | A*mC*mC* A* A* G. |
| 21723 | 2081 | 1133 | mU.mC. A.mU.mC. | 1134 | P.mU.fU. A. A.fC. |
| | | | A. G.mU. | | A.fC.fU. G. A.fU. |
| | | | G.mU.mU. A. A.Chl | | G.mA*mA*mC*mC*mA |
| | | | | | *mA* G. |
| 21724 | 2081 | 1135 | mU.mC. A.mU.mC. | 1136 | P.mU.fU. A. A.fC. |
| | | | A. G.mU. | | A.fC.fU. G. A.mU. |
| | | | G.mU.mU. A. A.Chl | | G.mA*mA*mC*mC*mA |
| | | | | | *mA* G. |
| 21725 | 2081 | 1137 | mU.mC. A.mU.mC. | 1138 | P.mU.fU. A. A.fC. |
| | | | A. G.mU. | | A.fC.fU. G. A.fU. G. A* |
| | | | G.mU.mU. A. A.Chl | | A*fC*fC*mA*mA* G. |
| 21726 | 2081 | 1139 | mU.mC. A.mU.mC. | 1140 | P.mU.fU. A. A.fC. |
| | | | A. G.mU. | | A.fC.fU. G. A.fU. |
| | | | G.mU.mU. A. A.Chl | | G.mA*mA*fC*fC*mA* |
| | | | | | mA* G. |
| 21727 | 2081 | 1141 | mU.mC. A.mU.mC. | 1142 | P.mU.fU. A. A.fC. |
| | | | A. G.mU. | | A.fC.fU. G. A.fU. G. A* |
| | | | G.mU.mU*mA*mA | | A*fC*fC* A* A* G. |
| | | | .TEG-Chl | | |
| 21728 | 2081 | 1143 | mU*mC* | 1144 | P.mU.fU. A. A.fC. |
| | | | A.mU.mC. A. | | A.fC.fU. G. A.fU. G. A* |
| | | | G.mU. | | A*fC*fC* A* A* G. |
| | | | G.mU.mU*mA*mA | | |
| | | | .TEG-Chl | | |
| 21729 | 2081 | 1145 | mU*mC*mA.mU.m | 1146 | P.mU.fU. A. A.fC. |
| | | | C.mA.mG.mU.mG. | | A.fC.fU. G. A.fU. G. A* |
| | | | mU.mU*mA*mA.T | | A*fC*fC* A* A* G. |
| | | | EG-Chl | | |
| 21375 | 2081 | 1147 | mU.mC. A.mU.mC. | 1148 | P.mU.fU. A. A.fC. |
| | | | A. G.mU. | | A.fC.fU. G. A.fU. G. A* |
| | | | G.mU.mU*mA*mA | | A*mC*mC* A* A* G. |
| | | | .TEG-Chl | | |
| 21376 | 2081 | 1149 | mU.mC. A.mU.mC. | 1150 | P.mU.fU. A. A.fC. |
| | | | A. G.mU. | | A.fC.fU. G. A.fU. G. A* |
| | | | G.mU.mU*mA*mA | | A*fC*fC*mA*mA* G. |
| | | | .TEG-Chl | | |
| 21377 | 2081 | 1151 | mU.mC. A.mU.mC. | 1152 | P.mU.fU. A. A.fC. |
| | | | A. G.mU. | | A.fC.fU. G. A.fU. |
| | | | G.mU.mU*mA*mA | | G.mA*mA*fC*fC*mA* |
| | | | .TEG-Chl | | mA* G. |
| 21378 | 2081 | 1153 | mU*mC*mA.mU.m | 1154 | P.mU.fU. A. A.fC. |
| | | | C.mA.mG.mU.mG. | | A.fC.fU. G. A.fU. G. A* |
| | | | mU.mU*mA*mA.T | | A*mC*mC* A* A* G. |
| | | | EG-Chl | | |
| 21379 | 2081 | 1155 | mU*mC*mA.mU.m | 1156 | P.mU.fU. A. A.fC. |
| | | | C.mA.mG.mU.mG. | | A.fC.fU. G. A.fU. G. A* |
| | | | mU.mU*mA*mA.T | | A*fC*fC*mA*mA* G. |
| | | | EG-Chl | | |
| 21380 | 2081 | 1157 | mU*mC*mA.mU.m | 1158 | P.mU.fU. A. A.fC. |
| | | | C.mA.mG.mU.mG. | | A.fC.fU. G. A.fU. |
| | | | mU.mU*mA*mA.T | | G.mA*mA*fC*fC*mA* |
| | | | EG-Chl | | mA* G. |

| | |
|---|---|
| Key | |
| Chl = | cholesterol with hydroxyprolinol linker |
| TEG-chl = | cholesterol with TEG linker |
| m = | 2′Ome |
| f = | 2′fluoro |
| * = | phosphorothioate llinkage |
| . = | phosphodiester linkage |

TABLE 7
TGFβ1 (Accession Number: NM_000660.3)

| Oligo | Start | SEQ ID | | SEQ ID | |
|---|---|---|---|---|---|
| Number | Site | NO | Sense sequence | NO | Antisense sequence |
| 14394 | 1194 | 1159 | G.mC.mU. A. | 1160 | P.mU.fU.fC.fC. A.fC.fC. |
| | | | A.mU. G. G.mU. G. | | A.fU.fU. A. G.mC* |
| | | | G. A. A.Chl | | A*mC* G*mC* G* G. |
| 14395 | 2006 | 1161 | mU. G. A.mU.mC. | 1162 | P.mG. A. G.fC. G.fC. |
| | | | G.mU. G.mC. | | A.fC. G. A.mU.mC. |
| | | | G.mC.mU.mC.Chl | | A*mU* G*mU*mU* G* |
| | | | | | G. |
| 14396 | 1389 | 1163 | mC. A. | 1164 | P.mU.fC. G.fC.fC. A. G. |
| | | | A.mU.mU.mC.mC. | | G. A. A.mU.mU. |
| | | | mU. G. G.mC. G. | | G*mU*mU* |
| | | | A.Chl | | G*mC*mU* G. |
| 14397 | 1787 | 1165 | A. G.mU. G. G. | 1166 | P.mU.fC. G.fU. G. G. |
| | | | A.mU.mC.mC. | | A.fU.fC.fC. |
| | | | A.mC. G. A.Chl | | A.mC.mU*mU*mC*mC |
| | | | | | * A* G* C. |
| 14398 | 1867 | 1167 | mU. A.mC. A. | 1168 | P.mG. G. A.fC.fC.fU.fU. |
| | | | G.mC. A. A. G. | | G.fC.fU. G.mU. |
| | | | G.mU.mC.mC.Chl | | A*mC*mU* G*mC* G* |
| | | | | | U. |
| 14399 | 2002 | 1169 | A. A.mC. A.mU. G. | 1170 | P.mG.fC. A.fC. G. |
| | | | A.mU.mC. G.mU. | | A.fU.fC. A.fU. |
| | | | G.mC.Chl | | G.mU.mU* G* G* |
| | | | | | A*mC* A* G. |
| 14400 | 2003 | 1171 | A.mC. A.mU. G. | 1172 | P.mC. G.fC. A.fC. G. |
| | | | A.mU.mC. G.mU. | | A.fU.fC. A.mU. |
| | | | G.mC. G.Chl | | G.mU*mU* G* G* |
| | | | | | A*mC* A. |
| 14401 | 1869 | 1173 | mC. A. G.mC. A. A. | 1174 | P.mC. A. G. G. |
| | | | G. | | A.fC.fC.fU.fU. |
| | | | G.mU.mC.mC.mU. | | G.mC.mU. G*mU* |
| | | | G.Chl | | A*mC*mU* G* C. |
| 14402 | 2000 | 1175 | mC.mC. A. A.mC. | 1176 | P.mA.fC. G. A.fU.fC. |
| | | | A.mU. G. A.mU.mC. | | A.fU. G.fU.mU. G. G* |
| | | | G.mU.Chl | | A*mC* A* G*mC* U. |
| 14403 | 986 | 1177 | A. G.mC. G. G. A. | 1178 | P.mA.fU. G.fC. |
| | | | A. G.mC. G.mC. | | G.fC.fU.fU.fC.fC. |
| | | | A.mU.Chl | | G.mC.mU*mU*mC* |
| | | | | | A*mC*mC* A. |
| 14404 | 995 | 1179 | G.mC. A.mU.mC. | 1180 | P.mA.fU. G. |
| | | | G. A. G. G.mC.mC. | | G.fC.fC.fU.fC. G. A.mU. |
| | | | A.mU.Chl | | G.mC* |
| | | | | | G*mC*mU*mU*mC* C. |
| 14405 | 963 | 1181 | G. A.mC.mU. | 1182 | P.mC. A.fU. G.fU.fC. G. |
| | | | A.mU.mC. G. A.mC. | | A.fU. A. |
| | | | A.mU. G.Chl | | G.mU.mC*mU*mU* |
| | | | | | G*mC* A* G. |
| 14406 | 955 | 1183 | A.mC.mC.mU. | 1184 | P.mU. A. G.fU.fC.fU.fU. |
| | | | G.mC. A. A. G. | | G.fC. A. G. G.mU* G* |
| | | | A.mC.mU. A.Chl | | G* A*mU* A* G. |
| 14407 | 1721 | 1185 | G.mC.mU.mC.mC. | 1186 | P.mU.fU.fC.fU.fC.fC. |
| | | | A.mC. G. G. A. G. A. | | G.fU. G. G. A. |
| | | | A.Chl | | G.mC*mU* G* A* A* |
| | | | | | G* C. |
| 18454 | 1246 | 1187 | mC. A.mC. A. G.mC. | 1188 | P.mU. A.fU. A.fU. A.fU. |
| | | | A.mU. A.mU. A.mU. | | G.fC.fU. G.fU. G*fU* |
| | | | A.Chl | | G*fU* A*fC* U. |
| 18455 | 1248 | 1189 | mC. A. G.mC. | 1190 | P.mU. A.fU. A.fU. A.fU. |
| | | | A.mU. A.mU. A.mU. | | A.fU. G.fC.fU. G*fU* |
| | | | A.mU. A.Chl | | G*fU* G*fU* A. |
| 18456 | 1755 | 1191 | G.mU. A.mC. | 1192 | P.mU. A. A. G.fU.fC. A. |
| | | | A.mU.mU. G. | | A.fU. G.fU. A.fC* A* |
| | | | A.mC.mU.mU. | | G*fC*fU* G* C. |
| | | | A.Chl | | |
| 18457 | 1755 | 1193 | mU. G.mU. A.mC. | 1194 | P.mU. A. A. G.fU.fC. A. |
| | | | A.mU.mU. G. | | A.fU. G.fU. A.fC* A* |
| | | | A.mC.mU.mU. | | G*fC*fU* G* C. |
| | | | A.Chl | | |
| 18458 | 1708 | 1195 | A. A.mC.mU. | 1196 | P.mU. G. A. A. G.fC. A. |
| | | | A.mU.mU. | | A.fU. A. G.fU.fU* G* |
| | | | G.mC.mU.mU.mC. | | G*fU* G*fU* C. |
| | | | A.Chl | | |
| 18459 | 1708 | 1197 | mC. A. A.mC.mU. | 1198 | P.mU. G. A. A. G.fC. A. |
| | | | A.mU.mU. | | A.fU. A. G.fU.fU* G* |
| | | | G.mC.mU.mU.mC. | | G*fU* G*fU* C. |
| | | | A.Chl | | |
| 18460 | 1250 | 1199 | G.mC. A.mU. | 1200 | P.mU. A.fC. A.fU. A.fU. |
| | | | A.mU. A.mU. A.mU. | | A.fU. A.fU. G.fC*fU* |
| | | | G.mU. A.Chl | | G*fU* G*fU* G. |
| 18461 | 1754 | 1201 | mU. G.mU. A.mC. | 1202 | P.mU. A. G.fU.fC. A. |
| | | | A.mU.mU. G. | | A.fU. G.fU. A.fC. A* |
| | | | A.mC.mU. A.Chl | | G*fC*fU* G*fC* C. |
| 18462 | 1754 | 1203 | mC.mU. G.mU. | 1204 | P.mU. A. G.fU.fC. A. |
| | | | A.mC. A.mU.mU. G. | | A.fU. G.fU. A.fC. A* |
| | | | A.mC.mU. A.Chl | | G*fC*fU* G*fC* C. |
| 18463 | 1249 | 1205 | A. G.mC. A.mU. | 1206 | P.mU.fC. A.fU. A.fU. |
| | | | A.mU. A.mU. A.mU. | | A.fU. A.fU. G.fC.fU* |
| | | | G. A.Chl | | G*fU* G*fU* G* U. |
| 18464 | 1383 | 1207 | mC. A. G.mC. A. | 1208 | P.mU. G. A. A.fU.fU. |
| | | | A.mC. A. | | G.fU.fU. G.fC.fU. G*fU* |
| | | | A.mU.mU.mC. | | A*fU*fU*fU* C. |
| | | | A.Chl | | |
| 18465 | 1251 | 1209 | mC. A.mU. A.mU. | 1210 | P.mU. A. A.fC. A.fU. |
| | | | A.mU. A.mU. | | A.fU. A.fU. A.fU. |
| | | | G.mU.mU. A.Chl | | G*fC*fU* G*fU* G* U. |
| 18466 | 1713 | 1211 | mU.mU. | 1212 | P.mU. G. A. G.fC.fU. G. |
| | | | G.mC.mU.mU.mC. | | A. A. G.fC. A. A*fU* A* |
| | | | A. G.mC.mU.mC. | | G*fU*fU* G. |
| | | | A.Chl | | |
| 18467 | 1713 | 1213 | A.mU.mU. | 1214 | P.mU. G. A. G.fC.fU. G. |
| | | | G.mC.mU.mU.mC. | | A. A. G.fC. A. A*fU* A* |
| | | | A. G.mC.mU.mC. | | G*fU*fU* G. |
| | | | A.Chl | | |
| 18468 | 1247 | 1215 | A.mC. A. G.mC. | 1216 | P.mU.fU. A.fU. A.fU. |
| | | | A.mU. A.mU. A.mU. | | A.fU. G.fC.fU. G.fU* |
| | | | A. A.Chl | | G*fU* G*fU* A* C. |
| 18469 | 1712 | 1217 | A.mU.mU. | 1218 | P.mU. A. G.fC.fU. G. A. |
| | | | G.mC.mU.mU.mC. | | A. G.fC. A. A.fU* A* |
| | | | A. G.mC.mU. A.Chl | | G*fU*fU* G* G. |
| 18470 | 1712 | 1219 | mU. A.mU.mU. | 1220 | P.mU. A. G.fC.fU. G. A. |
| | | | G.mC.mU.mU.mC. | | A. G.fC. A. A.fU* A* |
| | | | A. G.mC.mU. A.Chl | | G*fU*fU* G* G. |
| 18471 | 1212 | 1221 | mC. A. A. | 1222 | P.mU.fU. G.fC.fU.fU. G. |
| | | | G.mU.mU.mC. A. A. | | A. A.fC.fU.fU. G*fU*fC* |
| | | | G.mC. A. A.Chl | | A*fU* A* G. |
| 18472 | 1222 | 1223 | mC. A. G. A. G.mU. | 1224 | P.mU. G.fU. G.fU. G.fU. |
| | | | A.mC. A.mC. A.mC. | | A.fC.fU.fC.fU. G* |
| | | | A.Chl | | C*fU*fU* G* A* A. |
| 18473 | 1228 | 1225 | A.mC. A.mC. A.mC. | 1226 | P.mU.fU. A.fU. G.fC.fU. |
| | | | A. G.mC. A.mU. A. | | G.fU. G.fU. G.fU* |
| | | | A.Chl | | A*fC*fU*fC*fU* G. |
| 18474 | 1233 | 1227 | mC. A. G.mC. | 1228 | P.mU. A.fU. A.fU. A.fU. |
| | | | A.mU. A.mU. A.mU. | | A.fU. G.fC.fU. G*fU* |
| | | | A.mU. A.Chl | | G*fU* G*fU* A. |
| 18475 | 1218 | 1229 | mU.mC. A. A. | 1230 | P.mU.fU. A.fC.fU.fC.fU. |
| | | | G.mC. A. G. A. | | G.fC.fU.fU. G. A* |
| | | | G.mU. A. A.Chl | | A*fC*fU*fU* G* U. |
| 18476 | 1235 | 1231 | A. G.mC. A.mU. | 1232 | P.mU.fC. A.fU. A.fU. |
| | | | A.mU. A.mU. A.mU. | | A.fU. A.fU. G.fC.fU* |
| | | | G. A.Chl | | G*fU* G*fU* G* U. |
| 18477 | 1225 | 1233 | A. G. A. G.mU. | 1234 | P.mU.fU. G.fU. G.fU. |
| | | | A.mC. A.mC. A.mC. | | G.fU. A.fC.fU.fC.fU* |
| | | | A. A.Chl | | G*fC*fU*fU* G* A. |
| 18478 | 1221 | 1235 | A. A. G.mC. A. G. A. | 1236 | P.mU.fU. G.fU. |
| | | | G.mU. A.mC. A. | | A.fC.fU.fC.fU. |
| | | | A.Chl | | G.fC.fU.fU* G* A* |
| | | | | | A*fC*fU* U. |
| 18479 | 1244 | 1237 | mU.mU.mC. A. | 1238 | P.mU.fU. G. A.fU. G.fU. |
| | | | A.mC. A.mC. | | G.fU.fU. G. A. A* G* A* |
| | | | A.mU.mC. A. A.Chl | | A*fC* A* U. |
| 18480 | 1224 | 1239 | A. G.mC. A. G. A. | 1240 | P.mU. G.fU. G.fU. |
| | | | G.mU. A.mC. A.mC. | | A.fC.fU.fC.fU. |
| | | | A.Chl | | G.fC.fU*fU* G* A* |
| | | | | | A*fC* U. |
| 18481 | 1242 | 1241 | A.mU. A.mU. | 1242 | P.mU. A. A. G. A. A.fC. |
| | | | A.mU. | | A.fU. A.fU. A.fU* A*fU* |
| | | | G.mU.mU.mC.mU. | | G*fC*fU* G. |
| | | | mU. A.Chl | | |
| 18482 | 1213 | 1243 | G. A.mC. A. A. | 1244 | P.mU.fC.fU.fU. G. A. |
| | | | G.mU.mU.mC. A. A. | | A.fC.fU.fU. G.fU.fC* |
| | | | G. A.Chl | | A*fU* A* G* A* U. |
| 18483 | 1760 | 1245 | mU.mU. A. A. A. G. | 1246 | P.mU.fC.fU.fC.fC. |
| | | | A.mU. G. G. A. G. | | A.fU.fC.fU.fU.fU. A. |
| | | | A.Chl | | A*fU* G* G* G* G* C. |
| 18484 | 1211 | 1247 | mC.mU. A.mU. G. | 1248 | P.mU. A. A.fC.fU.fU. |
| | | | A.mC. A. A. | | G.fU.fC. A.fU. A. G* |
| | | | G.mU.mU. A.Chl | | A*fU*fU*fU*fC* G. |
| 19411 | 1212 | 1249 | mC. A. A.mC. G. A. | 1250 | P.mU.fU. A. G. |
| | | | A. A.mU.mC.mU. A. | | A.fU.fU.fU.fC. G.fU.fU. |
| | | | A.Chl | | G*fU* G* G* G*fU*fU. |
| 19412 | 1222 | 1251 | mU. A.mU. G. | 1252 | P.mU. G. A. A.fC.fU.fU. |
| | | | A.mC. A. A. | | G.fU.fC. A.fU. A* G* |
| | | | G.mU.mU.mC. | | A*fU*fU*fU*fC. |
| | | | A.Chl | | |
| 19413 | 1228 | 1253 | A. A. | 1254 | P.mU.fC.fU. G.fC.fU.fU. |
| | | | G.mU.mU.mC. A. A. | | G. A. A.fC.fU.fU* |
| | | | G.mC. A. G. A.Chl | | G*fU*fC* A*fU* A. |
| 19414 | 1233 | 1255 | mC. A. A. G.mC. A. | 1256 | P.mU. G.fU. |
| | | | G. A. G.mU. A.mC. | | A.fC.fU.fC.fU. |
| | | | A.Chl | | G.fC.fU.fU. G* A* |
| | | | | | A*fC*fU*fU* G. |
| 19415 | 1218 | 1257 | A. A.mU.mC.mU. | 1258 | P.mU.fU.fU. G.fU.fC. |
| | | | A.mU. G. A.mC. A. | | A.fU. A. G. |
| | | | A. A.Chl | | A.fU.fU*fU*fC* |
| | | | | | G*fU*fU* G. |
| 19416 | 1244 | 1259 | mC. A.mC. A.mC. A. | 1260 | P.mU. A.fU. A.fU. |
| | | | G.mC. A.mU. A.mU. | | G.fC.fU. G.fU. G.fU. |
| | | | A.Chl | | G*fU* A*fC*fU*fC*fU. |
| 19417 | 655 | 1261 | G. A. A. A.mU. | 1262 | P.mU.fU.fU. G.fC.fU. |
| | | | A.mU. A. G.mC. A. | | A.fU. A.fU.fU.fU.fC*fU* |
| | | | A. A.Chl | | G* G*fU* A* G. |
| 19418 | 644 | 1263 | G. A. | 1264 | P.mU.fC.fU. G. G.fU. A. |
| | | | A.mC.mU.mC.mU. | | G. A. G.fU.fU.fC*fU* |
| | | | A.mC.mC. A. G. | | A*fC* G*fU* G. |
| | | | A.Chl | | |
| 19419 | 819 | 1265 | G.mC. A. A. A. G. | 1266 | P.mU.fC. A.fU.fU. |
| | | | A.mU. A. A.mU. G. | | A.fU.fC.fU.fU.fU. |
| | | | A.Chl | | G.fC*fU* G*fU*fC* A* |
| | | | | | C. |
| 19420 | 645 | 1267 | A. | 1268 | P.mU.fU.fC.fU. G. G.fU. |
| | | | A.mC.mU.mC.mU. | | A. G. A. G.fU.fU*fC*fU* |
| | | | A.mC.mC. A. G. A. | | A*fC* G* U. |
| | | | A.Chl | | |
| 19421 | 646 | 1269 | A.mC.mU.mC.mU. | 1270 | P.mU.fU.fU.fC.fU. G. |
| | | | A.mC.mC. A. G. A. | | G.fU. A. G. A. |
| | | | A. A.Chl | | G.fU*fU*fC*fU* A*fC* |
| | | | | | G. |
| 19422 | 816 | 1271 | A.mC. A. G.mC. A. | 1272 | P.mU.fU. |
| | | | A. A. G. A.mU. A. | | A.fU.fC.fU.fU.fU. |
| | | | A.Chl | | G.fC.fU. G.fU*fC* A*fC* |
| | | | | | A* A* G. |
| 19423 | 495 | 1273 | mC. A. | 1274 | P.mU.fU. G.fU.fC. A.fU. |
| | | | A.mU.mC.mU. | | A. G. A.fU.fU. G*fC* |
| | | | A.mU. G. A.mC. A. | | G*fU*fU* G* U. |
| | | | A.Chl | | |
| 19424 | 614 | 1275 | A. G. | 1276 | P.mU.fU. G. A.fC.fU.fU. |
| | | | A.mU.mU.mC. A. A. | | G. A. A.fU.fC.fU*fC*fU* |
| | | | G.mU.mC. A. A.Chl | | G*fC* A* G. |
| 19425 | 627 | 1277 | mC.mU. G.mU. G. | 1278 | P.mU. G.fU.fU. |
| | | | G. A. G.mC. A. | | G.fC.fU.fC.fC. A.fC. A. |
| | | | A.mC. A.Chl | | G*fU*fU* G* A*fC* U. |
| 19426 | 814 | 1279 | mU. G. A.mC. A. | 1280 | P.mU.fU.fC.fU.fU.fU. |
| | | | G.mC. A. A. A. G. A. | | G.fC.fU. G.fU.fC. A*fC* |
| | | | A.Chl | | A* A* G* A* G. |
| 19427 | 501 | 1281 | A.mU. G. A.mC. A. | 1282 | P.mU.fU. G. |
| | | | A. A. A.mC.mC. A. | | G.fU.fU.fU.fU. G.fU.fC. |
| | | | A.Chl | | A.fU* A* G* A*fU*fU* |
| | | | | | G. |
| 19428 | 613 | 1283 | G. A. G. | 1284 | P.mU. G. A.fC.fU.fU. G. |
| | | | A.mU.mU.mC. A. A. | | A. A.fU.fC.fU.fC*fU* |
| | | | G.mU.mC. A.Chl | | G*fC* A* G* G. |
| 21240 | 1244 | 1285 | mC. A.mC. A.mC. A. | 1286 | P.mU. A.fU. A.fU. |
| | | | G.mC. A.mU. A.mU. | | G.fC.fU. G.fU. G.fU. |
| | | | A.Chl | | G*mU* |
| | | | | | A*mC*mU*mC* U. |
| 21241 | 1244 | 1287 | mC. A.mC. A.mC. A. | 1288 | P.mU. A.fU. A.fU. |
| | | | G.mC. A.mU. A.mU. | | G.fC.fU. G.fU. G.fU. |
| | | | A.Chl | | G*mU*mA*mC*mU*m |
| | | | | | C* U. |
| 21242 | 1244 | 1289 | mC. A.mC. A.mC. A. | 1290 | P.mU. A.fU. A.fU. |
| | | | G.mC. A.mU. A.mU. | | G.fC.fU. G.fU. |
| | | | A.Chl | | G.fU.mG*mU*mA*mC* |
| | | | | | mU*mC* U. |
| 21243 | 1244 | 1291 | mC. A.mC. A.mC. A. | 1292 | P.mU. A.fU. A.fU. |
| | | | G.mC. A.mU. A.mU. | | G.fC.fU. G.fU. |
| | | | A.Chl | | G.fU.mG*fU*mA*fC*m |
| | | | | | U*fC* U. |
| 21244 | 1244 | 1293 | mC. A.mC. A.mC. A. | 1294 | P.mU. A.fU. A.fU. |
| | | | G.mC. A.mU. A.mU. | | G.fC.fU. G.fU. G.fU. |
| | | | A.Chl | | G*fU* A*fC*mU*mC* |
| | | | | | U. |
| 21245 | 1244 | 1295 | mC. A.mC. A.mC. A. | 1296 | P.mU. A.fU. A.fU. |
| | | | G.mC. A.mU. | | G.fC.fU. G.fU. G.fU. |
| | | | A*mU*mA.TEG-Chl | | G*fU* A*fC*fU*fC*fU. |
| 21246 | 1244 | 1297 | mC*mA*mC. A.mC. | 1298 | P.mU. A.fU. A.fU. |
| | | | A. G.mC. A.mU. | | G.fC.fU. G.fU. G.fU. |
| | | | A*mU*mA.TEG-Chl | | G*fU* A*fC*fU*fC*fU. |
| 21247 | 1244 | 1299 | mC*mA*mC.mA.m | 1300 | P.mU. A.fU. A.fU. |
| | | | C.mA.mG.mC.mA. | | G.fC.fU. G.fU. G.fU. |
| | | | mU.mA*mU*mA.T | | G*fU* A*fC*fU*fC*fU. |
| | | | EG-Chl | | |
| 21248 | 614 | 1301 | mA. G. | 1302 | P.mU.fU. G. A.fC.fU.fU. |
| | | | A.mU.mU.mC. A. A. | | G. A. A.fU.fC.fU*fC*fU* |
| | | | G.mU.mC*mA*mA. | | G*fC*fU* U. |
| | | | TEG-Chl | | |
| | | 1303 | | 1304 | |
| 20608 | 1244 | 1305 | mC. A.mC. A.mC. A. | 1306 | P.mU. A.fU. A.fU. |
| | | | G.mC. A.mU. A.mU. | | G.fC.fU. G.fU. G.mU. |
| | | | A.Chl | | G*mU* |
| | | | | | A*mC*mU*mC* U. |
| 20609 | 1244 | 1307 | mC. A.mC. A.mC. A. | 1308 | P.mU. A.fU. A.fU. |
| | | | G.mC. A.mU. A.mU. | | G.fC.fU. G.fU. G.mU. |
| | | | A.Chl | | G*fU* A*mC*fU*mC* |
| | | | | | U. |
| 20610 | 1244 | 1309 | mC. A.mC. A.mC. A. | 1310 | P.mU. A. U. A. U. G. C. |
| | | | G.mC. A.mU. A.mU. | | U. G. U. G.mU. G*mU* |
| | | | A.Chl | | A*mC*mU*mC* U. |
| 20611 | 1244 | 1311 | mC. A.mC. A.mC. A. | 1312 | P.mU. A.fU. A.fU. |
| | | | G.mC. A.mU. A.mU. | | G.fC.fU. G.fU. |
| | | | A.Chl | | G.mU.mG*mU*mA*mC |
| | | | | | *mU*mC* U. |
| 21374 | 614 | 1313 | mC*mA*mC.mA.m | 1314 | P.mU. A.fU. A.fU. |
| | | | C.mA.mG.mC.mA. | | G.fC.fU. G.fU. |
| | | | mU.mA*mU*mA.T | | G.fU.mG*fU*mA*fC*m |
| | | | EG-Chl | | U*fC* U. |

| | |
|---|---|
| Key | |
| Chl = | cholesterol with hydroxyprolinol linker |
| TEG-chl = | cholesterol with TEG linker |
| m = | 2′Ome |
| f = | 2′fluoro |
| * = | phosphorothioate llinkage |
| . = | phosphodiester linkage |

TABLE 8
Examples of VEGF (Accession No.
NM_001171623.1) sd-rxRNA sequences

| Oligo | Gene | Ref | SEQ | Sense | SEQ | Antisense |
|---|---|---|---|---|---|---|
| ID | Region | Pos | ID | sequence | ID | sequence |
| 19850 | CDS | 1389 | 1315 | GAUGAGCUUCCUA | 1316 | UAGGAAGCUCAUCUCUCCU |
| 19851 | 3′UTR | 1853 | 1317 | AGAACAGUCCUUA | 1318 | UAAGGACUGUUCUGUCGAU |
| 19852 | 3′UTR | 1854 | 1319 | GAACAGUCCUUAA | 1320 | UUAAGGACUGUUCUGUCGA |
| 19853 | 3′UTR | 1857 | 1321 | CAGUCCUUAAUCA | 1322 | UGAUUAAGGACUGUUCUGU |
| 19854 | 3′UTR | 1859 | 1323 | GUCCUUAAUCCAA | 1324 | UUGGAUUAAGGACUGUUCU |
| 19855 | 3′UTR | 1863 | 1325 | UUAAUCCAGAAAA | 1326 | UUUUCUGGAUUAAGGACUG |
| 19856 | 3′UTR | 2183 | 1327 | UGUUAUUGGUGUA | 1328 | UACACCAAUAACAUUAGCA |
| 19857 | 3′UTR | 2790 | 1329 | UUGAAACCACUAA | 1330 | UUAGUGGUUUCAAUGGUGU |
| 19858 | 3′UTR | 2931 | 1331 | GAGAAAAGAGAAA | 1332 | UUUCUCUUUUCUCUGCCUC |
| 19859 | 3′UTR | 2932 | 1333 | AGAAAAGAGAAAA | 1334 | UUUUCUCUUUUCUCUGCCU |
| 19860 | 3′UTR | 2933 | 1335 | GAAAAGAGAAAGA | 1336 | UCUUUCUCUUUUCUCUGCC |
| 19861 | 3′UTR | 3199 | 1337 | ACACUCAGCUCUA | 1338 | UAGAGCUGAGUGUUAGCAA |
| 19862 | 3′UTR | 3252 | 1339 | AAAUAAGGUUUCA | 1340 | UGAAACCUUAUUUCAAAGG |
| 19863 | 3′UTR | 3427 | 1341 | AAUCUCUCUCCUA | 1342 | UAGGAGAGAGAUUUAGUAU |
| 19864 | 3′UTR | 3429 | 1343 | UCUCUCUCCUUUA | 1344 | UAAAGGAGAGAGAUUUAGU |
| 19865 | 3′UTR | 3430 | 1345 | CUCUCUCCUUUUA | 1346 | UAAAAGGAGAGAGAUUUAG |
| 19866 | 3′UTR | 3471 | 1347 | AUUGGUGCUACUA | 1348 | UAGUAGCACCAAUAAAUAA |
| 19867 | 3′UTR | 3476 | 1349 | UGCUACUGUUUAA | 1350 | UUAAACAGUAGCACCAAUA |
| 19868 | 3′UTR | 1852 | 1351 | CAGAACAGUCCUA | 1352 | UAGGACUGUUCUGUCGAUG |
| 19869 | CDS | 1343 | 1353 | UGCAGAUUAUGCA | 1354 | UGCAUAAUCUGCAUGGUGA |
| 19870 | CDS | 1346 | 1355 | GAUUAUGCGGAUA | 1356 | UAUCCGCAUAAUCUGCAUG |
| 19871 | CDS | 1352 | 1357 | UGCGGAUCAAACA | 1358 | UGUUUGAUCCGCAUAAUCU |
| 19872 | 3′UTR | 1985 | 1359 | GGAUUCGCCAUUA | 1360 | UAAUGGCGAAUCCAAUUCC |
| 19873 | 3′UTR | 2210 | 1361 | UUGACUGCUGUGA | 1362 | UCACAGCAGUCAAAUACAU |
| 19874 | 3′UTR | 2447 | 1363 | CAGAAAGACAGAA | 1364 | UUCUGUCUUUCUGUCCGUC |
| 19875 | 3′UTR | 2792 | 1365 | GAAACCACUAGUA | 1366 | UACUAGUGGUUUCAAUGGU |
| 19876 | 3′UTR | 2794 | 1367 | AACCACUAGUUCA | 1368 | UGAACUAGUGGUUUCAAUG |
| 19877 | 3′UTR | 3072 | 1369 | UAUCUUUUGCUCA | 1370 | UGAGCAAAAGAUACAUCUC |
| 19878 | 3′UTR | 3073 | 1371 | AUCUUUUGCUCUA | 1372 | UAGAGCAAAAGAUACAUCU |
| 19879 | 3′UTR | 3162 | 1373 | UCACUAGCUUAUA | 1374 | UAUAAGCUAGUGACUGUCA |
| 19880 | 3′UTR | 3163 | 1375 | CACUAGCUUAUCA | 1376 | UGAUAAGCUAGUGACUGUC |

TABLE 9
Examples of selected VEGF rxRNAori Sequences

| Oligo | Start | 25 mer Sense | 25 mer Anti- |
|---|---|---|---|
| ID | Site | Sequence | sense sequence |
| 18760 | 1853 | 5′-AUCACCAUCGAC | 5′-UAAGGACUGUUCUG |
| | | AGAACAGUCCUUA | UCGAUGGUGAU |
| | | (SEQ ID NO: 13) | (SEQ ID NO: 1377) |
| 18886 | 1352 | 5′-CCAUGCAGAUUA | 5′-UGUUUGAUCCGCAU |
| | | UGCGGAUCAAACA | AAUCUGCAUGG |
| | | (SEQ ID NO: 28) | (SEQ ID NO: 1378) |

TABLE 10
Optimized VEGF sd-rxRNA Sequences With Increased Stability

| Duplex | Oligo ID | SEQ ID NO | |
|---|---|---|---|
| 19851 | 19790 | 1379 | A. G. A. A.mC. A. G.mU.mC.mC.mU.mU. A.Chl |
| | 19791 | 1380 | P.mU. A. A. G. G. A.fC.fU. G.fU.fU.fC.fU* G*fU*fC* G* A* |
| | | | U |
| | Description | | |
| SS | 3′ Ome block | 1381 | A.G.A.A.mC.A.G.mU.mC.mC.mU*mU*mA-TEG-Chl |
| | Complete Ome | 1382 | mA.mG.mA.mA.mC.mA.mG.mU.mC.mC.mU*mU*mA- |
| | | | TEG-Chl |
| | 3′ and 5′ Ome | 1383 | mA.mG.A.A.mC.A.G.mU.mC.mC.mU*mU*mA-TEG-Chl |
| | block | | |
| AS - no >3 | Pos 5 2′Ome G | 1384 | P.mU.A.A.G.mG.A.fC.fU.G.fU.fU.fC.fU*G*fU*fC*G*A*U |
| 2′OH | Pos 4 2′Ome G | 1385 | P.mU.A.A.mG.G.A.fC.fU.G.fU.fU.fC.fU*G*fU*fC*G*A*U |
| | Pos 3 2′Ome A | 1386 | P.mU.A.mA.G.G.A.fC.fU.G.fU.fU.fC.fU*G*fU*fC*G*A*U |
| | Pos 4 2′F G | 1387 | P.mU.A.A.fG.G.A.fC.fU.G.fU.fU.fC.fU*G*fU*fC*G*A*U |
| Stabilizing | No 2′OH 3′ tail | 1388 | P.mU.A.A.mG.G.A.fC.fU.G.fU.fU.fC.fU*mG*fU*fC*mG*mA |
| 3′ end (no | | | *U |
| 2′OH | (1) 2′OH 3′ tail | 1389 | P.mU.A.A.mG.G.A.fC.fU.G.fU.fU.fC.fU*G*fU*fC*mG*mA* |
| | | | U |
| | No 2′OH 3′ tail | 1390 | P.mU.A.A.fG.G.A.fC.fU.G.fU.fU.fC.fU*mG*fU*fC*mG*mA* |
| | | | U |
| | (1) 2′OH 3′ tail | 1391 | P.mU.A.A.fG.G.A.fC.fU.G.fU.fU.fC.fU*G*fU*fC*mG*mA*U |
| | No 2′OH 3′ tail | 1392 | P.mU.A.A.fG.G.A.fC.fU.G.fU.fU.fC.fU*fG*fU*fC*mG*mA* |
| | | | U |
| 5 Methyl C | | 1393 | P.mY.A.A.fG.G.A.fX.fY.G.fY.fY.fX.fU*G*fY*fX*mG*mA*U |
| and U | | 1394 | P.mY.A.A.fG.G.A.fX.fY.G.fY.fY.fX.fU*mG*fY*fX*mG*mA*U |
| | | 1395 | P.mY.A.A.mG.G.A.fX.fY.G.fY.fY.fX.fU*G*fY*fX*mG*mA*U |
| | | 1396 | P.mY.A.A.mG.G.A.fX.fY.G.fY.fY.fX.fU*mG*fY*fX*mG*mA* |
| | | | U |
| 19871 | 19830 | 1397 | mU. G.mC. G. G. A.mU.mC. A. A. A.mC. A.Chl |
| | 19831 | 1398 | P.mU. G.fU.fU.fU. G. A.fU.fC.fC. G.fC. A*fU* A* A*fU*fC* |
| | | | U |

| | |
|---|---|
| Key | |
| Chl = | cholesterol with hydroxyprolinol linker |
| TEG-chl = | cholesterol with TEG linker |
| m = | 2′Ome |
| f = | 2′fluoro |
| * = | phosphorothioate llinkage |
| . = | phosphodiester linkage |

TABLE 11
Examples of MAP4K4 sd-rxRNA sequences

| | SEQ ID NO. for | | SEQ ID for | |
|---|---|---|---|---|
| | sense strand | Sense strand | antisense strand | Anti-sense strand |
| Oligo ID | sequence | sequence | sequence | sequence |
| MAP4K4.1 | 1399 | fC.fU.fG.fU.fG.fG.fA.fA | 1400 | P.fU.fA.fG.fA.fC.fU.fU.fC. |
| | | .fG.fU.fC*fU*fA-Chl | | fC.fA.fC*fA*fG*fA*fA*fC |
| | | | | *fU*fC*U |
| MAP4K4.2 | 1401 | fC.fU.fG.fU.fG.fG.fA.fA | 1402 | P.fU.fA.fG.fA.fC.fU.fU.fC. |
| | | .fG.fU.fC*fU*fA-Chl | | fC.fA.mC*fA*mG*fA*mA |
| | | | | *mC*mU*mC*U |
| MAP4K4.3 | 1403 | fC.fU.fG.fU.fG.fG.fA.fA | 1404 | P.fY.A.G.A.fX.fY.fY.fX.fC.A |
| | | .fG.fU.fC*fU*fA-Chl | | .mX*A*mG*A*mA*mX* |
| | | | | mY*mX*U |
| MAP4K4.4 | 1405 | fC.fU.fG.fU.fG.fG.fA.fA | 1406 | P.fY.fA.fG.fA.fX.fY.fY.fX.f |
| | | .fG.fU.fC*fU*fA-Chl | | X.fA.mX*fA*mG*fA*mA* |
| | | | | mX*mY*mX*U |
| MAP4K4.5 | 1407 | mX.mY.G.mY.G.G.A.A. | 1408 | P.fU.fA.fG.fA.fC.fU.fU.fC. |
| | | G.mY.mX*mY*A-Chl | | fC.fA.fC*fA*fG*fA*fA*fC |
| | | | | *fU*fC*U |
| MAP4K4.6 | 1409 | mX.mY.G.mY.G.G.A.A. | 1410 | P.fU.fA.fG.fA.fC.fU.fU.fC. |
| | | G.mY.mX*mY*A-Chl | | fC.fA.mC*fA*mG*fA*mA |
| | | | | *mC*mU*mC*U |
| MAP4K4.7 | 1411 | mX.mY.G.mY.G.G.A.A. | 1412 | P.fY.A.G.A.fX.fY.fY.fX.fC.A |
| | | G.mY.mX*mY*A-Chl | | .mX*A*mG*A*mA*mX* |
| | | | | mY*mX*U |
| MAP4K4.8 | 1413 | mX.mY.G.mY.G.G.A.A. | 1414 | P.fY.fA.fG.fA.fX.fY.fY.fX.f |
| | | G.mY.mX*mY*A-Chl | | X.fA.mX*fA*mG*fA*mA* |
| | | | | mX*mY*mX*U |
| MAP4K4.9 | 1415 | mC.mU. G.mU. G. G. | 1416 | P.fU.fA.fG.fA.fC.fU.fU.fC. |
| | | A. A. G.mU.mC*mU* | | fC.fA.fC*fA*fG*fA*fA*fC |
| | | A-Chl | | *fU*fC*U |
| MAP4K4.10 | 1417 | mC.mU. G.mU. G. G. | 1418 | P.fU.fA.fG.fA.fC.fU.fU.fC. |
| | | A. A. G.mU.mC*mU* | | fC.fA.mC*fA*mG*fA*mA |
| | | A-Chl | | *mC*mU*mC*U |
| MAP4K4.11 | 1419 | mC.mU. G.mU. G. G. | 1420 | P.fY.A.G.A.fX.fY.fY.fX.fC.A |
| | | A. A. G.mU.mC*mU* | | .mX*A*mG*A*mA*mX* |
| | | A-Chl | | mY*mX*U |
| MAP4K4.12 | 1421 | mC.mU. G.mU. G. G. | 1422 | P.fY.fA.fG.fA.fX.fY.fY.fX.f |
| | | A. A. G.mU.mC*mU* | | X.fA.mX*fA*mG*fA*mA* |
| | | A-Chl | | mX*mY*mX*U |
| MAP4K4.13 | 1423 | fC.fU.fG.fU.fG.fG.fA.fA | 1424 | P.fU. A. G. |
| | | .fG.fU.fC*fU*fA-Chl | | A.fC.fU.fU.fC.fC. A.mC. |
| | | | | A.mG* |
| | | | | A*mA*mC*mU*mC* U |
| MAP4K4.14 | 1425 | mX.mY.G.mY.G.G.A.A. | 1426 | P.fU. A. G. |
| | | G.mY.mX*mY*A-Chl | | A.fC.fU.fU.fC.fC. A.mC. |
| | | | | A.mG* |
| | | | | A*mA*mC*mU*mC* U |
| MAP4K4.15 | 1427 | mC.mU. G.mU. G. G. | 1428 | P.mU. A. G. |
| | | A. A. G.mU.mC.mU. | | A.fC.fU.fU.fC.fC. A.mC. A. |
| | | A.TEG-Chl | | G* A* A*mC*mU*mC* U |
| MAP4K4.16 | 1429 | mC.mU. G.mU. G. G. | 1430 | P.fU. A. G. |
| | | A. A. G.mU.mC*mU* | | A.fC.fU.fU.fC.fC. A.mC* |
| | | A-Chl | | A*mG* |
| | | | | A*mA*mC*mU*mC* U |

Key:
Chl = cholesterol
m = 2′ome
f = 2′fluoro
* = phosphorothioate
. = phosphorodiester linkage
X = 5-methyl cytosine
Y = 5-methyl uracil

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety. This application incorporates by reference the entire contents, including all the drawings and all parts of the specification (including sequence listing or amino acid/polynucleotide sequences) of US Patent Publication No. US2013/0131142, entitled “RNA Interference in Ocular Indications,” filed on Feb. 5, 2013, PCT Publication No. WO2010/033247 (Application No. PCT/US2009/005247), filed on Sep. 22, 2009, and entitled “REDUCED SIZE SELF-DELIVERING RNAI COMPOUNDS,” U.S. Pat. No. 8,796,443, issued on Aug. 5, 2014, published as US 2012/0040459 on Feb. 16, 2012, entitled “REDUCED SIZE SELF-DELIVERING RNAI COMPOUNDS,” PCT Publication No. WO2009/102427 (Application No. PCT/US2009/000852), filed on Feb. 11, 2009, and entitled, “MODIFIED RNAI POLYNUCLEOTIDES AND USES THEREOF,” and US Patent Publication No. 2011/0039914, published on Feb. 17, 2011 and entitled “MODIFIED RNAI POLYNUCLEOTIDES AND USES THEREOF.”

1. A method for treating an ocular disorder associated with the front of the eye, comprising
administering to the eye of a subject in need thereof a therapeutic RNA molecule, in an effective amount to treat an ocular disorder associated with the front of the eye.

2. The method of claim 1, wherein the ocular disorder associated with the front of the eye is selected from the group consisting of: Corneal scarring, corneal perforation, corneal dystrophies, corneal injury and/or trauma (including burns), corneal inflammation, corneal infection, opthalmia neonatorum, erythema multiform (Stevens-Johnson Syndrome), xerophthalmia (dry eye syndrome), trachoma, onchocerciasis (river blindness), corneal complications of leprosy, keratitis, persistent corneal epithelial defects, conjunctivitis, anterior uveitis, iridocorneal endothelial syndrome, Fuch's Dystrophy, trichiasis, ocular herpes, corneal grafting or transplant (including ex vivo treatment of a graft or transplant prior to surgery), corneal transplant failure and/or rejection.

3. The method of claim 1 or 2, wherein the therapeutic RNA molecule is delivered to an area of the eye other than the front of the eye.

4. The method of claim 1 or 2, wherein the therapeutic RNA molecule is delivered to the front of the eye.

5. The method of any one of claims 1-4, wherein the therapeutic RNA molecule is administered by a method selected from the group consisting of: intravitreal, subretinal, periocular (subconjunctival, sub-tenon, retrobulbar, peribulbar and posterior juxtascleral), topical, eye drops, corneal implants, biodegradable implants, non-biodegradable implants ocular inserts, thin-films, sustained release formulations, polymers and slow release polymers, iontophoresis, hydrogel contact lenses, reverse/thermal hydrogels and biodegradable pellets.

6. The method of any one of claims 1-5, wherein the therapeutic RNA molecule is directed against a gene encoding a protein selected from the group consisting of: CTGF, VEGF, MAP4K4, PDGF-B, SDF-1, IGTA5, ANG2, HIF-1α, mTOR, SDF-1, PDGF-B, SPP1, PTGS2 (COX-2), TGFβ1, TGFβ2, complement factors 3 and 5, PDGFRa, PPIB, IL-1 alpha, IL-1 beta, Icam-1, Tie 1, Tie 2, ANg 1, Ang 2, and myc, or a combination thereof.

7. The method of claim 6, wherein the therapeutic RNA molecule is directed against a gene encoding CTGF.

8. The method of claim 6, wherein the therapeutic RNA molecule is directed against a gene encoding VEGF.

9. The method of claim 6, wherein the therapeutic RNA molecule is directed against a gene encoding Map4K4.

10. The method of any one of claims 1-9, wherein two or more different therapeutic RNA molecules that are directed against genes encoding two or more different proteins are both administered to the eye of the subject.

11. The method of any one of claims 1-9, wherein two or more different therapeutic RNA molecules that are directed against genes encoding the same protein are both administered to the eye of the subject.

12. The method of any one of claims 1-11, wherein the therapeutic RNA molecule is an sd-rxRNA.

13. The method of claim 12, wherein the sd-rxRNA comprises at least 12 contiguous nucleotides of a sequence selected from the sequences within Tables 3-8, 10 or 11.

14. The method of claim 12, wherein the antisense strand of the sd-rxRNA comprises at least 12 contiguous nucleotides of the sequence of SEQ ID NO:948 or SEQ ID NO:964.

15. The method of claim 12, wherein the sense strand of the sd-rxRNA comprises at least 12 contiguous nucleotides of the sequence of SEQ ID NO:947 or SEQ ID NO:963.

16. The method of claim 12, wherein the sense strand of the sd-rxRNA comprises SEQ ID NO:947 and the antisense strand of the sd-rxRNA comprises SEQ ID NO:948.

17. The method of claim 12, wherein the sense strand of the sd-rxRNA comprises at least 12 contiguous nucleotides of the sequence of SEQ ID NO:1317 or SEQ ID NO:1357.

18. The method of claim 12, wherein the antisense strand of the sd-rxRNA comprises at least 12 contiguous nucleotides of the sequence of SEQ ID NO:1318 or SEQ ID NO:1358.

19. The method of claim 12, wherein the sense strand of the sd-rxRNA comprises SEQ ID NO:1317 and the antisense strand of the sd-rxRNA comprises SEQ ID NO:1318.

20. The method of claim 12, wherein the sense strand of the sd-rxRNA comprises SEQ ID NO:1357 and the antisense strand of the sd-rxRNA comprises SEQ ID NO:1358.

21. The method of claim 12, wherein the sense strand of the sd-rxRNA comprises SEQ ID NO:1379 and the antisense strand of the sd-rxRNA comprises SEQ ID NO:1380.

22. The method of claim 12, wherein the sense strand of the sd-rxRNA comprises SEQ ID NO:1397 and the antisense strand of the sd-rxRNA comprises SEQ ID NO:1398.

23. The method of any one of claims 12-22, wherein the sd-rxRNA is hydrophobically modified.

24. The method of claim 23, wherein the sd-rxRNA is linked to one or more hydrophobic conjugates.

25. The method of any one of claims 1-11, wherein the therapeutic RNA molecule is an rxRNAori.

26. An sd-rxRNA that is directed against a sequence comprising at least 12 contiguous nucleotides of a sequence within Table 11.

27. An sd-rxRNA that comprises at least 12 contiguous nucleotides of a sequence within Table 11.

28. The method of any one of claims 1-9, wherein the therapeutic RNA molecule is administered to an eye that is compromised and/or wounded.

29. The method of claim 28, wherein the cornea is compromised and/or wounded.

30. The method of claim 28 or claim 29, wherein the therapeutic RNA molecule is administered to the cornea.

31. The method of any one of claims 28-30, wherein the therapeutic RNA molecule is administered topically.


SEQUENCE LISTING (1434 sequences)

SEQ ID NO: 1 - RNA, 25 nt, Artificial Sequence
  Synthetic Polynucleotide
uaucauuuauuuauuggugcuacua

SEQ ID NO: 2 - RNA, 25 nt, Artificial Sequence
  Synthetic Polynucleotide
uuaauuuugcuaacacucagcucua

SEQ ID NO: 3 - RNA, 25 nt, Artificial Sequence
  Synthetic Polynucleotide
ccucacaccauugaaaccacuagua

SEQ ID NO: 4 - RNA, 25 nt, Artificial Sequence
  Synthetic Polynucleotide
cuacauacuaaaucucucuccuuua

SEQ ID NO: 5 - RNA, 25 nt, Artificial Sequence
  Synthetic Polynucleotide
ccaacaucaccaugcagauuaugca

SEQ ID NO: 6 - RNA, 25 nt, Artificial Sequence
  Synthetic Polynucleotide
gcacauaggagagaugagcuuccua

SEQ ID NO: 7 - RNA, 25 nt, Artificial Sequence
  Synthetic Polynucleotide
aucggugacagucacuagcuuauca

SEQ ID NO: 8 - RNA, 25 nt, Artificial Sequence
  Synthetic Polynucleotide
uuuaugagauguaucuuuugcucua

SEQ ID NO: 9 - RNA, 25 nt, Artificial Sequence
  Synthetic Polynucleotide
uacauacuaaaucucucuccuuuua

SEQ ID NO: 10 - RNA, 25 nt, Artificial Sequence
  Synthetic Polynucleotide
uaacagugcuaauguuauuggugua

SEQ ID NO: 11 - RNA, 25 nt, Artificial Sequence
  Synthetic Polynucleotide
uuguggaggcagagaaaagagaaaa

SEQ ID NO: 12 - RNA, 25 nt, Artificial Sequence
  Synthetic Polynucleotide
cacuggauguauuugacugcuguga

SEQ ID NO: 13 - RNA, 25 nt, Artificial Sequence
  Synthetic Polynucleotide
aucaccaucgacagaacaguccuua

SEQ ID NO: 14 - RNA, 25 nt, Artificial Sequence
  Synthetic Polynucleotide
aucgacagaacaguccuuaauccaa

SEQ ID NO: 15 - RNA, 25 nt, Artificial Sequence
  Synthetic Polynucleotide
auuuaugagauguaucuuuugcuca

SEQ ID NO: 16 - RNA, 25 nt, Artificial Sequence
  Synthetic Polynucleotide
ucacaccauugaaaccacuaguuca

SEQ ID NO: 17 - RNA, 25 nt, Artificial Sequence
  Synthetic Polynucleotide
uuuauuuauuggugcuacuguuuaa

SEQ ID NO: 18 - RNA, 25 nt, Artificial Sequence
  Synthetic Polynucleotide
uucuacauacuaaaucucucuccua

SEQ ID NO: 19 - RNA, 25 nt, Artificial Sequence
  Synthetic Polynucleotide
ucaccaucgacagaacaguccuuaa

SEQ ID NO: 20 - RNA, 25 nt, Artificial Sequence
  Synthetic Polynucleotide
ccucuuggaauuggauucgccauua

SEQ ID NO: 21 - RNA, 25 nt, Artificial Sequence
  Synthetic Polynucleotide
ccaucgacagaacaguccuuaauca

SEQ ID NO: 22 - RNA, 25 nt, Artificial Sequence
  Synthetic Polynucleotide
caucaccaugcagauuaugcggaua

SEQ ID NO: 23 - RNA, 25 nt, Artificial Sequence
  Synthetic Polynucleotide
guccucacaccauugaaaccacuaa

SEQ ID NO: 24 - RNA, 25 nt, Artificial Sequence
  Synthetic Polynucleotide
gaucggugacagucacuagcuuaua

SEQ ID NO: 25 - RNA, 25 nt, Artificial Sequence
  Synthetic Polynucleotide
uguggaggcagagaaaagagaaaga

SEQ ID NO: 26 - RNA, 25 nt, Artificial Sequence
  Synthetic Polynucleotide
aggucagacggacagaaagacagaa

SEQ ID NO: 27 - RNA, 25 nt, Artificial Sequence
  Synthetic Polynucleotide
auuguggaggcagagaaaagagaaa

SEQ ID NO: 28 - RNA, 25 nt, Artificial Sequence
  Synthetic Polynucleotide
ccaugcagauuaugcggaucaaaca

SEQ ID NO: 29 - RNA, 25 nt, Artificial Sequence
  Synthetic Polynucleotide
acagaacaguccuuaauccagaaaa

SEQ ID NO: 30 - RNA, 25 nt, Artificial Sequence
  Synthetic Polynucleotide
cacauuccuuugaaauaagguuuca

SEQ ID NO: 31 - RNA, 25 nt, Artificial Sequence
  Synthetic Polynucleotide
caucaccaucgacagaacaguccua

SEQ ID NO: 32 - RNA, 25 nt, Artificial Sequence
  Synthetic Polynucleotide
uccaacaucaccaugcagauuauga

SEQ ID NO: 33 - RNA, 25 nt, Artificial Sequence
  Synthetic Polynucleotide
ugcccauuguggaggcagagaaaaa

SEQ ID NO: 34 - RNA, 25 nt, Artificial Sequence
  Synthetic Polynucleotide
gcagauuaugcggaucaaaccucaa

SEQ ID NO: 35 - RNA, 25 nt, Artificial Sequence
  Synthetic Polynucleotide
acuggauguauuugacugcugugga

SEQ ID NO: 36 - RNA, 25 nt, Artificial Sequence
  Synthetic Polynucleotide
caccaucgacagaacaguccuuaaa

SEQ ID NO: 37 - RNA, 25 nt, Artificial Sequence
  Synthetic Polynucleotide
uuaacagugcuaauguuauugguga

SEQ ID NO: 38 - RNA, 25 nt, Artificial Sequence
  Synthetic Polynucleotide
caugcagauuaugcggaucaaacca

SEQ ID NO: 39 - RNA, 25 nt, Artificial Sequence
  Synthetic Polynucleotide
ggaaaagauauuaacaucacgucua

SEQ ID NO: 40 - RNA, 25 nt, Artificial Sequence
  Synthetic Polynucleotide
aguccaacaucaccaugcagauuaa

SEQ ID NO: 41 - RNA, 25 nt, Artificial Sequence
  Synthetic Polynucleotide
ccagcacacauuccuuugaaauaaa

SEQ ID NO: 42 - RNA, 25 nt, Artificial Sequence
  Synthetic Polynucleotide
auuuaauuuugcuaacacucagcua

SEQ ID NO: 43 - RNA, 25 nt, Artificial Sequence
  Synthetic Polynucleotide
agagaaaguguuuuauauacgguaa

SEQ ID NO: 44 - RNA, 25 nt, Artificial Sequence
  Synthetic Polynucleotide
ggagagaugagcuuccuacagcaca

SEQ ID NO: 45 - RNA, 25 nt, Artificial Sequence
  Synthetic Polynucleotide
uggaggcagagaaaagagaaaguga

SEQ ID NO: 46 - RNA, 25 nt, Artificial Sequence
  Synthetic Polynucleotide
ugauaaaauagacauugcuauucua

SEQ ID NO: 47 - RNA, 25 nt, Artificial Sequence
  Synthetic Polynucleotide
gugacagucacuagcuuaucuugaa

SEQ ID NO: 48 - RNA, 25 nt, Artificial Sequence
  Synthetic Polynucleotide
uauuuauuggugcuacuguuuauca

SEQ ID NO: 49 - RNA, 25 nt, Artificial Sequence
  Synthetic Polynucleotide
cuaauguuauuggugucuucacuga

SEQ ID NO: 50 - RNA, 25 nt, Artificial Sequence
  Synthetic Polynucleotide
aggaguccaacaucaccaugcagaa

SEQ ID NO: 51 - RNA, 25 nt, Artificial Sequence
  Synthetic Polynucleotide
augcagauuaugcggaucaaaccua

SEQ ID NO: 52 - RNA, 25 nt, Artificial Sequence
  Synthetic Polynucleotide
guccaacaucaccaugcagauuaua

SEQ ID NO: 53 - RNA, 25 nt, Artificial Sequence
  Synthetic Polynucleotide
cauuguggaggcagagaaaagagaa

SEQ ID NO: 54 - RNA, 25 nt, Artificial Sequence
  Synthetic Polynucleotide
aaaccugaaaugaaggaagaggaga

SEQ ID NO: 55 - RNA, 25 nt, Artificial Sequence
  Synthetic Polynucleotide
auuaacagugcuaauguuauuggua

SEQ ID NO: 56 - RNA, 25 nt, Artificial Sequence
  Synthetic Polynucleotide
acagugcuaauguuauuggugucua

SEQ ID NO: 57 - RNA, 25 nt, Artificial Sequence
  Synthetic Polynucleotide
ucucccugaucggugacagucacua

SEQ ID NO: 58 - RNA, 25 nt, Artificial Sequence
  Synthetic Polynucleotide
ucuacauacuaaaucucucuccuua

SEQ ID NO: 59 - RNA, 25 nt, Artificial Sequence
  Synthetic Polynucleotide
ucgacagaacaguccuuaauccaga

SEQ ID NO: 60 - RNA, 25 nt, Artificial Sequence
  Synthetic Polynucleotide
uauuuaauuuugcuaacacucagca

SEQ ID NO: 61 - RNA, 25 nt, Artificial Sequence
  Synthetic Polynucleotide
acacauuccuuugaaauaagguuua

SEQ ID NO: 62 - RNA, 25 nt, Artificial Sequence
  Synthetic Polynucleotide
cccugguggacaucuuccaggagua

SEQ ID NO: 63 - RNA, 25 nt, Artificial Sequence
  Synthetic Polynucleotide
ucuuggaauuggauucgccauuuua

SEQ ID NO: 64 - RNA, 25 nt, Artificial Sequence
  Synthetic Polynucleotide
acaucaccaugcagauuaugcggaa

SEQ ID NO: 65 - RNA, 25 nt, Artificial Sequence
  Synthetic Polynucleotide
caccauugaaaccacuaguucugua

SEQ ID NO: 66 - RNA, 25 nt, Artificial Sequence
  Synthetic Polynucleotide
gccagcacauaggagagaugagcua

SEQ ID NO: 67 - RNA, 25 nt, Artificial Sequence
  Synthetic Polynucleotide
uaaaauagacauugcuauucuguua

SEQ ID NO: 68 - RNA, 25 nt, Artificial Sequence
  Synthetic Polynucleotide
accaucgacagaacaguccuuaaua

SEQ ID NO: 69 - RNA, 25 nt, Artificial Sequence
  Synthetic Polynucleotide
uaaacaacgacaaagaaauacagaa

SEQ ID NO: 70 - RNA, 25 nt, Artificial Sequence
  Synthetic Polynucleotide
uuauuuauuggugcuacuguuuaua

SEQ ID NO: 71 - RNA, 25 nt, Artificial Sequence
  Synthetic Polynucleotide
uuauuuuucuugcugcuaaaucaca

SEQ ID NO: 72 - RNA, 25 nt, Artificial Sequence
  Synthetic Polynucleotide
aaccugaaaugaaggaagaggagaa

SEQ ID NO: 73 - RNA, 25 nt, Artificial Sequence
  Synthetic Polynucleotide
uauuuuucuugcugcuaaaucacca

SEQ ID NO: 74 - RNA, 25 nt, Artificial Sequence
  Synthetic Polynucleotide
caucgacagaacaguccuuaaucca

SEQ ID NO: 75 - RNA, 25 nt, Artificial Sequence
  Synthetic Polynucleotide
ggaggcagagaaaagagaaagugua

SEQ ID NO: 76 - RNA, 25 nt, Artificial Sequence
  Synthetic Polynucleotide
aaaagagaaaguguuuuauauacga

SEQ ID NO: 77 - RNA, 25 nt, Artificial Sequence
  Synthetic Polynucleotide
auaaaauagacauugcuauucugua

SEQ ID NO: 78 - RNA, 25 nt, Artificial Sequence
  Synthetic Polynucleotide
aaaauagacauugcuauucuguuua

SEQ ID NO: 79 - RNA, 25 nt, Artificial Sequence
  Synthetic Polynucleotide
caggaguacccugaugagaucgaga

SEQ ID NO: 80 - RNA, 25 nt, Artificial Sequence
  Synthetic Polynucleotide
agugcuaauguuauuggugucuuca

SEQ ID NO: 81 - RNA, 25 nt, Artificial Sequence
  Synthetic Polynucleotide
aauuaacagugcuaauguuauugga

SEQ ID NO: 82 - RNA, 25 nt, Artificial Sequence
  Synthetic Polynucleotide
aggaguacccugaugagaucgagua

SEQ ID NO: 83 - RNA, 25 nt, Artificial Sequence
  Synthetic Polynucleotide
agcacacauuccuuugaaauaagga

SEQ ID NO: 84 - RNA, 25 nt, Artificial Sequence
  Synthetic Polynucleotide
auucauguuuccaaucucucucuca

SEQ ID NO: 85 - RNA, 25 nt, Artificial Sequence
  Synthetic Polynucleotide
gagaaaguguuuuauauacgguaca

SEQ ID NO: 86 - RNA, 25 nt, Artificial Sequence
  Synthetic Polynucleotide
aacagugcuaauguuauugguguca

SEQ ID NO: 87 - RNA, 25 nt, Artificial Sequence
  Synthetic Polynucleotide
ucuagugcaguuuuucgagauauua

SEQ ID NO: 88 - RNA, 25 nt, Artificial Sequence
  Synthetic Polynucleotide
uaggagagaugagcuuccuacagca

SEQ ID NO: 89 - RNA, 25 nt, Artificial Sequence
  Synthetic Polynucleotide
gugcuaauguuauuggugucuucaa

SEQ ID NO: 90 - RNA, 25 nt, Artificial Sequence
  Synthetic Polynucleotide
auuuauuuauuggugcuacuguuua

SEQ ID NO: 91 - RNA, 25 nt, Artificial Sequence
  Synthetic Polynucleotide
ucucucuugcucucuuauuuguaca

SEQ ID NO: 92 - RNA, 25 nt, Artificial Sequence
  Synthetic Polynucleotide
cacaccauugaaaccacuaguucua

SEQ ID NO: 93 - RNA, 25 nt, Artificial Sequence
  Synthetic Polynucleotide
gggaaaagauauuaacaucacguca

SEQ ID NO: 94 - RNA, 25 nt, Artificial Sequence
  Synthetic Polynucleotide
aucaccaugcagauuaugcggauca

SEQ ID NO: 95 - RNA, 25 nt, Artificial Sequence
  Synthetic Polynucleotide
gugauucugauaaaauagacauuga

SEQ ID NO: 96 - RNA, 25 nt, Artificial Sequence
  Synthetic Polynucleotide
cagcacacauuccuuugaaauaaga

SEQ ID NO: 97 - RNA, 25 nt, Artificial Sequence
  Synthetic Polynucleotide
uaaaauucauguuuccaaucucuca

SEQ ID NO: 98 - RNA, 25 nt, Artificial Sequence
  Synthetic Polynucleotide
guacccugaugagaucgaguacaua

SEQ ID NO: 99 - RNA, 25 nt, Artificial Sequence
  Synthetic Polynucleotide
aaauucauguuuccaaucucucuca

SEQ ID NO: 100 - RNA, 25 nt, Artificial Sequence
  Synthetic Polynucleotide
cuggauguauuugacugcuguggaa

SEQ ID NO: 101 - RNA, 25 nt, Artificial Sequence
  Synthetic Polynucleotide
auuaacaucacgucuuugucucuaa

SEQ ID NO: 102 - RNA, 25 nt, Artificial Sequence
  Synthetic Polynucleotide
uccucacaccauugaaaccacuaga

SEQ ID NO: 103 - RNA, 25 nt, Artificial Sequence
  Synthetic Polynucleotide
ggccagcacauaggagagaugagca

SEQ ID NO: 104 - RNA, 25 nt, Artificial Sequence
  Synthetic Polynucleotide
gauaaaauagacauugcuauucuga

SEQ ID NO: 105 - RNA, 25 nt, Artificial Sequence
  Synthetic Polynucleotide
gauauuuaauuuugcuaacacucaa

SEQ ID NO: 106 - RNA, 25 nt, Artificial Sequence
  Synthetic Polynucleotide
accugaaaugaaggaagaggagaca

SEQ ID NO: 107 - RNA, 25 nt, Artificial Sequence
  Synthetic Polynucleotide
aauucauguuuccaaucucucucua

SEQ ID NO: 108 - RNA, 25 nt, Artificial Sequence
  Synthetic Polynucleotide
ggggaaaagauauuaacaucacgua

SEQ ID NO: 109 - RNA, 25 nt, Artificial Sequence
  Synthetic Polynucleotide
cuacagcacaacaaaugugaaugca

SEQ ID NO: 110 - RNA, 25 nt, Artificial Sequence
  Synthetic Polynucleotide
acacacccacccacauacauacaua

SEQ ID NO: 111 - RNA, 25 nt, Artificial Sequence
  Synthetic Polynucleotide
aguuuuucgagauauuccguaguaa

SEQ ID NO: 112 - RNA, 25 nt, Artificial Sequence
  Synthetic Polynucleotide
ggucccucuuggaauuggauucgca

SEQ ID NO: 113 - RNA, 25 nt, Artificial Sequence
  Synthetic Polynucleotide
gcaguuuuucgagauauuccguaga

SEQ ID NO: 114 - RNA, 25 nt, Artificial Sequence
  Synthetic Polynucleotide
uuaaacaacgacaaagaaauacaga

SEQ ID NO: 115 - RNA, 25 nt, Artificial Sequence
  Synthetic Polynucleotide
auauuuaauuuugcuaacacucaga

SEQ ID NO: 116 - RNA, 25 nt, Artificial Sequence
  Synthetic Polynucleotide
agaauucuacauacuaaaucucuca

SEQ ID NO: 117 - RNA, 25 nt, Artificial Sequence
  Synthetic Polynucleotide
aaacaacgacaaagaaauacagaua

SEQ ID NO: 118 - RNA, 25 nt, Artificial Sequence
  Synthetic Polynucleotide
aacaucaccaugcagauuaugcgga

SEQ ID NO: 119 - RNA, 25 nt, Artificial Sequence
  Synthetic Polynucleotide
accauugaaaccacuaguucuguca

SEQ ID NO: 120 - RNA, 25 nt, Artificial Sequence
  Synthetic Polynucleotide
uccaggaguacccugaugagaucga

SEQ ID NO: 121 - RNA, 25 nt, Artificial Sequence
  Synthetic Polynucleotide
guuauuggugucuucacuggaugua

SEQ ID NO: 122 - RNA, 25 nt, Artificial Sequence
  Synthetic Polynucleotide
uggauguauuugacugcuguggaca

SEQ ID NO: 123 - RNA, 25 nt, Artificial Sequence
  Synthetic Polynucleotide
uguuauuggugucuucacuggauga

SEQ ID NO: 124 - RNA, 25 nt, Artificial Sequence
  Synthetic Polynucleotide
gcuaauguuauuggugucuucacua

SEQ ID NO: 125 - RNA, 25 nt, Artificial Sequence
  Synthetic Polynucleotide
aaaagauauuaacaucacgucuuua

SEQ ID NO: 126 - RNA, 25 nt, Artificial Sequence
  Synthetic Polynucleotide
auauuaacaucacgucuuugucuca

SEQ ID NO: 127 - RNA, 25 nt, Artificial Sequence
  Synthetic Polynucleotide
agauauuaacaucacgucuuuguca

SEQ ID NO: 128 - RNA, 25 nt, Artificial Sequence
  Synthetic Polynucleotide
aaauaagguuucaauauacaucuaa

SEQ ID NO: 129 - RNA, 25 nt, Artificial Sequence
  Synthetic Polynucleotide
gaguacccugaugagaucgaguaca

SEQ ID NO: 130 - RNA, 25 nt, Artificial Sequence
  Synthetic Polynucleotide
auuuauuggugcuacuguuuaucca

SEQ ID NO: 131 - RNA, 25 nt, Artificial Sequence
  Synthetic Polynucleotide
auaaaauucauguuuccaaucucua

SEQ ID NO: 132 - RNA, 25 nt, Artificial Sequence
  Synthetic Polynucleotide
ggaguccaacaucaccaugcagaua

SEQ ID NO: 133 - RNA, 25 nt, Artificial Sequence
  Synthetic Polynucleotide
cucuagugcaguuuuucgagauaua

SEQ ID NO: 134 - RNA, 25 nt, Artificial Sequence
  Synthetic Polynucleotide
uauuaacaucacgucuuugucucua

SEQ ID NO: 135 - RNA, 25 nt, Artificial Sequence
  Synthetic Polynucleotide
gaguccaacaucaccaugcagauua

SEQ ID NO: 136 - RNA, 25 nt, Artificial Sequence
  Synthetic Polynucleotide
gagaauucuacauacuaaaucucua

SEQ ID NO: 137 - RNA, 25 nt, Artificial Sequence
  Synthetic Polynucleotide
gacagaacaguccuuaauccagaaa

SEQ ID NO: 138 - RNA, 25 nt, Artificial Sequence
  Synthetic Polynucleotide
ucccucuuggaauuggauucgccaa

SEQ ID NO: 139 - RNA, 25 nt, Artificial Sequence
  Synthetic Polynucleotide
uuggugcuacuguuuauccguaaua

SEQ ID NO: 140 - RNA, 25 nt, Artificial Sequence
  Synthetic Polynucleotide
uuuauuggugcuacuguuuauccga

SEQ ID NO: 141 - RNA, 25 nt, Artificial Sequence
  Synthetic Polynucleotide
cuagugcaguuuuucgagauauuca

SEQ ID NO: 142 - RNA, 25 nt, Artificial Sequence
  Synthetic Polynucleotide
guuuuucgagauauuccguaguaca

SEQ ID NO: 143 - RNA, 25 nt, Artificial Sequence
  Synthetic Polynucleotide
accaugcagauuaugcggaucaaaa

SEQ ID NO: 144 - RNA, 25 nt, Artificial Sequence
  Synthetic Polynucleotide
caguuuuucgagauauuccguagua

SEQ ID NO: 145 - RNA, 25 nt, Artificial Sequence
  Synthetic Polynucleotide
aagauauuaacaucacgucuuugua

SEQ ID NO: 146 - RNA, 25 nt, Artificial Sequence
  Synthetic Polynucleotide
cagcacauaggagagaugagcuuca

SEQ ID NO: 147 - RNA, 25 nt, Artificial Sequence
  Synthetic Polynucleotide
agugcaguuuuucgagauauuccga

SEQ ID NO: 148 - RNA, 25 nt, Artificial Sequence
  Synthetic Polynucleotide
auguuauuggugucuucacuggaua

SEQ ID NO: 149 - RNA, 25 nt, Artificial Sequence
  Synthetic Polynucleotide
uagugcaguuuuucgagauauucca

SEQ ID NO: 150 - RNA, 25 nt, Artificial Sequence
  Synthetic Polynucleotide
gugcaguuuuucgagauauuccgua

SEQ ID NO: 151 - RNA, 25 nt, Artificial Sequence
  Synthetic Polynucleotide
acauaggagagaugagcuuccuaca

SEQ ID NO: 152 - RNA, 25 nt, Artificial Sequence
  Synthetic Polynucleotide
aaagauauuaacaucacgucuuuga

SEQ ID NO: 153 - RNA, 25 nt, Artificial Sequence
  Synthetic Polynucleotide
gucucuagugcaguuuuucgagaua

SEQ ID NO: 154 - RNA, 25 nt, Artificial Sequence
  Synthetic Polynucleotide
cuauuuaugagauguaucuuuugca

SEQ ID NO: 155 - RNA, 25 nt, Artificial Sequence
  Synthetic Polynucleotide
ugcuaauguuauuggugucuucaca

SEQ ID NO: 156 - RNA, 25 nt, Artificial Sequence
  Synthetic Polynucleotide
auauauauuuggcaacuuguauuua

SEQ ID NO: 157 - RNA, 25 nt, Artificial Sequence
  Synthetic Polynucleotide
gauauuaacaucacgucuuugucua

SEQ ID NO: 158 - RNA, 25 nt, Artificial Sequence
  Synthetic Polynucleotide
gucccucuuggaauuggauucgcca

SEQ ID NO: 159 - RNA, 25 nt, Artificial Sequence
  Synthetic Polynucleotide
aauucuacauacuaaaucucucuca

SEQ ID NO: 160 - RNA, 25 nt, Artificial Sequence
  Synthetic Polynucleotide
gcuccccagcacacauuccuuugaa

SEQ ID NO: 161 - RNA, 25 nt, Artificial Sequence
  Synthetic Polynucleotide
ccugaaaugaaggaagaggagacua

SEQ ID NO: 162 - RNA, 25 nt, Artificial Sequence
  Synthetic Polynucleotide
uuaacaucacgucuuugucucuaga

SEQ ID NO: 163 - RNA, 25 nt, Artificial Sequence
  Synthetic Polynucleotide
ggauguauuugacugcuguggacua

SEQ ID NO: 164 - RNA, 25 nt, Artificial Sequence
  Synthetic Polynucleotide
gaauucuacauacuaaaucucucua

SEQ ID NO: 165 - RNA, 25 nt, Artificial Sequence
  Synthetic Polynucleotide
uauauauuuggcaacuuguauuuga

SEQ ID NO: 166 - RNA, 25 nt, Artificial Sequence
  Synthetic Polynucleotide
caaggccagcacauaggagagauga

SEQ ID NO: 167 - RNA, 25 nt, Artificial Sequence
  Synthetic Polynucleotide
cggugacagucacuagcuuaucuua

SEQ ID NO: 168 - RNA, 25 nt, Artificial Sequence
  Synthetic Polynucleotide
uuuaaacaacgacaaagaaauacaa

SEQ ID NO: 169 - RNA, 25 nt, Artificial Sequence
  Synthetic Polynucleotide
ugaucggugacagucacuagcuuaa

SEQ ID NO: 170 - RNA, 25 nt, Artificial Sequence
  Synthetic Polynucleotide
aguacccugaugagaucgaguacaa

SEQ ID NO: 171 - RNA, 25 nt, Artificial Sequence
  Synthetic Polynucleotide
ugcaguuuuucgagauauuccguaa

SEQ ID NO: 172 - RNA, 25 nt, Artificial Sequence
  Synthetic Polynucleotide
uuuuucgagauauuccguaguacaa

SEQ ID NO: 173 - RNA, 25 nt, Artificial Sequence
  Synthetic Polynucleotide
uuauuggugcuacuguuuauccgua

SEQ ID NO: 174 - RNA, 25 nt, Artificial Sequence
  Synthetic Polynucleotide
cugaucggugacagucacuagcuua

SEQ ID NO: 175 - RNA, 25 nt, Artificial Sequence
  Synthetic Polynucleotide
uacuguuuauccguaauaauuguga

SEQ ID NO: 176 - RNA, 25 nt, Artificial Sequence
  Synthetic Polynucleotide
uuuucgagauauuccguaguacaua

SEQ ID NO: 177 - RNA, 25 nt, Artificial Sequence
  Synthetic Polynucleotide
auuggugcuacuguuuauccguaaa

SEQ ID NO: 178 - RNA, 25 nt, Artificial Sequence
  Synthetic Polynucleotide
ucucuagugcaguuuuucgagauaa

SEQ ID NO: 179 - RNA, 25 nt, Artificial Sequence
  Synthetic Polynucleotide
ggugacagucacuagcuuaucuuga

SEQ ID NO: 180 - RNA, 25 nt, Artificial Sequence
  Synthetic Polynucleotide
auauauuuggcaacuuguauuugua

SEQ ID NO: 181 - RNA, 25 nt, Artificial Sequence
  Synthetic Polynucleotide
uauuggugcuacuguuuauccguaa

SEQ ID NO: 182 - RNA, 25 nt, Artificial Sequence
  Synthetic Polynucleotide
uuucgagauauuccguaguacauaa

SEQ ID NO: 183 - RNA, 13 nt, Artificial Sequence
  Synthetic Polynucleotide
cucaugaauuaga

SEQ ID NO: 184 - RNA, 19 nt, Artificial Sequence
  Synthetic Polynucleotide
ucuaauucaugagaaauac

SEQ ID NO: 185 - RNA, 13 nt, Artificial Sequence
  Synthetic Polynucleotide
cugaggucaauua

SEQ ID NO: 186 - RNA, 19 nt, Artificial Sequence
  Synthetic Polynucleotide
uaauugaccucagaagaug

SEQ ID NO: 187 - RNA, 13 nt, Artificial Sequence
  Synthetic Polynucleotide
gaggucaauuaaa

SEQ ID NO: 188 - RNA, 19 nt, Artificial Sequence
  Synthetic Polynucleotide
uuuaauugaccucagaaga

SEQ ID NO: 189 - RNA, 13 nt, Artificial Sequence
  Synthetic Polynucleotide
ucugaggucaauu

SEQ ID NO: 190 - RNA, 19 nt, Artificial Sequence
  Synthetic Polynucleotide
aauugaccucagaagaugc

SEQ ID NO: 191 - RNA, 13 nt, Artificial Sequence
  Synthetic Polynucleotide
ugaggucaauuaa

SEQ ID NO: 192 - RNA, 19 nt, Artificial Sequence
  Synthetic Polynucleotide
uuaauugaccucagaagau

SEQ ID NO: 193 - RNA, 13 nt, Artificial Sequence
  Synthetic Polynucleotide
uucugaggucaau

SEQ ID NO: 194 - RNA, 19 nt, Artificial Sequence
  Synthetic Polynucleotide
auugaccucagaagaugca

SEQ ID NO: 195 - RNA, 13 nt, Artificial Sequence
  Synthetic Polynucleotide
gucagcuggauga

SEQ ID NO: 196 - RNA, 19 nt, Artificial Sequence
  Synthetic Polynucleotide
ucauccagcugacucguuu

SEQ ID NO: 197 - RNA, 13 nt, Artificial Sequence
  Synthetic Polynucleotide
uucugaugaaucu

SEQ ID NO: 198 - RNA, 19 nt, Artificial Sequence
  Synthetic Polynucleotide
agauucaucagaaugguga

SEQ ID NO: 199 - RNA, 13 nt, Artificial Sequence
  Synthetic Polynucleotide
uggacugagguca

SEQ ID NO: 200 - RNA, 19 nt, Artificial Sequence
  Synthetic Polynucleotide
ugaccucaguccauaaacc

SEQ ID NO: 201 - RNA, 13 nt, Artificial Sequence
  Synthetic Polynucleotide
gagucucaccauu

SEQ ID NO: 202 - RNA, 19 nt, Artificial Sequence
  Synthetic Polynucleotide
aauggugagacucaucaga

SEQ ID NO: 203 - RNA, 13 nt, Artificial Sequence
  Synthetic Polynucleotide
gacugaggucaaa

SEQ ID NO: 204 - RNA, 19 nt, Artificial Sequence
  Synthetic Polynucleotide
uuugaccucaguccauaaa

SEQ ID NO: 205 - RNA, 13 nt, Artificial Sequence
  Synthetic Polynucleotide
ucacagccaugaa

SEQ ID NO: 206 - RNA, 19 nt, Artificial Sequence
  Synthetic Polynucleotide
uucauggcugugaaauuca

SEQ ID NO: 207 - RNA, 13 nt, Artificial Sequence
  Synthetic Polynucleotide
agucucaccauuc

SEQ ID NO: 208 - RNA, 19 nt, Artificial Sequence
  Synthetic Polynucleotide
gaauggugagacucaucag

SEQ ID NO: 209 - RNA, 13 nt, Artificial Sequence
  Synthetic Polynucleotide
aagcggaaagcca

SEQ ID NO: 210 - RNA, 19 nt, Artificial Sequence
  Synthetic Polynucleotide
uggcuuuccgcuuauauaa

SEQ ID NO: 211 - RNA, 13 nt, Artificial Sequence
  Synthetic Polynucleotide
agcggaaagccaa

SEQ ID NO: 212 - RNA, 19 nt, Artificial Sequence
  Synthetic Polynucleotide
uuggcuuuccgcuuauaua

SEQ ID NO: 213 - RNA, 13 nt, Artificial Sequence
  Synthetic Polynucleotide
accacauggauga

SEQ ID NO: 214 - RNA, 19 nt, Artificial Sequence
  Synthetic Polynucleotide
ucauccauguggucauggc

SEQ ID NO: 215 - RNA, 13 nt, Artificial Sequence
  Synthetic Polynucleotide
gccaugaccacau

SEQ ID NO: 216 - RNA, 19 nt, Artificial Sequence
  Synthetic Polynucleotide
auguggucauggcuuucgu

SEQ ID NO: 217 - RNA, 13 nt, Artificial Sequence
  Synthetic Polynucleotide
aagccaugaccac

SEQ ID NO: 218 - RNA, 19 nt, Artificial Sequence
  Synthetic Polynucleotide
guggucauggcuuucguug

SEQ ID NO: 219 - RNA, 13 nt, Artificial Sequence
  Synthetic Polynucleotide
gcggaaagccaau

SEQ ID NO: 220 - RNA, 19 nt, Artificial Sequence
  Synthetic Polynucleotide
auuggcuuuccgcuuauau

SEQ ID NO: 221 - RNA, 13 nt, Artificial Sequence
  Synthetic Polynucleotide
aaauuucguauuu

SEQ ID NO: 222 - RNA, 19 nt, Artificial Sequence
  Synthetic Polynucleotide
aaauacgaaauuucaggug

SEQ ID NO: 223 - RNA, 13 nt, Artificial Sequence
  Synthetic Polynucleotide
auuucguauuucu

SEQ ID NO: 224 - RNA, 19 nt, Artificial Sequence
  Synthetic Polynucleotide
agaaauacgaaauuucagg

SEQ ID NO: 225 - RNA, 13 nt, Artificial Sequence
  Synthetic Polynucleotide
aaagccaugacca

SEQ ID NO: 226 - RNA, 19 nt, Artificial Sequence
  Synthetic Polynucleotide
uggucauggcuuucguugg

SEQ ID NO: 227 - RNA, 13 nt, Artificial Sequence
  Synthetic Polynucleotide
acauggaugauau

SEQ ID NO: 228 - RNA, 19 nt, Artificial Sequence
  Synthetic Polynucleotide
auaucauccauguggucau

SEQ ID NO: 229 - RNA, 13 nt, Artificial Sequence
  Synthetic Polynucleotide
gaaauuucguauu

SEQ ID NO: 230 - RNA, 19 nt, Artificial Sequence
  Synthetic Polynucleotide
aauacgaaauuucaggugu

SEQ ID NO: 231 - RNA, 13 nt, Artificial Sequence
  Synthetic Polynucleotide
gcgccuucugauu

SEQ ID NO: 232 - RNA, 19 nt, Artificial Sequence
  Synthetic Polynucleotide
aaucagaaggcgcguucag

SEQ ID NO: 233 - RNA, 13 nt, Artificial Sequence
  Synthetic Polynucleotide
auuucucaugaau

SEQ ID NO: 234 - RNA, 19 nt, Artificial Sequence
  Synthetic Polynucleotide
auucaugagaaauacgaaa

SEQ ID NO: 235 - RNA, 13 nt, Artificial Sequence
  Synthetic Polynucleotide
cucucaugaauag

SEQ ID NO: 236 - RNA, 19 nt, Artificial Sequence
  Synthetic Polynucleotide
cuauucaugagagaauaac

SEQ ID NO: 237 - RNA, 13 nt, Artificial Sequence
  Synthetic Polynucleotide
aaguccaacgaaa

SEQ ID NO: 238 - RNA, 19 nt, Artificial Sequence
  Synthetic Polynucleotide
uuucguuggacuuacuugg

SEQ ID NO: 239 - RNA, 13 nt, Artificial Sequence
  Synthetic Polynucleotide
augaugagagcaa

SEQ ID NO: 240 - RNA, 19 nt, Artificial Sequence
  Synthetic Polynucleotide
uugcucucaucauuggcuu

SEQ ID NO: 241 - RNA, 13 nt, Artificial Sequence
  Synthetic Polynucleotide
gcgaggaguugaa

SEQ ID NO: 242 - RNA, 19 nt, Artificial Sequence
  Synthetic Polynucleotide
uucaacuccucgcuuucca

SEQ ID NO: 243 - RNA, 13 nt, Artificial Sequence
  Synthetic Polynucleotide
ugauugauaguca

SEQ ID NO: 244 - RNA, 19 nt, Artificial Sequence
  Synthetic Polynucleotide
ugacuaucaaucacaucgg

SEQ ID NO: 245 - RNA, 13 nt, Artificial Sequence
  Synthetic Polynucleotide
agauagugcaucu

SEQ ID NO: 246 - RNA, 19 nt, Artificial Sequence
  Synthetic Polynucleotide
agaugcacuaucuaauuca

SEQ ID NO: 247 - RNA, 13 nt, Artificial Sequence
  Synthetic Polynucleotide
auguguaucuauu

SEQ ID NO: 248 - RNA, 19 nt, Artificial Sequence
  Synthetic Polynucleotide
aauagauacacauucaacc

SEQ ID NO: 249 - RNA, 13 nt, Artificial Sequence
  Synthetic Polynucleotide
uucuauagaagaa

SEQ ID NO: 250 - RNA, 19 nt, Artificial Sequence
  Synthetic Polynucleotide
uucuucuauagaaugaaca

SEQ ID NO: 251 - RNA, 13 nt, Artificial Sequence
  Synthetic Polynucleotide
uuguccagcaauu

SEQ ID NO: 252 - RNA, 19 nt, Artificial Sequence
  Synthetic Polynucleotide
aauugcuggacaaccgugg

SEQ ID NO: 253 - RNA, 13 nt, Artificial Sequence
  Synthetic Polynucleotide
acauggaaagcga

SEQ ID NO: 254 - RNA, 19 nt, Artificial Sequence
  Synthetic Polynucleotide
ucgcuuuccaugugugagg

SEQ ID NO: 255 - RNA, 13 nt, Artificial Sequence
  Synthetic Polynucleotide
gcaguccagauua

SEQ ID NO: 256 - RNA, 19 nt, Artificial Sequence
  Synthetic Polynucleotide
uaaucuggacugcuugugg

SEQ ID NO: 257 - RNA, 13 nt, Artificial Sequence
  Synthetic Polynucleotide
ugguugaaugugu

SEQ ID NO: 258 - RNA, 19 nt, Artificial Sequence
  Synthetic Polynucleotide
acacauucaaccaauaaac

SEQ ID NO: 259 - RNA, 13 nt, Artificial Sequence
  Synthetic Polynucleotide
uuaugaaacgagu

SEQ ID NO: 260 - RNA, 19 nt, Artificial Sequence
  Synthetic Polynucleotide
acucguuucauaacugucc

SEQ ID NO: 261 - RNA, 13 nt, Artificial Sequence
  Synthetic Polynucleotide
caguccagauuau

SEQ ID NO: 262 - RNA, 19 nt, Artificial Sequence
  Synthetic Polynucleotide
auaaucuggacugcuugug

SEQ ID NO: 263 - RNA, 13 nt, Artificial Sequence
  Synthetic Polynucleotide
auauaagcggaaa

SEQ ID NO: 264 - RNA, 19 nt, Artificial Sequence
  Synthetic Polynucleotide
uuuccgcuuauauaaucug

SEQ ID NO: 265 - RNA, 13 nt, Artificial Sequence
  Synthetic Polynucleotide
uaccaguuaaaca

SEQ ID NO: 266 - RNA, 19 nt, Artificial Sequence
  Synthetic Polynucleotide
uguuuaacugguauggcac

SEQ ID NO: 267 - RNA, 13 nt, Artificial Sequence
  Synthetic Polynucleotide
uguucauucuaua

SEQ ID NO: 268 - RNA, 19 nt, Artificial Sequence
  Synthetic Polynucleotide
uauagaaugaacauagaca

SEQ ID NO: 269 - RNA, 13 nt, Artificial Sequence
  Synthetic Polynucleotide
ccgaccaaggaaa

SEQ ID NO: 270 - RNA, 19 nt, Artificial Sequence
  Synthetic Polynucleotide
uuuccuuggucggcguuug

SEQ ID NO: 271 - RNA, 13 nt, Artificial Sequence
  Synthetic Polynucleotide
gaauggugcauac

SEQ ID NO: 272 - RNA, 19 nt, Artificial Sequence
  Synthetic Polynucleotide
guaugcaccauucaacucc

SEQ ID NO: 273 - RNA, 13 nt, Artificial Sequence
  Synthetic Polynucleotide
auaugauggccga

SEQ ID NO: 274 - RNA, 19 nt, Artificial Sequence
  Synthetic Polynucleotide
ucggccaucauaugugucu

SEQ ID NO: 275 - RNA, 13 nt, Artificial Sequence
  Synthetic Polynucleotide
agcaguccagauu

SEQ ID NO: 276 - RNA, 19 nt, Artificial Sequence
  Synthetic Polynucleotide
aaucuggacugcuuguggc

SEQ ID NO: 277 - RNA, 13 nt, Artificial Sequence
  Synthetic Polynucleotide
gcauuuagucaaa

SEQ ID NO: 278 - RNA, 19 nt, Artificial Sequence
  Synthetic Polynucleotide
uuugacuaaaugcaaagug

SEQ ID NO: 279 - RNA, 13 nt, Artificial Sequence
  Synthetic Polynucleotide
agcauuccgaugu

SEQ ID NO: 280 - RNA, 19 nt, Artificial Sequence
  Synthetic Polynucleotide
acaucggaaugcucauugc

SEQ ID NO: 281 - RNA, 13 nt, Artificial Sequence
  Synthetic Polynucleotide
uagucaggaacuu

SEQ ID NO: 282 - RNA, 19 nt, Artificial Sequence
  Synthetic Polynucleotide
aaguuccugacuaucaauc

SEQ ID NO: 283 - RNA, 13 nt, Artificial Sequence
  Synthetic Polynucleotide
ugcauuuagucaa

SEQ ID NO: 284 - RNA, 19 nt, Artificial Sequence
  Synthetic Polynucleotide
uugacuaaaugcaaaguga

SEQ ID NO: 285 - RNA, 13 nt, Artificial Sequence
  Synthetic Polynucleotide
gucugaugagucu

SEQ ID NO: 286 - RNA, 19 nt, Artificial Sequence
  Synthetic Polynucleotide
agacucaucagacugguga

SEQ ID NO: 287 - RNA, 13 nt, Artificial Sequence
  Synthetic Polynucleotide
uagacacauauga

SEQ ID NO: 288 - RNA, 19 nt, Artificial Sequence
  Synthetic Polynucleotide
ucauaugugucuacugugg

SEQ ID NO: 289 - RNA, 13 nt, Artificial Sequence
  Synthetic Polynucleotide
cagacgaggacau

SEQ ID NO: 290 - RNA, 19 nt, Artificial Sequence
  Synthetic Polynucleotide
auguccucgucuguagcau

SEQ ID NO: 291 - RNA, 13 nt, Artificial Sequence
  Synthetic Polynucleotide
cagccgugaauuc

SEQ ID NO: 292 - RNA, 19 nt, Artificial Sequence
  Synthetic Polynucleotide
gaauucacggcugacuuug

SEQ ID NO: 293 - RNA, 13 nt, Artificial Sequence
  Synthetic Polynucleotide
agucuggaaauaa

SEQ ID NO: 294 - RNA, 19 nt, Artificial Sequence
  Synthetic Polynucleotide
uuauuuccagacucaaaua

SEQ ID NO: 295 - RNA, 13 nt, Artificial Sequence
  Synthetic Polynucleotide
aguuuguggcuuc

SEQ ID NO: 296 - RNA, 19 nt, Artificial Sequence
  Synthetic Polynucleotide
gaagccacaaacuaaacua

SEQ ID NO: 297 - RNA, 13 nt, Artificial Sequence
  Synthetic Polynucleotide
aguccaacgaaag

SEQ ID NO: 298 - RNA, 19 nt, Artificial Sequence
  Synthetic Polynucleotide
cuuucguuggacuuacuug

SEQ ID NO: 299 - RNA, 13 nt, Artificial Sequence
  Synthetic Polynucleotide
aaguuucgcagac

SEQ ID NO: 300 - RNA, 19 nt, Artificial Sequence
  Synthetic Polynucleotide
gucugcgaaacuucuuaga

SEQ ID NO: 301 - RNA, 13 nt, Artificial Sequence
  Synthetic Polynucleotide
agcaaugagcauu

SEQ ID NO: 302 - RNA, 19 nt, Artificial Sequence
  Synthetic Polynucleotide
aaugcucauugcucucauc

SEQ ID NO: 303 - RNA, 13 nt, Artificial Sequence
  Synthetic Polynucleotide
uuagauagugcau

SEQ ID NO: 304 - RNA, 19 nt, Artificial Sequence
  Synthetic Polynucleotide
augcacuaucuaauucaug

SEQ ID NO: 305 - RNA, 13 nt, Artificial Sequence
  Synthetic Polynucleotide
uggugcauacaag

SEQ ID NO: 306 - RNA, 19 nt, Artificial Sequence
  Synthetic Polynucleotide
cuuguaugcaccauucaac

SEQ ID NO: 307 - RNA, 13 nt, Artificial Sequence
  Synthetic Polynucleotide
augaaacgaguca

SEQ ID NO: 308 - RNA, 19 nt, Artificial Sequence
  Synthetic Polynucleotide
ugacucguuucauaacugu

SEQ ID NO: 309 - RNA, 13 nt, Artificial Sequence
  Synthetic Polynucleotide
ccagagugcugaa

SEQ ID NO: 310 - RNA, 19 nt, Artificial Sequence
  Synthetic Polynucleotide
uucagcacucuggucaucc

SEQ ID NO: 311 - RNA, 13 nt, Artificial Sequence
  Synthetic Polynucleotide
cagccaugaauuu

SEQ ID NO: 312 - RNA, 19 nt, Artificial Sequence
  Synthetic Polynucleotide
aaauucauggcuguggaau

SEQ ID NO: 313 - RNA, 13 nt, Artificial Sequence
  Synthetic Polynucleotide
auugguugaaugu

SEQ ID NO: 314 - RNA, 19 nt, Artificial Sequence
  Synthetic Polynucleotide
acauucaaccaauaaacug

SEQ ID NO: 315 - RNA, 13 nt, Artificial Sequence
  Synthetic Polynucleotide
gguugaaugugua

SEQ ID NO: 316 - RNA, 19 nt, Artificial Sequence
  Synthetic Polynucleotide
uacacauucaaccaauaaa

SEQ ID NO: 317 - RNA, 13 nt, Artificial Sequence
  Synthetic Polynucleotide
ggaaauaacuaau

SEQ ID NO: 318 - RNA, 19 nt, Artificial Sequence
  Synthetic Polynucleotide
auuaguuauuuccagacuc

SEQ ID NO: 319 - RNA, 13 nt, Artificial Sequence
  Synthetic Polynucleotide
ucaugaauagaaa

SEQ ID NO: 320 - RNA, 19 nt, Artificial Sequence
  Synthetic Polynucleotide
uuucuauucaugagagaau

SEQ ID NO: 321 - RNA, 13 nt, Artificial Sequence
  Synthetic Polynucleotide
gccagcaaccgaa

SEQ ID NO: 322 - RNA, 19 nt, Artificial Sequence
  Synthetic Polynucleotide
uucgguugcuggcaggucc

SEQ ID NO: 323 - RNA, 13 nt, Artificial Sequence
  Synthetic Polynucleotide
caccucacacaug

SEQ ID NO: 324 - RNA, 19 nt, Artificial Sequence
  Synthetic Polynucleotide
caugugugaggugaugucc

SEQ ID NO: 325 - RNA, 13 nt, Artificial Sequence
  Synthetic Polynucleotide
aguugaauggugc

SEQ ID NO: 326 - RNA, 19 nt, Artificial Sequence
  Synthetic Polynucleotide
gcaccauucaacuccucgc

SEQ ID NO: 327 - RNA, 13 nt, Artificial Sequence
  Synthetic Polynucleotide
agucagcuggaug

SEQ ID NO: 328 - RNA, 19 nt, Artificial Sequence
  Synthetic Polynucleotide
cauccagcugacucguuuc

SEQ ID NO: 329 - RNA, 13 nt, Artificial Sequence
  Synthetic Polynucleotide
uauaagcggaaag

SEQ ID NO: 330 - RNA, 19 nt, Artificial Sequence
  Synthetic Polynucleotide
cuuuccgcuuauauaaucu

SEQ ID NO: 331 - RNA, 13 nt, Artificial Sequence
  Synthetic Polynucleotide
uuccgaugugauu

SEQ ID NO: 332 - RNA, 19 nt, Artificial Sequence
  Synthetic Polynucleotide
aaucacaucggaaugcuca

SEQ ID NO: 333 - RNA, 13 nt, Artificial Sequence
  Synthetic Polynucleotide
auaacuaaugugu

SEQ ID NO: 334 - RNA, 19 nt, Artificial Sequence
  Synthetic Polynucleotide
acacauuaguuauuuccag

SEQ ID NO: 335 - RNA, 13 nt, Artificial Sequence
  Synthetic Polynucleotide
ucauucuauagaa

SEQ ID NO: 336 - RNA, 19 nt, Artificial Sequence
  Synthetic Polynucleotide
uucuauagaaugaacauag

SEQ ID NO: 337 - RNA, 13 nt, Artificial Sequence
  Synthetic Polynucleotide
aacuaucacugua

SEQ ID NO: 338 - RNA, 19 nt, Artificial Sequence
  Synthetic Polynucleotide
uacagugauaguuugcauu

SEQ ID NO: 339 - RNA, 13 nt, Artificial Sequence
  Synthetic Polynucleotide
gucaauugcuuau

SEQ ID NO: 340 - RNA, 19 nt, Artificial Sequence
  Synthetic Polynucleotide
auaagcaauugacaccacc

SEQ ID NO: 341 - RNA, 13 nt, Artificial Sequence
  Synthetic Polynucleotide
agcaauuaauaaa

SEQ ID NO: 342 - RNA, 19 nt, Artificial Sequence
  Synthetic Polynucleotide
uuuauuaauugcuggacaa

SEQ ID NO: 343 - RNA, 13 nt, Artificial Sequence
  Synthetic Polynucleotide
acgacucugauga

SEQ ID NO: 344 - RNA, 19 nt, Artificial Sequence
  Synthetic Polynucleotide
ucaucagagucguucgagu

SEQ ID NO: 345 - RNA, 13 nt, Artificial Sequence
  Synthetic Polynucleotide
uagugugguuuau

SEQ ID NO: 346 - RNA, 19 nt, Artificial Sequence
  Synthetic Polynucleotide
auaaaccacacuaucaccu

SEQ ID NO: 347 - RNA, 13 nt, Artificial Sequence
  Synthetic Polynucleotide
aagccaaugauga

SEQ ID NO: 348 - RNA, 19 nt, Artificial Sequence
  Synthetic Polynucleotide
ucaucauuggcuuuccgcu

SEQ ID NO: 349 - RNA, 13 nt, Artificial Sequence
  Synthetic Polynucleotide
auagucaggaacu

SEQ ID NO: 350 - RNA, 19 nt, Artificial Sequence
  Synthetic Polynucleotide
aguuccugacuaucaauca

SEQ ID NO: 351 - RNA, 13 nt, Artificial Sequence
  Synthetic Polynucleotide
agucagccgugaa

SEQ ID NO: 352 - RNA, 19 nt, Artificial Sequence
  Synthetic Polynucleotide
uucacggcugacuuuggaa

SEQ ID NO: 353 - RNA, 13 nt, Artificial Sequence
  Synthetic Polynucleotide
acuaccaugagaa

SEQ ID NO: 354 - RNA, 19 nt, Artificial Sequence
  Synthetic Polynucleotide
uucucaugguagugaguuu

SEQ ID NO: 355 - RNA, 13 nt, Artificial Sequence
  Synthetic Polynucleotide
aaacaggcugauu

SEQ ID NO: 356 - RNA, 19 nt, Artificial Sequence
  Synthetic Polynucleotide
aaucagccuguuuaacugg

SEQ ID NO: 357 - RNA, 13 nt, Artificial Sequence
  Synthetic Polynucleotide
gagugcugaaacc

SEQ ID NO: 358 - RNA, 19 nt, Artificial Sequence
  Synthetic Polynucleotide
gguuucagcacucugguca

SEQ ID NO: 359 - RNA, 13 nt, Artificial Sequence
  Synthetic Polynucleotide
ugagcauuccgau

SEQ ID NO: 360 - RNA, 19 nt, Artificial Sequence
  Synthetic Polynucleotide
aucggaaugcucauugcuc

SEQ ID NO: 361 - RNA, 13 nt, Artificial Sequence
  Synthetic Polynucleotide
aauuccacagcca

SEQ ID NO: 362 - RNA, 19 nt, Artificial Sequence
  Synthetic Polynucleotide
uggcuguggaauucacggc

SEQ ID NO: 363 - RNA, 13 nt, Artificial Sequence
  Synthetic Polynucleotide
ugucaauugcuua

SEQ ID NO: 364 - RNA, 19 nt, Artificial Sequence
  Synthetic Polynucleotide
uaagcaauugacaccacca

SEQ ID NO: 365 - RNA, 13 nt, Artificial Sequence
  Synthetic Polynucleotide
accaugagaauug

SEQ ID NO: 366 - RNA, 19 nt, Artificial Sequence
  Synthetic Polynucleotide
caauucucaugguagugag

SEQ ID NO: 367 - RNA, 13 nt, Artificial Sequence
  Synthetic Polynucleotide
ccaacgaaagcca

SEQ ID NO: 368 - RNA, 19 nt, Artificial Sequence
  Synthetic Polynucleotide
uggcuuucguuggacuuac

SEQ ID NO: 369 - RNA, 13 nt, Artificial Sequence
  Synthetic Polynucleotide
cuggucacugauu

SEQ ID NO: 370 - RNA, 19 nt, Artificial Sequence
  Synthetic Polynucleotide
aaucagugaccaguucauc

SEQ ID NO: 371 - RNA, 13 nt, Artificial Sequence
  Synthetic Polynucleotide
ugguuuauggacu

SEQ ID NO: 372 - RNA, 19 nt, Artificial Sequence
  Synthetic Polynucleotide
aguccauaaaccacacuau

SEQ ID NO: 373 - RNA, 13 nt, Artificial Sequence
  Synthetic Polynucleotide
gaccagagugcug

SEQ ID NO: 374 - RNA, 19 nt, Artificial Sequence
  Synthetic Polynucleotide
cagcacucuggucauccag

SEQ ID NO: 375 - RNA, 13 nt, Artificial Sequence
  Synthetic Polynucleotide
gaugugauugaua

SEQ ID NO: 376 - RNA, 19 nt, Artificial Sequence
  Synthetic Polynucleotide
uaucaaucacaucggaaug

SEQ ID NO: 377 - RNA, 13 nt, Artificial Sequence
  Synthetic Polynucleotide
gucagccgugaau

SEQ ID NO: 378 - RNA, 19 nt, Artificial Sequence
  Synthetic Polynucleotide
auucacggcugacuuugga

SEQ ID NO: 379 - RNA, 13 nt, Artificial Sequence
  Synthetic Polynucleotide
aauguguaucuau

SEQ ID NO: 380 - RNA, 19 nt, Artificial Sequence
  Synthetic Polynucleotide
auagauacacauucaacca

SEQ ID NO: 381 - RNA, 13 nt, Artificial Sequence
  Synthetic Polynucleotide
uugagucuggaaa

SEQ ID NO: 382 - RNA, 19 nt, Artificial Sequence
  Synthetic Polynucleotide
uuuccagacucaaauagau

SEQ ID NO: 383 - RNA, 13 nt, Artificial Sequence
  Synthetic Polynucleotide
guccagcaauuaa

SEQ ID NO: 384 - RNA, 19 nt, Artificial Sequence
  Synthetic Polynucleotide
uuaauugcuggacaaccgu

SEQ ID NO: 385 - RNA, 13 nt, Artificial Sequence
  Synthetic Polynucleotide
ccagcaauuaaua

SEQ ID NO: 386 - RNA, 19 nt, Artificial Sequence
  Synthetic Polynucleotide
uauuaauugcuggacaacc

SEQ ID NO: 387 - RNA, 13 nt, Artificial Sequence
  Synthetic Polynucleotide
gacucgaacgacu

SEQ ID NO: 388 - RNA, 19 nt, Artificial Sequence
  Synthetic Polynucleotide
agucguucgagucaaugga

SEQ ID NO: 389 - RNA, 13 nt, Artificial Sequence
  Synthetic Polynucleotide
accugccagcaac

SEQ ID NO: 390 - RNA, 19 nt, Artificial Sequence
  Synthetic Polynucleotide
guugcuggcagguccgugg

SEQ ID NO: 391 - RNA, 13 nt, Artificial Sequence
  Synthetic Polynucleotide
gaugaaucugaua

SEQ ID NO: 392 - RNA, 19 nt, Artificial Sequence
  Synthetic Polynucleotide
uaucagauucaucagaaug

SEQ ID NO: 393 - RNA, 14 nt, Artificial Sequence
  Synthetic Polynucleotide
ugaugaaucugaua

SEQ ID NO: 394 - RNA, 19 nt, Artificial Sequence
  Synthetic Polynucleotide
uaucagauucaucagaaug

SEQ ID NO: 395 - RNA, 13 nt, Artificial Sequence
  Synthetic Polynucleotide
auuugcuuuugca

SEQ ID NO: 396 - RNA, 19 nt, Artificial Sequence
  Synthetic Polynucleotide
ugcaaaagcaaaucacugc

SEQ ID NO: 397 - RNA, 14 nt, Artificial Sequence
  Synthetic Polynucleotide
gauuugcuuuugca

SEQ ID NO: 398 - RNA, 19 nt, Artificial Sequence
  Synthetic Polynucleotide
ugcaaaagcaaaucacugc

SEQ ID NO: 399 - RNA, 13 nt, Artificial Sequence
  Synthetic Polynucleotide
gugauuugcuuua

SEQ ID NO: 400 - RNA, 19 nt, Artificial Sequence
  Synthetic Polynucleotide
uaaagcaaaucacugcaau

SEQ ID NO: 401 - RNA, 14 nt, Artificial Sequence
  Synthetic Polynucleotide
agugauuugcuuua

SEQ ID NO: 402 - RNA, 19 nt, Artificial Sequence
  Synthetic Polynucleotide
uaaagcaaaucacugcaau

SEQ ID NO: 403 - RNA, 13 nt, Artificial Sequence
  Synthetic Polynucleotide
aauuucguauuua

SEQ ID NO: 404 - RNA, 19 nt, Artificial Sequence
  Synthetic Polynucleotide
uaaauacgaaauuucaggu

SEQ ID NO: 405 - RNA, 14 nt, Artificial Sequence
  Synthetic Polynucleotide
aaauuucguauuua

SEQ ID NO: 406 - RNA, 19 nt, Artificial Sequence
  Synthetic Polynucleotide
uaaauacgaaauuucaggu

SEQ ID NO: 407 - RNA, 13 nt, Artificial Sequence
  Synthetic Polynucleotide
cacagccaugaaa

SEQ ID NO: 408 - RNA, 19 nt, Artificial Sequence
  Synthetic Polynucleotide
uuucauggcugugaaauuc

SEQ ID NO: 409 - RNA, 14 nt, Artificial Sequence
  Synthetic Polynucleotide
ucacagccaugaaa

SEQ ID NO: 410 - RNA, 19 nt, Artificial Sequence
  Synthetic Polynucleotide
uuucauggcugugaaauuc

SEQ ID NO: 411 - RNA, 13 nt, Artificial Sequence
  Synthetic Polynucleotide
gauuugcuuuuga

SEQ ID NO: 412 - RNA, 19 nt, Artificial Sequence
  Synthetic Polynucleotide
ucaaaagcaaaucacugca

SEQ ID NO: 413 - RNA, 14 nt, Artificial Sequence
  Synthetic Polynucleotide
ugauuugcuuuuga

SEQ ID NO: 414 - RNA, 19 nt, Artificial Sequence
  Synthetic Polynucleotide
ucaaaagcaaaucacugca

SEQ ID NO: 415 - RNA, 13 nt, Artificial Sequence
  Synthetic Polynucleotide
uugcuuuugccua

SEQ ID NO: 416 - RNA, 19 nt, Artificial Sequence
  Synthetic Polynucleotide
uaggcaaaagcaaaucacu

SEQ ID NO: 417 - RNA, 14 nt, Artificial Sequence
  Synthetic Polynucleotide
uuugcuuuugccua

SEQ ID NO: 418 - RNA, 19 nt, Artificial Sequence
  Synthetic Polynucleotide
uaggcaaaagcaaaucacu

SEQ ID NO: 419 - RNA, 13 nt, Artificial Sequence
  Synthetic Polynucleotide
uuucucaguuuaa

SEQ ID NO: 420 - RNA, 19 nt, Artificial Sequence
  Synthetic Polynucleotide
uuaaacugagaaagaagca

SEQ ID NO: 421 - RNA, 13 nt, Artificial Sequence
  Synthetic Polynucleotide
uugcauuuaguca

SEQ ID NO: 422 - RNA, 19 nt, Artificial Sequence
  Synthetic Polynucleotide
ugacuaaaugcaaagugag

SEQ ID NO: 423 - RNA, 13 nt, Artificial Sequence
  Synthetic Polynucleotide
acuuugcauuuaa

SEQ ID NO: 424 - RNA, 19 nt, Artificial Sequence
  Synthetic Polynucleotide
uuaaaugcaaagugagaaa

SEQ ID NO: 425 - RNA, 13 nt, Artificial Sequence
  Synthetic Polynucleotide
auuuagucaaaaa

SEQ ID NO: 426 - RNA, 19 nt, Artificial Sequence
  Synthetic Polynucleotide
uuuuugacuaaaugcaaag

SEQ ID NO: 427 - RNA, 13 nt, Artificial Sequence
  Synthetic Polynucleotide
uucuuucucagua

SEQ ID NO: 428 - RNA, 19 nt, Artificial Sequence
  Synthetic Polynucleotide
uacugagaaagaagcauuu

SEQ ID NO: 429 - RNA, 13 nt, Artificial Sequence
  Synthetic Polynucleotide
ucuuucucaguua

SEQ ID NO: 430 - RNA, 19 nt, Artificial Sequence
  Synthetic Polynucleotide
uaacugagaaagaagcauu

SEQ ID NO: 431 - RNA, 13 nt, Artificial Sequence
  Synthetic Polynucleotide
gaaagagaacaua

SEQ ID NO: 432 - RNA, 19 nt, Artificial Sequence
  Synthetic Polynucleotide
uauguucucuuucauuuug

SEQ ID NO: 433 - RNA, 13 nt, Artificial Sequence
  Synthetic Polynucleotide
cuuugcauuuaga

SEQ ID NO: 434 - RNA, 19 nt, Artificial Sequence
  Synthetic Polynucleotide
ucuaaaugcaaagugagaa

SEQ ID NO: 435 - RNA, 13 nt, Artificial Sequence
  Synthetic Polynucleotide
uuugcauuuagua

SEQ ID NO: 436 - RNA, 19 nt, Artificial Sequence
  Synthetic Polynucleotide
uacuaaaugcaaagugaga

SEQ ID NO: 437 - RNA, 13 nt, Artificial Sequence
  Synthetic Polynucleotide
cucacuuugcaua

SEQ ID NO: 438 - RNA, 19 nt, Artificial Sequence
  Synthetic Polynucleotide
uaugcaaagugagaaauug

SEQ ID NO: 439 - RNA, 13 nt, Artificial Sequence
  Synthetic Polynucleotide
uucucacuuugca

SEQ ID NO: 440 - RNA, 19 nt, Artificial Sequence
  Synthetic Polynucleotide
ugcaaagugagaaauugua

SEQ ID NO: 441 - RNA, 13 nt, Artificial Sequence
  Synthetic Polynucleotide
cacuccaguugua

SEQ ID NO: 442 - RNA, 19 nt, Artificial Sequence
  Synthetic Polynucleotide
uacaacuggagugaaaacu

SEQ ID NO: 443 - RNA, 13 nt, Artificial Sequence
  Synthetic Polynucleotide
aaugaaagagaaa

SEQ ID NO: 444 - RNA, 19 nt, Artificial Sequence
  Synthetic Polynucleotide
uuucucuuucauuuugcua

SEQ ID NO: 445 - RNA, 13 nt, Artificial Sequence
  Synthetic Polynucleotide
ugcagugauuuga

SEQ ID NO: 446 - RNA, 19 nt, Artificial Sequence
  Synthetic Polynucleotide
ucaaaucacugcaauucuc

SEQ ID NO: 447 - RNA, 13 nt, Artificial Sequence
  Synthetic Polynucleotide
ugaaagagaacaa

SEQ ID NO: 448 - RNA, 19 nt, Artificial Sequence
  Synthetic Polynucleotide
uuguucucuuucauuuugc

SEQ ID NO: 449 - RNA, 13 nt, Artificial Sequence
  Synthetic Polynucleotide
accugaaauuuca

SEQ ID NO: 450 - RNA, 19 nt, Artificial Sequence
  Synthetic Polynucleotide
ugaaauuucagguguuuau

SEQ ID NO: 451 - RNA, 13 nt, Artificial Sequence
  Synthetic Polynucleotide
gaauugcagugaa

SEQ ID NO: 452 - RNA, 19 nt, Artificial Sequence
  Synthetic Polynucleotide
uucacugcaauucucaugg

SEQ ID NO: 453 - RNA, 13 nt, Artificial Sequence
  Synthetic Polynucleotide
ggcugauucugga

SEQ ID NO: 454 - RNA, 19 nt, Artificial Sequence
  Synthetic Polynucleotide
uccagaaucagccuguuua

SEQ ID NO: 455 - RNA, 14 nt, Artificial Sequence
  Synthetic Polynucleotide
agugauuugcuuua

SEQ ID NO: 456 - RNA, 19 nt, Artificial Sequence
  Synthetic Polynucleotide
uaaagcaaaucacugcaau

SEQ ID NO: 457 - RNA, 14 nt, Artificial Sequence
  Synthetic Polynucleotide
agugauuugcuuua

SEQ ID NO: 458 - RNA, 19 nt, Artificial Sequence
  Synthetic Polynucleotide
uaaagcaaaucacugcaau

SEQ ID NO: 459 - RNA, 14 nt, Artificial Sequence
  Synthetic Polynucleotide
agugauuugcuuua

SEQ ID NO: 460 - RNA, 19 nt, Artificial Sequence
  Synthetic Polynucleotide
uaaagcaaaucacugcaau

SEQ ID NO: 461 - RNA, 14 nt, Artificial Sequence
  Synthetic Polynucleotide
agugauuugcuuua

SEQ ID NO: 462 - RNA, 19 nt, Artificial Sequence
  Synthetic Polynucleotide
uaaagcaaaucacugcaau

SEQ ID NO: 463 - RNA, 13 nt, Artificial Sequence
  Synthetic Polynucleotide
cacauuugauuga

SEQ ID NO: 464 - RNA, 19 nt, Artificial Sequence
  Synthetic Polynucleotide
ucaaucaaaugugaucugg

SEQ ID NO: 465 - RNA, 13 nt, Artificial Sequence
  Synthetic Polynucleotide
cacugccucaauu

SEQ ID NO: 466 - RNA, 19 nt, Artificial Sequence
  Synthetic Polynucleotide
aauugaggcaguguugaug

SEQ ID NO: 467 - RNA, 13 nt, Artificial Sequence
  Synthetic Polynucleotide
aaauaccagucuu

SEQ ID NO: 468 - RNA, 19 nt, Artificial Sequence
  Synthetic Polynucleotide
aagacugguauuucaucug

SEQ ID NO: 469 - RNA, 13 nt, Artificial Sequence
  Synthetic Polynucleotide
cauuugauugaca

SEQ ID NO: 470 - RNA, 19 nt, Artificial Sequence
  Synthetic Polynucleotide
ugucaaucaaaugugaucu

SEQ ID NO: 471 - RNA, 13 nt, Artificial Sequence
  Synthetic Polynucleotide
gaaaacugcucaa

SEQ ID NO: 472 - RNA, 19 nt, Artificial Sequence
  Synthetic Polynucleotide
uugagcaguuuucuccaua

SEQ ID NO: 473 - RNA, 13 nt, Artificial Sequence
  Synthetic Polynucleotide
accucuccuauua

SEQ ID NO: 474 - RNA, 19 nt, Artificial Sequence
  Synthetic Polynucleotide
uaauaggagagguuagaga

SEQ ID NO: 475 - RNA, 13 nt, Artificial Sequence
  Synthetic Polynucleotide
uccaccaacuuaa

SEQ ID NO: 476 - RNA, 19 nt, Artificial Sequence
  Synthetic Polynucleotide
uuaaguugguggacuguca

SEQ ID NO: 477 - RNA, 14 nt, Artificial Sequence
  Synthetic Polynucleotide
guccaccaacuuaa

SEQ ID NO: 478 - RNA, 19 nt, Artificial Sequence
  Synthetic Polynucleotide
uuaaguugguggacuguca

SEQ ID NO: 479 - RNA, 13 nt, Artificial Sequence
  Synthetic Polynucleotide
cuccuauuauaca

SEQ ID NO: 480 - RNA, 19 nt, Artificial Sequence
  Synthetic Polynucleotide
uguauaauaggagagguua

SEQ ID NO: 481 - RNA, 13 nt, Artificial Sequence
  Synthetic Polynucleotide
gaucacauuugaa

SEQ ID NO: 482 - RNA, 19 nt, Artificial Sequence
  Synthetic Polynucleotide
uucaaaugugaucuggaug

SEQ ID NO: 483 - RNA, 14 nt, Artificial Sequence
  Synthetic Polynucleotide
agaucacauuugaa

SEQ ID NO: 484 - RNA, 19 nt, Artificial Sequence
  Synthetic Polynucleotide
uucaaaugugaucuggaug

SEQ ID NO: 485 - RNA, 13 nt, Artificial Sequence
  Synthetic Polynucleotide
aaccucuccuaua

SEQ ID NO: 486 - RNA, 19 nt, Artificial Sequence
  Synthetic Polynucleotide
uauaggagagguuagagaa

SEQ ID NO: 487 - RNA, 13 nt, Artificial Sequence
  Synthetic Polynucleotide
guugacauccaga

SEQ ID NO: 488 - RNA, 19 nt, Artificial Sequence
  Synthetic Polynucleotide
ucuggaugucaacacauaa

SEQ ID NO: 489 - RNA, 13 nt, Artificial Sequence
  Synthetic Polynucleotide
ccuuccuucgaaa

SEQ ID NO: 490 - RNA, 19 nt, Artificial Sequence
  Synthetic Polynucleotide
uuucgaaggaagggaaugu

SEQ ID NO: 491 - RNA, 13 nt, Artificial Sequence
  Synthetic Polynucleotide
acuccaaacacaa

SEQ ID NO: 492 - RNA, 19 nt, Artificial Sequence
  Synthetic Polynucleotide
uuguguuuggaguggguuu

SEQ ID NO: 493 - RNA, 14 nt, Artificial Sequence
  Synthetic Polynucleotide
cacuccaaacacaa

SEQ ID NO: 494 - RNA, 19 nt, Artificial Sequence
  Synthetic Polynucleotide
uuguguuuggaguggguuu

SEQ ID NO: 495 - RNA, 13 nt, Artificial Sequence
  Synthetic Polynucleotide
cacuccaaacaca

SEQ ID NO: 496 - RNA, 19 nt, Artificial Sequence
  Synthetic Polynucleotide
uguguuuggaguggguuuc

SEQ ID NO: 497 - RNA, 13 nt, Artificial Sequence
  Synthetic Polynucleotide
ccaccaacuuaca

SEQ ID NO: 498 - RNA, 19 nt, Artificial Sequence
  Synthetic Polynucleotide
uguaaguugguggacuguc

SEQ ID NO: 499 - RNA, 14 nt, Artificial Sequence
  Synthetic Polynucleotide
uccaccaacuuaca

SEQ ID NO: 500 - RNA, 19 nt, Artificial Sequence
  Synthetic Polynucleotide
uguaaguugguggacuguc

SEQ ID NO: 501 - RNA, 13 nt, Artificial Sequence
  Synthetic Polynucleotide
aauaccagucuua

SEQ ID NO: 502 - RNA, 19 nt, Artificial Sequence
  Synthetic Polynucleotide
uaagacugguauuucaucu

SEQ ID NO: 503 - RNA, 13 nt, Artificial Sequence
  Synthetic Polynucleotide
gaccaguauaaga

SEQ ID NO: 504 - RNA, 19 nt, Artificial Sequence
  Synthetic Polynucleotide
ucuuauacuggucaaaucc

SEQ ID NO: 505 - RNA, 13 nt, Artificial Sequence
  Synthetic Polynucleotide
gucuuuuaaugaa

SEQ ID NO: 506 - RNA, 19 nt, Artificial Sequence
  Synthetic Polynucleotide
uucauuaaaagacugguau

SEQ ID NO: 507 - RNA, 13 nt, Artificial Sequence
  Synthetic Polynucleotide
aauuucaugucua

SEQ ID NO: 508 - RNA, 19 nt, Artificial Sequence
  Synthetic Polynucleotide
uagacaugaaauuacuggu

SEQ ID NO: 509 - RNA, 13 nt, Artificial Sequence
  Synthetic Polynucleotide
aucacauuugaua

SEQ ID NO: 510 - RNA, 19 nt, Artificial Sequence
  Synthetic Polynucleotide
uaucaaaugugaucuggau

SEQ ID NO: 511 - RNA, 14 nt, Artificial Sequence
  Synthetic Polynucleotide
gaucacauuugaua

SEQ ID NO: 512 - RNA, 19 nt, Artificial Sequence
  Synthetic Polynucleotide
uaucaaaugugaucuggau

SEQ ID NO: 513 - RNA, 13 nt, Artificial Sequence
  Synthetic Polynucleotide
uccagaucacaua

SEQ ID NO: 514 - RNA, 19 nt, Artificial Sequence
  Synthetic Polynucleotide
uaugugaucuggaugucaa

SEQ ID NO: 515 - RNA, 13 nt, Artificial Sequence
  Synthetic Polynucleotide
uacugauaggaga

SEQ ID NO: 516 - RNA, 19 nt, Artificial Sequence
  Synthetic Polynucleotide
ucuccuaucaguauuagcc

SEQ ID NO: 517 - RNA, 13 nt, Artificial Sequence
  Synthetic Polynucleotide
gugcaacacuuga

SEQ ID NO: 518 - RNA, 19 nt, Artificial Sequence
  Synthetic Polynucleotide
ucaaguguugcacauaauc

SEQ ID NO: 519 - RNA, 13 nt, Artificial Sequence
  Synthetic Polynucleotide
accaguauaagua

SEQ ID NO: 520 - RNA, 19 nt, Artificial Sequence
  Synthetic Polynucleotide
uacuuauacuggucaaauc

SEQ ID NO: 521 - RNA, 13 nt, Artificial Sequence
  Synthetic Polynucleotide
gaagucuaaugaa

SEQ ID NO: 522 - RNA, 19 nt, Artificial Sequence
  Synthetic Polynucleotide
uucauuagacuucuacagu

SEQ ID NO: 523 - RNA, 13 nt, Artificial Sequence
  Synthetic Polynucleotide
aagaagaaaguua

SEQ ID NO: 524 - RNA, 19 nt, Artificial Sequence
  Synthetic Polynucleotide
uaacuuucuucuuagaagc

SEQ ID NO: 525 - RNA, 13 nt, Artificial Sequence
  Synthetic Polynucleotide
ucacauuugauua

SEQ ID NO: 526 - RNA, 19 nt, Artificial Sequence
  Synthetic Polynucleotide
uaaucaaaugugaucugga

SEQ ID NO: 527 - RNA, 14 nt, Artificial Sequence
  Synthetic Polynucleotide
aucacauuugauua

SEQ ID NO: 528 - RNA, 19 nt, Artificial Sequence
  Synthetic Polynucleotide
uaaucaaaugugaucugga

SEQ ID NO: 529 - RNA, 13 nt, Artificial Sequence
  Synthetic Polynucleotide
acauuugauugaa

SEQ ID NO: 530 - RNA, 19 nt, Artificial Sequence
  Synthetic Polynucleotide
uucaaucaaaugugaucug

SEQ ID NO: 531 - RNA, 14 nt, Artificial Sequence
  Synthetic Polynucleotide
cacauuugauugaa

SEQ ID NO: 532 - RNA, 19 nt, Artificial Sequence
  Synthetic Polynucleotide
uucaaucaaaugugaucug

SEQ ID NO: 533 - RNA, 13 nt, Artificial Sequence
  Synthetic Polynucleotide
auuugauugacaa

SEQ ID NO: 534 - RNA, 19 nt, Artificial Sequence
  Synthetic Polynucleotide
uugucaaucaaaugugauc

SEQ ID NO: 535 - RNA, 14 nt, Artificial Sequence
  Synthetic Polynucleotide
cauuugauugacaa

SEQ ID NO: 536 - RNA, 19 nt, Artificial Sequence
  Synthetic Polynucleotide
uugucaaucaaaugugauc

SEQ ID NO: 537 - RNA, 13 nt, Artificial Sequence
  Synthetic Polynucleotide
caucugcaauaaa

SEQ ID NO: 538 - RNA, 19 nt, Artificial Sequence
  Synthetic Polynucleotide
uuuauugcagaugagagac

SEQ ID NO: 539 - RNA, 14 nt, Artificial Sequence
  Synthetic Polynucleotide
ucaucugcaauaaa

SEQ ID NO: 540 - RNA, 19 nt, Artificial Sequence
  Synthetic Polynucleotide
uuuauugcagaugagagac

SEQ ID NO: 541 - RNA, 13 nt, Artificial Sequence
  Synthetic Polynucleotide
gaucacauuugaa

SEQ ID NO: 542 - RNA, 19 nt, Artificial Sequence
  Synthetic Polynucleotide
uucaaaugugaucuggaug

SEQ ID NO: 543 - RNA, 13 nt, Artificial Sequence
  Synthetic Polynucleotide
gaucacauuugaa

SEQ ID NO: 544 - RNA, 19 nt, Artificial Sequence
  Synthetic Polynucleotide
uucaaaugugaucuggaug

SEQ ID NO: 545 - RNA, 13 nt, Artificial Sequence
  Synthetic Polynucleotide
  misc_feature (3)..(4): Modified with 2′Ome
  misc_feature (6)..(6): Modified with 2′Ome
  misc_feature (8)..(10): Modified with 2′Ome
  misc_feature (12)..(13): Modified with 2′Ome
  misc_feature (13)..(13): Modified with cholesterol with TEG linker
gaucacauuugaa

SEQ ID NO: 546 - RNA, 19 nt, Artificial Sequence
  Synthetic Polynucleotide
uucaaaugugaucuggaug

SEQ ID NO: 547 - RNA, 13 nt, Artificial Sequence
  Synthetic Polynucleotide
gaucacauuugaa

SEQ ID NO: 548 - RNA, 19 nt, Artificial Sequence
  Synthetic Polynucleotide
uucaaaugugaucuggaug

SEQ ID NO: 549 - RNA, 13 nt, Artificial Sequence
  Synthetic Polynucleotide
gaucacauuugaa

SEQ ID NO: 550 - RNA, 19 nt, Artificial Sequence
  Synthetic Polynucleotide
uucaaaugugaucuggaug

SEQ ID NO: 551 - RNA, 14 nt, Artificial Sequence
  Synthetic Polynucleotide
gaucacauuugaua

SEQ ID NO: 552 - RNA, 20 nt, Artificial Sequence
  Synthetic Polynucleotide
  misc_feature (1)..(1): Modified with P
  misc_feature (1)..(1): Modified with 2′Ome
  misc_feature (3)..(4): Modified with 2′fluoro
  misc_feature (8)..(8): Modified with 2′fluoro
  misc_feature (10)..(10): Modified with 2′fluoro
  misc_feature (13)..(15): Modified with 2′fluoro
  misc_feature (19)..(19): Modified with 2′fluoro
  misc_feature (14)..(20): Phosphorothioate internucleotide bond
uaucaaaugugaucuggaug

SEQ ID NO: 553 - RNA, 14 nt, Artificial Sequence
  Synthetic Polynucleotide
gaucacauuugaua

SEQ ID NO: 554 - RNA, 20 nt, Artificial Sequence
  Synthetic Polynucleotide
uaucaaaugugaucuggaug

SEQ ID NO: 555 - RNA, 14 nt, Artificial Sequence
  Synthetic Polynucleotide
gaucacauuugaua

SEQ ID NO: 556 - RNA, 20 nt, Artificial Sequence
  Synthetic Polynucleotide
uaucaaaugugaucuggaug

SEQ ID NO: 557 - RNA, 14 nt, Artificial Sequence
  Synthetic Polynucleotide
gaucacauuugaua

SEQ ID NO: 558 - RNA, 20 nt, Artificial Sequence
  Synthetic Polynucleotide
uaucaaaugugaucuggaug

SEQ ID NO: 559 - RNA, 14 nt, Artificial Sequence
  Synthetic Polynucleotide
gaucacauuugaua

SEQ ID NO: 560 - RNA, 20 nt, Artificial Sequence
  Synthetic Polynucleotide
uaucaaaugugaucuggaug

SEQ ID NO: 561 - RNA, 14 nt, Artificial Sequence
  Synthetic Polynucleotide
gaucacauuugaua

SEQ ID NO: 562 - RNA, 20 nt, Artificial Sequence
  Synthetic Polynucleotide
  misc_feature (1)..(1): Modified with P
  misc_feature (1)..(1): Modified with 2′Ome
  misc_feature (3)..(4): Modified with 2′fluoro
  misc_feature (8)..(8): Modified with 2′fluoro
  misc_feature (10)..(10): Modified with 2′fluoro
  misc_feature (13)..(15): Modified with 2′fluoro
  misc_feature (16)..(18): Modified with 2′Ome
  misc_feature (19)..(19): Modified with 2′fluoro
  misc_feature (14)..(20): Phosphorothioate internucleotide bond
uaucaaaugugaucuggaug

SEQ ID NO: 563 - RNA, 14 nt, Artificial Sequence
  Synthetic Polynucleotide
gaucacauuugaua

SEQ ID NO: 564 - RNA, 20 nt, Artificial Sequence
  Synthetic Polynucleotide
uaucaaaugugaucuggaug

SEQ ID NO: 565 - RNA, 14 nt, Artificial Sequence
  Synthetic Polynucleotide
gaucacauuugaua

SEQ ID NO: 566 - RNA, 20 nt, Artificial Sequence
  Synthetic Polynucleotide
uaucaaaugugaucuggaug

SEQ ID NO: 567 - RNA, 14 nt, Artificial Sequence
  Synthetic Polynucleotide
  misc_feature (3)..(4): Modified with 2′Ome
  misc_feature (6)..(6): Modified with 2′Ome
  misc_feature (8)..(10): Modified with 2′Ome
  misc_feature (13)..(14): Modified with 2′Ome
  misc_feature (12)..(14): Phosphorothioate internucleotide bond
  misc_feature (14)..(14): Modified with cholesterol with TEG linker
gaucacauuugaua

SEQ ID NO: 568 - RNA, 19 nt, Artificial Sequence
  Synthetic Polynucleotide
uaucaaaugugaucuggau

SEQ ID NO: 569 - RNA, 14 nt, Artificial Sequence
  Synthetic Polynucleotide
gaucacauuugaua

SEQ ID NO: 570 - RNA, 19 nt, Artificial Sequence
  Synthetic Polynucleotide
uaucaaaugugaucuggau

SEQ ID NO: 571 - RNA, 14 nt, Artificial Sequence
  Synthetic Polynucleotide
gaucacauuugaua

SEQ ID NO: 572 - RNA, 19 nt, Artificial Sequence
  Synthetic Polynucleotide
uaucaaaugugaucuggau

SEQ ID NO: 573 - RNA, 14 nt, Artificial Sequence
  Synthetic Polynucleotide
gaucacauuugaua

SEQ ID NO: 574 - RNA, 19 nt, Artificial Sequence
  Synthetic Polynucleotide
uaucaaaugugaucuggau

SEQ ID NO: 575 - RNA, 14 nt, Artificial Sequence
  Synthetic Polynucleotide
gaucacauuugaua

SEQ ID NO: 576 - RNA, 19 nt, Artificial Sequence
  Synthetic Polynucleotide
uaucaaaugugaucuggau

SEQ ID NO: 577 - RNA, 14 nt, Artificial Sequence
  Synthetic Polynucleotide
gaucacauuugaua

SEQ ID NO: 578 - RNA, 19 nt, Artificial Sequence
  Synthetic Polynucleotide
uaucaaaugugaucuggau

SEQ ID NO: 579 - RNA, 14 nt, Artificial Sequence
  Synthetic Polynucleotide
gaucacauuugaua

SEQ ID NO: 580 - RNA, 19 nt, Artificial Sequence
  Synthetic Polynucleotide
uaucaaaugugaucuggau

SEQ ID NO: 581 - RNA, 13 nt, Artificial Sequence
  Synthetic Polynucleotide
gaucacauuugaa

SEQ ID NO: 582 - RNA, 19 nt, Artificial Sequence
  Synthetic Polynucleotide
  misc_feature (1)..(1): Modified with P
  misc_feature (1)..(1): Modified with 2′Ome
  misc_feature (2)..(3): Modified with 2′fluoro
  misc_feature (7)..(7): Modified with 2′fluoro
  misc_feature (9)..(9): Modified with 2′fluoro
  misc_feature (12)..(14): Modified with 2′Ome
  misc_feature (18)..(18): Modified with 2′Ome
  misc_feature (13)..(19): Phosphorothioate internucleotide bond
uucaaaugugaucuggaug

SEQ ID NO: 583 - RNA, 13 nt, Artificial Sequence
  Synthetic Polynucleotide
gaucacauuugaa

SEQ ID NO: 584 - RNA, 19 nt, Artificial Sequence
  Synthetic Polynucleotide
uucaaaugugaucuggaug

SEQ ID NO: 585 - RNA, 13 nt, Artificial Sequence
  Synthetic Polynucleotide
gaucacauuugaa

SEQ ID NO: 586 - RNA, 19 nt, Artificial Sequence
  Synthetic Polynucleotide
uucaaaugugaucuggaug

SEQ ID NO: 587 - RNA, 13 nt, Artificial Sequence
  Synthetic Polynucleotide
gaucacauuugaa

SEQ ID NO: 588 - RNA, 19 nt, Artificial Sequence
  Synthetic Polynucleotide
uucaaaugugaucuggaug

SEQ ID NO: 589 - RNA, 13 nt, Artificial Sequence
  Synthetic Polynucleotide
acaggaagaugua

SEQ ID NO: 590 - RNA, 19 nt, Artificial Sequence
  Synthetic Polynucleotide
uacaucuuccuguaguaca

SEQ ID NO: 591 - RNA, 13 nt, Artificial Sequence
  Synthetic Polynucleotide
gaguggagcgccu

SEQ ID NO: 592 - RNA, 19 nt, Artificial Sequence
  Synthetic Polynucleotide
aggcgcuccacucuguggu

SEQ ID NO: 593 - RNA, 13 nt, Artificial Sequence
  Synthetic Polynucleotide
cgacuggaagaca

SEQ ID NO: 594 - RNA, 19 nt, Artificial Sequence
  Synthetic Polynucleotide
ugucuuccagucgguaagc

SEQ ID NO: 595 - RNA, 13 nt, Artificial Sequence
  Synthetic Polynucleotide
ggagcgccuguuc

SEQ ID NO: 596 - RNA, 19 nt, Artificial Sequence
  Synthetic Polynucleotide
gaacaggcgcuccacucug

SEQ ID NO: 597 - RNA, 13 nt, Artificial Sequence
  Synthetic Polynucleotide
gccauuacaacug

SEQ ID NO: 598 - RNA, 19 nt, Artificial Sequence
  Synthetic Polynucleotide
caguuguaauggcaggcac

SEQ ID NO: 599 - RNA, 13 nt, Artificial Sequence
  Synthetic Polynucleotide
gagcuuucuggcu

SEQ ID NO: 600 - RNA, 19 nt, Artificial Sequence
  Synthetic Polynucleotide
agccagaaagcucaaacuu

SEQ ID NO: 601 - RNA, 13 nt, Artificial Sequence
  Synthetic Polynucleotide
aguggagcgccug

SEQ ID NO: 602 - RNA, 19 nt, Artificial Sequence
  Synthetic Polynucleotide
caggcgcuccacucugugg

SEQ ID NO: 603 - RNA, 13 nt, Artificial Sequence
  Synthetic Polynucleotide
uggagcgccuguu

SEQ ID NO: 604 - RNA, 19 nt, Artificial Sequence
  Synthetic Polynucleotide
aacaggcgcuccacucugu

SEQ ID NO: 605 - RNA, 13 nt, Artificial Sequence
  Synthetic Polynucleotide
guuugagcuuucu

SEQ ID NO: 606 - RNA, 19 nt, Artificial Sequence
  Synthetic Polynucleotide
agaaagcucaaacuugaua

SEQ ID NO: 607 - RNA, 13 nt, Artificial Sequence
  Synthetic Polynucleotide
ugccauuacaacu

SEQ ID NO: 608 - RNA, 19 nt, Artificial Sequence
  Synthetic Polynucleotide
aguuguaauggcaggcaca

SEQ ID NO: 609 - RNA, 13 nt, Artificial Sequence
  Synthetic Polynucleotide
acuggaagacacg

SEQ ID NO: 610 - RNA, 19 nt, Artificial Sequence
  Synthetic Polynucleotide
cgugucuuccagucgguaa

SEQ ID NO: 611 - RNA, 13 nt, Artificial Sequence
  Synthetic Polynucleotide
aacugccuggucc

SEQ ID NO: 612 - RNA, 19 nt, Artificial Sequence
  Synthetic Polynucleotide
ggaccaggcaguuggcucu

SEQ ID NO: 613 - RNA, 13 nt, Artificial Sequence
  Synthetic Polynucleotide
agaccugugccug

SEQ ID NO: 614 - RNA, 19 nt, Artificial Sequence
  Synthetic Polynucleotide
caggcacaggucuugauga

SEQ ID NO: 615 - RNA, 13 nt, Artificial Sequence
  Synthetic Polynucleotide
cagaguggagcgc

SEQ ID NO: 616 - RNA, 19 nt, Artificial Sequence
  Synthetic Polynucleotide
gcgcuccacucuguggucu

SEQ ID NO: 617 - RNA, 13 nt, Artificial Sequence
  Synthetic Polynucleotide
ccugguccagacc

SEQ ID NO: 618 - RNA, 19 nt, Artificial Sequence
  Synthetic Polynucleotide
ggucuggaccaggcaguug

SEQ ID NO: 619 - RNA, 13 nt, Artificial Sequence
  Synthetic Polynucleotide
ccauuacaacugu

SEQ ID NO: 620 - RNA, 19 nt, Artificial Sequence
  Synthetic Polynucleotide
acaguuguaauggcaggca

SEQ ID NO: 621 - RNA, 13 nt, Artificial Sequence
  Synthetic Polynucleotide
cugccauuacaac

SEQ ID NO: 622 - RNA, 19 nt, Artificial Sequence
  Synthetic Polynucleotide
guuguaauggcaggcacag

SEQ ID NO: 623 - RNA, 13 nt, Artificial Sequence
  Synthetic Polynucleotide
auuacaacugucc

SEQ ID NO: 624 - RNA, 19 nt, Artificial Sequence
  Synthetic Polynucleotide
ggacaguuguaauggcagg

SEQ ID NO: 625 - RNA, 13 nt, Artificial Sequence
  Synthetic Polynucleotide
cauuacaacuguc

SEQ ID NO: 626 - RNA, 19 nt, Artificial Sequence
  Synthetic Polynucleotide
gacaguuguaauggcaggc

SEQ ID NO: 627 - RNA, 13 nt, Artificial Sequence
  Synthetic Polynucleotide
agaguggagcgcc

SEQ ID NO: 628 - RNA, 19 nt, Artificial Sequence
  Synthetic Polynucleotide
ggcgcuccacucugugguc

SEQ ID NO: 629 - RNA, 13 nt, Artificial Sequence
  Synthetic Polynucleotide
accgacuggaaga

SEQ ID NO: 630 - RNA, 19 nt, Artificial Sequence
  Synthetic Polynucleotide
ucuuccagucgguaagccg

SEQ ID NO: 631 - RNA, 13 nt, Artificial Sequence
  Synthetic Polynucleotide
auguacggagaca

SEQ ID NO: 632 - RNA, 19 nt, Artificial Sequence
  Synthetic Polynucleotide
ugucuccguacaucuuccu

SEQ ID NO: 633 - RNA, 13 nt, Artificial Sequence
  Synthetic Polynucleotide
gccuugcgaagcu

SEQ ID NO: 634 - RNA, 19 nt, Artificial Sequence
  Synthetic Polynucleotide
agcuucgcaaggccugacc

SEQ ID NO: 635 - RNA, 13 nt, Artificial Sequence
  Synthetic Polynucleotide
gcugcgaggagug

SEQ ID NO: 636 - RNA, 19 nt, Artificial Sequence
  Synthetic Polynucleotide
cacuccucgcagcauuucc

SEQ ID NO: 637 - RNA, 13 nt, Artificial Sequence
  Synthetic Polynucleotide
gccuaucaaguuu

SEQ ID NO: 638 - RNA, 19 nt, Artificial Sequence
  Synthetic Polynucleotide
aaacuugauaggcuuggag

SEQ ID NO: 639 - RNA, 13 nt, Artificial Sequence
  Synthetic Polynucleotide
aauucuguggagu

SEQ ID NO: 640 - RNA, 19 nt, Artificial Sequence
  Synthetic Polynucleotide
acuccacagaauuuagcuc

SEQ ID NO: 641 - RNA, 13 nt, Artificial Sequence
  Synthetic Polynucleotide
uguacggagacau

SEQ ID NO: 642 - RNA, 19 nt, Artificial Sequence
  Synthetic Polynucleotide
augucuccguacaucuucc

SEQ ID NO: 643 - RNA, 13 nt, Artificial Sequence
  Synthetic Polynucleotide
agccuaucaaguu

SEQ ID NO: 644 - RNA, 19 nt, Artificial Sequence
  Synthetic Polynucleotide
aacuugauaggcuuggaga

SEQ ID NO: 645 - RNA, 13 nt, Artificial Sequence
  Synthetic Polynucleotide
caaguuugagcuu

SEQ ID NO: 646 - RNA, 19 nt, Artificial Sequence
  Synthetic Polynucleotide
aagcucaaacuugauaggc

SEQ ID NO: 647 - RNA, 13 nt, Artificial Sequence
  Synthetic Polynucleotide
cuguggaguaugu

SEQ ID NO: 648 - RNA, 19 nt, Artificial Sequence
  Synthetic Polynucleotide
acauacuccacagaauuua

SEQ ID NO: 649 - RNA, 13 nt, Artificial Sequence
  Synthetic Polynucleotide
aaauucuguggag

SEQ ID NO: 650 - RNA, 19 nt, Artificial Sequence
  Synthetic Polynucleotide
cuccacagaauuuagcucg

SEQ ID NO: 651 - RNA, 13 nt, Artificial Sequence
  Synthetic Polynucleotide
uuucaguagcaca

SEQ ID NO: 652 - RNA, 19 nt, Artificial Sequence
  Synthetic Polynucleotide
ugugcuacugaaaucauuu

SEQ ID NO: 653 - RNA, 13 nt, Artificial Sequence
  Synthetic Polynucleotide
caaugacaucuuu

SEQ ID NO: 654 - RNA, 19 nt, Artificial Sequence
  Synthetic Polynucleotide
aaagaugucauugucuccg

SEQ ID NO: 655 - RNA, 13 nt, Artificial Sequence
  Synthetic Polynucleotide
aguaccagugcac

SEQ ID NO: 656 - RNA, 19 nt, Artificial Sequence
  Synthetic Polynucleotide
gugcacugguacuugcagc

SEQ ID NO: 657 - RNA, 13 nt, Artificial Sequence
  Synthetic Polynucleotide
ggaagacacguuu

SEQ ID NO: 658 - RNA, 19 nt, Artificial Sequence
  Synthetic Polynucleotide
aaacgugucuuccagucgg

SEQ ID NO: 659 - RNA, 13 nt, Artificial Sequence
  Synthetic Polynucleotide
cuaucaaguuuga

SEQ ID NO: 660 - RNA, 19 nt, Artificial Sequence
  Synthetic Polynucleotide
ucaaacuugauaggcuugg

SEQ ID NO: 661 - RNA, 13 nt, Artificial Sequence
  Synthetic Polynucleotide
agcuaaauucugu

SEQ ID NO: 662 - RNA, 19 nt, Artificial Sequence
  Synthetic Polynucleotide
acagaauuuagcucgguau

SEQ ID NO: 663 - RNA, 13 nt, Artificial Sequence
  Synthetic Polynucleotide
agguagaauguaa

SEQ ID NO: 664 - RNA, 19 nt, Artificial Sequence
  Synthetic Polynucleotide
uuacauucuaccuauggug

SEQ ID NO: 665 - RNA, 13 nt, Artificial Sequence
  Synthetic Polynucleotide
agcugaucaguuu

SEQ ID NO: 666 - RNA, 19 nt, Artificial Sequence
  Synthetic Polynucleotide
aaacugaucagcuauauag

SEQ ID NO: 667 - RNA, 13 nt, Artificial Sequence
  Synthetic Polynucleotide
uucugcucagaua

SEQ ID NO: 668 - RNA, 19 nt, Artificial Sequence
  Synthetic Polynucleotide
uaucugagcagaauuucca

SEQ ID NO: 669 - RNA, 13 nt, Artificial Sequence
  Synthetic Polynucleotide
uuaucuaaguuaa

SEQ ID NO: 670 - RNA, 19 nt, Artificial Sequence
  Synthetic Polynucleotide
uuaacuuagauaacuguac

SEQ ID NO: 671 - RNA, 13 nt, Artificial Sequence
  Synthetic Polynucleotide
uauacgaguaaua

SEQ ID NO: 672 - RNA, 19 nt, Artificial Sequence
  Synthetic Polynucleotide
uauuacucguauaagaugc

SEQ ID NO: 673 - RNA, 13 nt, Artificial Sequence
  Synthetic Polynucleotide
gacuggacagcuu

SEQ ID NO: 674 - RNA, 19 nt, Artificial Sequence
  Synthetic Polynucleotide
aagcuguccagucuaaucg

SEQ ID NO: 675 - RNA, 13 nt, Artificial Sequence
  Synthetic Polynucleotide
auggccuuuauua

SEQ ID NO: 676 - RNA, 19 nt, Artificial Sequence
  Synthetic Polynucleotide
uaauaaaggccauuuguuc

SEQ ID NO: 677 - RNA, 13 nt, Artificial Sequence
  Synthetic Polynucleotide
auaccgagcuaaa

SEQ ID NO: 678 - RNA, 19 nt, Artificial Sequence
  Synthetic Polynucleotide
uuuagcucgguaugucuuc

SEQ ID NO: 679 - RNA, 13 nt, Artificial Sequence
  Synthetic Polynucleotide
uuguugagagugu

SEQ ID NO: 680 - RNA, 19 nt, Artificial Sequence
  Synthetic Polynucleotide
acacucucaacaaauaaac

SEQ ID NO: 681 - RNA, 13 nt, Artificial Sequence
  Synthetic Polynucleotide
acauaccgagcua

SEQ ID NO: 682 - RNA, 19 nt, Artificial Sequence
  Synthetic Polynucleotide
uagcucgguaugucuucau

SEQ ID NO: 683 - RNA, 13 nt, Artificial Sequence
  Synthetic Polynucleotide
agcagaaagguua

SEQ ID NO: 684 - RNA, 19 nt, Artificial Sequence
  Synthetic Polynucleotide
uaaccuuucugcugguacc

SEQ ID NO: 685 - RNA, 13 nt, Artificial Sequence
  Synthetic Polynucleotide
aguuguuccuuaa

SEQ ID NO: 686 - RNA, 19 nt, Artificial Sequence
  Synthetic Polynucleotide
uuaaggaacaacuugacuc

SEQ ID NO: 687 - RNA, 13 nt, Artificial Sequence
  Synthetic Polynucleotide
auuugaaguguaa

SEQ ID NO: 688 - RNA, 19 nt, Artificial Sequence
  Synthetic Polynucleotide
uuacacuucaaauagcagg

SEQ ID NO: 689 - RNA, 13 nt, Artificial Sequence
  Synthetic Polynucleotide
aagcugaccugga

SEQ ID NO: 690 - RNA, 19 nt, Artificial Sequence
  Synthetic Polynucleotide
uccaggucagcuucgcaag

SEQ ID NO: 691 - RNA, 13 nt, Artificial Sequence
  Synthetic Polynucleotide
ggucaugaagaag

SEQ ID NO: 692 - RNA, 19 nt, Artificial Sequence
  Synthetic Polynucleotide
cuucuucaugaccucgccg

SEQ ID NO: 693 - RNA, 13 nt, Artificial Sequence
  Synthetic Polynucleotide
auggucaggccuu

SEQ ID NO: 694 - RNA, 19 nt, Artificial Sequence
  Synthetic Polynucleotide
aaggccugaccaugcacag

SEQ ID NO: 695 - RNA, 13 nt, Artificial Sequence
  Synthetic Polynucleotide
gaagacacguuug

SEQ ID NO: 696 - RNA, 19 nt, Artificial Sequence
  Synthetic Polynucleotide
caaacgugucuuccagucg

SEQ ID NO: 697 - RNA, 13 nt, Artificial Sequence
  Synthetic Polynucleotide
aggccuugcgaag

SEQ ID NO: 698 - RNA, 19 nt, Artificial Sequence
  Synthetic Polynucleotide
cuucgcaaggccugaccau

SEQ ID NO: 699 - RNA, 13 nt, Artificial Sequence
  Synthetic Polynucleotide
uaccgacuggaag

SEQ ID NO: 700 - RNA, 19 nt, Artificial Sequence
  Synthetic Polynucleotide
cuuccagucgguaagccgc

SEQ ID NO: 701 - RNA, 13 nt, Artificial Sequence
  Synthetic Polynucleotide
accgcaagaucgg

SEQ ID NO: 702 - RNA, 19 nt, Artificial Sequence
  Synthetic Polynucleotide
ccgaucuugcgguuggccg

SEQ ID NO: 703 - RNA, 13 nt, Artificial Sequence
  Synthetic Polynucleotide
caggccuugcgaa

SEQ ID NO: 704 - RNA, 19 nt, Artificial Sequence
  Synthetic Polynucleotide
uucgcaaggccugaccaug

SEQ ID NO: 705 - RNA, 13 nt, Artificial Sequence
  Synthetic Polynucleotide
cgagcuaaauucu

SEQ ID NO: 706 - RNA, 19 nt, Artificial Sequence
  Synthetic Polynucleotide
agaauuuagcucgguaugu

SEQ ID NO: 707 - RNA, 13 nt, Artificial Sequence
  Synthetic Polynucleotide
ucuguggaguaug

SEQ ID NO: 708 - RNA, 19 nt, Artificial Sequence
  Synthetic Polynucleotide
cauacuccacagaauuuag

SEQ ID NO: 709 - RNA, 13 nt, Artificial Sequence
  Synthetic Polynucleotide
cggagacauggca

SEQ ID NO: 710 - RNA, 19 nt, Artificial Sequence
  Synthetic Polynucleotide
ugccaugucuccguacauc

SEQ ID NO: 711 - RNA, 13 nt, Artificial Sequence
  Synthetic Polynucleotide
augacaacgccuc

SEQ ID NO: 712 - RNA, 19 nt, Artificial Sequence
  Synthetic Polynucleotide
gaggcguugucauugguaa

SEQ ID NO: 713 - RNA, 13 nt, Artificial Sequence
  Synthetic Polynucleotide
gaggucaugaaga

SEQ ID NO: 714 - RNA, 19 nt, Artificial Sequence
  Synthetic Polynucleotide
ucuucaugaccucgccguc

SEQ ID NO: 715 - RNA, 13 nt, Artificial Sequence
  Synthetic Polynucleotide
uaaauucugugga

SEQ ID NO: 716 - RNA, 19 nt, Artificial Sequence
  Synthetic Polynucleotide
uccacagaauuuagcucgg

SEQ ID NO: 717 - RNA, 13 nt, Artificial Sequence
  Synthetic Polynucleotide
uggaagacacguu

SEQ ID NO: 718 - RNA, 19 nt, Artificial Sequence
  Synthetic Polynucleotide
aacgugucuuccagucggu

SEQ ID NO: 719 - RNA, 13 nt, Artificial Sequence
  Synthetic Polynucleotide
aagauguacggag

SEQ ID NO: 720 - RNA, 19 nt, Artificial Sequence
  Synthetic Polynucleotide
cuccguacaucuuccugua

SEQ ID NO: 721 - RNA, 13 nt, Artificial Sequence
  Synthetic Polynucleotide
aaugacaacgccu

SEQ ID NO: 722 - RNA, 19 nt, Artificial Sequence
  Synthetic Polynucleotide
aggcguugucauugguaac

SEQ ID NO: 723 - RNA, 13 nt, Artificial Sequence
  Synthetic Polynucleotide
ggcgaggucauga

SEQ ID NO: 724 - RNA, 19 nt, Artificial Sequence
  Synthetic Polynucleotide
ucaugaccucgccgucagg

SEQ ID NO: 725 - RNA, 13 nt, Artificial Sequence
  Synthetic Polynucleotide
gacacguuuggcc

SEQ ID NO: 726 - RNA, 19 nt, Artificial Sequence
  Synthetic Polynucleotide
ggccaaacgugucuuccag

SEQ ID NO: 727 - RNA, 13 nt, Artificial Sequence
  Synthetic Polynucleotide
acggagacauggc

SEQ ID NO: 728 - RNA, 19 nt, Artificial Sequence
  Synthetic Polynucleotide
gccaugucuccguacaucu

SEQ ID NO: 729 - RNA, 13 nt, Artificial Sequence
  Synthetic Polynucleotide
ucaggccuugcga

SEQ ID NO: 730 - RNA, 19 nt, Artificial Sequence
  Synthetic Polynucleotide
ucgcaaggccugaccaugc

SEQ ID NO: 731 - RNA, 13 nt, Artificial Sequence
  Synthetic Polynucleotide
gcgaagcugaccu

SEQ ID NO: 732 - RNA, 19 nt, Artificial Sequence
  Synthetic Polynucleotide
aggucagcuucgcaaggcc

SEQ ID NO: 733 - RNA, 13 nt, Artificial Sequence
  Synthetic Polynucleotide
ggaagauguacgg

SEQ ID NO: 734 - RNA, 19 nt, Artificial Sequence
  Synthetic Polynucleotide
ccguacaucuuccuguagu

SEQ ID NO: 735 - RNA, 13 nt, Artificial Sequence
  Synthetic Polynucleotide
gugacuucggcuc

SEQ ID NO: 736 - RNA, 19 nt, Artificial Sequence
  Synthetic Polynucleotide
gagccgaagucacagaaga

SEQ ID NO: 737 - RNA, 13 nt, Artificial Sequence
  Synthetic Polynucleotide
ugacuucggcucc

SEQ ID NO: 738 - RNA, 19 nt, Artificial Sequence
  Synthetic Polynucleotide
ggagccgaagucacagaag

SEQ ID NO: 739 - RNA, 13 nt, Artificial Sequence
  Synthetic Polynucleotide
uggucaggccuug

SEQ ID NO: 740 - RNA, 19 nt, Artificial Sequence
  Synthetic Polynucleotide
caaggccugaccaugcaca

SEQ ID NO: 741 - RNA, 13 nt, Artificial Sequence
  Synthetic Polynucleotide
ucaaguuugagcu

SEQ ID NO: 742 - RNA, 19 nt, Artificial Sequence
  Synthetic Polynucleotide
agcucaaacuugauaggcu

SEQ ID NO: 743 - RNA, 13 nt, Artificial Sequence
  Synthetic Polynucleotide
gccagaacugcag

SEQ ID NO: 744 - RNA, 19 nt, Artificial Sequence
  Synthetic Polynucleotide
cugcaguucuggccgacgg

SEQ ID NO: 745 - RNA, 13 nt, Artificial Sequence
  Synthetic Polynucleotide
uggaguauguacc

SEQ ID NO: 746 - RNA, 19 nt, Artificial Sequence
  Synthetic Polynucleotide
gguacauacuccacagaau

SEQ ID NO: 747 - RNA, 13 nt, Artificial Sequence
  Synthetic Polynucleotide
gcuagagaagcag

SEQ ID NO: 748 - RNA, 19 nt, Artificial Sequence
  Synthetic Polynucleotide
cugcuucucuagccugcag

SEQ ID NO: 749 - RNA, 13 nt, Artificial Sequence
  Synthetic Polynucleotide
ggucaggccuugc

SEQ ID NO: 750 - RNA, 19 nt, Artificial Sequence
  Synthetic Polynucleotide
gcaaggccugaccaugcac

SEQ ID NO: 751 - RNA, 13 nt, Artificial Sequence
  Synthetic Polynucleotide
gagcuaaauucug

SEQ ID NO: 752 - RNA, 19 nt, Artificial Sequence
  Synthetic Polynucleotide
cagaauuuagcucgguaug

SEQ ID NO: 753 - RNA, 13 nt, Artificial Sequence
  Synthetic Polynucleotide
aagacacguuugg

SEQ ID NO: 754 - RNA, 19 nt, Artificial Sequence
  Synthetic Polynucleotide
ccaaacgugucuuccaguc

SEQ ID NO: 755 - RNA, 13 nt, Artificial Sequence
  Synthetic Polynucleotide
cgaggucaugaag

SEQ ID NO: 756 - RNA, 19 nt, Artificial Sequence
  Synthetic Polynucleotide
cuucaugaccucgccguca

SEQ ID NO: 757 - RNA, 13 nt, Artificial Sequence
  Synthetic Polynucleotide
ggccuugcgaagc

SEQ ID NO: 758 - RNA, 19 nt, Artificial Sequence
  Synthetic Polynucleotide
gcuucgcaaggccugacca

SEQ ID NO: 759 - RNA, 13 nt, Artificial Sequence
  Synthetic Polynucleotide
cuugcgaagcuga

SEQ ID NO: 760 - RNA, 19 nt, Artificial Sequence
  Synthetic Polynucleotide
ucagcuucgcaaggccuga

SEQ ID NO: 761 - RNA, 13 nt, Artificial Sequence
  Synthetic Polynucleotide
ccgacuggaagac

SEQ ID NO: 762 - RNA, 19 nt, Artificial Sequence
  Synthetic Polynucleotide
gucuuccagucgguaagcc

SEQ ID NO: 763 - RNA, 13 nt, Artificial Sequence
  Synthetic Polynucleotide
ccuaucaaguuug

SEQ ID NO: 764 - RNA, 19 nt, Artificial Sequence
  Synthetic Polynucleotide
caaacuugauaggcuugga

SEQ ID NO: 765 - RNA, 13 nt, Artificial Sequence
  Synthetic Polynucleotide
uguuccaagaccu

SEQ ID NO: 766 - RNA, 19 nt, Artificial Sequence
  Synthetic Polynucleotide
aggucuuggaacaggcgcu

SEQ ID NO: 767 - RNA, 13 nt, Artificial Sequence
  Synthetic Polynucleotide
cgaagcugaccug

SEQ ID NO: 768 - RNA, 19 nt, Artificial Sequence
  Synthetic Polynucleotide
caggucagcuucgcaaggc

SEQ ID NO: 769 - RNA, 13 nt, Artificial Sequence
  Synthetic Polynucleotide
uugcgaagcugac

SEQ ID NO: 770 - RNA, 19 nt, Artificial Sequence
  Synthetic Polynucleotide
gucagcuucgcaaggccug

SEQ ID NO: 771 - RNA, 13 nt, Artificial Sequence
  Synthetic Polynucleotide
caaugacaacgcc

SEQ ID NO: 772 - RNA, 19 nt, Artificial Sequence
  Synthetic Polynucleotide
ggcguugucauugguaacc

SEQ ID NO: 773 - RNA, 13 nt, Artificial Sequence
  Synthetic Polynucleotide
guaccagugcacg

SEQ ID NO: 774 - RNA, 19 nt, Artificial Sequence
  Synthetic Polynucleotide
cgugcacugguacuugcag

SEQ ID NO: 775 - RNA, 13 nt, Artificial Sequence
  Synthetic Polynucleotide
ccuguuccaagac

SEQ ID NO: 776 - RNA, 19 nt, Artificial Sequence
  Synthetic Polynucleotide
gucuuggaacaggcgcucc

SEQ ID NO: 777 - RNA, 13 nt, Artificial Sequence
  Synthetic Polynucleotide
uacggagacaugg

SEQ ID NO: 778 - RNA, 19 nt, Artificial Sequence
  Synthetic Polynucleotide
ccaugucuccguacaucuu

SEQ ID NO: 779 - RNA, 13 nt, Artificial Sequence
  Synthetic Polynucleotide
ugcgaagcugacc

SEQ ID NO: 780 - RNA, 19 nt, Artificial Sequence
  Synthetic Polynucleotide
ggucagcuucgcaaggccu

SEQ ID NO: 781 - RNA, 13 nt, Artificial Sequence
  Synthetic Polynucleotide
ccuugcgaagcug

SEQ ID NO: 782 - RNA, 19 nt, Artificial Sequence
  Synthetic Polynucleotide
cagcuucgcaaggccugac

SEQ ID NO: 783 - RNA, 13 nt, Artificial Sequence
  Synthetic Polynucleotide
cugugacuucggc

SEQ ID NO: 784 - RNA, 19 nt, Artificial Sequence
  Synthetic Polynucleotide
gccgaagucacagaagagg

SEQ ID NO: 785 - RNA, 13 nt, Artificial Sequence
  Synthetic Polynucleotide
gcuaaauucugug

SEQ ID NO: 786 - RNA, 19 nt, Artificial Sequence
  Synthetic Polynucleotide
cacagaauuuagcucggua

SEQ ID NO: 787 - RNA, 13 nt, Artificial Sequence
  Synthetic Polynucleotide
cuaaauucugugg

SEQ ID NO: 788 - RNA, 19 nt, Artificial Sequence
  Synthetic Polynucleotide
ccacagaauuuagcucggu

SEQ ID NO: 789 - RNA, 13 nt, Artificial Sequence
  Synthetic Polynucleotide
agacacguuuggc

SEQ ID NO: 790 - RNA, 19 nt, Artificial Sequence
  Synthetic Polynucleotide
gccaaacgugucuuccagu

SEQ ID NO: 791 - RNA, 13 nt, Artificial Sequence
  Synthetic Polynucleotide
ccgcaagaucggc

SEQ ID NO: 792 - RNA, 19 nt, Artificial Sequence
  Synthetic Polynucleotide
gccgaucuugcgguuggcc

SEQ ID NO: 793 - RNA, 13 nt, Artificial Sequence
  Synthetic Polynucleotide
uaucaaguuugag

SEQ ID NO: 794 - RNA, 19 nt, Artificial Sequence
  Synthetic Polynucleotide
cucaaacuugauaggcuug

SEQ ID NO: 795 - RNA, 13 nt, Artificial Sequence
  Synthetic Polynucleotide
gaagcugaccugg

SEQ ID NO: 796 - RNA, 19 nt, Artificial Sequence
  Synthetic Polynucleotide
ccaggucagcuucgcaagg

SEQ ID NO: 797 - RNA, 13 nt, Artificial Sequence
  Synthetic Polynucleotide
acauuaacucaua

SEQ ID NO: 798 - RNA, 19 nt, Artificial Sequence
  Synthetic Polynucleotide
uaugaguuaaugucucuca

SEQ ID NO: 799 - RNA, 14 nt, Artificial Sequence
  Synthetic Polynucleotide
gacauuaacucaua

SEQ ID NO: 800 - RNA, 19 nt, Artificial Sequence
  Synthetic Polynucleotide
uaugaguuaaugucucuca

SEQ ID NO: 801 - RNA, 13 nt, Artificial Sequence
  Synthetic Polynucleotide
ugaagaauguuaa

SEQ ID NO: 802 - RNA, 19 nt, Artificial Sequence
  Synthetic Polynucleotide
uuaacauucuucaaaccag

SEQ ID NO: 803 - RNA, 14 nt, Artificial Sequence
  Synthetic Polynucleotide
uugaagaauguuaa

SEQ ID NO: 804 - RNA, 19 nt, Artificial Sequence
  Synthetic Polynucleotide
uuaacauucuucaaaccag

SEQ ID NO: 805 - RNA, 13 nt, Artificial Sequence
  Synthetic Polynucleotide
gauagcaucuuaa

SEQ ID NO: 806 - RNA, 19 nt, Artificial Sequence
  Synthetic Polynucleotide
uuaagaugcuaucugauga

SEQ ID NO: 807 - RNA, 14 nt, Artificial Sequence
  Synthetic Polynucleotide
agauagcaucuuaa

SEQ ID NO: 808 - RNA, 19 nt, Artificial Sequence
  Synthetic Polynucleotide
uuaagaugcuaucugauga

SEQ ID NO: 809 - RNA, 13 nt, Artificial Sequence
  Synthetic Polynucleotide
ugaaguguaauua

SEQ ID NO: 810 - RNA, 19 nt, Artificial Sequence
  Synthetic Polynucleotide
uaauuacacuucaaauagc

SEQ ID NO: 811 - RNA, 13 nt, Artificial Sequence
  Synthetic Polynucleotide
aauugagaaggaa

SEQ ID NO: 812 - RNA, 19 nt, Artificial Sequence
  Synthetic Polynucleotide
uuccuucucaauuacacuu

SEQ ID NO: 813 - RNA, 13 nt, Artificial Sequence
  Synthetic Polynucleotide
uugagaaggaaaa

SEQ ID NO: 814 - RNA, 19 nt, Artificial Sequence
  Synthetic Polynucleotide
uuuuccuucucaauuacac

SEQ ID NO: 815 - RNA, 13 nt, Artificial Sequence
  Synthetic Polynucleotide
cauucugauucga

SEQ ID NO: 816 - RNA, 19 nt, Artificial Sequence
  Synthetic Polynucleotide
ucgaaucagaaugucagag

SEQ ID NO: 817 - RNA, 13 nt, Artificial Sequence
  Synthetic Polynucleotide
uucugauucgaaa

SEQ ID NO: 818 - RNA, 19 nt, Artificial Sequence
  Synthetic Polynucleotide
uuucgaaucagaaugucag

SEQ ID NO: 819 - RNA, 13 nt, Artificial Sequence
  Synthetic Polynucleotide
cugucgauuagaa

SEQ ID NO: 820 - RNA, 19 nt, Artificial Sequence
  Synthetic Polynucleotide
uucuaaucgacaggauucc

SEQ ID NO: 821 - RNA, 13 nt, Artificial Sequence
  Synthetic Polynucleotide
uuugccuguaaca

SEQ ID NO: 822 - RNA, 19 nt, Artificial Sequence
  Synthetic Polynucleotide
uguuacaggcaaauucacu

SEQ ID NO: 823 - RNA, 14 nt, Artificial Sequence
  Synthetic Polynucleotide
auuugccuguaaca

SEQ ID NO: 824 - RNA, 19 nt, Artificial Sequence
  Synthetic Polynucleotide
uguuacaggcaaauucacu

SEQ ID NO: 825 - RNA, 13 nt, Artificial Sequence
  Synthetic Polynucleotide
acaagccagauua

SEQ ID NO: 826 - RNA, 19 nt, Artificial Sequence
  Synthetic Polynucleotide
uaaucuggcuuguuacagg

SEQ ID NO: 827 - RNA, 14 nt, Artificial Sequence
  Synthetic Polynucleotide
aacaagccagauua

SEQ ID NO: 828 - RNA, 19 nt, Artificial Sequence
  Synthetic Polynucleotide
uaaucuggcuuguuacagg

SEQ ID NO: 829 - RNA, 13 nt, Artificial Sequence
  Synthetic Polynucleotide
caguuuauuugua

SEQ ID NO: 830 - RNA, 19 nt, Artificial Sequence
  Synthetic Polynucleotide
uacaaauaaacuguccgaa

SEQ ID NO: 831 - RNA, 13 nt, Artificial Sequence
  Synthetic Polynucleotide
uguugagagugua

SEQ ID NO: 832 - RNA, 19 nt, Artificial Sequence
  Synthetic Polynucleotide
uacacucucaacaaauaaa

SEQ ID NO: 833 - RNA, 14 nt, Artificial Sequence
  Synthetic Polynucleotide
uuguugagagugua

SEQ ID NO: 834 - RNA, 19 nt, Artificial Sequence
  Synthetic Polynucleotide
uacacucucaacaaauaaa

SEQ ID NO: 835 - RNA, 13 nt, Artificial Sequence
  Synthetic Polynucleotide
ugcaccuuucuaa

SEQ ID NO: 836 - RNA, 19 nt, Artificial Sequence
  Synthetic Polynucleotide
uuagaaaggugcaaacaug

SEQ ID NO: 837 - RNA, 14 nt, Artificial Sequence
  Synthetic Polynucleotide
uugcaccuuucuaa

SEQ ID NO: 838 - RNA, 19 nt, Artificial Sequence
  Synthetic Polynucleotide
uuagaaaggugcaaacaug

SEQ ID NO: 839 - RNA, 13 nt, Artificial Sequence
  Synthetic Polynucleotide
uugagcuuucuga

SEQ ID NO: 840 - RNA, 19 nt, Artificial Sequence
  Synthetic Polynucleotide
ucagaaagcucaaacuuga

SEQ ID NO: 841 - RNA, 13 nt, Artificial Sequence
  Synthetic Polynucleotide
ugagagugugaca

SEQ ID NO: 842 - RNA, 19 nt, Artificial Sequence
  Synthetic Polynucleotide
ugucacacucucaacaaau

SEQ ID NO: 843 - RNA, 13 nt, Artificial Sequence
  Synthetic Polynucleotide
agugugaccaaaa

SEQ ID NO: 844 - RNA, 19 nt, Artificial Sequence
  Synthetic Polynucleotide
uuuuggucacacucucaac

SEQ ID NO: 845 - RNA, 14 nt, Artificial Sequence
  Synthetic Polynucleotide
gagugugaccaaaa

SEQ ID NO: 846 - RNA, 19 nt, Artificial Sequence
  Synthetic Polynucleotide
uuuuggucacacucucaac

SEQ ID NO: 847 - RNA, 13 nt, Artificial Sequence
  Synthetic Polynucleotide
gugugaccaaaaa

SEQ ID NO: 848 - RNA, 19 nt, Artificial Sequence
  Synthetic Polynucleotide
uuuuuggucacacucucaa

SEQ ID NO: 849 - RNA, 13 nt, Artificial Sequence
  Synthetic Polynucleotide
ugugaccaaaaga

SEQ ID NO: 850 - RNA, 19 nt, Artificial Sequence
  Synthetic Polynucleotide
ucuuuuggucacacucuca

SEQ ID NO: 851 - RNA, 14 nt, Artificial Sequence
  Synthetic Polynucleotide
gugugaccaaaaga

SEQ ID NO: 852 - RNA, 19 nt, Artificial Sequence
  Synthetic Polynucleotide
ucuuuuggucacacucuca

SEQ ID NO: 853 - RNA, 13 nt, Artificial Sequence
  Synthetic Polynucleotide
gugaccaaaagua

SEQ ID NO: 854 - RNA, 19 nt, Artificial Sequence
  Synthetic Polynucleotide
uacuuuuggucacacucuc

SEQ ID NO: 855 - RNA, 13 nt, Artificial Sequence
  Synthetic Polynucleotide
gaccaaaaguuaa

SEQ ID NO: 856 - RNA, 19 nt, Artificial Sequence
  Synthetic Polynucleotide
uuaacuuuuggucacacuc

SEQ ID NO: 857 - RNA, 13 nt, Artificial Sequence
  Synthetic Polynucleotide
gcaccuuucuaga

SEQ ID NO: 858 - RNA, 19 nt, Artificial Sequence
  Synthetic Polynucleotide
ucuagaaaggugcaaacau

SEQ ID NO: 859 - RNA, 13 nt, Artificial Sequence
  Synthetic Polynucleotide
ccuuucuaguuga

SEQ ID NO: 860 - RNA, 19 nt, Artificial Sequence
  Synthetic Polynucleotide
ucaacuagaaaggugcaaa

SEQ ID NO: 861 - RNA, 13 nt, Artificial Sequence
  Synthetic Polynucleotide
gcaccuuucuaga

SEQ ID NO: 862 - RNA, 19 nt, Artificial Sequence
  Synthetic Polynucleotide
ucuagaaaggugcaaacau

SEQ ID NO: 863 - RNA, 13 nt, Artificial Sequence
  Synthetic Polynucleotide
gcaccuuucuaga

SEQ ID NO: 864 - RNA, 19 nt, Artificial Sequence
  Synthetic Polynucleotide
ucuagaaaggugcaaacau

SEQ ID NO: 865 - RNA, 13 nt, Artificial Sequence
  Synthetic Polynucleotide
gcaccuuucuaga

SEQ ID NO: 866 - RNA, 19 nt, Artificial Sequence
  Synthetic Polynucleotide
ucuagaaaggugcaaacau

SEQ ID NO: 867 - RNA, 13 nt, Artificial Sequence
  Synthetic Polynucleotide
gcaccuuucuaga

SEQ ID NO: 868 - RNA, 19 nt, Artificial Sequence
  Synthetic Polynucleotide
ucuagaaaggugcaaacau

SEQ ID NO: 869 - RNA, 13 nt, Artificial Sequence
  Synthetic Polynucleotide
gcaccuuucuaga

SEQ ID NO: 870 - RNA, 19 nt, Artificial Sequence
  Synthetic Polynucleotide
ucuagaaaggugcaaacau

SEQ ID NO: 871 - RNA, 13 nt, Artificial Sequence
  Synthetic Polynucleotide
gcaccuuucuaga

SEQ ID NO: 872 - RNA, 19 nt, Artificial Sequence
  Synthetic Polynucleotide
ucuagaaaggugcaaacau

SEQ ID NO: 873 - RNA, 13 nt, Artificial Sequence
  Synthetic Polynucleotide
gcaccuuucuaga

SEQ ID NO: 874 - RNA, 19 nt, Artificial Sequence
  Synthetic Polynucleotide
ucuagaaaggugcaaacau

SEQ ID NO: 875 - RNA, 13 nt, Artificial Sequence
  Synthetic Polynucleotide
gcaccuuucuaga

SEQ ID NO: 876 - RNA, 19 nt, Artificial Sequence
  Synthetic Polynucleotide
ucuagaaaggugcaaacau

SEQ ID NO: 877 - RNA, 13 nt, Artificial Sequence
  Synthetic Polynucleotide
gcaccuuucuaga

SEQ ID NO: 878 - RNA, 19 nt, Artificial Sequence
  Synthetic Polynucleotide
ucuagaaaggugcaaacau

SEQ ID NO: 879 - RNA, 13 nt, Artificial Sequence
  Synthetic Polynucleotide
gcaccuuucuaga

SEQ ID NO: 880 - RNA, 19 nt, Artificial Sequence
  Synthetic Polynucleotide
ucuagaaaggugcaaacau

SEQ ID NO: 881 - RNA, 13 nt, Artificial Sequence
  Synthetic Polynucleotide
gcaccuuucuaga

SEQ ID NO: 882 - RNA, 19 nt, Artificial Sequence
  Synthetic Polynucleotide
ucuagaaaggugcaaacau

SEQ ID NO: 883 - RNA, 13 nt, Artificial Sequence
  Synthetic Polynucleotide
gugaccaaaagua

SEQ ID NO: 884 - RNA, 19 nt, Artificial Sequence
  Synthetic Polynucleotide
uacuuuuggucacacucuc

SEQ ID NO: 885 - RNA, 13 nt, Artificial Sequence
  Synthetic Polynucleotide
gugaccaaaagua

SEQ ID NO: 886 - RNA, 19 nt, Artificial Sequence
  Synthetic Polynucleotide
uacuuuuggucacacucuc

SEQ ID NO: 887 - RNA, 13 nt, Artificial Sequence
  Synthetic Polynucleotide
gugaccaaaagua

SEQ ID NO: 888 - RNA, 19 nt, Artificial Sequence
  Synthetic Polynucleotide
uacuuuuggucacacucuc

SEQ ID NO: 889 - RNA, 14 nt, Artificial Sequence
  Synthetic Polynucleotide
uugcaccuuucuaa

SEQ ID NO: 890 - RNA, 20 nt, Artificial Sequence
  Synthetic Polynucleotide
uuagaaaggugcaaacaagg

SEQ ID NO: 891 - RNA, 14 nt, Artificial Sequence
  Synthetic Polynucleotide
uugcaccuuucuaa

SEQ ID NO: 892 - RNA, 20 nt, Artificial Sequence
  Synthetic Polynucleotide
uuagaaaggugcaaacaagg

SEQ ID NO: 893 - RNA, 14 nt, Artificial Sequence
  Synthetic Polynucleotide
uugcaccuuucuaa

SEQ ID NO: 894 - RNA, 20 nt, Artificial Sequence
  Synthetic Polynucleotide
uuagaaaggugcaaacaagg

SEQ ID NO: 895 - RNA, 14 nt, Artificial Sequence
  Synthetic Polynucleotide
uugcaccuuucuaa

SEQ ID NO: 896 - RNA, 20 nt, Artificial Sequence
  Synthetic Polynucleotide
uuagaaaggugcaaacaagg

SEQ ID NO: 897 - RNA, 14 nt, Artificial Sequence
  Synthetic Polynucleotide
uugcaccuuucuaa

SEQ ID NO: 898 - RNA, 20 nt, Artificial Sequence
  Synthetic Polynucleotide
uuagaaaggugcaaacaagg

SEQ ID NO: 899 - RNA, 14 nt, Artificial Sequence
  Synthetic Polynucleotide
uugcaccuuucuaa

SEQ ID NO: 900 - RNA, 20 nt, Artificial Sequence
  Synthetic Polynucleotide
uuagaaaggugcaaacaagg

SEQ ID NO: 901 - RNA, 14 nt, Artificial Sequence
  Synthetic Polynucleotide
uugcaccuuucuaa

SEQ ID NO: 902 - RNA, 20 nt, Artificial Sequence
  Synthetic Polynucleotide
uuagaaaggugcaaacaagg

SEQ ID NO: 903 - RNA, 14 nt, Artificial Sequence
  Synthetic Polynucleotide
uugcaccuuucuaa

SEQ ID NO: 904 - RNA, 20 nt, Artificial Sequence
  Synthetic Polynucleotide
uuagaaaggugcaaacaagg

SEQ ID NO: 905 - RNA, 14 nt, Artificial Sequence
  Synthetic Polynucleotide
uugcaccuuucuaa

SEQ ID NO: 906 - RNA, 20 nt, Artificial Sequence
  Synthetic Polynucleotide
uuagaaaggugcaaacaagg

SEQ ID NO: 907 - RNA, 14 nt, Artificial Sequence
  Synthetic Polynucleotide
uugcaccuuucuaa

SEQ ID NO: 908 - RNA, 20 nt, Artificial Sequence
  Synthetic Polynucleotide
uuagaaaggugcaaacaagg

SEQ ID NO: 909 - RNA, 14 nt, Artificial Sequence
  Synthetic Polynucleotide
uugcaccuuucuaa

SEQ ID NO: 910 - RNA, 20 nt, Artificial Sequence
  Synthetic Polynucleotide
uuagaaaggugcaaacaagg

SEQ ID NO: 911 - RNA, 14 nt, Artificial Sequence
  Synthetic Polynucleotide
uugcaccuuucuaa

SEQ ID NO: 912 - RNA, 20 nt, Artificial Sequence
  Synthetic Polynucleotide
uuagaaaggugcaaacaagg

SEQ ID NO: 913 - RNA, 14 nt, Artificial Sequence
  Synthetic Polynucleotide
uugcaccuuucuaa

SEQ ID NO: 914 - RNA, 20 nt, Artificial Sequence
  Synthetic Polynucleotide
uuagaaaggugcaaacaagg

SEQ ID NO: 915 - RNA, 14 nt, Artificial Sequence
  Synthetic Polynucleotide
uugcaccuuucuaa

SEQ ID NO: 916 - RNA, 20 nt, Artificial Sequence
  Synthetic Polynucleotide
uuagaaaggugcaaacaagg

SEQ ID NO: 917 - RNA, 14 nt, Artificial Sequence
  Synthetic Polynucleotide
uugcaccuuucuaa

SEQ ID NO: 918 - RNA, 20 nt, Artificial Sequence
  Synthetic Polynucleotide
uuagaaaggugcaaacaagg

SEQ ID NO: 919 - RNA, 13 nt, Artificial Sequence
  Synthetic Polynucleotide
ccuuucuaguuga

SEQ ID NO: 920 - RNA, 19 nt, Artificial Sequence
  Synthetic Polynucleotide
ucaacuagaaaggugcaaa

SEQ ID NO: 921 - RNA, 13 nt, Artificial Sequence
  Synthetic Polynucleotide
ccuuucuaguuga

SEQ ID NO: 922 - RNA, 19 nt, Artificial Sequence
  Synthetic Polynucleotide
ucaacuagaaaggugcaaa

SEQ ID NO: 923 - RNA, 13 nt, Artificial Sequence
  Synthetic Polynucleotide
ccuuucuaguuga

SEQ ID NO: 924 - RNA, 19 nt, Artificial Sequence
  Synthetic Polynucleotide
ucaacuagaaaggugcaaa

SEQ ID NO: 925 - RNA, 13 nt, Artificial Sequence
  Synthetic Polynucleotide
ccuuucuaguuga

SEQ ID NO: 926 - RNA, 19 nt, Artificial Sequence
  Synthetic Polynucleotide
ucaacuagaaaggugcaaa

SEQ ID NO: 927 - RNA, 13 nt, Artificial Sequence
  Synthetic Polynucleotide
ccuuucuaguuga

SEQ ID NO: 928 - RNA, 19 nt, Artificial Sequence
  Synthetic Polynucleotide
ucaacuagaaaggugcaaa

SEQ ID NO: 929 - RNA, 13 nt, Artificial Sequence
  Synthetic Polynucleotide
ccuuucuaguuga

SEQ ID NO: 930 - RNA, 19 nt, Artificial Sequence
  Synthetic Polynucleotide
ucaacuagaaaggugcaaa

SEQ ID NO: 931 - RNA, 13 nt, Artificial Sequence
  Synthetic Polynucleotide
ccuuucuaguuga

SEQ ID NO: 932 - RNA, 19 nt, Artificial Sequence
  Synthetic Polynucleotide
ucaacuagaaaggugcaaa

SEQ ID NO: 933 - RNA, 13 nt, Artificial Sequence
  Synthetic Polynucleotide
ccuuucuaguuga

SEQ ID NO: 934 - RNA, 19 nt, Artificial Sequence
  Synthetic Polynucleotide
ucaacuagaaaggugcaaa

SEQ ID NO: 935 - RNA, 13 nt, Artificial Sequence
  Synthetic Polynucleotide
ccuuucuaguuga

SEQ ID NO: 936 - RNA, 19 nt, Artificial Sequence
  Synthetic Polynucleotide
ucaacuagaaaggugcaaa

SEQ ID NO: 937 - RNA, 13 nt, Artificial Sequence
  Synthetic Polynucleotide
ccuuucuaguuga

SEQ ID NO: 938 - RNA, 19 nt, Artificial Sequence
  Synthetic Polynucleotide
ucaacuagaaaggugcaaa

SEQ ID NO: 939 - RNA, 13 nt, Artificial Sequence
  Synthetic Polynucleotide
ccuuucuaguuga

SEQ ID NO: 940 - RNA, 19 nt, Artificial Sequence
  Synthetic Polynucleotide
ucaacuagaaaggugcaaa

SEQ ID NO: 941 - RNA, 13 nt, Artificial Sequence
  Synthetic Polynucleotide
ccuuucuaguuga

SEQ ID NO: 942 - RNA, 19 nt, Artificial Sequence
  Synthetic Polynucleotide
ucaacuagaaaggugcaaa

SEQ ID NO: 943 - RNA, 13 nt, Artificial Sequence
  Synthetic Polynucleotide
ccuuucuaguuga

SEQ ID NO: 944 - RNA, 19 nt, Artificial Sequence
  Synthetic Polynucleotide
ucaacuagaaaggugcaaa

SEQ ID NO: 945 - RNA, 13 nt, Artificial Sequence
  Synthetic Polynucleotide
gcaccuuucuaga

SEQ ID NO: 946 - RNA, 19 nt, Artificial Sequence
  Synthetic Polynucleotide
ucuagaaaggugcaaacau

SEQ ID NO: 947 - RNA, 13 nt, Artificial Sequence
  Synthetic Polynucleotide
  misc_feature (2)..(2): Modified with 2′Ome
  misc_feature (4)..(10): Modified with 2′Ome
  misc_feature (12)..(13): Modified with 2′Ome
  misc_feature (13)..(13): Modified with cholesterol with TEG linker
gcaccuuucuaga

SEQ ID NO: 948 - RNA, 19 nt, Artificial Sequence
  Synthetic Polynucleotide
  misc_feature (1)..(1): Modified with P
  misc_feature (1)..(1): Modified with 2′Ome
  misc_feature (2)..(3): Modified with 2′fluoro
  misc_feature (6)..(6): Modified with 2′Ome
  misc_feature (8)..(8): Modified with 2′Ome
  misc_feature (11)..(11): Modified with 2′fluoro
  misc_feature (13)..(13): Modified with 2′Ome
  misc_feature (17)..(17): Modified with 2′Ome
  misc_feature (13)..(19): Phosphorothioate internucleotide bond
ucuagaaaggugcaaacau

SEQ ID NO: 949 - RNA, 13 nt, Artificial Sequence
  Synthetic Polynucleotide
gcaccuuucuaga

SEQ ID NO: 950 - RNA, 19 nt, Artificial Sequence
  Synthetic Polynucleotide
ucuagaaaggugcaaacau

SEQ ID NO: 951 - RNA, 13 nt, Artificial Sequence
  Synthetic Polynucleotide
gcaccuuucuaga

SEQ ID NO: 952 - RNA, 19 nt, Artificial Sequence
  Synthetic Polynucleotide
ucuagaaaggugcaaacau

SEQ ID NO: 953 - RNA, 13 nt, Artificial Sequence
  Synthetic Polynucleotide
gcaccuuucuaga

SEQ ID NO: 954 - RNA, 19 nt, Artificial Sequence
  Synthetic Polynucleotide
ucuagaaaggugcaaacau

SEQ ID NO: 955 - RNA, 13 nt, Artificial Sequence
  Synthetic Polynucleotide
gcaccuuucuaga

SEQ ID NO: 956 - RNA, 19 nt, Artificial Sequence
  Synthetic Polynucleotide
ucuagaaaggugcaaacau

SEQ ID NO: 957 - RNA, 13 nt, Artificial Sequence
  Synthetic Polynucleotide
gcaccuuucuaga

SEQ ID NO: 958 - RNA, 19 nt, Artificial Sequence
  Synthetic Polynucleotide
ucuagaaaggugcaaacau

SEQ ID NO: 959 - RNA, 13 nt, Artificial Sequence
  Synthetic Polynucleotide
gcaccuuucuaga

SEQ ID NO: 960 - RNA, 19 nt, Artificial Sequence
  Synthetic Polynucleotide
ucuagaaaggugcaaacau

SEQ ID NO: 961 - RNA, 13 nt, Artificial Sequence
  Synthetic Polynucleotide
gcaccuuucuaga

SEQ ID NO: 962 - RNA, 19 nt, Artificial Sequence
  Synthetic Polynucleotide
ucuagaaaggugcaaacau

SEQ ID NO: 963 - RNA, 14 nt, Artificial Sequence
  Synthetic Polynucleotide
  misc_feature (1)..(2): Modified with 2′Ome
  misc_feature (4)..(4): Modified with 2′Ome
  misc_feature (6)..(14): Modified with 2′Ome
  misc_feature (12)..(14): Phosphorothioate internucleotide bond
  misc_feature (14)..(14): Modified with cholesterol with TEG linker
uugcaccuuucuaa

SEQ ID NO: 964 - RNA, 20 nt, Artificial Sequence
  Synthetic Polynucleotide
  misc_feature (1)..(1): Modified with P
  misc_feature (1)..(1): Modified with 2′Ome
  misc_feature (2)..(2): Modified with 2′fluoro
  misc_feature (6)..(6): Modified with 2′Ome
  misc_feature (10)..(10): Modified with 2′fluoro
  misc_feature (12)..(12): Modified with 2′fluoro
  misc_feature (13)..(15): Modified with 2′Ome
  misc_feature (16)..(16): Modified with 2′fluoro
  misc_feature (17)..(19): Modified with 2′Ome
  misc_feature (14)..(20): Phosphorothioate internucleotide bond
uuagaaaggugcaaacaagg

SEQ ID NO: 965 - RNA, 14 nt, Artificial Sequence
  Synthetic Polynucleotide
uugcaccuuucuaa

SEQ ID NO: 966 - RNA, 20 nt, Artificial Sequence
  Synthetic Polynucleotide
uuagaaaggugcaaacaagg

SEQ ID NO: 967 - RNA, 14 nt, Artificial Sequence
  Synthetic Polynucleotide
uugcaccuuucuaa

SEQ ID NO: 968 - RNA, 20 nt, Artificial Sequence
  Synthetic Polynucleotide
uuagaaaggugcaaacaagg

SEQ ID NO: 969 - RNA, 14 nt, Artificial Sequence
  Synthetic Polynucleotide
uugcaccuuucuaa

SEQ ID NO: 970 - RNA, 20 nt, Artificial Sequence
  Synthetic Polynucleotide
uuagaaaggugcaaacaagg

SEQ ID NO: 971 - RNA, 14 nt, Artificial Sequence
  Synthetic Polynucleotide
uugcaccuuucuaa

SEQ ID NO: 972 - RNA, 20 nt, Artificial Sequence
  Synthetic Polynucleotide
uuagaaaggugcaaacaagg

SEQ ID NO: 973 - RNA, 14 nt, Artificial Sequence
  Synthetic Polynucleotide
uugcaccuuucuaa

SEQ ID NO: 974 - RNA, 20 nt, Artificial Sequence
  Synthetic Polynucleotide
uuagaaaggugcaaacaagg

SEQ ID NO: 975 - RNA, 14 nt, Artificial Sequence
  Synthetic Polynucleotide
uugcaccuuucuaa

SEQ ID NO: 976 - RNA, 20 nt, Artificial Sequence
  Synthetic Polynucleotide
uuagaaaggugcaaacaagg

SEQ ID NO: 977 - RNA, 14 nt, Artificial Sequence
  Synthetic Polynucleotide
uugcaccuuucuaa

SEQ ID NO: 978 - RNA, 20 nt, Artificial Sequence
  Synthetic Polynucleotide
uuagaaaggugcaaacaagg

SEQ ID NO: 979 - RNA, 14 nt, Artificial Sequence
  Synthetic Polynucleotide
uugcaccuuucuaa

SEQ ID NO: 980 - RNA, 20 nt, Artificial Sequence
  Synthetic Polynucleotide
uuagaaaggugcaaacaagg

SEQ ID NO: 981 - RNA, 14 nt, Artificial Sequence
  Synthetic Polynucleotide
uugcaccuuucuaa

SEQ ID NO: 982 - RNA, 20 nt, Artificial Sequence
  Synthetic Polynucleotide
uuagaaaggugcaaacaagg

SEQ ID NO: 983 - RNA, 14 nt, Artificial Sequence
  Synthetic Polynucleotide
uugcaccuuucuaa

SEQ ID NO: 984 - RNA, 20 nt, Artificial Sequence
  Synthetic Polynucleotide
uuagaaaggugcaaacaagg

SEQ ID NO: 985 - RNA, 14 nt, Artificial Sequence
  Synthetic Polynucleotide
uugcaccuuucuaa

SEQ ID NO: 986 - RNA, 20 nt, Artificial Sequence
  Synthetic Polynucleotide
uuagaaaggugcaaacaagg

SEQ ID NO: 987 - RNA, 13 nt, Artificial Sequence
  Synthetic Polynucleotide
ccuuucuaguuga

SEQ ID NO: 988 - RNA, 19 nt, Artificial Sequence
  Synthetic Polynucleotide
ucaacuagaaaggugcaaa

SEQ ID NO: 989 - RNA, 13 nt, Artificial Sequence
  Synthetic Polynucleotide
ccuuucuaguuga

SEQ ID NO: 990 - RNA, 19 nt, Artificial Sequence
  Synthetic Polynucleotide
ucaacuagaaaggugcaaa

SEQ ID NO: 991 - RNA, 13 nt, Artificial Sequence
  Synthetic Polynucleotide
ccuuucuaguuga

SEQ ID NO: 992 - RNA, 19 nt, Artificial Sequence
  Synthetic Polynucleotide
ucaacuagaaaggugcaaa

SEQ ID NO: 993 - RNA, 13 nt, Artificial Sequence
  Synthetic Polynucleotide
  misc_feature (2)..(2): Modified with 2′Ome
  misc_feature (4)..(10): Modified with 2′Ome
  misc_feature (12)..(13): Modified with 2′Ome
  misc_feature (11)..(13): Phosphorothioate internucleotide bond
  misc_feature (13)..(13): Modified with cholesterol with TEG linker
gcaccuuucuaga

SEQ ID NO: 994 - RNA, 19 nt, Artificial Sequence
  Synthetic Polynucleotide
  misc_feature (1)..(1): Modified with P
  misc_feature (1)..(1): Modified with 2′Ome
  misc_feature (2)..(3): Modified with 2′fluoro
  misc_feature (6)..(6): Modified with 2′Ome
  misc_feature (8)..(8): Modified with 2′Ome
  misc_feature (11)..(11): Modified with 2′fluoro
  misc_feature (13)..(13): Modified with 2′fluoro
  misc_feature (14)..(16): Modified with 2′Ome
  misc_feature (17)..(17): Modified with 2′fluoro
  misc_feature (18)..(18): Modified with 2′Ome
  misc_feature (13)..(19): Phosphorothioate internucleotide bond
ucuagaaaggugcaaacau

SEQ ID NO: 995 - RNA, 13 nt, Artificial Sequence
  Synthetic Polynucleotide
gcaccuuucuaga

SEQ ID NO: 996 - RNA, 19 nt, Artificial Sequence
  Synthetic Polynucleotide
ucuagaaaggugcaaacau

SEQ ID NO: 997 - RNA, 13 nt, Artificial Sequence
  Synthetic Polynucleotide
gcaccuuucuaga

SEQ ID NO: 998 - RNA, 19 nt, Artificial Sequence
  Synthetic Polynucleotide
ucuagaaaggugcaaacau

SEQ ID NO: 999 - RNA, 13 nt, Artificial Sequence
  Synthetic Polynucleotide
gcaccuuucuaga

SEQ ID NO: 1000 - RNA, 19 nt, Artificial Sequence
  Synthetic Polynucleotide
ucuagaaaggugcaaacau

SEQ ID NO: 1001 - RNA, 13 nt, Artificial Sequence
  Synthetic Polynucleotide
gcaccuuucuaga

SEQ ID NO: 1002 - RNA, 19 nt, Artificial Sequence
  Synthetic Polynucleotide
ucuagaaaggugcaaacau

SEQ ID NO: 1003 - RNA, 13 nt, Artificial Sequence
  Synthetic Polynucleotide
gugaccaaaagua

SEQ ID NO: 1004 - RNA, 19 nt, Artificial Sequence
  Synthetic Polynucleotide
uacuuuuggucacacucuc

SEQ ID NO: 1005 - RNA, 13 nt, Artificial Sequence
  Synthetic Polynucleotide
gugaccaaaagua

SEQ ID NO: 1006 - RNA, 19 nt, Artificial Sequence
  Synthetic Polynucleotide
uacuuuuggucacacucuc

SEQ ID NO: 1007 - RNA, 13 nt, Artificial Sequence
  Synthetic Polynucleotide
gugaccaaaagua

SEQ ID NO: 1008 - RNA, 19 nt, Artificial Sequence
  Synthetic Polynucleotide
uacuuuuggucacacucuc

SEQ ID NO: 1009 - RNA, 13 nt, Artificial Sequence
  Synthetic Polynucleotide
gugaccaaaagua

SEQ ID NO: 1010 - RNA, 19 nt, Artificial Sequence
  Synthetic Polynucleotide
uacuuuuggucacacucuc

SEQ ID NO: 1011 - RNA, 13 nt, Artificial Sequence
  Synthetic Polynucleotide
gugaccaaaagua

SEQ ID NO: 1012 - RNA, 19 nt, Artificial Sequence
  Synthetic Polynucleotide
uacuuuuggucacacucuc

SEQ ID NO: 1013 - RNA, 13 nt, Artificial Sequence
  Synthetic Polynucleotide
gugaccaaaagua

SEQ ID NO: 1014 - RNA, 19 nt, Artificial Sequence
  Synthetic Polynucleotide
uacuuuuggucacacucuc

SEQ ID NO: 1015 - RNA, 13 nt, Artificial Sequence
  Synthetic Polynucleotide
gugaccaaaagua

SEQ ID NO: 1016 - RNA, 19 nt, Artificial Sequence
  Synthetic Polynucleotide
uacuuuuggucacacucuc

SEQ ID NO: 1017 - RNA, 13 nt, Artificial Sequence
  Synthetic Polynucleotide
gugaccaaaagua

SEQ ID NO: 1018 - RNA, 19 nt, Artificial Sequence
  Synthetic Polynucleotide
uacuuuuggucacacucuc

SEQ ID NO: 1019 - RNA, 13 nt, Artificial Sequence
  Synthetic Polynucleotide
gugaccaaaagua

SEQ ID NO: 1020 - RNA, 19 nt, Artificial Sequence
  Synthetic Polynucleotide
uacuuuuggucacacucuc

SEQ ID NO: 1021 - RNA, 13 nt, Artificial Sequence
  Synthetic Polynucleotide
gcaccuuucuaga

SEQ ID NO: 1022 - RNA, 19 nt, Artificial Sequence
  Synthetic Polynucleotide
ucuagaaaggugcaaacau

SEQ ID NO: 1023 - RNA, 13 nt, Artificial Sequence
  Synthetic Polynucleotide
gugaccaaaagua

SEQ ID NO: 1024 - RNA, 19 nt, Artificial Sequence
  Synthetic Polynucleotide
uacuuuuggucacacucuc

SEQ ID NO: 1025 - RNA, 13 nt, Artificial Sequence
  Synthetic Polynucleotide
gugaccaaaagua

SEQ ID NO: 1026 - RNA, 19 nt, Artificial Sequence
  Synthetic Polynucleotide
uacuuuuggucacacucuc

SEQ ID NO: 1027 - RNA, 13 nt, Artificial Sequence
  Synthetic Polynucleotide
ggcucuccuucga

SEQ ID NO: 1028 - RNA, 19 nt, Artificial Sequence
  Synthetic Polynucleotide
ucgaaggagagccauucgc

SEQ ID NO: 1029 - RNA, 13 nt, Artificial Sequence
  Synthetic Polynucleotide
gacaggaaccugg

SEQ ID NO: 1030 - RNA, 19 nt, Artificial Sequence
  Synthetic Polynucleotide
ccagguuccugucuuuaug

SEQ ID NO: 1031 - RNA, 13 nt, Artificial Sequence
  Synthetic Polynucleotide
ccaaggagguuua

SEQ ID NO: 1032 - RNA, 19 nt, Artificial Sequence
  Synthetic Polynucleotide
uaaaccuccuuggcguagu

SEQ ID NO: 1033 - RNA, 13 nt, Artificial Sequence
  Synthetic Polynucleotide
auuuccaucuaca

SEQ ID NO: 1034 - RNA, 19 nt, Artificial Sequence
  Synthetic Polynucleotide
uguagauggaaaucaccuc

SEQ ID NO: 1035 - RNA, 13 nt, Artificial Sequence
  Synthetic Polynucleotide
uccaucuacaaca

SEQ ID NO: 1036 - RNA, 19 nt, Artificial Sequence
  Synthetic Polynucleotide
uguuguagauggaaaucac

SEQ ID NO: 1037 - RNA, 13 nt, Artificial Sequence
  Synthetic Polynucleotide
uuuccaucuacaa

SEQ ID NO: 1038 - RNA, 19 nt, Artificial Sequence
  Synthetic Polynucleotide
uuguagauggaaaucaccu

SEQ ID NO: 1039 - RNA, 13 nt, Artificial Sequence
  Synthetic Polynucleotide
cgccaaggagguu

SEQ ID NO: 1040 - RNA, 19 nt, Artificial Sequence
  Synthetic Polynucleotide
aaccuccuuggcguaguac

SEQ ID NO: 1041 - RNA, 13 nt, Artificial Sequence
  Synthetic Polynucleotide
guggugaucagaa

SEQ ID NO: 1042 - RNA, 19 nt, Artificial Sequence
  Synthetic Polynucleotide
uucugaucaccacugguau

SEQ ID NO: 1043 - RNA, 13 nt, Artificial Sequence
  Synthetic Polynucleotide
cuccugcuaaugu

SEQ ID NO: 1044 - RNA, 19 nt, Artificial Sequence
  Synthetic Polynucleotide
acauuagcaggagaugugg

SEQ ID NO: 1045 - RNA, 13 nt, Artificial Sequence
  Synthetic Polynucleotide
accuccacauaua

SEQ ID NO: 1046 - RNA, 19 nt, Artificial Sequence
  Synthetic Polynucleotide
uauauguggaggugccauc

SEQ ID NO: 1047 - RNA, 13 nt, Artificial Sequence
  Synthetic Polynucleotide
aaguccacuagga

SEQ ID NO: 1048 - RNA, 19 nt, Artificial Sequence
  Synthetic Polynucleotide
uccuaguggacuuuauagu

SEQ ID NO: 1049 - RNA, 13 nt, Artificial Sequence
  Synthetic Polynucleotide
uggugaucagaaa

SEQ ID NO: 1050 - RNA, 19 nt, Artificial Sequence
  Synthetic Polynucleotide
uuucugaucaccacuggua

SEQ ID NO: 1051 - RNA, 13 nt, Artificial Sequence
  Synthetic Polynucleotide
aguccacuaggaa

SEQ ID NO: 1052 - RNA, 19 nt, Artificial Sequence
  Synthetic Polynucleotide
uuccuaguggacuuuauag

SEQ ID NO: 1053 - RNA, 13 nt, Artificial Sequence
  Synthetic Polynucleotide
acgccaaggaggu

SEQ ID NO: 1054 - RNA, 19 nt, Artificial Sequence
  Synthetic Polynucleotide
accuccuuggcguaguacu

SEQ ID NO: 1055 - RNA, 13 nt, Artificial Sequence
  Synthetic Polynucleotide
uauuuauugugua

SEQ ID NO: 1056 - RNA, 19 nt, Artificial Sequence
  Synthetic Polynucleotide
uacacaauaaauaacucac

SEQ ID NO: 1057 - RNA, 14 nt, Artificial Sequence
  Synthetic Polynucleotide
uuauuuauugugua

SEQ ID NO: 1058 - RNA, 19 nt, Artificial Sequence
  Synthetic Polynucleotide
uacacaauaaauaacucac

SEQ ID NO: 1059 - RNA, 13 nt, Artificial Sequence
  Synthetic Polynucleotide
aucaguguuaaaa

SEQ ID NO: 1060 - RNA, 19 nt, Artificial Sequence
  Synthetic Polynucleotide
uuuuaacacugaugaacca

SEQ ID NO: 1061 - RNA, 14 nt, Artificial Sequence
  Synthetic Polynucleotide
caucaguguuaaaa

SEQ ID NO: 1062 - RNA, 19 nt, Artificial Sequence
  Synthetic Polynucleotide
uuuuaacacugaugaacca

SEQ ID NO: 1063 - RNA, 13 nt, Artificial Sequence
  Synthetic Polynucleotide
auggcuuaaggaa

SEQ ID NO: 1064 - RNA, 19 nt, Artificial Sequence
  Synthetic Polynucleotide
uuccuuaagccauccauga

SEQ ID NO: 1065 - RNA, 14 nt, Artificial Sequence
  Synthetic Polynucleotide
gauggcuuaaggaa

SEQ ID NO: 1066 - RNA, 19 nt, Artificial Sequence
  Synthetic Polynucleotide
uuccuuaagccauccauga

SEQ ID NO: 1067 - RNA, 13 nt, Artificial Sequence
  Synthetic Polynucleotide
uuguguucuguua

SEQ ID NO: 1068 - RNA, 19 nt, Artificial Sequence
  Synthetic Polynucleotide
uaacagaacacaaacuucc

SEQ ID NO: 1069 - RNA, 14 nt, Artificial Sequence
  Synthetic Polynucleotide
uuuguguucuguua

SEQ ID NO: 1070 - RNA, 19 nt, Artificial Sequence
  Synthetic Polynucleotide
uaacagaacacaaacuucc

SEQ ID NO: 1071 - RNA, 13 nt, Artificial Sequence
  Synthetic Polynucleotide
aaauacuuugcca

SEQ ID NO: 1072 - RNA, 19 nt, Artificial Sequence
  Synthetic Polynucleotide
uggcaaaguauuuggucuc

SEQ ID NO: 1073 - RNA, 14 nt, Artificial Sequence
  Synthetic Polynucleotide
caaauacuuugcca

SEQ ID NO: 1074 - RNA, 19 nt, Artificial Sequence
  Synthetic Polynucleotide
uggcaaaguauuuggucuc

SEQ ID NO: 1075 - RNA, 13 nt, Artificial Sequence
  Synthetic Polynucleotide
cuugcacuacaaa

SEQ ID NO: 1076 - RNA, 19 nt, Artificial Sequence
  Synthetic Polynucleotide
uuuguagugcaagucaaac

SEQ ID NO: 1077 - RNA, 14 nt, Artificial Sequence
  Synthetic Polynucleotide
acuugcacuacaaa

SEQ ID NO: 1078 - RNA, 19 nt, Artificial Sequence
  Synthetic Polynucleotide
uuuguagugcaagucaaac

SEQ ID NO: 1079 - RNA, 13 nt, Artificial Sequence
  Synthetic Polynucleotide
gaauuuauuagua

SEQ ID NO: 1080 - RNA, 19 nt, Artificial Sequence
  Synthetic Polynucleotide
uacuaauaaauucuuccag

SEQ ID NO: 1081 - RNA, 14 nt, Artificial Sequence
  Synthetic Polynucleotide
agaauuuauuagua

SEQ ID NO: 1082 - RNA, 19 nt, Artificial Sequence
  Synthetic Polynucleotide
uacuaauaaauucuuccag

SEQ ID NO: 1083 - RNA, 13 nt, Artificial Sequence
  Synthetic Polynucleotide
uugcacuacaaaa

SEQ ID NO: 1084 - RNA, 19 nt, Artificial Sequence
  Synthetic Polynucleotide
uuuuguagugcaagucaaa

SEQ ID NO: 1085 - RNA, 14 nt, Artificial Sequence
  Synthetic Polynucleotide
cuugcacuacaaaa

SEQ ID NO: 1086 - RNA, 19 nt, Artificial Sequence
  Synthetic Polynucleotide
uuuuguagugcaagucaaa

SEQ ID NO: 1087 - RNA, 13 nt, Artificial Sequence
  Synthetic Polynucleotide
auaaaacagguga

SEQ ID NO: 1088 - RNA, 19 nt, Artificial Sequence
  Synthetic Polynucleotide
ucaccuguuuuauuuucca

SEQ ID NO: 1089 - RNA, 14 nt, Artificial Sequence
  Synthetic Polynucleotide
aauaaaacagguga

SEQ ID NO: 1090 - RNA, 19 nt, Artificial Sequence
  Synthetic Polynucleotide
ucaccuguuuuauuuucca

SEQ ID NO: 1091 - RNA, 13 nt, Artificial Sequence
  Synthetic Polynucleotide
gacaacaacaaca

SEQ ID NO: 1092 - RNA, 19 nt, Artificial Sequence
  Synthetic Polynucleotide
uguuguuguugucguuguu

SEQ ID NO: 1093 - RNA, 13 nt, Artificial Sequence
  Synthetic Polynucleotide
augcuuguaacaa

SEQ ID NO: 1094 - RNA, 19 nt, Artificial Sequence
  Synthetic Polynucleotide
uuguuacaagcaucaucgu

SEQ ID NO: 1095 - RNA, 13 nt, Artificial Sequence
  Synthetic Polynucleotide
cagaaacucauga

SEQ ID NO: 1096 - RNA, 19 nt, Artificial Sequence
  Synthetic Polynucleotide
ucaugaguuucuggcaaag

SEQ ID NO: 1097 - RNA, 13 nt, Artificial Sequence
  Synthetic Polynucleotide
guauugcuaugca

SEQ ID NO: 1098 - RNA, 19 nt, Artificial Sequence
  Synthetic Polynucleotide
ugcauagcaauacagaaaa

SEQ ID NO: 1099 - RNA, 13 nt, Artificial Sequence
  Synthetic Polynucleotide
ccagaaacucaua

SEQ ID NO: 1100 - RNA, 19 nt, Artificial Sequence
  Synthetic Polynucleotide
uaugaguuucuggcaaagu

SEQ ID NO: 1101 - RNA, 13 nt, Artificial Sequence
  Synthetic Polynucleotide
acucaaacgagca

SEQ ID NO: 1102 - RNA, 19 nt, Artificial Sequence
  Synthetic Polynucleotide
ugcucguuugaguucaagu

SEQ ID NO: 1103 - RNA, 13 nt, Artificial Sequence
  Synthetic Polynucleotide
auaugaccgagaa

SEQ ID NO: 1104 - RNA, 19 nt, Artificial Sequence
  Synthetic Polynucleotide
uucucggucauauaauaac

SEQ ID NO: 1105 - RNA, 13 nt, Artificial Sequence
  Synthetic Polynucleotide
cgacgacaacgaa

SEQ ID NO: 1106 - RNA, 19 nt, Artificial Sequence
  Synthetic Polynucleotide
uucguugucgucgucauca

SEQ ID NO: 1107 - RNA, 13 nt, Artificial Sequence
  Synthetic Polynucleotide
guaaaccagugaa

SEQ ID NO: 1108 - RNA, 19 nt, Artificial Sequence
  Synthetic Polynucleotide
uucacugguuuacuaaacu

SEQ ID NO: 1109 - RNA, 13 nt, Artificial Sequence
  Synthetic Polynucleotide
uugucaguuuaga

SEQ ID NO: 1110 - RNA, 19 nt, Artificial Sequence
  Synthetic Polynucleotide
ucuaaacugacaaagaacc

SEQ ID NO: 1111 - RNA, 13 nt, Artificial Sequence
  Synthetic Polynucleotide
ucaucaguguuaa

SEQ ID NO: 1112 - RNA, 19 nt, Artificial Sequence
  Synthetic Polynucleotide
uuaacacugaugaaccaag

SEQ ID NO: 1113 - RNA, 13 nt, Artificial Sequence
  Synthetic Polynucleotide
aacucaaacgaga

SEQ ID NO: 1114 - RNA, 19 nt, Artificial Sequence
  Synthetic Polynucleotide
ucucguuugaguucaaguu

SEQ ID NO: 1115 - RNA, 13 nt, Artificial Sequence
  Synthetic Polynucleotide
cgacaacaacaaa

SEQ ID NO: 1116 - RNA, 19 nt, Artificial Sequence
  Synthetic Polynucleotide
uuuguuguugucguuguuc

SEQ ID NO: 1117 - RNA, 13 nt, Artificial Sequence
  Synthetic Polynucleotide
acgacaacgauga

SEQ ID NO: 1118 - RNA, 19 nt, Artificial Sequence
  Synthetic Polynucleotide
ucaucguugucgucgucau

SEQ ID NO: 1119 - RNA, 13 nt, Artificial Sequence
  Synthetic Polynucleotide
gcugccuaaggaa

SEQ ID NO: 1120 - RNA, 19 nt, Artificial Sequence
  Synthetic Polynucleotide
uuccuuaggcagcugauac

SEQ ID NO: 1121 - RNA, 13 nt, Artificial Sequence
  Synthetic Polynucleotide
auucuacauuuca

SEQ ID NO: 1122 - RNA, 19 nt, Artificial Sequence
  Synthetic Polynucleotide
ugaaauguagaauaaggcc

SEQ ID NO: 1123 - RNA, 14 nt, Artificial Sequence
  Synthetic Polynucleotide
caucaguguuaaaa

SEQ ID NO: 1124 - RNA, 19 nt, Artificial Sequence
  Synthetic Polynucleotide
uuuuaacacugaugaacca

SEQ ID NO: 1125 - RNA, 14 nt, Artificial Sequence
  Synthetic Polynucleotide
caucaguguuaaaa

SEQ ID NO: 1126 - RNA, 19 nt, Artificial Sequence
  Synthetic Polynucleotide
uuuuaacacugaugaacca

SEQ ID NO: 1127 - RNA, 14 nt, Artificial Sequence
  Synthetic Polynucleotide
caucaguguuaaaa

SEQ ID NO: 1128 - RNA, 19 nt, Artificial Sequence
  Synthetic Polynucleotide
uuuuaacacugaugaacca

SEQ ID NO: 1129 - RNA, 14 nt, Artificial Sequence
  Synthetic Polynucleotide
caucaguguuaaaa

SEQ ID NO: 1130 - RNA, 19 nt, Artificial Sequence
  Synthetic Polynucleotide
uuuuaacacugaugaacca

SEQ ID NO: 1131 - RNA, 13 nt, Artificial Sequence
  Synthetic Polynucleotide
ucaucaguguuaa

SEQ ID NO: 1132 - RNA, 19 nt, Artificial Sequence
  Synthetic Polynucleotide
uuaacacugaugaaccaag

SEQ ID NO: 1133 - RNA, 13 nt, Artificial Sequence
  Synthetic Polynucleotide
ucaucaguguuaa

SEQ ID NO: 1134 - RNA, 19 nt, Artificial Sequence
  Synthetic Polynucleotide
uuaacacugaugaaccaag

SEQ ID NO: 1135 - RNA, 13 nt, Artificial Sequence
  Synthetic Polynucleotide
ucaucaguguuaa

SEQ ID NO: 1136 - RNA, 19 nt, Artificial Sequence
  Synthetic Polynucleotide
uuaacacugaugaaccaag

SEQ ID NO: 1137 - RNA, 13 nt, Artificial Sequence
  Synthetic Polynucleotide
ucaucaguguuaa

SEQ ID NO: 1138 - RNA, 19 nt, Artificial Sequence
  Synthetic Polynucleotide
uuaacacugaugaaccaag

SEQ ID NO: 1139 - RNA, 13 nt, Artificial Sequence
  Synthetic Polynucleotide
ucaucaguguuaa

SEQ ID NO: 1140 - RNA, 19 nt, Artificial Sequence
  Synthetic Polynucleotide
uuaacacugaugaaccaag

SEQ ID NO: 1141 - RNA, 13 nt, Artificial Sequence
  Synthetic Polynucleotide
ucaucaguguuaa

SEQ ID NO: 1142 - RNA, 19 nt, Artificial Sequence
  Synthetic Polynucleotide
uuaacacugaugaaccaag

SEQ ID NO: 1143 - RNA, 13 nt, Artificial Sequence
  Synthetic Polynucleotide
ucaucaguguuaa

SEQ ID NO: 1144 - RNA, 19 nt, Artificial Sequence
  Synthetic Polynucleotide
uuaacacugaugaaccaag

SEQ ID NO: 1145 - RNA, 13 nt, Artificial Sequence
  Synthetic Polynucleotide
ucaucaguguuaa

SEQ ID NO: 1146 - RNA, 19 nt, Artificial Sequence
  Synthetic Polynucleotide
uuaacacugaugaaccaag

SEQ ID NO: 1147 - RNA, 13 nt, Artificial Sequence
  Synthetic Polynucleotide
ucaucaguguuaa

SEQ ID NO: 1148 - RNA, 19 nt, Artificial Sequence
  Synthetic Polynucleotide
uuaacacugaugaaccaag

SEQ ID NO: 1149 - RNA, 13 nt, Artificial Sequence
  Synthetic Polynucleotide
ucaucaguguuaa

SEQ ID NO: 1150 - RNA, 19 nt, Artificial Sequence
  Synthetic Polynucleotide
uuaacacugaugaaccaag

SEQ ID NO: 1151 - RNA, 13 nt, Artificial Sequence
  Synthetic Polynucleotide
ucaucaguguuaa

SEQ ID NO: 1152 - RNA, 19 nt, Artificial Sequence
  Synthetic Polynucleotide
uuaacacugaugaaccaag

SEQ ID NO: 1153 - RNA, 13 nt, Artificial Sequence
  Synthetic Polynucleotide
ucaucaguguuaa

SEQ ID NO: 1154 - RNA, 19 nt, Artificial Sequence
  Synthetic Polynucleotide
uuaacacugaugaaccaag

SEQ ID NO: 1155 - RNA, 13 nt, Artificial Sequence
  Synthetic Polynucleotide
  misc_feature (1)..(13): Modified with 2′Ome
  misc_feature (13)..(13): Modified with cholesterol with TEG linker
ucaucaguguuaa

SEQ ID NO: 1156 - RNA, 19 nt, Artificial Sequence
  Synthetic Polynucleotide
  misc_feature (1)..(1): Modified with P
  misc_feature (1)..(1): Modified with 2′Ome
  misc_feature (2)..(2): Modified with 2′fluoro
  misc_feature (5)..(5): Modified with 2′fluoro
  misc_feature (7)..(8): Modified with 2′fluoro
  misc_feature (11)..(11): Modified with 2′fluoro
  misc_feature (15)..(16): Modified with 2′fluoro
  misc_feature (17)..(18): Modified with 2′Ome
  misc_feature (13)..(19): Phosphorothioate internucleotide bond
uuaacacugaugaaccaag

SEQ ID NO: 1157 - RNA, 13 nt, Artificial Sequence
  Synthetic Polynucleotide
  misc_feature (1)..(13): Modified with 2′Ome
  misc_feature (13)..(13): Modified with cholesterol with TEG linker
ucaucaguguuaa

SEQ ID NO: 1158 - RNA, 19 nt, Artificial Sequence
  Synthetic Polynucleotide
  misc_feature (1)..(1): Modified with P
  misc_feature (1)..(1): Modified with 2′Ome
  misc_feature (2)..(2): Modified with 2′fluoro
  misc_feature (5)..(5): Modified with 2′fluoro
  misc_feature (7)..(8): Modified with 2′fluoro
  misc_feature (11)..(11): Modified with 2′fluoro
  misc_feature (13)..(14): Modified with 2′Ome
  misc_feature (15)..(16): Modified with 2′fluoro
  misc_feature (17)..(18): Modified with 2′Ome
  misc_feature (13)..(19): Phosphorothioate internucleotide bond
uuaacacugaugaaccaag

SEQ ID NO: 1159 - RNA, 15 nt, Artificial Sequence
  Synthetic Polynucleotide
gcuaaugguggaaab

SEQ ID NO: 1160 - RNA, 19 nt, Artificial Sequence
  Synthetic Polynucleotide
uuccaccauuagcacgcgg

SEQ ID NO: 1161 - RNA, 13 nt, Artificial Sequence
  Synthetic Polynucleotide
ugaucgugcgcuc

SEQ ID NO: 1162 - RNA, 19 nt, Artificial Sequence
  Synthetic Polynucleotide
gagcgcacgaucauguugg

SEQ ID NO: 1163 - RNA, 13 nt, Artificial Sequence
  Synthetic Polynucleotide
caauuccuggcga

SEQ ID NO: 1164 - RNA, 19 nt, Artificial Sequence
  Synthetic Polynucleotide
ucgccaggaauuguugcug

SEQ ID NO: 1165 - RNA, 13 nt, Artificial Sequence
  Synthetic Polynucleotide
aguggauccacga

SEQ ID NO: 1166 - RNA, 19 nt, Artificial Sequence
  Synthetic Polynucleotide
ucguggauccacuuccagc

SEQ ID NO: 1167 - RNA, 13 nt, Artificial Sequence
  Synthetic Polynucleotide
uacagcaaggucc

SEQ ID NO: 1168 - RNA, 19 nt, Artificial Sequence
  Synthetic Polynucleotide
ggaccuugcuguacugcgu

SEQ ID NO: 1169 - RNA, 13 nt, Artificial Sequence
  Synthetic Polynucleotide
aacaugaucgugc

SEQ ID NO: 1170 - RNA, 19 nt, Artificial Sequence
  Synthetic Polynucleotide
gcacgaucauguuggacag

SEQ ID NO: 1171 - RNA, 13 nt, Artificial Sequence
  Synthetic Polynucleotide
acaugaucgugcg

SEQ ID NO: 1172 - RNA, 19 nt, Artificial Sequence
  Synthetic Polynucleotide
cgcacgaucauguuggaca

SEQ ID NO: 1173 - RNA, 13 nt, Artificial Sequence
  Synthetic Polynucleotide
cagcaagguccug

SEQ ID NO: 1174 - RNA, 19 nt, Artificial Sequence
  Synthetic Polynucleotide
caggaccuugcuguacugc

SEQ ID NO: 1175 - RNA, 13 nt, Artificial Sequence
  Synthetic Polynucleotide
ccaacaugaucgu

SEQ ID NO: 1176 - RNA, 19 nt, Artificial Sequence
  Synthetic Polynucleotide
acgaucauguuggacagcu

SEQ ID NO: 1177 - RNA, 13 nt, Artificial Sequence
  Synthetic Polynucleotide
agcggaagcgcau

SEQ ID NO: 1178 - RNA, 19 nt, Artificial Sequence
  Synthetic Polynucleotide
augcgcuuccgcuucacca

SEQ ID NO: 1179 - RNA, 13 nt, Artificial Sequence
  Synthetic Polynucleotide
gcaucgaggccau

SEQ ID NO: 1180 - RNA, 19 nt, Artificial Sequence
  Synthetic Polynucleotide
auggccucgaugcgcuucc

SEQ ID NO: 1181 - RNA, 13 nt, Artificial Sequence
  Synthetic Polynucleotide
gacuaucgacaug

SEQ ID NO: 1182 - RNA, 19 nt, Artificial Sequence
  Synthetic Polynucleotide
caugucgauagucuugcag

SEQ ID NO: 1183 - RNA, 13 nt, Artificial Sequence
  Synthetic Polynucleotide
accugcaagacua

SEQ ID NO: 1184 - RNA, 19 nt, Artificial Sequence
  Synthetic Polynucleotide
uagucuugcagguggauag

SEQ ID NO: 1185 - RNA, 13 nt, Artificial Sequence
  Synthetic Polynucleotide
gcuccacggagaa

SEQ ID NO: 1186 - RNA, 19 nt, Artificial Sequence
  Synthetic Polynucleotide
uucuccguggagcugaagc

SEQ ID NO: 1187 - RNA, 13 nt, Artificial Sequence
  Synthetic Polynucleotide
cacagcauauaua

SEQ ID NO: 1188 - RNA, 19 nt, Artificial Sequence
  Synthetic Polynucleotide
uauauaugcuguguguacu

SEQ ID NO: 1189 - RNA, 13 nt, Artificial Sequence
  Synthetic Polynucleotide
cagcauauauaua

SEQ ID NO: 1190 - RNA, 19 nt, Artificial Sequence
  Synthetic Polynucleotide
uauauauaugcugugugua

SEQ ID NO: 1191 - RNA, 13 nt, Artificial Sequence
  Synthetic Polynucleotide
guacauugacuua

SEQ ID NO: 1192 - RNA, 19 nt, Artificial Sequence
  Synthetic Polynucleotide
uaagucaauguacagcugc

SEQ ID NO: 1193 - RNA, 14 nt, Artificial Sequence
  Synthetic Polynucleotide
uguacauugacuua

SEQ ID NO: 1194 - RNA, 19 nt, Artificial Sequence
  Synthetic Polynucleotide
uaagucaauguacagcugc

SEQ ID NO: 1195 - RNA, 13 nt, Artificial Sequence
  Synthetic Polynucleotide
aacuauugcuuca

SEQ ID NO: 1196 - RNA, 19 nt, Artificial Sequence
  Synthetic Polynucleotide
ugaagcaauaguugguguc

SEQ ID NO: 1197 - RNA, 14 nt, Artificial Sequence
  Synthetic Polynucleotide
caacuauugcuuca

SEQ ID NO: 1198 - RNA, 19 nt, Artificial Sequence
  Synthetic Polynucleotide
ugaagcaauaguugguguc

SEQ ID NO: 1199 - RNA, 13 nt, Artificial Sequence
  Synthetic Polynucleotide
gcauauauaugua

SEQ ID NO: 1200 - RNA, 19 nt, Artificial Sequence
  Synthetic Polynucleotide
uacauauauaugcugugug

SEQ ID NO: 1201 - RNA, 13 nt, Artificial Sequence
  Synthetic Polynucleotide
uguacauugacua

SEQ ID NO: 1202 - RNA, 19 nt, Artificial Sequence
  Synthetic Polynucleotide
uagucaauguacagcugcc

SEQ ID NO: 1203 - RNA, 14 nt, Artificial Sequence
  Synthetic Polynucleotide
cuguacauugacua

SEQ ID NO: 1204 - RNA, 19 nt, Artificial Sequence
  Synthetic Polynucleotide
uagucaauguacagcugcc

SEQ ID NO: 1205 - RNA, 13 nt, Artificial Sequence
  Synthetic Polynucleotide
agcauauauauga

SEQ ID NO: 1206 - RNA, 19 nt, Artificial Sequence
  Synthetic Polynucleotide
ucauauauaugcugugugu

SEQ ID NO: 1207 - RNA, 13 nt, Artificial Sequence
  Synthetic Polynucleotide
cagcaacaauuca

SEQ ID NO: 1208 - RNA, 19 nt, Artificial Sequence
  Synthetic Polynucleotide
ugaauuguugcuguauuuc

SEQ ID NO: 1209 - RNA, 13 nt, Artificial Sequence
  Synthetic Polynucleotide
cauauauauguua

SEQ ID NO: 1210 - RNA, 19 nt, Artificial Sequence
  Synthetic Polynucleotide
uaacauauauaugcugugu

SEQ ID NO: 1211 - RNA, 13 nt, Artificial Sequence
  Synthetic Polynucleotide
uugcuucagcuca

SEQ ID NO: 1212 - RNA, 19 nt, Artificial Sequence
  Synthetic Polynucleotide
ugagcugaagcaauaguug

SEQ ID NO: 1213 - RNA, 14 nt, Artificial Sequence
  Synthetic Polynucleotide
auugcuucagcuca

SEQ ID NO: 1214 - RNA, 19 nt, Artificial Sequence
  Synthetic Polynucleotide
ugagcugaagcaauaguug

SEQ ID NO: 1215 - RNA, 13 nt, Artificial Sequence
  Synthetic Polynucleotide
acagcauauauaa

SEQ ID NO: 1216 - RNA, 19 nt, Artificial Sequence
  Synthetic Polynucleotide
uuauauaugcuguguguac

SEQ ID NO: 1217 - RNA, 13 nt, Artificial Sequence
  Synthetic Polynucleotide
auugcuucagcua

SEQ ID NO: 1218 - RNA, 19 nt, Artificial Sequence
  Synthetic Polynucleotide
uagcugaagcaauaguugg

SEQ ID NO: 1219 - RNA, 14 nt, Artificial Sequence
  Synthetic Polynucleotide
uauugcuucagcua

SEQ ID NO: 1220 - RNA, 19 nt, Artificial Sequence
  Synthetic Polynucleotide
uagcugaagcaauaguugg

SEQ ID NO: 1221 - RNA, 13 nt, Artificial Sequence
  Synthetic Polynucleotide
caaguucaagcaa

SEQ ID NO: 1222 - RNA, 19 nt, Artificial Sequence
  Synthetic Polynucleotide
uugcuugaacuugucauag

SEQ ID NO: 1223 - RNA, 13 nt, Artificial Sequence
  Synthetic Polynucleotide
cagaguacacaca

SEQ ID NO: 1224 - RNA, 19 nt, Artificial Sequence
  Synthetic Polynucleotide
uguguguacucugcuugaa

SEQ ID NO: 1225 - RNA, 13 nt, Artificial Sequence
  Synthetic Polynucleotide
acacacagcauaa

SEQ ID NO: 1226 - RNA, 19 nt, Artificial Sequence
  Synthetic Polynucleotide
uuaugcuguguguacucug

SEQ ID NO: 1227 - RNA, 13 nt, Artificial Sequence
  Synthetic Polynucleotide
cagcauauauaua

SEQ ID NO: 1228 - RNA, 19 nt, Artificial Sequence
  Synthetic Polynucleotide
uauauauaugcugugugua

SEQ ID NO: 1229 - RNA, 13 nt, Artificial Sequence
  Synthetic Polynucleotide
ucaagcagaguaa

SEQ ID NO: 1230 - RNA, 19 nt, Artificial Sequence
  Synthetic Polynucleotide
uuacucugcuugaacuugu

SEQ ID NO: 1231 - RNA, 13 nt, Artificial Sequence
  Synthetic Polynucleotide
agcauauauauga

SEQ ID NO: 1232 - RNA, 19 nt, Artificial Sequence
  Synthetic Polynucleotide
ucauauauaugcugugugu

SEQ ID NO: 1233 - RNA, 13 nt, Artificial Sequence
  Synthetic Polynucleotide
agaguacacacaa

SEQ ID NO: 1234 - RNA, 19 nt, Artificial Sequence
  Synthetic Polynucleotide
uuguguguacucugcuuga

SEQ ID NO: 1235 - RNA, 13 nt, Artificial Sequence
  Synthetic Polynucleotide
aagcagaguacaa

SEQ ID NO: 1236 - RNA, 19 nt, Artificial Sequence
  Synthetic Polynucleotide
uuguacucugcuugaacuu

SEQ ID NO: 1237 - RNA, 13 nt, Artificial Sequence
  Synthetic Polynucleotide
uucaacacaucaa

SEQ ID NO: 1238 - RNA, 19 nt, Artificial Sequence
  Synthetic Polynucleotide
uugauguguugaagaacau

SEQ ID NO: 1239 - RNA, 13 nt, Artificial Sequence
  Synthetic Polynucleotide
agcagaguacaca

SEQ ID NO: 1240 - RNA, 19 nt, Artificial Sequence
  Synthetic Polynucleotide
uguguacucugcuugaacu

SEQ ID NO: 1241 - RNA, 13 nt, Artificial Sequence
  Synthetic Polynucleotide
auauauguucuua

SEQ ID NO: 1242 - RNA, 19 nt, Artificial Sequence
  Synthetic Polynucleotide
uaagaacauauauaugcug

SEQ ID NO: 1243 - RNA, 13 nt, Artificial Sequence
  Synthetic Polynucleotide
gacaaguucaaga

SEQ ID NO: 1244 - RNA, 19 nt, Artificial Sequence
  Synthetic Polynucleotide
ucuugaacuugucauagau

SEQ ID NO: 1245 - RNA, 13 nt, Artificial Sequence
  Synthetic Polynucleotide
uuaaagauggaga

SEQ ID NO: 1246 - RNA, 19 nt, Artificial Sequence
  Synthetic Polynucleotide
ucuccaucuuuaauggggc

SEQ ID NO: 1247 - RNA, 13 nt, Artificial Sequence
  Synthetic Polynucleotide
cuaugacaaguua

SEQ ID NO: 1248 - RNA, 19 nt, Artificial Sequence
  Synthetic Polynucleotide
uaacuugucauagauuucg

SEQ ID NO: 1249 - RNA, 13 nt, Artificial Sequence
  Synthetic Polynucleotide
caacgaaaucuaa

SEQ ID NO: 1250 - RNA, 19 nt, Artificial Sequence
  Synthetic Polynucleotide
uuagauuucguuguggguu

SEQ ID NO: 1251 - RNA, 13 nt, Artificial Sequence
  Synthetic Polynucleotide
uaugacaaguuca

SEQ ID NO: 1252 - RNA, 19 nt, Artificial Sequence
  Synthetic Polynucleotide
ugaacuugucauagauuuc

SEQ ID NO: 1253 - RNA, 13 nt, Artificial Sequence
  Synthetic Polynucleotide
aaguucaagcaga

SEQ ID NO: 1254 - RNA, 19 nt, Artificial Sequence
  Synthetic Polynucleotide
ucugcuugaacuugucaua

SEQ ID NO: 1255 - RNA, 13 nt, Artificial Sequence
  Synthetic Polynucleotide
caagcagaguaca

SEQ ID NO: 1256 - RNA, 19 nt, Artificial Sequence
  Synthetic Polynucleotide
uguacucugcuugaacuug

SEQ ID NO: 1257 - RNA, 13 nt, Artificial Sequence
  Synthetic Polynucleotide
aaucuaugacaaa

SEQ ID NO: 1258 - RNA, 19 nt, Artificial Sequence
  Synthetic Polynucleotide
uuugucauagauuucguug

SEQ ID NO: 1259 - RNA, 13 nt, Artificial Sequence
  Synthetic Polynucleotide
cacacagcauaua

SEQ ID NO: 1260 - RNA, 19 nt, Artificial Sequence
  Synthetic Polynucleotide
uauaugcuguguguacucu

SEQ ID NO: 1261 - RNA, 13 nt, Artificial Sequence
  Synthetic Polynucleotide
gaaauauagcaaa

SEQ ID NO: 1262 - RNA, 19 nt, Artificial Sequence
  Synthetic Polynucleotide
uuugcuauauuucugguag

SEQ ID NO: 1263 - RNA, 13 nt, Artificial Sequence
  Synthetic Polynucleotide
gaacucuaccaga

SEQ ID NO: 1264 - RNA, 19 nt, Artificial Sequence
  Synthetic Polynucleotide
ucugguagaguucuacgug

SEQ ID NO: 1265 - RNA, 13 nt, Artificial Sequence
  Synthetic Polynucleotide
gcaaagauaauga

SEQ ID NO: 1266 - RNA, 19 nt, Artificial Sequence
  Synthetic Polynucleotide
ucauuaucuuugcugucac

SEQ ID NO: 1267 - RNA, 13 nt, Artificial Sequence
  Synthetic Polynucleotide
aacucuaccagaa

SEQ ID NO: 1268 - RNA, 19 nt, Artificial Sequence
  Synthetic Polynucleotide
uucugguagaguucuacgu

SEQ ID NO: 1269 - RNA, 13 nt, Artificial Sequence
  Synthetic Polynucleotide
acucuaccagaaa

SEQ ID NO: 1270 - RNA, 19 nt, Artificial Sequence
  Synthetic Polynucleotide
uuucugguagaguucuacg

SEQ ID NO: 1271 - RNA, 13 nt, Artificial Sequence
  Synthetic Polynucleotide
acagcaaagauaa

SEQ ID NO: 1272 - RNA, 19 nt, Artificial Sequence
  Synthetic Polynucleotide
uuaucuuugcugucacaag

SEQ ID NO: 1273 - RNA, 13 nt, Artificial Sequence
  Synthetic Polynucleotide
caaucuaugacaa

SEQ ID NO: 1274 - RNA, 19 nt, Artificial Sequence
  Synthetic Polynucleotide
uugucauagauugcguugu

SEQ ID NO: 1275 - RNA, 13 nt, Artificial Sequence
  Synthetic Polynucleotide
agauucaagucaa

SEQ ID NO: 1276 - RNA, 19 nt, Artificial Sequence
  Synthetic Polynucleotide
uugacuugaaucucugcag

SEQ ID NO: 1277 - RNA, 13 nt, Artificial Sequence
  Synthetic Polynucleotide
cuguggagcaaca

SEQ ID NO: 1278 - RNA, 19 nt, Artificial Sequence
  Synthetic Polynucleotide
uguugcuccacaguugacu

SEQ ID NO: 1279 - RNA, 13 nt, Artificial Sequence
  Synthetic Polynucleotide
ugacagcaaagaa

SEQ ID NO: 1280 - RNA, 19 nt, Artificial Sequence
  Synthetic Polynucleotide
uucuuugcugucacaagag

SEQ ID NO: 1281 - RNA, 13 nt, Artificial Sequence
  Synthetic Polynucleotide
augacaaaaccaa

SEQ ID NO: 1282 - RNA, 19 nt, Artificial Sequence
  Synthetic Polynucleotide
uugguuuugucauagauug

SEQ ID NO: 1283 - RNA, 13 nt, Artificial Sequence
  Synthetic Polynucleotide
gagauucaaguca

SEQ ID NO: 1284 - RNA, 19 nt, Artificial Sequence
  Synthetic Polynucleotide
ugacuugaaucucugcagg

SEQ ID NO: 1285 - RNA, 13 nt, Artificial Sequence
  Synthetic Polynucleotide
cacacagcauaua

SEQ ID NO: 1286 - RNA, 19 nt, Artificial Sequence
  Synthetic Polynucleotide
uauaugcuguguguacucu

SEQ ID NO: 1287 - RNA, 13 nt, Artificial Sequence
  Synthetic Polynucleotide
cacacagcauaua

SEQ ID NO: 1288 - RNA, 19 nt, Artificial Sequence
  Synthetic Polynucleotide
uauaugcuguguguacucu

SEQ ID NO: 1289 - RNA, 13 nt, Artificial Sequence
  Synthetic Polynucleotide
cacacagcauaua

SEQ ID NO: 1290 - RNA, 19 nt, Artificial Sequence
  Synthetic Polynucleotide
uauaugcuguguguacucu

SEQ ID NO: 1291 - RNA, 13 nt, Artificial Sequence
  Synthetic Polynucleotide
cacacagcauaua

SEQ ID NO: 1292 - RNA, 19 nt, Artificial Sequence
  Synthetic Polynucleotide
uauaugcuguguguacucu

SEQ ID NO: 1293 - RNA, 13 nt, Artificial Sequence
  Synthetic Polynucleotide
cacacagcauaua

SEQ ID NO: 1294 - RNA, 19 nt, Artificial Sequence
  Synthetic Polynucleotide
uauaugcuguguguacucu

SEQ ID NO: 1295 - RNA, 13 nt, Artificial Sequence
  Synthetic Polynucleotide
cacacagcauaua

SEQ ID NO: 1296 - RNA, 19 nt, Artificial Sequence
  Synthetic Polynucleotide
uauaugcuguguguacucu

SEQ ID NO: 1297 - RNA, 13 nt, Artificial Sequence
  Synthetic Polynucleotide
cacacagcauaua

SEQ ID NO: 1298 - RNA, 19 nt, Artificial Sequence
  Synthetic Polynucleotide
uauaugcuguguguacucu

SEQ ID NO: 1299 - RNA, 13 nt, Artificial Sequence
  Synthetic Polynucleotide
cacacagcauaua

SEQ ID NO: 1300 - RNA, 19 nt, Artificial Sequence
  Synthetic Polynucleotide
uauaugcuguguguacucu

SEQ ID NO: 1301 - RNA, 13 nt, Artificial Sequence
  Synthetic Polynucleotide
agauucaagucaa

SEQ ID NO: 1302 - RNA, 19 nt, Artificial Sequence
  Synthetic Polynucleotide
uugacuugaaucucugcuu

SEQ ID NO: 1303-1304 - skipped (2 entries)

SEQ ID NO: 1305 - RNA, 13 nt, Artificial Sequence
  Synthetic Polynucleotide
cacacagcauaua

SEQ ID NO: 1306 - RNA, 19 nt, Artificial Sequence
  Synthetic Polynucleotide
uauaugcuguguguacucu

SEQ ID NO: 1307 - RNA, 13 nt, Artificial Sequence
  Synthetic Polynucleotide
cacacagcauaua

SEQ ID NO: 1308 - RNA, 19 nt, Artificial Sequence
  Synthetic Polynucleotide
uauaugcuguguguacucu

SEQ ID NO: 1309 - RNA, 13 nt, Artificial Sequence
  Synthetic Polynucleotide
cacacagcauaua

SEQ ID NO: 1310 - RNA, 19 nt, Artificial Sequence
  Synthetic Polynucleotide
uauaugcuguguguacucu

SEQ ID NO: 1311 - RNA, 13 nt, Artificial Sequence
  Synthetic Polynucleotide
cacacagcauaua

SEQ ID NO: 1312 - RNA, 19 nt, Artificial Sequence
  Synthetic Polynucleotide
uauaugcuguguguacucu

SEQ ID NO: 1313 - RNA, 13 nt, Artificial Sequence
  Synthetic Polynucleotide
  misc_feature (1)..(13): Modified with 2′Ome
  misc_feature (13)..(13): Modified with cholesterol with TEG linker
cacacagcauaua

SEQ ID NO: 1314 - RNA, 19 nt, Artificial Sequence
  Synthetic Polynucleotide
  misc_feature (1)..(1): Modified with P
  misc_feature (1)..(1): Modified with 2′Ome
  misc_feature (3)..(3): Modified with 2′fluoro
  misc_feature (5)..(5): Modified with 2′fluoro
  misc_feature (7)..(8): Modified with 2′fluoro
  misc_feature (10)..(10): Modified with 2′fluoro
  misc_feature (12)..(12): Modified with 2′fluoro
  misc_feature (13)..(13): Modified with 2′Ome
  misc_feature (14)..(14): Modified with 2′fluoro
  misc_feature (15)..(15): Modified with 2′Ome
  misc_feature (16)..(16): Modified with 2′fluoro
  misc_feature (17)..(17): Modified with 2′Ome
  misc_feature (18)..(18): Modified with 2′fluoro
  misc_feature (13)..(19): Phosphorothioate internucleotide bond
uauaugcuguguguacucu

SEQ ID NO: 1315 - RNA, 13 nt, Artificial Sequence
  Synthetic Polynucleotide
gaugagcuuccua

SEQ ID NO: 1316 - RNA, 19 nt, Artificial Sequence
  Synthetic Polynucleotide
uaggaagcucaucucuccu

SEQ ID NO: 1317 - RNA, 13 nt, Artificial Sequence
  Synthetic Polynucleotide
agaacaguccuua

SEQ ID NO: 1318 - RNA, 19 nt, Artificial Sequence
  Synthetic Polynucleotide
uaaggacuguucugucgau

SEQ ID NO: 1319 - RNA, 13 nt, Artificial Sequence
  Synthetic Polynucleotide
gaacaguccuuaa

SEQ ID NO: 1320 - RNA, 19 nt, Artificial Sequence
  Synthetic Polynucleotide
uuaaggacuguucugucga

SEQ ID NO: 1321 - RNA, 13 nt, Artificial Sequence
  Synthetic Polynucleotide
caguccuuaauca

SEQ ID NO: 1322 - RNA, 19 nt, Artificial Sequence
  Synthetic Polynucleotide
ugauuaaggacuguucugu

SEQ ID NO: 1323 - RNA, 13 nt, Artificial Sequence
  Synthetic Polynucleotide
guccuuaauccaa

SEQ ID NO: 1324 - RNA, 19 nt, Artificial Sequence
  Synthetic Polynucleotide
uuggauuaaggacuguucu

SEQ ID NO: 1325 - RNA, 13 nt, Artificial Sequence
  Synthetic Polynucleotide
uuaauccagaaaa

SEQ ID NO: 1326 - RNA, 19 nt, Artificial Sequence
  Synthetic Polynucleotide
uuuucuggauuaaggacug

SEQ ID NO: 1327 - RNA, 13 nt, Artificial Sequence
  Synthetic Polynucleotide
uguuauuggugua

SEQ ID NO: 1328 - RNA, 19 nt, Artificial Sequence
  Synthetic Polynucleotide
uacaccaauaacauuagca

SEQ ID NO: 1329 - RNA, 13 nt, Artificial Sequence
  Synthetic Polynucleotide
uugaaaccacuaa

SEQ ID NO: 1330 - RNA, 19 nt, Artificial Sequence
  Synthetic Polynucleotide
uuagugguuucaauggugu

SEQ ID NO: 1331 - RNA, 13 nt, Artificial Sequence
  Synthetic Polynucleotide
gagaaaagagaaa

SEQ ID NO: 1332 - RNA, 19 nt, Artificial Sequence
  Synthetic Polynucleotide
uuucucuuuucucugccuc

SEQ ID NO: 1333 - RNA, 13 nt, Artificial Sequence
  Synthetic Polynucleotide
agaaaagagaaaa

SEQ ID NO: 1334 - RNA, 19 nt, Artificial Sequence
  Synthetic Polynucleotide
uuuucucuuuucucugccu

SEQ ID NO: 1335 - RNA, 13 nt, Artificial Sequence
  Synthetic Polynucleotide
gaaaagagaaaga

SEQ ID NO: 1336 - RNA, 19 nt, Artificial Sequence
  Synthetic Polynucleotide
ucuuucucuuuucucugcc

SEQ ID NO: 1337 - RNA, 13 nt, Artificial Sequence
  Synthetic Polynucleotide
acacucagcucua

SEQ ID NO: 1338 - RNA, 19 nt, Artificial Sequence
  Synthetic Polynucleotide
uagagcugaguguuagcaa

SEQ ID NO: 1339 - RNA, 13 nt, Artificial Sequence
  Synthetic Polynucleotide
aaauaagguuuca

SEQ ID NO: 1340 - RNA, 19 nt, Artificial Sequence
  Synthetic Polynucleotide
ugaaaccuuauuucaaagg

SEQ ID NO: 1341 - RNA, 13 nt, Artificial Sequence
  Synthetic Polynucleotide
aaucucucuccua

SEQ ID NO: 1342 - RNA, 19 nt, Artificial Sequence
  Synthetic Polynucleotide
uaggagagagauuuaguau

SEQ ID NO: 1343 - RNA, 13 nt, Artificial Sequence
  Synthetic Polynucleotide
ucucucuccuuua

SEQ ID NO: 1344 - RNA, 19 nt, Artificial Sequence
  Synthetic Polynucleotide
uaaaggagagagauuuagu

SEQ ID NO: 1345 - RNA, 13 nt, Artificial Sequence
  Synthetic Polynucleotide
cucucuccuuuua

SEQ ID NO: 1346 - RNA, 19 nt, Artificial Sequence
  Synthetic Polynucleotide
uaaaaggagagagauuuag

SEQ ID NO: 1347 - RNA, 13 nt, Artificial Sequence
  Synthetic Polynucleotide
auuggugcuacua

SEQ ID NO: 1348 - RNA, 19 nt, Artificial Sequence
  Synthetic Polynucleotide
uaguagcaccaauaaauaa

SEQ ID NO: 1349 - RNA, 13 nt, Artificial Sequence
  Synthetic Polynucleotide
ugcuacuguuuaa

SEQ ID NO: 1350 - RNA, 19 nt, Artificial Sequence
  Synthetic Polynucleotide
uuaaacaguagcaccaaua

SEQ ID NO: 1351 - RNA, 13 nt, Artificial Sequence
  Synthetic Polynucleotide
cagaacaguccua

SEQ ID NO: 1352 - RNA, 19 nt, Artificial Sequence
  Synthetic Polynucleotide
uaggacuguucugucgaug

SEQ ID NO: 1353 - RNA, 13 nt, Artificial Sequence
  Synthetic Polynucleotide
ugcagauuaugca

SEQ ID NO: 1354 - RNA, 19 nt, Artificial Sequence
  Synthetic Polynucleotide
ugcauaaucugcaugguga

SEQ ID NO: 1355 - RNA, 13 nt, Artificial Sequence
  Synthetic Polynucleotide
gauuaugcggaua

SEQ ID NO: 1356 - RNA, 19 nt, Artificial Sequence
  Synthetic Polynucleotide
uauccgcauaaucugcaug

SEQ ID NO: 1357 - RNA, 13 nt, Artificial Sequence
  Synthetic Polynucleotide
ugcggaucaaaca

SEQ ID NO: 1358 - RNA, 19 nt, Artificial Sequence
  Synthetic Polynucleotide
uguuugauccgcauaaucu

SEQ ID NO: 1359 - RNA, 13 nt, Artificial Sequence
  Synthetic Polynucleotide
ggauucgccauua

SEQ ID NO: 1360 - RNA, 19 nt, Artificial Sequence
  Synthetic Polynucleotide
uaauggcgaauccaauucc

SEQ ID NO: 1361 - RNA, 13 nt, Artificial Sequence
  Synthetic Polynucleotide
uugacugcuguga

SEQ ID NO: 1362 - RNA, 19 nt, Artificial Sequence
  Synthetic Polynucleotide
ucacagcagucaaauacau

SEQ ID NO: 1363 - RNA, 13 nt, Artificial Sequence
  Synthetic Polynucleotide
cagaaagacagaa

SEQ ID NO: 1364 - RNA, 19 nt, Artificial Sequence
  Synthetic Polynucleotide
uucugucuuucuguccguc

SEQ ID NO: 1365 - RNA, 13 nt, Artificial Sequence
  Synthetic Polynucleotide
gaaaccacuagua

SEQ ID NO: 1366 - RNA, 19 nt, Artificial Sequence
  Synthetic Polynucleotide
uacuagugguuucaauggu

SEQ ID NO: 1367 - RNA, 13 nt, Artificial Sequence
  Synthetic Polynucleotide
aaccacuaguuca

SEQ ID NO: 1368 - RNA, 19 nt, Artificial Sequence
  Synthetic Polynucleotide
ugaacuagugguuucaaug

SEQ ID NO: 1369 - RNA, 13 nt, Artificial Sequence
  Synthetic Polynucleotide
uaucuuuugcuca

SEQ ID NO: 1370 - RNA, 19 nt, Artificial Sequence
  Synthetic Polynucleotide
ugagcaaaagauacaucuc

SEQ ID NO: 1371 - RNA, 13 nt, Artificial Sequence
  Synthetic Polynucleotide
aucuuuugcucua

SEQ ID NO: 1372 - RNA, 19 nt, Artificial Sequence
  Synthetic Polynucleotide
uagagcaaaagauacaucu

SEQ ID NO: 1373 - RNA, 13 nt, Artificial Sequence
  Synthetic Polynucleotide
ucacuagcuuaua

SEQ ID NO: 1374 - RNA, 19 nt, Artificial Sequence
  Synthetic Polynucleotide
uauaagcuagugacuguca

SEQ ID NO: 1375 - RNA, 13 nt, Artificial Sequence
  Synthetic Polynucleotide
cacuagcuuauca

SEQ ID NO: 1376 - RNA, 19 nt, Artificial Sequence
  Synthetic Polynucleotide
ugauaagcuagugacuguc

SEQ ID NO: 1377 - RNA, 25 nt, Artificial Sequence
  Synthetic Polynucleotide
uaaggacuguucugucgauggugau

SEQ ID NO: 1378 - RNA, 25 nt, Artificial Sequence
  Synthetic Polynucleotide
uguuugauccgcauaaucugcaugg

SEQ ID NO: 1379 - RNA, 13 nt, Artificial Sequence
  Synthetic Polynucleotide
  misc_feature (5)..(5): Modified with 2′Ome
  misc_feature (8)..(12): Modified with 2′Ome
  misc_feature (13)..(13): Modified with cholesterol with hydroxyprolinol linker
agaacaguccuua

SEQ ID NO: 1380 - RNA, 19 nt, Artificial Sequence
  Synthetic Polynucleotide
  misc_feature (1)..(1): Modified with P
  misc_feature (1)..(1): Modified with 2′Ome
  misc_feature (7)..(8): Modified with 2′fluoro
  misc_feature (10)..(13): Modified with 2′fluoro
  misc_feature (15)..(16): Modified with 2′fluoro
  misc_feature (13)..(19): Phosphorothioate internucleotide bond
uaaggacuguucugucgau

SEQ ID NO: 1381 - RNA, 13 nt, Artificial Sequence
  Synthetic Polynucleotide
agaacaguccuua

SEQ ID NO: 1382 - RNA, 13 nt, Artificial Sequence
  Synthetic Polynucleotide
agaacaguccuua

SEQ ID NO: 1383 - RNA, 13 nt, Artificial Sequence
  Synthetic Polynucleotide
agaacaguccuua

SEQ ID NO: 1384 - RNA, 19 nt, Artificial Sequence
  Synthetic Polynucleotide
uaaggacuguucugucgau

SEQ ID NO: 1385 - RNA, 19 nt, Artificial Sequence
  Synthetic Polynucleotide
uaaggacuguucugucgau

SEQ ID NO: 1386 - RNA, 19 nt, Artificial Sequence
  Synthetic Polynucleotide
uaaggacuguucugucgau

SEQ ID NO: 1387 - RNA, 19 nt, Artificial Sequence
  Synthetic Polynucleotide
uaaggacuguucugucgau

SEQ ID NO: 1388 - RNA, 19 nt, Artificial Sequence
  Synthetic Polynucleotide
uaaggacuguucugucgau

SEQ ID NO: 1389 - RNA, 19 nt, Artificial Sequence
  Synthetic Polynucleotide
uaaggacuguucugucgau

SEQ ID NO: 1390 - RNA, 19 nt, Artificial Sequence
  Synthetic Polynucleotide
uaaggacuguucugucgau

SEQ ID NO: 1391 - RNA, 19 nt, Artificial Sequence
  Synthetic Polynucleotide
uaaggacuguucugucgau

SEQ ID NO: 1392 - RNA, 19 nt, Artificial Sequence
  Synthetic Polynucleotide
uaaggacuguucugucgau

SEQ ID NO: 1393 - RNA, 19 nt, Artificial Sequence
  Synthetic Polynucleotide
  misc_feature (1)..(1): n is 5-methyl uracil
  misc_feature (7)..(7): n is 5-methyl cytosine
  misc_feature (8)..(8): n is 5-methyl uracil
  misc_feature (10)..(11): n is 5-methyl uracil
  misc_feature (12)..(12): n is 5-methyl cytosine
  misc_feature (15)..(15): n is 5-methyl uracil
  misc_feature (16)..(16): n is 5-methyl cytosine
naagganngnnnugnngau

SEQ ID NO: 1394 - RNA, 19 nt, Artificial Sequence
  Synthetic Polynucleotide
  misc_feature (1)..(1): n is 5-methyl uracil
  misc_feature (7)..(7): n is 5-methyl cytosine
  misc_feature (8)..(8): n is 5-methyl uracil
  misc_feature (10)..(11): n is 5-methyl uracil
  misc_feature (12)..(12): n is 5-methyl cytosine
  misc_feature (15)..(15): n is 5-methyl uracil
  misc_feature (16)..(16): n is 5-methyl cytosine
naagganngnnnugnngau

SEQ ID NO: 1395 - RNA, 19 nt, Artificial Sequence
  Synthetic Polynucleotide
  misc_feature (1)..(1): n is 5-methyl uracil
  misc_feature (7)..(7): n is 5-methyl cytosine
  misc_feature (8)..(8): n is 5-methyl uracil
  misc_feature (10)..(11): n is 5-methyl uracil
  misc_feature (12)..(12): n is 5-methyl cytosine
  misc_feature (15)..(15): n is 5-methyl uracil
  misc_feature (16)..(16): n is 5-methyl cytosine
naagganngnnnugnngau

SEQ ID NO: 1396 - RNA, 19 nt, Artificial Sequence
  Synthetic Polynucleotide
  misc_feature (1)..(1): n is 5-methyl uracil
  misc_feature (7)..(7): n is 5-methyl cytosine
  misc_feature (8)..(8): n is 5-methyl uracil
  misc_feature (10)..(11): n is 5-methyl uracil
  misc_feature (12)..(12): n is 5-methyl cytosine
  misc_feature (15)..(15): n is 5-methyl uracil
  misc_feature (16)..(16): n is 5-methyl cytosine
naagganngnnnugnngau

SEQ ID NO: 1397 - RNA, 13 nt, Artificial Sequence
  Synthetic Polynucleotide
  misc_feature (1)..(1): Modified with 2′Ome
  misc_feature (3)..(3): Modified with 2′Ome
  misc_feature (7)..(8): Modified with 2′Ome
  misc_feature (12)..(12): Modified with 2′Ome
  misc_feature (13)..(13): Modified with cholesterol with hydroxyprolinol linker
ugcggaucaaaca

SEQ ID NO: 1398 - RNA, 19 nt, Artificial Sequence
  Synthetic Polynucleotide
  misc_feature (1)..(1): Modified with P
  misc_feature (1)..(1): Modified with 2′Ome
  misc_feature (3)..(5): Modified with 2′fluoro
  misc_feature (8)..(10): Modified with 2′fluoro
  misc_feature (12)..(12): Modified with 2′fluoro
  misc_feature (14)..(14): Modified with 2′fluoro
  misc_feature (17)..(18): Modified with 2′fluoro
  misc_feature (13)..(19): Phosphorothioate internucleotide bond
uguuugauccgcauaaucu

SEQ ID NO: 1399 - RNA, 15 nt, Artificial Sequence
  Synthetic Polynucleotide
  misc_feature (1)..(13): Modified with 2′fluoro
  misc_feature (11)..(13): Phosphorothioate internucleotide bond
  misc_feature (13)..(13): Modified with cholesterol with hydroxyprolinol linker
cuguggaagucuaab

SEQ ID NO: 1400 - RNA, 19 nt, Artificial Sequence
  Synthetic Polynucleotide
  misc_feature (1)..(1): Modified with P
  misc_feature (1)..(18): Modified with 2′fluoro
  misc_feature (11)..(19): Phosphorothioate internucleotide bond
uagacuuccacagaacucu

SEQ ID NO: 1401 - RNA, 13 nt, Artificial Sequence
  Synthetic Polynucleotide
  misc_feature (1)..(13): Modified with 2′fluoro
  misc_feature (11)..(13): Phosphorothioate internucleotide bond
  misc_feature (13)..(13): Modified with cholesterol with hydroxyprolinol linker
cuguggaagucua

SEQ ID NO: 1402 - RNA, 19 nt, Artificial Sequence
  Synthetic Polynucleotide
  misc_feature (1)..(1): Modified with P
  misc_feature (1)..(10): Modified with 2′fluoro
  misc_feature (11)..(11): Modified with 2′Ome
  misc_feature (12)..(12): Modified with 2′fluoro
  misc_feature (13)..(13): Modified with 2′Ome
  misc_feature (14)..(14): Modified with 2′fluoro
  misc_feature (15)..(18): Modified with 2′Ome
  misc_feature (11)..(19): Phosphorothioate internucleotide bond
uagacuuccacagaacucu

SEQ ID NO: 1403 - RNA, 13 nt, Artificial Sequence
  Synthetic Polynucleotide
  misc_feature (1)..(13): Modified with 2′fluoro
  misc_feature (11)..(13): Phosphorothioate internucleotide bond
  misc_feature (13)..(13): Modified with cholesterol with hydroxyprolinol linker
cuguggaagucua

SEQ ID NO: 1404 - RNA, 19 nt, Artificial Sequence
  Synthetic Polynucleotide
  misc_feature (1)..(1): Modified with P
  misc_feature (1)..(1): n is 5-methyl uracil
  misc_feature (1)..(1): Modified with 2′fluoro
  misc_feature (5)..(5): n is 5-methyl cytosine
  misc_feature (6)..(7): n is 5-methyl uracil
  misc_feature (8)..(8): n is 5-methyl cytosine
  misc_feature (5)..(9): Modified with 2′fluoro
  misc_feature (11)..(11): n is 5-methyl cytosine
  misc_feature (11)..(11): Modified with 2′Ome
  misc_feature (13)..(13): Modified with 2′Ome
  misc_feature (16)..(16): n is 5-methyl cytosine
  misc_feature (17)..(17): n is 5-methyl uracil
  misc_feature (18)..(18): n is 5-methyl cytosine
  misc_feature (15)..(18): Modified with 2′Ome
  misc_feature (11)..(19): Phosphorothioate internucleotide bond
nagannnncanagaannnu

SEQ ID NO: 1405 - RNA, 13 nt, Artificial Sequence
  Synthetic Polynucleotide
  misc_feature (1)..(13): Modified with 2′fluoro
  misc_feature (11)..(13): Phosphorothioate internucleotide bond
  misc_feature (13)..(13): Modified with cholesterol with hydroxyprolinol linker
cuguggaagucua

SEQ ID NO: 1406 - RNA, 19 nt, Artificial Sequence
  Synthetic Polynucleotide
  misc_feature (1)..(1): Modified with P
  misc_feature (1)..(1): n is 5-methyl uracil
  misc_feature (5)..(5): n is 5-methyl cytosine
  misc_feature (6)..(7): n is 5-methyl uracil
  misc_feature (8)..(9): n is 5-methyl cytosine
  misc_feature (1)..(10): Modified with 2′fluoro
  misc_feature (11)..(11): n is 5-methyl cytosine
  misc_feature (11)..(11): Modified with 2′Ome
  misc_feature (12)..(12): Modified with 2′fluoro
  misc_feature (13)..(13): Modified with 2′Ome
  misc_feature (14)..(14): Modified with 2′fluoro
  misc_feature (16)..(16): n is 5-methyl cytosine
  misc_feature (17)..(17): n is 5-methyl uracil
  misc_feature (18)..(18): n is 5-methyl cytosine
  misc_feature (15)..(18): Modified with 2′Ome
  misc_feature (11)..(19): Phosphorothioate internucleotide bond
nagannnnnanagaannnu

SEQ ID NO: 1407 - RNA, 13 nt, Artificial Sequence
  Synthetic Polynucleotide
  misc_feature (1)..(1): n is 5-methyl cytosine
  misc_feature (2)..(2): n is 5-methyl uracil
  misc_feature (1)..(2): Modified with 2′Ome
  misc_feature (4)..(4): n is 5-methyl uracil
  misc_feature (4)..(4): Modified with 2′Ome
  misc_feature (10)..(10): n is 5-methyl uracil
  misc_feature (11)..(11): n is 5-methyl cytosine
  misc_feature (12)..(12): n is 5-methyl uracil
  misc_feature (10)..(12): Modified with 2′Ome
  misc_feature (11)..(13): Phosphorothioate internucleotide bond
  misc_feature (13)..(13): Modified with cholesterol
nngnggaagnnna

SEQ ID NO: 1408 - RNA, 19 nt, Artificial Sequence
  Synthetic Polynucleotide
  misc_feature (1)..(1): Modified with P
  misc_feature (1)..(18): Modified with 2′fluoro
  misc_feature (11)..(19): Phosphorothioate internucleotide bond
uagacuuccacagaacucu

SEQ ID NO: 1409 - RNA, 13 nt, Artificial Sequence
  Synthetic Polynucleotide
  misc_feature (1)..(1): n is 5-methyl cytosine
  misc_feature (2)..(2): n is 5-methyl uracil
  misc_feature (1)..(2): Modified with 2′Ome
  misc_feature (4)..(4): n is 5-methyl uracil
  misc_feature (4)..(4): Modified with 2′Ome
  misc_feature (10)..(10): n is 5-methyl uracil
  misc_feature (11)..(11): n is 5-methyl cytosine
  misc_feature (12)..(12): n is 5-methyl uracil
  misc_feature (10)..(12): Modified with 2′Ome
  misc_feature (11)..(13): Phosphorothioate internucleotide bond
  misc_feature (13)..(13): Modified with cholesterol
nngnggaagnnna

SEQ ID NO: 1410 - RNA, 19 nt, Artificial Sequence
  Synthetic Polynucleotide
  misc_feature (1)..(1): Modified with P
  misc_feature (1)..(10): Modified with 2′fluoro
  misc_feature (11)..(11): Modified with 2′Ome
  misc_feature (12)..(12): Modified with 2′fluoro
  misc_feature (13)..(13): Modified with 2′Ome
  misc_feature (14)..(14): Modified with 2′fluoro
  misc_feature (15)..(18): Modified with 2′Ome
  misc_feature (11)..(19): Phosphorothioate internucleotide bond
uagacuuccacagaacucu

SEQ ID NO: 1411 - RNA, 13 nt, Artificial Sequence
  Synthetic Polynucleotide
  misc_feature (1)..(1): n is 5-methyl cytosine
  misc_feature (2)..(2): n is 5-methyl uracil
  misc_feature (1)..(2): Modified with 2′Ome
  misc_feature (4)..(4): n is 5-methyl uracil
  misc_feature (4)..(4): Modified with 2′Ome
  misc_feature (10)..(10): n is 5-methyl uracil
  misc_feature (11)..(11): n is 5-methyl cytosine
  misc_feature (12)..(12): n is 5-methyl uracil
  misc_feature (10)..(12): Modified with 2′Ome
  misc_feature (11)..(13): Phosphorothioate internucleotide bond
  misc_feature (13)..(13): Modified with cholesterol
nngnggaagnnna

SEQ ID NO: 1412 - RNA, 19 nt, Artificial Sequence
  Synthetic Polynucleotide
  misc_feature (1)..(1): n is 5-methyl uracil
  misc_feature (1)..(1): Modified with P
  misc_feature (1)..(1): Modified with 2′fluoro
  misc_feature (5)..(5): n is 5-methyl cytosine
  misc_feature (6)..(7): n is 5-methyl uracil
  misc_feature (8)..(8): n is 5-methyl cytosine
  misc_feature (5)..(9): Modified with 2′fluoro
  misc_feature (11)..(11): n is 5-methyl cytosine
  misc_feature (11)..(11): Modified with 2′Ome
  misc_feature (13)..(13): Modified with 2′Ome
  misc_feature (16)..(16): n is 5-methyl cytosine
  misc_feature (17)..(17): n is 5-methyl uracil
  misc_feature (18)..(18): n is 5-methyl cytosine
  misc_feature (15)..(18): Modified with 2′Ome
  misc_feature (11)..(19): Phosphorothioate internucleotide bond
nagannnncanagaannnu

SEQ ID NO: 1413 - RNA, 13 nt, Artificial Sequence
  Synthetic Polynucleotide
  misc_feature (1)..(1): n is 5-methyl cytosine
  misc_feature (2)..(2): n is 5-methyl uracil
  misc_feature (1)..(2): Modified with 2′Ome
  misc_feature (4)..(4): n is 5-methyl uracil
  misc_feature (4)..(4): Modified with 2′Ome
  misc_feature (10)..(10): n is 5-methyl uracil
  misc_feature (11)..(11): n is 5-methyl cytosine
  misc_feature (12)..(12): n is 5-methyl uracil
  misc_feature (10)..(12): Modified with 2′Ome
  misc_feature (11)..(13): Phosphorothioate internucleotide bond
  misc_feature (13)..(13): Modified with cholesterol
nngnggaagnnna

SEQ ID NO: 1414 - RNA, 19 nt, Artificial Sequence
  Synthetic Polynucleotide
  misc_feature (1)..(1): Modified with P
  misc_feature (1)..(1): Modified with 2′fluoro
  misc_feature (1)..(1): n is 5-methyl uracil
  misc_feature (5)..(5): n is 5-methyl cytosine
  misc_feature (6)..(7): n is 5-methyl uracil
  misc_feature (8)..(9): n is 5-methyl cytosine
  misc_feature (1)..(10): Modified with 2′fluoro
  misc_feature (11)..(11): n is 5-methyl cytosine
  misc_feature (11)..(11): Modified with 2′Ome
  misc_feature (12)..(12): Modified with 2′fluoro
  misc_feature (13)..(13): Modified with 2′Ome
  misc_feature (14)..(14): Modified with 2′fluoro
  misc_feature (16)..(16): n is 5-methyl cytosine
  misc_feature (17)..(17): n is 5-methyl uracil
  misc_feature (18)..(18): n is 5-methyl cytosine
  misc_feature (15)..(18): Modified with 2′Ome
  misc_feature (11)..(19): Phosphorothioate internucleotide bond
nagannnnnanagaannnu

SEQ ID NO: 1415 - RNA, 13 nt, Artificial Sequence
  Synthetic Polynucleotide
  misc_feature (1)..(2): Modified with 2′Ome
  misc_feature (4)..(4): Modified with 2′Ome
  misc_feature (10)..(12): Modified with 2′Ome
  misc_feature (11)..(13): Phosphorothioate internucleotide bond
  misc_feature (13)..(13): Modified with cholesterol
cuguggaagucua

SEQ ID NO: 1416 - RNA, 19 nt, Artificial Sequence
  Synthetic Polynucleotide
  misc_feature (1)..(1): Modified with P
  misc_feature (1)..(18): Modified with 2′fluoro
  misc_feature (11)..(19): Phosphorothioate internucleotide bond
uagacuuccacagaacucu

SEQ ID NO: 1417 - RNA, 13 nt, Artificial Sequence
  Synthetic Polynucleotide
  misc_feature (1)..(2): Modified with 2′Ome
  misc_feature (4)..(4): Modified with 2′Ome
  misc_feature (10)..(12): Modified with 2′Ome
  misc_feature (11)..(13): Phosphorothioate internucleotide bond
  misc_feature (13)..(13): Modified with cholesterol
cuguggaagucua

SEQ ID NO: 1418 - RNA, 19 nt, Artificial Sequence
  Synthetic Polynucleotide
  misc_feature (1)..(1): Modified with P
  misc_feature (1)..(10): Modified with 2′fluoro
  misc_feature (11)..(11): Modified with 2′Ome
  misc_feature (12)..(12): Modified with 2′fluoro
  misc_feature (13)..(13): Modified with 2′Ome
  misc_feature (14)..(14): Modified with 2′fluoro
  misc_feature (15)..(18): Modified with 2′Ome
  misc_feature (11)..(19): Phosphorothioate internucleotide bond
uagacuuccacagaacucu

SEQ ID NO: 1419 - RNA, 13 nt, Artificial Sequence
  Synthetic Polynucleotide
  misc_feature (1)..(2): Modified with 2′Ome
  misc_feature (4)..(4): Modified with 2′Ome
  misc_feature (10)..(12): Modified with 2′Ome
  misc_feature (11)..(13): Phosphorothioate internucleotide bond
  misc_feature (13)..(13): Modified with cholesterol
cuguggaagucua

SEQ ID NO: 1420 - RNA, 19 nt, Artificial Sequence
  Synthetic Polynucleotide
  misc_feature (1)..(1): Modified with P
  misc_feature (1)..(1): Modified with 2′fluoro
  misc_feature (1)..(1): n is 5-methyl uracil
  misc_feature (5)..(5): n is 5-methyl cytosine
  misc_feature (6)..(7): n is 5-methyl uracil
  misc_feature (8)..(8): n is 5-methyl cytosine
  misc_feature (5)..(9): Modified with 2′fluoro
  misc_feature (11)..(11): n is 5-methyl cytosine
  misc_feature (11)..(11): Modified with 2′Ome
  misc_feature (13)..(13): Modified with 2′Ome
  misc_feature (16)..(16): n is 5-methyl cytosine
  misc_feature (17)..(17): n is 5-methyl uracil
  misc_feature (18)..(18): n is 5-methyl cytosine
  misc_feature (15)..(18): Modified with 2′Ome
  misc_feature (11)..(19): Phosphorothioate internucleotide bond
nagannnncanagaannnu

SEQ ID NO: 1421 - RNA, 13 nt, Artificial Sequence
  Synthetic Polynucleotide
  misc_feature (1)..(2): Modified with 2′Ome
  misc_feature (4)..(4): Modified with 2′Ome
  misc_feature (10)..(12): Modified with 2′Ome
  misc_feature (11)..(13): Phosphorothioate internucleotide bond
  misc_feature (13)..(13): Modified with cholesterol
cuguggaagucua

SEQ ID NO: 1422 - RNA, 19 nt, Artificial Sequence
  Synthetic Polynucleotide
  misc_feature (1)..(1): Modified with P
  misc_feature (1)..(1): Modified with 2′fluoro
  misc_feature (1)..(1): n is 5-methyl uracil
  misc_feature (5)..(5): n is 5-methyl cytosine
  misc_feature (6)..(7): n is 5-methyl uracil
  misc_feature (8)..(9): n is 5-methyl cytosine
  misc_feature (1)..(10): Modified with 2′fluoro
  misc_feature (11)..(11): n is 5-methyl cytosine
  misc_feature (11)..(11): Modified with 2′Ome
  misc_feature (12)..(12): Modified with 2′fluoro
  misc_feature (13)..(13): Modified with 2′Ome
  misc_feature (14)..(14): Modified with 2′fluoro
  misc_feature (16)..(16): n is 5-methyl cytosine
  misc_feature (17)..(17): n is 5-methyl uracil
  misc_feature (18)..(18): n is 5-methyl cytosine
  misc_feature (15)..(18): Modified with 2′Ome
  misc_feature (11)..(19): Phosphorothioate internucleotide bond
nagannnnnanagaannnu

SEQ ID NO: 1423 - RNA, 13 nt, Artificial Sequence
  Synthetic Polynucleotide
  misc_feature (1)..(13): Modified with 2′fluoro
  misc_feature (11)..(13): Phosphorothioate internucleotide bond
  misc_feature (13)..(13): Modified with cholesterol with hydroxyprolinol linker
cuguggaagucua

SEQ ID NO: 1424 - RNA, 19 nt, Artificial Sequence
  Synthetic Polynucleotide
  misc_feature (1)..(1): Modified with P
  misc_feature (1)..(1): Modified with 2′fluoro
  misc_feature (5)..(9): Modified with 2′fluoro
  misc_feature (11)..(11): Modified with 2′Ome
  misc_feature (13)..(13): Modified with 2′Ome
  misc_feature (15)..(18): Modified with 2′Ome
  misc_feature (13)..(19): Phosphorothioate internucleotide bond
uagacuuccacagaacucu

SEQ ID NO: 1425 - RNA, 13 nt, Artificial Sequence
  Synthetic Polynucleotide
  misc_feature (1)..(1): n is 5-methyl cytosine
  misc_feature (2)..(2): n is 5-methyl uracil
  misc_feature (1)..(2): Modified with 2′Ome
  misc_feature (4)..(4): n is 5-methyl uracil
  misc_feature (4)..(4): Modified with 2′Ome
  misc_feature (10)..(10): n is 5-methyl uracil
  misc_feature (11)..(11): n is 5-methyl cytosine
  misc_feature (12)..(12): n is 5-methyl uracil
  misc_feature (10)..(12): Modified with 2′Ome
  misc_feature (11)..(13): Phosphorothioate internucleotide bond
  misc_feature (13)..(13): Modified with cholesterol
nngnggaagnnna

SEQ ID NO: 1426 - RNA, 19 nt, Artificial Sequence
  Synthetic Polynucleotide
  misc_feature (1)..(1): Modified with P
  misc_feature (1)..(1): Modified with 2′fluoro
  misc_feature (5)..(9): Modified with 2′fluoro
  misc_feature (11)..(11): Modified with 2′Ome
  misc_feature (13)..(13): Modified with 2′Ome
  misc_feature (15)..(18): Modified with 2′Ome
  misc_feature (13)..(19): Phosphorothioate internucleotide bond
uagacuuccacagaacucu

SEQ ID NO: 1427 - RNA, 13 nt, Artificial Sequence
  Synthetic Polynucleotide
  misc_feature (1)..(2): Modified with 2′Ome
  misc_feature (4)..(4): Modified with 2′Ome
  misc_feature (10)..(12): Modified with 2′Ome
  misc_feature (11)..(13): Phosphorothioate internucleotide bond
  misc_feature (13)..(13): Modified with TEG-Chl
cuguggaagucua

SEQ ID NO: 1428 - RNA, 19 nt, Artificial Sequence
  Synthetic Polynucleotide
  misc_feature (1)..(1): Modified with P
  misc_feature (1)..(1): Modified with 2′Ome
  misc_feature (5)..(9): Modified with 2′fluoro
  misc_feature (11)..(11): Modified with 2′Ome
  misc_feature (16)..(18): Modified with 2′Ome
  misc_feature (13)..(19): Phosphorothioate internucleotide bond
uagacuuccacagaacucu

SEQ ID NO: 1429 - RNA, 13 nt, Artificial Sequence
  Synthetic Polynucleotide
  misc_feature (1)..(2): Modified with 2′Ome
  misc_feature (4)..(4): Modified with 2′Ome
  misc_feature (10)..(12): Modified with 2′Ome
  misc_feature (11)..(13): Phosphorothioate internucleotide bond
  misc_feature (13)..(13): Modified with cholesterol
cuguggaagucua

SEQ ID NO: 1430 - RNA, 19 nt, Artificial Sequence
  Synthetic Polynucleotide
  misc_feature (1)..(1): Modified with P
  misc_feature (1)..(1): Modified with 2′fluoro
  misc_feature (5)..(9): Modified with 2′fluoro
  misc_feature (11)..(11): Modified with 2′Ome
  misc_feature (13)..(13): Modified with 2′Ome
  misc_feature (15)..(18): Modified with 2′Ome
  misc_feature (11)..(19): Phosphorothioate internucleotide bond
uagacuuccacagaacucu

SEQ ID NO: 1431 - RNA, 13 nt, Artificial Sequence
  Synthetic Polynucleotide
gcaccuuucuaga

SEQ ID NO: 1432 - RNA, 19 nt, Artificial Sequence
  Synthetic Polynucleotide
ucuagaaaggugcaaacau

SEQ ID NO: 1433 - RNA, 14 nt, Artificial Sequence
  Synthetic Polynucleotide
uugcaccuuucuaa

SEQ ID NO: 1434 - RNA, 20 nt, Artificial Sequence
  Synthetic Polynucleotide
uuagaaaggugcaaacaagg